(19) United States
(12) Patent Application Publication
(10) Pub. No.: US 2005/0003502 A1
(43) Pub. Date: Jan. 6, 2005

(54) Structures and methods for designing topoisomerase I inhibitors

(75) Inventors: Alex Burgin, Bainbridge Island, WA (US); Kathyrn Hjerrild, Bainbridge Island, WA (US); Hidong Kim, Bainbridge Island, WA (US); Bart Lee Staker, Kingston, WA (US); Lance Stewart, Bainbridge Island, WA (US); Craig Behnke, Shoreline, WA (US); Michael Feese, Seattle, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: Emerald BioStructures, Inc., Brainbridge Island, WA (US)

(21) Appl. No.: 09/993,245

(22) Filed: Nov. 14, 2001

Related U.S. Application Data
(60) Provisional application No. 60/248,474, filed on Nov. 14, 2000.

(51) Int. Cl. G06F 19/00; C12N 9/99

(52) U.S. Cl. 435/184; 702/19

(57) ABSTRACT

This invention relates to crystalline structures of the topoisomerase I and their use in designing new anti-cancer agents anti-viral agents and anti-microbial agents.

This application claims the priority of provisional application Ser. No. 60/248,474 filed Nov. 14, 2000 which is incorporated herein by reference.

### BACKGROUND OF THE INVENTION

1. Field of the Invention

This invention is in the field of identifying interactions of biomolecules by examining crystal structures of complexes of a compound and a biomolecule as a means for designing active biological compounds.

2. Description of the Art

WO 99/45379 describes the use of x-ray crystallography to screen compounds that are not known ligands of a target biomolecule for their ability to bind the target biomolecule. This publication illustrates using x-ray crystallography to determine the binding of potential inhibitors of RNA methyltransferase.

WO 00/14105 describes a crystal structure of a protein construct containing catalytic kinase domain of vascular endothelial growth factor receptor 2, a key enzyme in angiogenesis.

U.S. Pat. No. 5,856,116 describes the use of a crystal structure to design active biological compounds. This publication describes the process of identifying potential inhibitor molecules given a crystal structure of a biomolecule and is incorporated herein by reference.

Topoisomerase I (Topo I) is an essential nuclear enzyme that facilitates DNA replication and transcription by relaxing the torsional stress generated in the wake of moving polymerase complexes. Topo I mediates DNA relaxation by introducing a transient break in the phosphodiester backbone of a single strand, allowing for unwinding of positively supercoiled DNA or rewinding of negatively supercoiled DNA. Strand cleavage involves a transesterification reaction catalyzed by a Tyr, Arg, Arg, His tetrad of conserved residues, and does not require any divalent metal cation or energy cofactor. The Tyr 0 oxygen mediates nucelophilic attack on the scissile phosphodiester bond, which culminates in the formation of a covalent bond between the enzyme and the 3′ end of the broken strand. Reversal of the transesterification restores the phosphodiester bond and liberates the enzyme. Human topo I belongs to the highly conserved euckaryotic topoisomerase I family of enzymes. The human topoisomerase I gene has been cloned and is described in, D'Arpa, P., et al., Proc Natl Acad Sci USA, 85, pp. 2543-2547 (1988). U.S. Pat. No. 5,070,192 describes recombinant human topoisomerase 1, cDNA coding and expression. This patent is incorporated herein by reference. Human topoisomerase I is the sole intracellular target of camptothecin (CPT) and other “topo I poisons,” some of which are among the most promising anticancer drugs ever identified. FIG. 8 illustrates domains of human topoisomerase I.

Burgin Jr., A. B., Huizenga, B. N., Nash, H. A., Nucleic Acids Res., 23, pp. 2973-2979 (1995) describes the synthesis of oligonucleotide substrates that contain a 5′-briding phosphorothiolate positioned at the cleavage site in duplex DNA for eukaryotic topo I. This substrate was defined as a “suicide substrate” because it was shown that topo I was capable of cleaving the substrate at the 5′-bridging phosphorothiolate, but that the cleavage was irreversible since the resulting 5′-sulfhydryl was not a sufficient nucleophile to reverse the cleavage reaction. Hence, upon cleaving the suicide substrate, topo I becomes irreversibly trapped in covalent complex with the 3′ end of the broken strand.

The X-ray crystal structures of Topo I, i.e., Crystal Form 4, Crystal Form 2, and Crystal Form I are described in Stewart, L., et al., Science, 729, pp. 1534-1541 (1998), and in Redinbo, M. R., Stewart, L., Kuhn, P., Champoux, J. J., Hol, W. G. J., Science, 279, pp. 1504-1513 (1998). Also see Stewart, L., et al., J. Mol. Biol., 269, pp. 355-372 (1997). These references describe crystallized topo I constructs with a 22 bp DNA structure having a 5′-phosphorothiolate at the topo I cleavage site. X-ray crystallography reveals the three-dimensional interaction between the DNA and the topoisomerase I enzyme. However, these crystal structures do not contain a desciption of the three dimensional interactions of inhibitor molecules to complexes of topoisomerase I and DNA. Because of the complicated interactions between the binary complex of topoisomerase I and DNA it is not obvious to a practitioner of the art how to design potential inhibitors based solely on the crystal structures of topoisomerase I and DNA in the absence of bound biologically active compound. In addition previous structures of Topo I in complex with DNA, do not contain a fully active construct of the Topoisomerase I protein.

### SUMMARY OF THE INVENTION

This invention solves the above problems by providing methods to crystallize the ternary complex of topoisomerase I with DNA and with known biologically active compounds. The spacial information obtained from these results permits one skilled in the art to design new inhibitor compounds.

It is an object of this invention to solve the three-dimensional crystal structure of topoisomerase I (Topol) in covalent complex with DNA and inhibitor compounds.

It is an object of this invention to solve the three-dimensional crystal structure of a fully active form of topoisomerase I in complex with DNA.

The invention relates to methods for identifying and designing Topol inhibitors which involves forming a crystal structure from the test agent and topoisomerase I covalently linked to duplex DNA at the topoisomerase I cleavage site and determining the crystal structure of the complex to determine the spacial relationship of the topoisomerase UDNA construct and the anti-cancer drug.

The invention includes methods for designing Topol inhibitors which involves utilizing the crystal structure described above to design modified compounds.

The invention also includes methods for making crystal structures.

### BRIEF DESCRIPTION OF THE FIGURES

FIG. 1 lists the atomic structure coordinates of human topo 70 in covalent complex with duplex 22 mer DNA (Form 7). The following abbreviations are used in FIG. 1. “Atom type” refers to the elembent whose coordinates are measured. The first letter in the column defines the element. “X, Y, Z” crystallographically define the atomic position of the element measured. “B” is a thermal factor that measures movement of the atom around its atomic center. Structure coordinates of Form7 according to FIG. 1 may be modified from this original set by mathematical manipulation. Such manipulations include, but are not limited to, crystallographic permutations of the raw structure coordinates, fractionalization of the raw structure coordinates, integer additions or subtractions to sets of the raw structure coordinates, inversion of the raw structure coordinates, and any combination of the above.

FIG. 2 lists the atomic structure coordinates of human topo 70 in covalent complex with duplex 22 mer DNA in complex with the compound topotecan (Form 9-TTC). The following abbreviations are used in FIG. 2. “Atom type” refers to the elembent whose coordinates are measured. The first letter in the column defines the element. “X, Y, Z” crystallographically define the atomic position of the element measured. “B” is a thermal factor that measures movement of the atom around its atomic center.

Structure coordinates of Form9-TTC according to FIG. 2 may be modified from this original set by mathematical manipulation. Such manipulations include, but are not limited to, crystallographic permutations of the raw structure coordinates, fractionalization of the raw structure coordinates, integer additions or subtractions to sets of the raw structure coordinates, inversion of the raw structure coordinates, and any combination of the above.

Residues 175-199 are not included in the coordinate set as they were not visible in the crystal structure.

FIG. 3 lists the atomic structure coordinates of human topo 70 in covalent complex with duplex 22 mer DNA in complex with the compound Ag260 (Form 9-AG260). The following abbreviations are used in FIG. 3. “Atom type” refers to the elembent whose coordinates are measured. The first letter in the column defines the element. “X, Y, Z” crystallographically define the atomic position of the element measured. “B” is a thermal factor that measures movement of the atom around its atomic center.

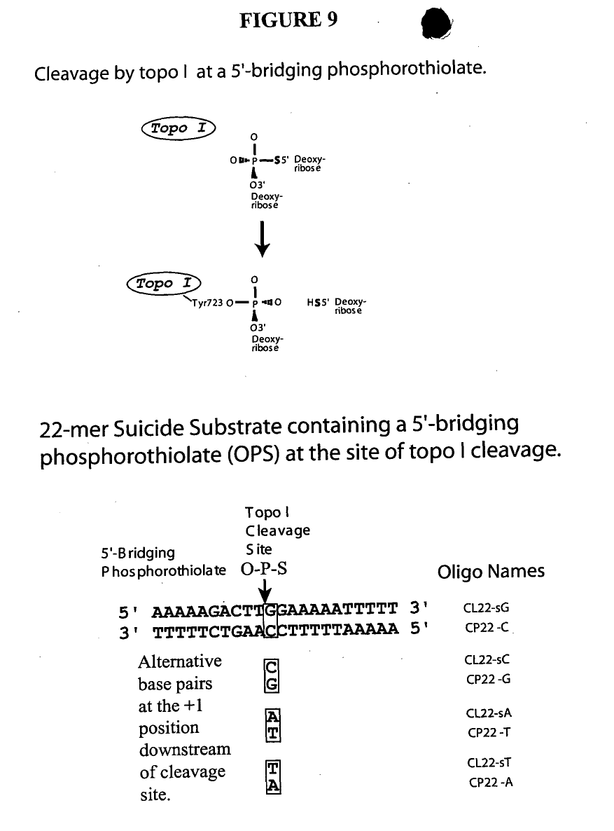

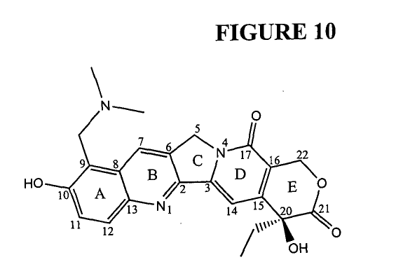

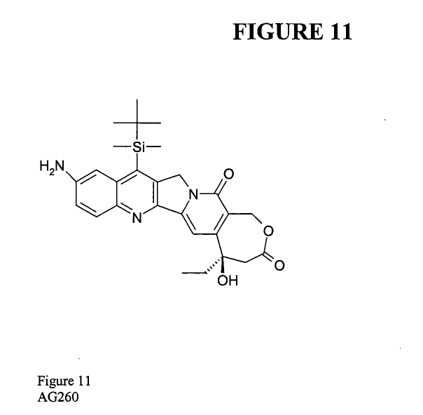

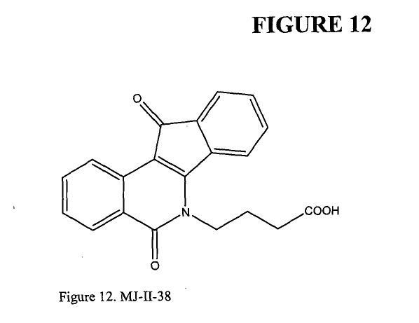

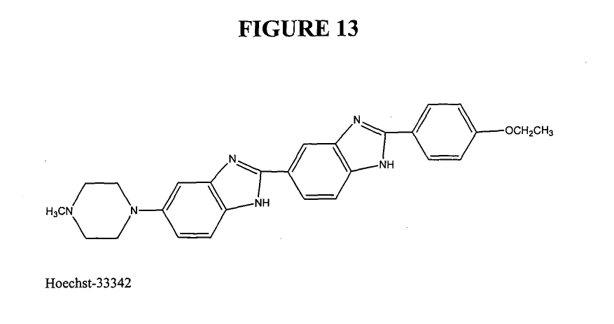

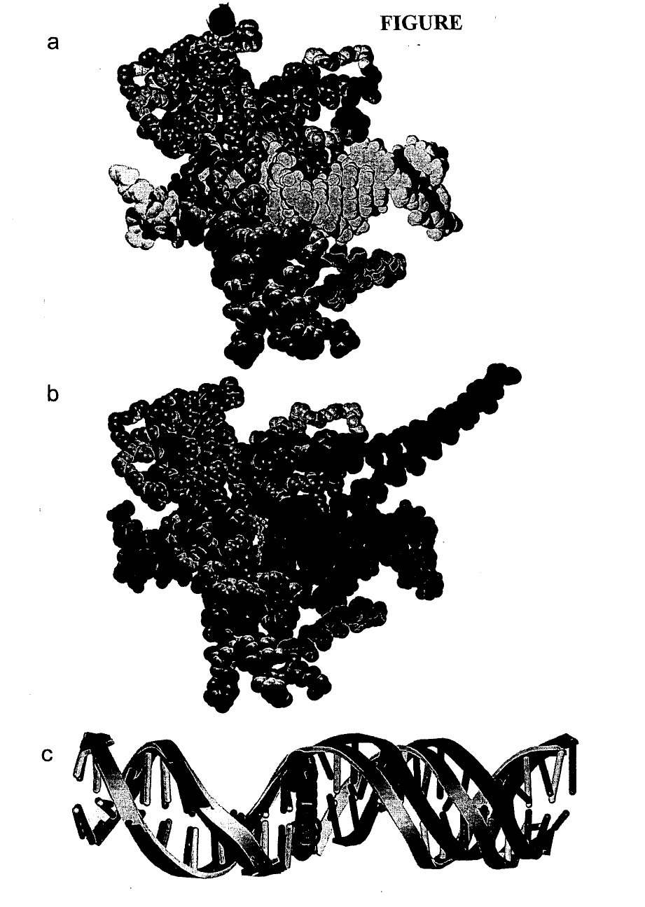

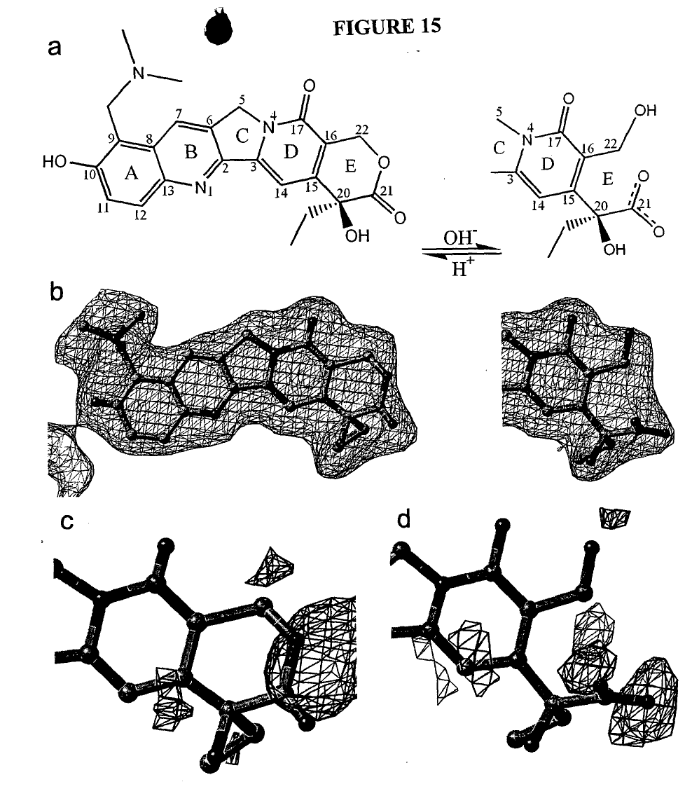

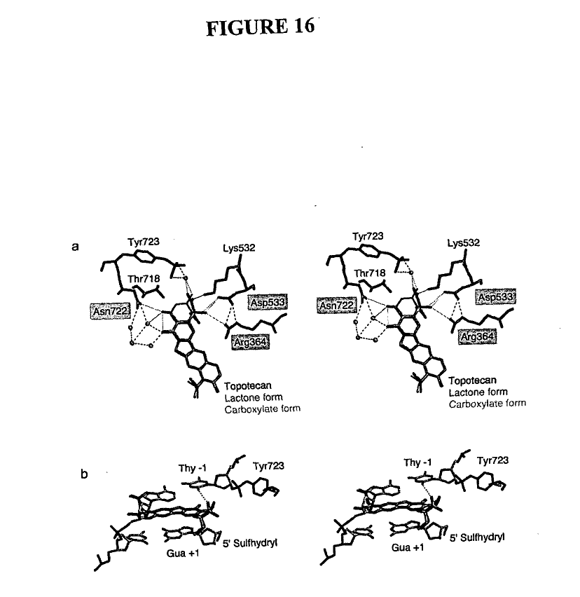

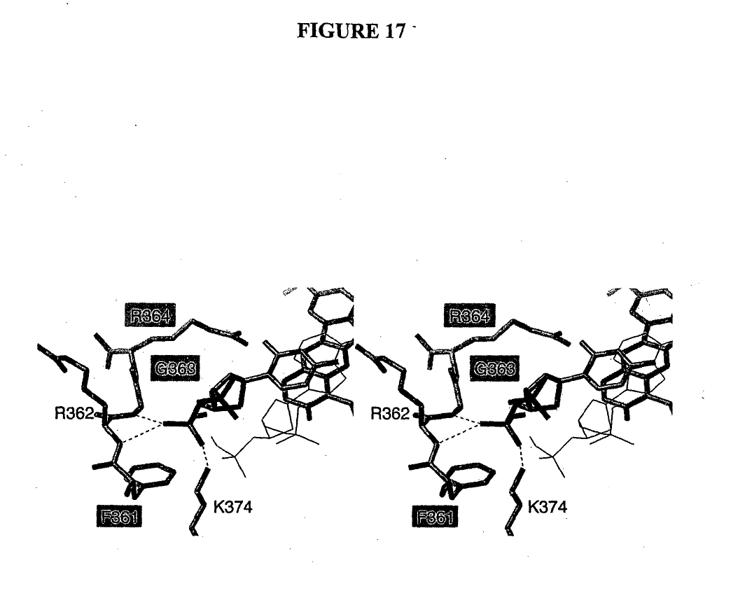

Structure coordinates of Form9-AG260 according to FIG. 3 may be modified from this original set by mathematical manipulation. Such manipulations include, but are not limited to, crystallographic permutations of the raw structure coordinates, fractionalization of the raw structure coordinates, integer additions or subtractions to sets of the raw structure coordinates, inversion of the raw structure coordinates, and any combination of the above.

Residue numbers 198-202 and 634-640 were modeled as alanine residues. Residues 175-197 are not included in the coordinate set as they were not visible in the crystal structure.

FIG. 4 lists the atomic structure coordinates of human topo 70 in covalent complex with duplex 22 mer DNA in complex with the compound MJ-II-38 (Form 10). The following abbreviations are used in FIG. 4. “Atom type” refers to the elembent whose coordinates are measured. The first letter in the column defines the element. “X, Y, Z” crystallographically define the atomic position of the element measured. “B” is a thermal factor that measures movement of the atom around its atomic center.

Structure coordinates of Form 10 according to FIG. 4 may be modified from this original set by mathematical manipulation. Such manipulations include, but are not limited to, crystallographic permutations of the raw structure coordinates, fractionalization of the raw structure coordinates, integer additions or subtractions to sets of the raw structure coordinates, inversion of the raw structure coordinates, and any combination of the above.

Residue numbers 201-202, and 634 were modeled as alanine residues. Residues 175-200 are not included in the coordinate set as they were not visible in the crystal structure.

FIG. 5 lists the atomic structure coordinates of human topo 70 in covalent complex with duplex 22 mer DNA in complex with the compound Hoechst-33342 (Form 11). The following abbreviations are used in FIG. 5. “Atom type” refers to the elembent whose coordinates are measured. The first letter in the column defines the element. “X, Y, Z” crystallographically define the atomic position of the element measured. “B” is a thermal factor that measures movement of the atom around its atomic center.

Structure coordinates of Form 11 according to FIG. 5 may be modified from this original set by mathematical manipulation. Such manipulations include, but are not limited to, crystallographic permutations of the raw structure coordinates, fractionalization of the raw structure coordinates, integer additions or subtractions to sets of the raw structure coordinates, inversion of the raw structure coordinates, and any combination of the above.

FIG. 6 illustrates a ribbon diagram of crystal Form 7.

FIG. 7 illustrates a ribbon diagram of crystal Form 9.

FIG. 8 illustrates the domain organization of human topo 1. A schematic representation of the domain organization for full-length human topo I is shown (line 1). Other human topo I constructs include the N-terminally truncated topo70 (line 2), reconstituted topo58/6.3 (line 3), and N-terminally truncated topo65 (line 4). Circles indicate residues that can be mutated to confer resistance to CPT. The Core domain is comprised of the “Cap” (black) and “Catalytic” (red) regions with helices α5 and a6 forming the “nose cone.” The “Linker” domain (orange).

FIG. 9 illustrates the phosphorthiolate DNA.

FIG. 10 is a chemical drawing of topotecan.

FIG. 11 is a chemical drawing of AG260.

FIG. 12 is a chemical drawing of MJ-11-38.

FIG. 13 is a chemical drawing of Hoechst-33342.

FIG. 14. Covalent Topo I-DNA complexes without (Panel A) and with (Panel B) bound topotecan. Protein main chain atoms are represented in grey CPK, with the linker domain residues Glu641-Asn711 colored blue, nose cone residues Phe302-Tyr338 colored green, and connector residues Pro635-Phe640 colored red. DNA is represented in full atom CPK, and colored yellow in the non-drug bound structure or purple in the drug bound structure. Topotecan is represented as orange ball-and-stick. Comparison of the 22 mer duplex of both structures (Panel C, shown with protein removed and DNAs rotated 180 degrees about the helix axis) demonstrates that topotecan (orange CPK) binds to the enzyme-substrate complex by intercalating at the site of DNA breakage.

FIG. 15. Topotecan electron density. Panel A depicts a schematic of topotecan with reversible hydrolysis of the base-labile E-ring from the closed lactone conformation to the open carboxylate form. Panel B displays a 3.0 σ |Fₒ|-|F_{c}| omit map of electron density for topotecan. The electron density map reveals that both the lactone and carboxylate forms of the E-ring are present in the crystal structure. The E-ring of topotecan is oriented towards the phosphotyrosine. The c-9-dimethylamine group of topotecan projects into the major groove of the B-form DNA duplex, whereas the c-20-ethylene group of the E-ring faces into the minor groove. Panel C displays the 3.0 σ |Fₒ|-|F_{c}| electron density map calculated with the lactone form of topotecan (100% closed E-ring). Negative electron density (red) is seen in the vicinity of the lactone oxygen, and positive (blue) electron density peaks are located nearby. Panel D displays the 3.0 σ |Fₒ|-|F_{c}| electron density calculated with the carboxylate form of topotecan (100% open E-ring). Negative electron density (red) surrounds the terminal hydroxyl and carboxylic acid moieties, while a positive (blue) electron density peak is in the location of what would be the lactone oxygen in the closed E-ring conformation.

FIG. 16. Mode of topotecan binding. Stereoviews of topotecan interactions with protein side chains (Panel A) and DNA (Panel B) are shown for both the carboxylate (thick gold) and lactone (thin green) forms of the drug. Hydrogen bonds that are nearly identical between the two forms are shown as thick dashed lines. Hydrogen bonds that differ between the two forms are shown as thin black dashed lines for the lactone and thin blue solid lines for the carboxylate. Labels for residues that if mutated produce a camptothecin resistant enzyme are highlighted in yellow. One potential electrostatic interaction between the carboxylate form and the O2 of the −1 thymidine of the cleaved strand (Thy-1) is shown as a thin dashed line. The oxygen atoms of water molecules are depicted as light blue spheres. Protein side chains are thick green and non-carbon atoms are colored red for oxygen, blue for nitrogen, and magenta for phosphorus.

FIG. 17. Topotecan inhibits relaxation via a “hinge-lock” mechanism. A stereoview of the ternary enzyme-DNA-topotecan complex demonstrates a binding pocket for the −1/+1 phosphodiester linkage of the intact DNA strand. Dashed lines represent hydrogen bonds. For reference, the ˜1/+1 phosphodiester linkage and associated +1 base (adenine) of the non-drug bound structure is also shown in grey stick. Atom coloring is green for carbon, red for oxygen, blue for nitrogen, and magenta for phosphorus. Labels for residues that if mutated produce a camptothecin resistant enzyme are highlighted in yellow. Topotecan (orange) is shown intercalated into the ternary complex.

### DEFINITIONS

The term “topoisomerase I” and “Topol” includes eukaryotic topoisomerase I, human topoisomerase I including constructs shown in FIG. 8. Those skilled in the genetic eingineering arts will recognize that from the cDNA disclosed in U.S. Pat. No. 5,070,192 many variations of topoisomerase I suitable for practicing the invention can be made.

The term “topo70” represents the fully active construct of the human topoisomerase I protein containing residues 175-765.

The term “Form 7” represents the crystal structure of topo70 bound in covalent complex with duplex.’

The term “Form 9-TTC” represents the crystal structure of topo70 bound in covalent complex with duplex DNA and the compound topotecan.

The term “Form 9-AG260” represents the crystal structure of topo70 bound in covalent complex with duplex DNA and the compound AG260.

The term “Form 10” represents the crystal structure of topo70 bound in covalent complex with duplex DNA and the compound MJ-II-38.

The term “Form 11” represents the crystal structure of topo70 bound in covalent complex with duplex DNA and the compound Hoechst-33342.

### DETAILED DESCRIPTION OF THE INVENTION

In order that the invention described herein may be more fully understood, the following detailed description is set forth.

Topoisomerase I (Topo I) is an essential eukaryotic enzyme that acts to relax torsional stress in supercoiled DNA generated during transcription and replication. Champoux, J. J., Ann. Rev. Biochem., 70, pp. 369-413 (2001). Topo I mediates DNA relaxation by creating a transient single strand break, allowing the broken strand to rotate around its intact complement. This nicking results from the transesterification of an active-site tyrosine at a DNA phosphodiester bond forming a 3′-phosphotyrosine covalent enzyme-DNA complex. After DNA relaxation, the covalent intermediate is reversed when the released 5′-OH of the broken strand re-attacks the phosphotyrosine intermediate in a second transesterification reaction Champoux, J. J., Ann. Rev. Biochem., 70, pp. 369-413 (2001). Topo I is the sole molecular target of a family of anti-cancer compounds called camptothecins, Wall, M. E., et al., J. Am. Chem. Soc., 88, pp. 3888-3890 (1966), Hsiang, Y. H., et al., J. Biol. Chem., 260, pp. 14873-14878 (1985), Nitiss, J. L. and Wang, J. C., Proc. Natl. Acad. Sci. U.S.A., 85, pp. 7501-7505 (1988) (CPTs). It is generally believed that CPTs act as uncompetitive inhibitors by binding to the covalent Topo I-DNA intermediate and blocking the second transesterification reaction, Hertzberg, R. P., et al., Biochem., 28, pp. 4629-4638 (1989), thus converting the enzyme into a molecular poison. Chen, A. Y. and Liu, L. F., Rev. Pharmacol. Toxicol., 34, pp. 191-218 (1994). Several other families of compounds exist which are known to inhibit topoisomerase I and are believed to bind at the same site as the camptothecin family of compounds. These compounds includes Indolacarbazoles, such as the anti-microbial marcellomycin; Indenoisoquinolines, such as the experimental anti-cancer compound MJ-II-38; silatecan derivatives which are camptothecin compounds with silicon derivitizations, such as AG260. Additionally, other compounds have been shown to inhibit topoisomerase I, but it is not known if they bind at the same site as the camptothecin compounds. These compounds include minor groove binding compounds such as Hoecsht-33342. We have shown that these compounds do not bind at the same site as camptothecin.

To determine the structural basis for the mechanism of inhibitory activity, we have solved several new crystal structures of a fully active version of human Topo I covalently joined to duplex DNA in the absence (Form7) and presence of topotecan, a camptothecin derivative (Form 9-TTC); AG260, a sialyl-tecan compound (Form 9-AG260); MJ-II-38, an indenoisoquinoline compound (Form 10); and hoechst-33342, a DNA minor groove binding compound (Form 11). Examination of the Form9-TTC, Form9-AG260, and Form 10, structures reveals that theses compounds intercalate at the site of DNA cleavage, forming base-stacking interactions with both the −1 (upstream) and +1 (downstream) base pairs. A detailed examination of the topotecan structure follows.

The planar five-membered ring system of topotecan mimics a base pair in the DNA duplex, and occupies the same space as the +1 base pair in the structure without drug bound (FIG. 14). Approximately 61% of the topotecan surface is covered by base stacking interactions, and 27% is covered by protein contacts. The intercalation pocket is stabilized by several protein-DNA interactions. The hydroxyl of Thr718 makes a hydrogen bond contact with the non-bridging phosphodiester oxygen of guanosine at position +1 of the cleaved strand, and Arg364 makes a hydrogen bond contact with N3 of adenosine at position −1 of the uncleaved strand. Consistent with this binding mode, mutation at position 364 is expected to destabilize the binding site and results in camptothecin resistance. Li, X. G., et al., Biochem. Pharmacol., 53, pp. 1019-1027 (1997). The intercalation also results in a 3.4 Å shift of the downstream duplex that displaces the reactive 5′-OH of the cleaved strand 10 Å away from the phosphotyrosine. In order for a religation event to occur, the topotecan molecule must be released from the nicked DNA and diffuse out of the complex.

The E-ring of camptothecin is known to be in equilibrium between a closed lactone form and a hydrolyzed open carboxylate form. Wall, M. E., et al., J. Am. Chem. Soc., 88, pp. 3888-3890 (1966) (FIG. 15). It is widely believed that the closed lactone E-ring is essential for inhibition of Topo I. Kehrer, D. F. S., et al., Anti-Cancer Drugs, 12, pp. 89-105 (2001). However, there is experimental evidence for E-ring opening upon formation of the ternary protein-DNA-drug complex. Chourpa, I., R10u, J. F., Millot, J., Pommier, Y., Manfait, M., Biochem., 37, pp. 7284-7291 (1998). In addition, despite a general belief that the carboxylate form is inactive, it has been shown that the sodium carboxylate form of camptothecin does have Topo I inhibitory activity in vitro Hsiang, Y. H., et al., Cancer Res, 49, pp. 4385-9 (1989) and in in vivo cell killing assays. Giovanella, B. C., et al., Science, 246, pp. 1046-8 (1989). Close inspection of the topotecan electron density allowed positioning of both the open and closed E-ring conformers (FIG. 15b). An unrestrained full matrix refinement of occupancy factors Sheldrick, G. M., pp. (1997) (with all positional and thermal parameters fixed) for the closed lactone and open carboxylate versions of topotecan converged to an occupancy of 63% (standard uncertainty 7%) closed lactone and 37% (standard uncertainty 7%) open carboxylate forms of topotecan. As another test, each conformer of topotecan was then placed into the structure and refined independently. Analysis of the difference Fourier maps demonstrates the presence of both the lactone and carboxylate forms of topotecan (FIGS. 15c and 15d). These results are typical of crystallographic structures in which multiple conformations of an amino acid side chain are present in a protein structure Smith, J. L., Hendrickson, W. A., Honzatko, R. B., Sheriff, S., Biochem., 25, pp. 5018-5027 (1986).

Surprisingly, there is only one protein-drug interaction stabilizing the lactone (E-ring closed) form of topotecan (FIG. 16). Asp533 hydrogen bonds to the 20(S) hydroxyl of topotecan. In turn, Asp533 is coordinated by Arg364, which is positioned only 4 Å from the B-ring nitrogen. Additionally, there are two water-mediated hydrogen bonds that assist in coordinating the topotecan into the cleaved DNA intermediate. The oxygen of the D-ring pyridone makes a water mediated contact to Asn722, and the C-21 oxygen of the E-ring is bridged by a water molecule to the phosphotyrosine and catalytic residues Arg488, Arg590 and His632 Champoux, J. J., Ann. Rev. Biochem., 70, pp. 369-413 (2001). Consistent with the structural model, mutations at residues Asp533, Arg364 and Asn722 would be expected to destabilize the bound drug and are known to result in camptothecin resistance Li, X. G., et al., Biochem. Pharmacol., 53, pp. 1019-1027 (1997), Tamura, H., et al., Nucleic Acids Res., 19, pp. 69-75 (1991), Fertala, J., et al., J. Biol. Chem, 275, pp. 15246-15253 (2000).

It is not possible to determine the relative affinities of open (carboxylate) vs. closed (lactone) forms of topotecan based on the crystal structures, however the carboxylate form of topotecan would be expected to have a slower rate of dissociation since three additional direct hydrogen bonds are possible between the open E-ring and the protein-DNA complex (FIG. 16). In the carboxylate model, the 22-hydroxyl is 2.7 Å from the R-group of Asn722. The 21-carboxylate oxygen is 2.8 Å from Lys532, a known catalytic residue, Krogh, B. O., Shuman, S., Mol. Cell, 5, pp. 1034-1041 (2000). The 20(S)-hydroxyl still coordinates Asp533, and makes an additional hydrogen bond contact (3.1 Å) to the 1-nitrogen of Arg364, a residue known to be involved in camptothecin sensitivity, Li, X. G., et al., Biochem. Pharmacol., 53, pp. 1019-1027 (1997). Finally, it is also important to note that in the carboxylate structure, one of the 21-carboxylate oxygens is 2.7 Å from the O2 of the −1 thymidine of the cleaved strand and the second carboxylate oxygen makes a water mediated contact with the phosphotyrosine phosphodiester. Topotecan therefore appears to inhibit religation by displacing the reactive 5′-OH and by simultaneously coordinating several active-site functional groups.

In addition to preventing DNA religation, Topo I poisons such as camptothecin have been shown to inhibit the rotation/relaxation process in vitro Champoux, J. J., Ann. N.Y. Acad. Sci., 922, pp. 56-64 (2000). It has been a mystery why camptothecins stabilize the nicked complex but prevent DNA relaxation—nicked DNA should be able to rotate and allow DNA relaxation Champoux, J. J., Ann. N.Y. Acad. Sci., 922, pp. 56-64 (2000). Topoisomerase I has been proposed to relax DNA via a mechanism of “controlled rotation,” in which the DNA duplex located downstream of the cleavage site rotates around the −1/+1 phosphodiester linkage of the intact strand, effectively passing the unbroken strand through the single strand break with each complete rotation event Stewart, L., et al., Science, 729, pp. 1534-1541 (1998). A comparison of the unbound and topotecan-bound structures shows that topotecan displaces the critical −1/+1 phosphodiester linkage of the non-scissile strand into a binding pocket, producing several interactions that are predicted to inhibit rotation (FIG. 17). One non-bridging oxygen of the −1/+1 phosphodiester is hydrogen bonded to the main chain nitrogen atoms of Arg362 and Gly363. The other non-bridging oxygen forms a hydrogen bond to the terminal nitrogen of Lys374. The hydrogen bond contact to Lys374 is also present in the non-drug bound structure, indicating that this side chain can move to accommodate a shift in position of the −1/+1 phosphodiester. The shifted −1/+1 phosphodiester is also positioned close to the Phe361 side chain which would provide and additional steric block to rotation. The tight positioning of the −1/+1 intact phosphodiester against the peptide backbone, together with support from Phe361 and a molecular clamping of the upstream duplex by Topo I Redinbo, M. R., Stewart, L., Kuhn, P., Champoux, J. J., Hol, W. G. J., Science, 279, pp. 1504-1513 (1998), effectively restrains 3 (α, β, γ) Saenger, W., Springer Advanced Texts in Chemistry, pp. 556 (1984) of the 5 potentially rotatable backbone bonds. This tight packing arrangement is expected to interfere with the conformational changes in the DNA required to complete a 360 degree rotation of the downstream DNA about the −1/+1 intact phosphodiester in what we propose is a “hinge-lock” mechanism. This model provides a rationale for understanding how camptothecins can inhibit DNA relaxation through an intercalative binding mode, and is consistent with the observations that Phe361, Gly363, and Arg364 are required for sensitivity to camptothecin Li, X. G., et al., Biochem. Pharmacol., 53, pp. 1019-1027 (1997), Rubin, E., et al., J Biol Chem, 269, pp. 2433-2439 (1994), Fiorani, P., et al., Mol Pharmacol, 56, pp. 1105-1115 (1999).

The hinge-lock mechanism would not eliminate all possible DNA rotation. For example, rotation could still occur at the +2 (or +3, etc.) phosphodiester. However, additional base-pair hydrogen bond interactions would have to be broken to allow this rotation. Alternatively, rotation could still occur at +1 since two rotatable bonds are not hindered. However in both cases, the trajectory of the rotating DNA would be significantly altered and this would require conformational flexibility that is not likely to be present in the protein. The protein encircles the DNA, and both the linker and nose cone domains of Topo I contain a positively charged residues that are likely to contact the DNA during rotation Stewart, L., et al., Science, 729, pp. 1534-1541 (1998). This may at least partially explain why reconstituted “linker-less” human Topo I is resistant to the relaxation-inhibition effect of topotecan Stewart, L., et al., J. Biol. Chem., 274, pp. 32950-32960 (1999), as well as the camptothecin resistant phenotype of an Ala653Pro mutation which destabilizes the linker domain, Fiorani, P., et al., Mol Pharmacol, 56, pp. 1105-1115 (1999).

A. Preparation of Recombinant topo70 and topo58/6.3 Protein.

The coding sequences for wild type human topo70 (residues 175 to 765 of the natural protein plus an N-terminal initiating methionine) were derived from plasmid pGST-topo70 wt Biochemical and biophysical analyses of recombinant forms of human topoisomerase I described in, Stewart, L., et al., J. Biol. Chem., 271, pp. 7593-7601 (1996). A BamHI-EcOR1 restriction fragment from pGST-topo70 wt was transferred into linear pFastBac baculovirus transfer vector (Life Technologies, Inc.) that was prepared by cleavage with BamHI and EcOR1. The resulting plasmid called “pFastBac-topo70 wt” was used, according to standard protocol (Life Technologies, Inc.), to generate recombinant baculovirus stock that expresses the recombinant topo70.

Recombinant baculoviruses were used to produce topo70 in insect cells and the protein was purified according to known procedures for purification of baculovirus expressed human DNA topoisomerase I. In protocols for DNA topoisomerases: I. DNA topology and enzyme purification, Stewart, L., et al., J. Biol. Chem., 271, pp. 7593-7601 (1996).

The topo58/6.3 protein was prepared as described previously with minor modification. Stewart, L., et al., J. Mol. Biol., 269, pp. 355-372 (1997).

B. Preparation of Oligonucleotides that Contain 5′-Bridging Phosphorothiolate.

The purification of oligonucleotides and the hybridization of complementary oligonucleotides to generate duplex oligonucleotide substrates was described. Stewart, L., et al., Science, 729, pp. 1534-1541 (1998). Three-dimensional structures of reconstituted human topoisomerase I in covalent and non-covalent complex with DNA is described in Redinbo, M. R., Stewart, L., Kuhn, P., Champoux, J. J., Hol, W. G. J., Science, 279, pp. 1504-1513 (1998).

The synthesis of suicide substrates that contain a 5′-bridging phosphorothiolate at the site of topo I cleavage wherein the base immediately downstream of the cleavage site is a thymidine as described, Burgin Jr., A. B., Huizenga, B. N., Nash, H. A., Nucleic Acids Res., 23, pp. 2973-2979 (1995). These synthetic routes have been used to produce oligonucleotides containing a 5′-bridging phosphorothiolate at the site of topo I cleavage immediately preceding a thymidine, adenine, guanine, or cytocine. FIG. 9 illustrates the process. The synthetic routes of 5′-bridging phosphorthiolate at the site of topo I cleavage wherein the base immediately downstream of the cleavage site is a adenine, guanine or cytosine are described in U.S. patent application Ser. No. 09/882,274 (Burgin, SDSU patent application). While a 22 mer duplex DNA is preferred, those skilled in the art will recognize that larger DNA sequences having 15-40 bp are operative and the DNA sequence can very as long as the DNA is linked to the topo I cleavage site.

C. Sources of Anti-Cancer Compounds.

Topotecan is a trade name for the structure shown in FIG. 10. Related compounds are described in U.S. Pat. No. 5,004,758. These compounds are water soluble camptothecin analogs useful for inhibiting growth of animal tumor cells. Synthesis of cytotoxic indenoisoquinoline topoisomerase I poisons. are described in, Strumberg, D., et al., J Med Chem, 11, pp. 446-457 (1999), and the synthesis of new Indeno[1,2-c]isoquinolines: cytotoxic non-camptothecin topoisomerase I inhibitors are described in, Cushman, M., et al., J Med Chem, 5, pp. 3688-3698 (2000).

D. Combinatorial Crystallization Screening to Identify Ternary Topo I-DNA-Inhibitor Crystallization Conditions.

In order to identify crystallization conditions that generate crystals comprised of topo70 in covalent complex with DNA and bound to anti-cancer compounds such as topotecan, numerous crystallization conditions that had salt concentrations less than 400 mM and buffered pHs between 4 and 9 were screened. The crystallant buffer; salt (CBS) cross optimization strategy is shown in disclosed in U.S. Pat. No. 6,039,804 and is incorporated herein by reference. The screening system utilized a combinatorial approach involving the set up of parallel crystallization conditions asdescribed in U.S. Pat. No. 6,039,804. Issued Mar. 21, 2000. The screened mixtures contained topo I (topo70 or topo58/6.3), suicide substrate 5′-bridging oligonucleotide duplex, and various inhibitors.

In order to identify crystallization conditions that depended on the presence of topotecan or other compounds, a novel approach to crystallization screening wherein was developed. A large number of novel crystallization conditions using all combinations of crystallants, buffers, and salts from all known crystallization conditions for topo70 and topo58/6.3. These recombinant crystallization conditions were screened with enzyme (topo70 or topo58/6.3), topotecan, and suicide substrate that contained a 5-bidging phosphorothiolate at the site of topo I breakage wherein the base immediately downstream of the break site on the cleaved strand was a guanine (G) which was base paired to its complementary cytosine (C) on the complementary strand. This approach proved to be successful in producing novel crystal forms of human topo70, wherein the crystal growth absolutely depended on the presence of the topotecan.

E. Buffers

The stock solutions of buffers were prepared as follows.

Tris-HCl pH 7.0 or 8.0

Tris base (Sigma Cat. # T1503, CAS # 77-86-1) stock solutions were made pH 7.0 or 8.0 with concentrated HCl (Sigma Cat. # H7020, CAS # 7647-01-0), and the volumes adjusted to 1 M final concentration of Tris base.

Na/K Phosphate pH 6.2

0.5 M Na₂HPO₄ (Sigma Cat. # S7907, CAS # 7558-79-4) and 0.5 M KH₂PO₄ (Sigma Cat. # PO662, CAS # 7778-77-0) solutions were mixed together to make a pH 6.2 Na/K phosphate stock solution.

MES pH 6.4

A MES (Sigma Cat. # M8250, CAS # 4432-31-9) stock solution was made pH 6.4 with 50% NaOH (Sigrna Cat. #S0899, CAS #1310-73-2), and the volume adjusted to 1 M MES.

ADA pH 6.5

A ADA (Sigma Cat. # A9883, CAS # 26239-55-4) stock solution was made pH 6.5 with 50% NaOH (Sigma Cat. #S0899, CAS #1310-73-2), and the volume adjusted to 1 M ADA.

TABLE I
Table I lists the crystal form space group parameters for crystals made in accordance with the
present invention.
Crystal Form Space Group Parameters

| | | | Alternative | | Space | ######## UNIT CELL PARAMETERS ########## | | | | | |
|---|---|---|---|---|---|---|---|---|---|---|---|
| Crystal Form | Protein | Oligo | Oligo | Drug | Group | a | b | c | alpha | beta | gamma |
| Crystal Form 7 | topo70 | CL22-sT:CP22-A | CL22-sA:CP22-T | None | P32 | 72.0 | 72.8 | 185.5 | 90.0 | 90.0 | 120.0 |
| Crystal Form 8 | topo70 | CL22-sG:CP22-C | Yet to be attempted | Topotecan | P1 | 76.2 | 76.2 | 103.8 | 107.8 | 96.1 | 113.0 |
| Crystal Form 9 TTC | topo70 | CL22-sG:CP22-C | CL22-sC:CP22-G | Topotecan | P21 | 57.7 | 115.9 | 75.4 | 90.0 | 97.3 | 90.0 |
| Crystal Form 9 AG260 | topo70 | CL22-sG:CP22-C | Yet to be attempted | AG260 | P21 | 57.7 | 115.9 | 75.4 | 90.0 | 97.3 | 90.0 |
| Crystal Form 10 | topo70 | CL22-sG:CP22-C | Yet to be attempted | MJ-11-38 | C2 | 260.9 | 74.6 | 57.5 | 90.0 | 96.9 | 113.0 |
| Crystal Form 11 | topo70 | CL22-sA:CP22T | CL22sC:CP22G | Hoechst- | P21212 | 270.9 | 71.1 | 57.6 | 90.0 | 90.0 | 90.0 |
| | | | | 33342 | | | | | | | |

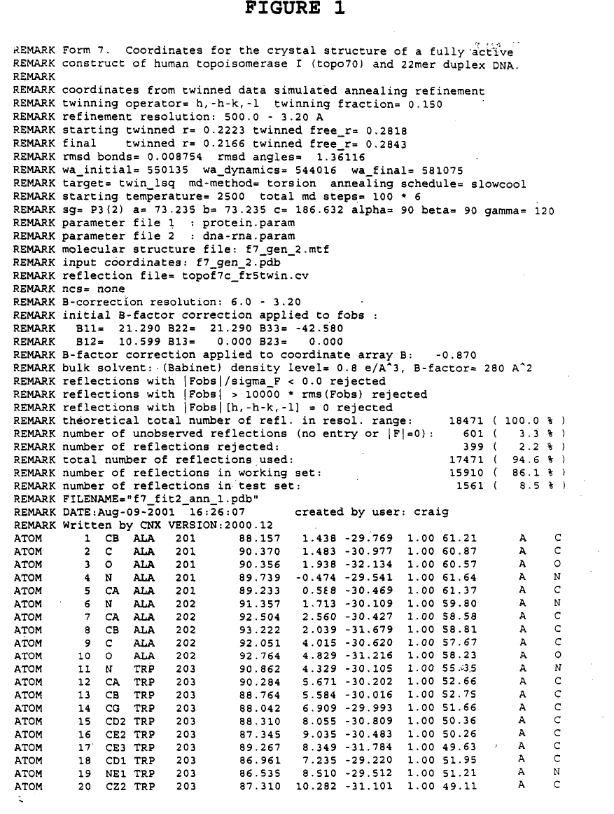

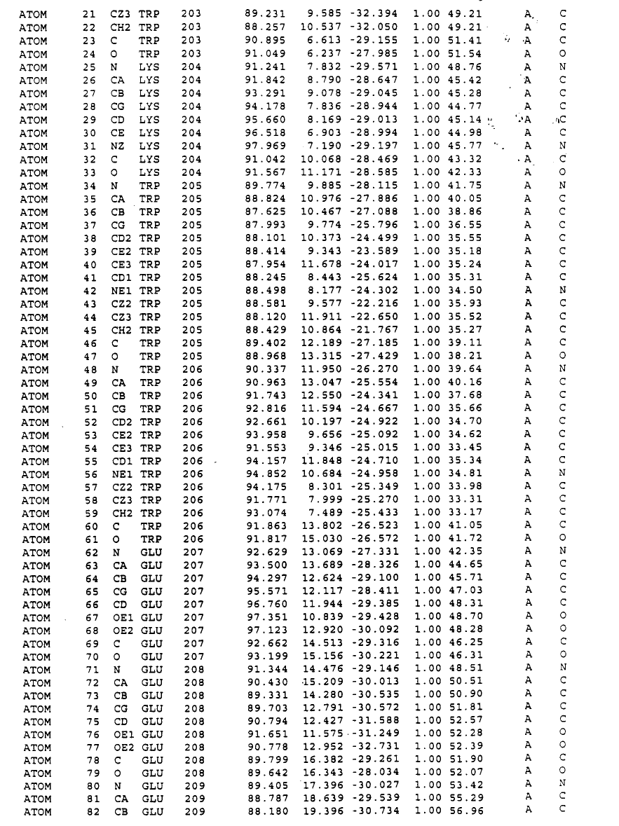

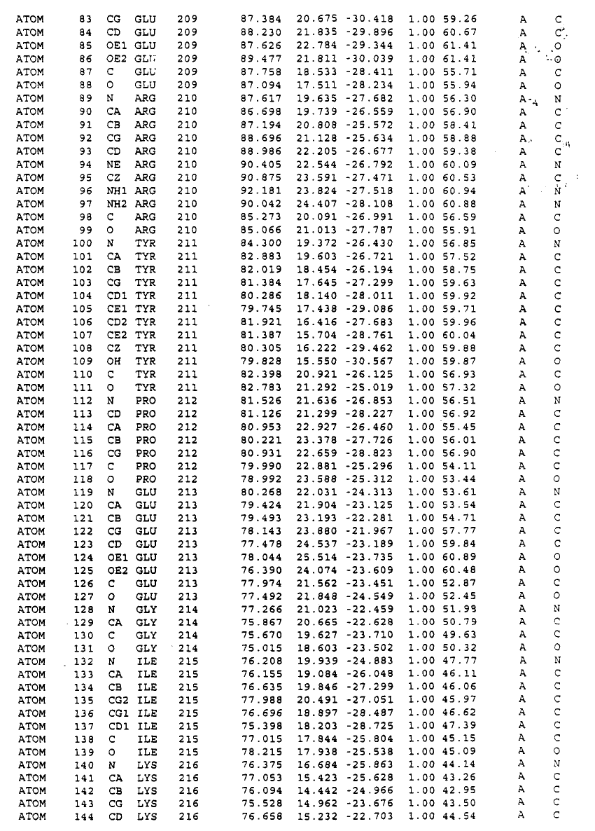

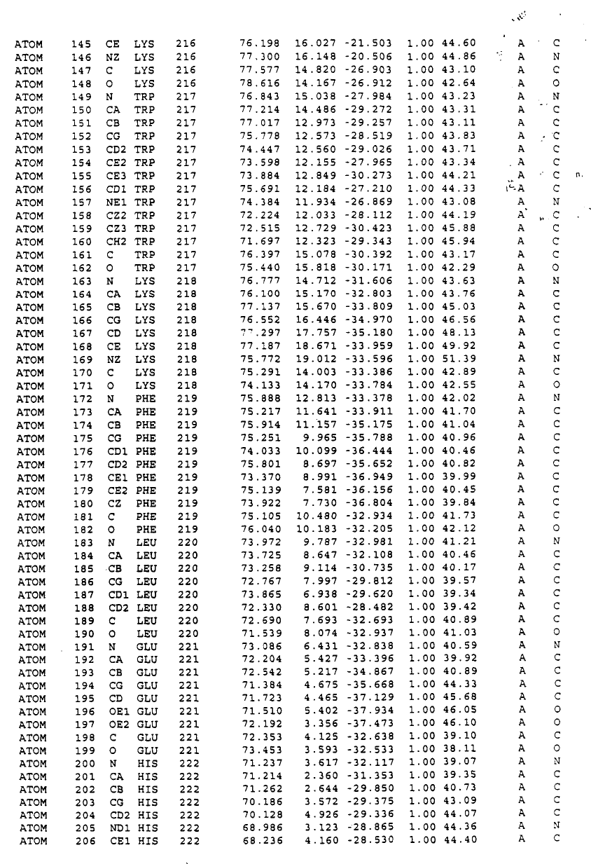

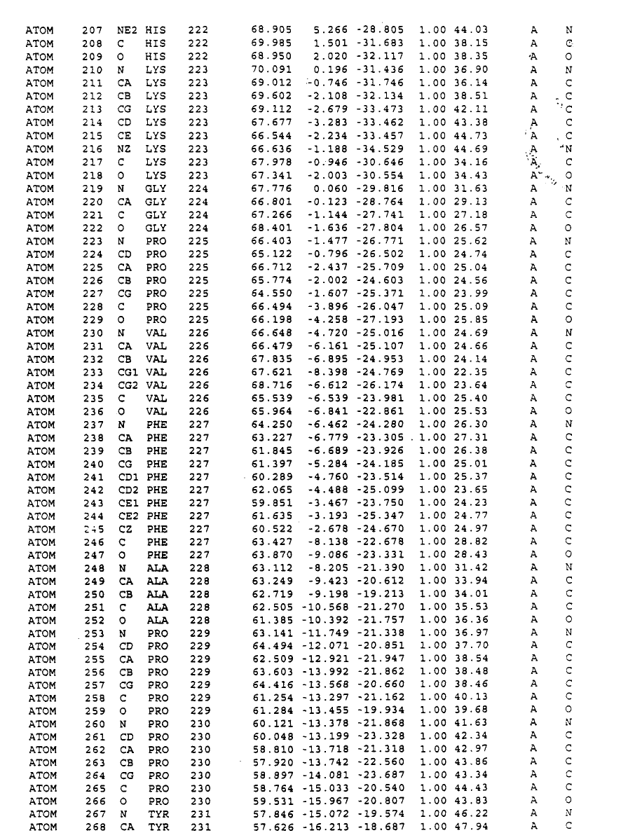

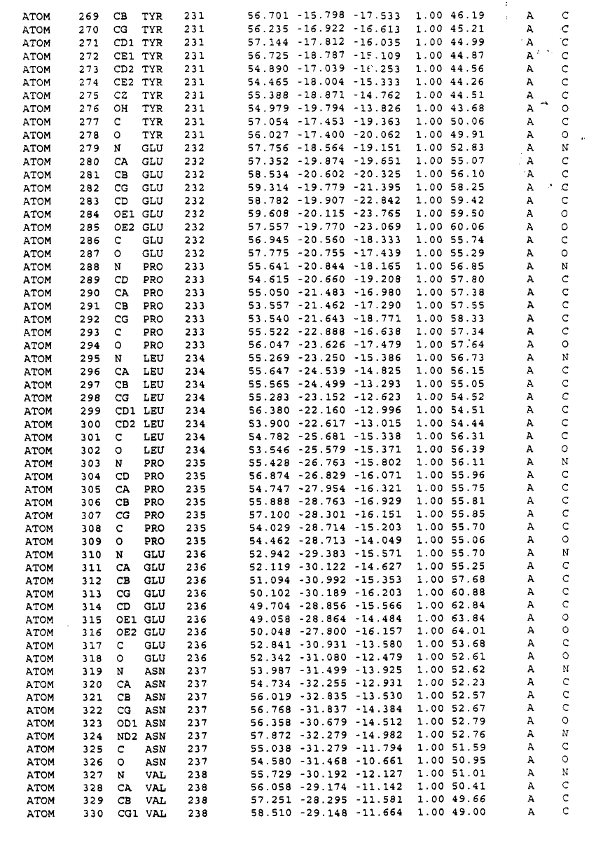

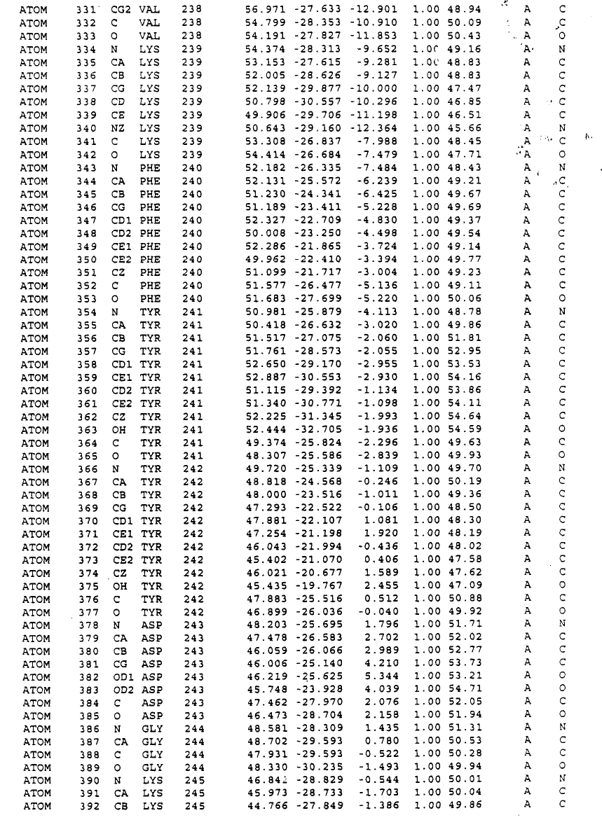

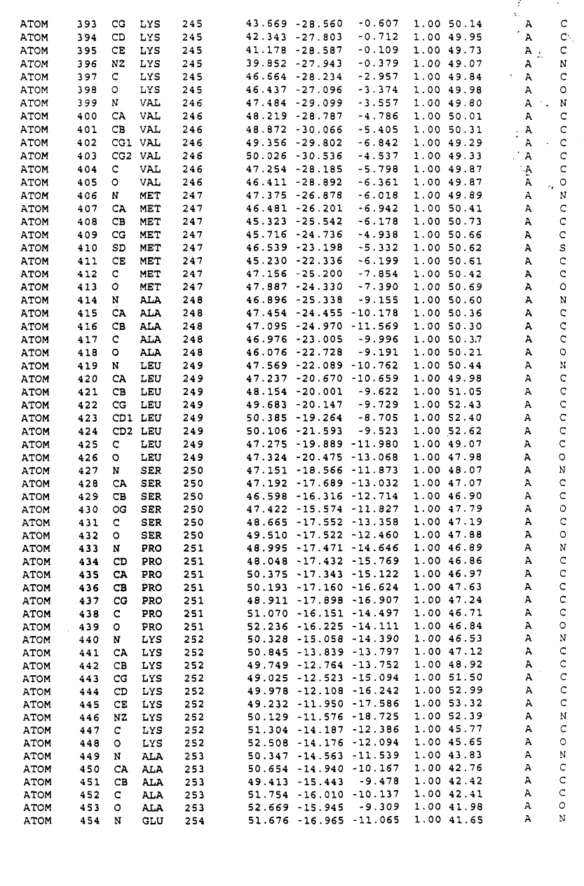

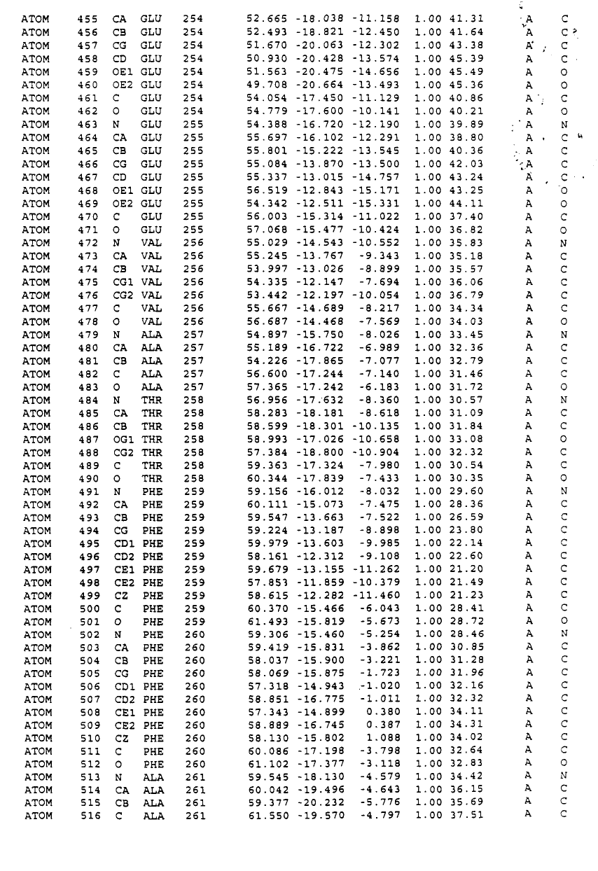

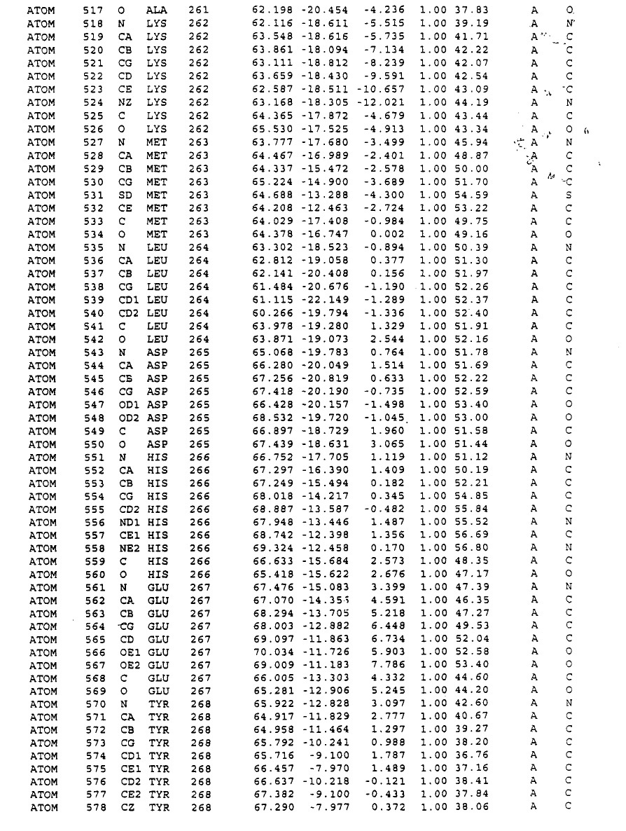

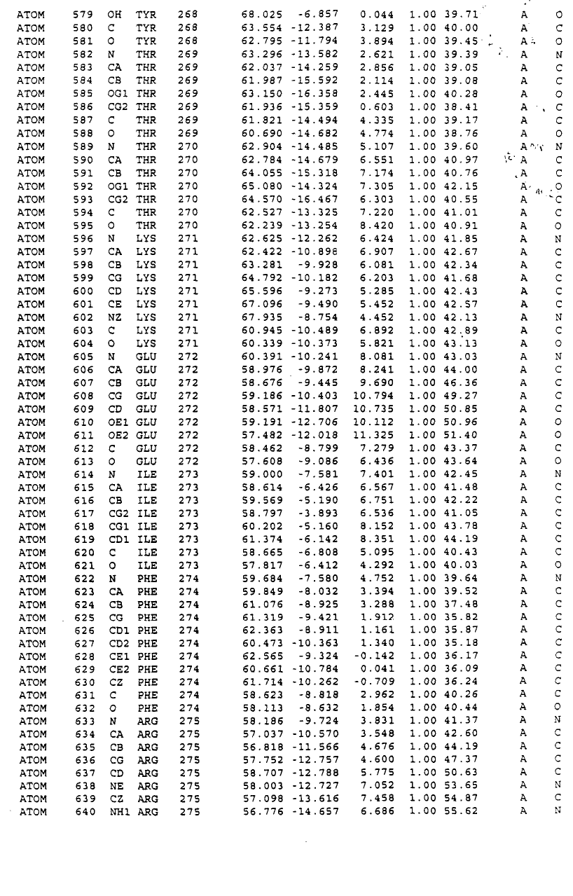

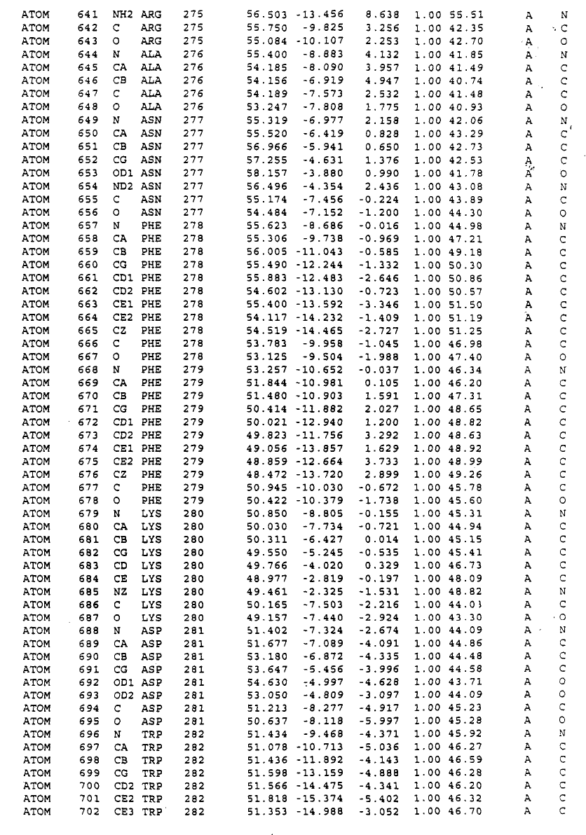

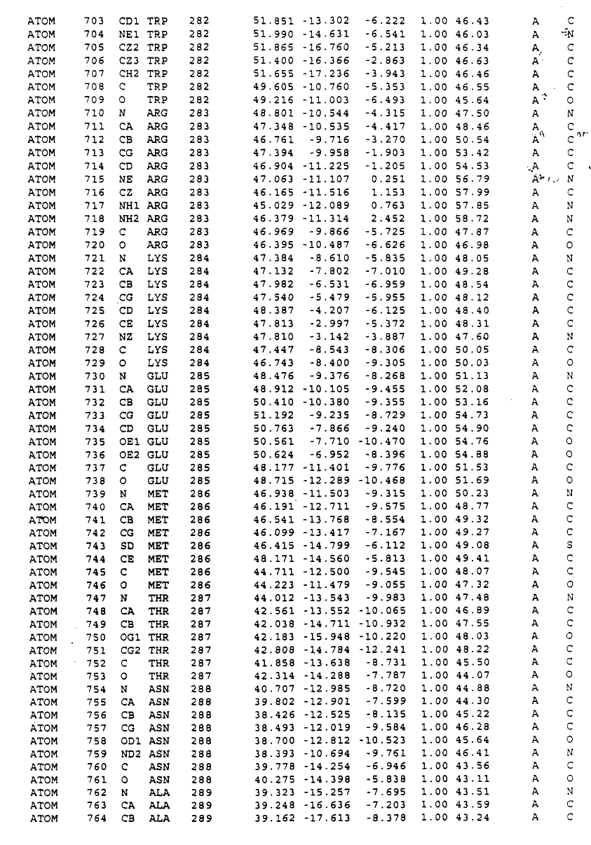

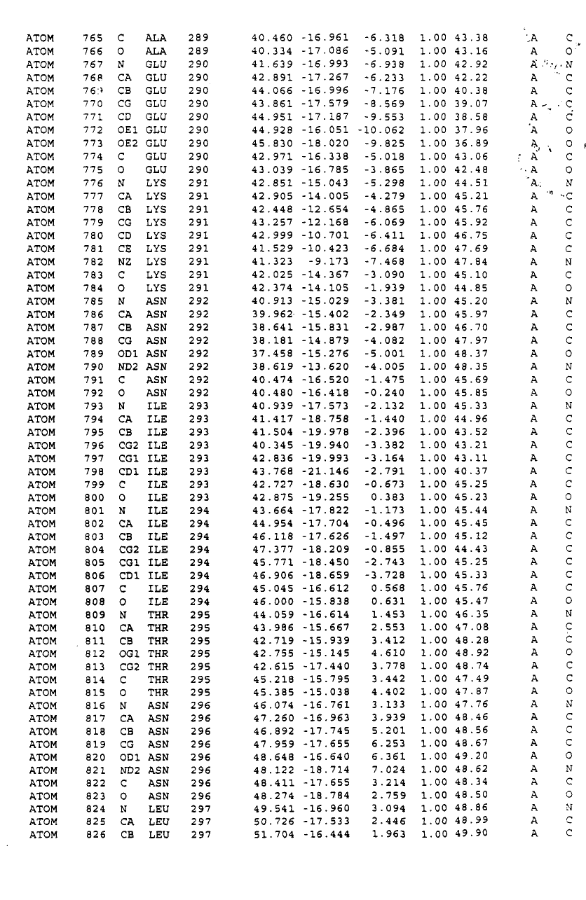

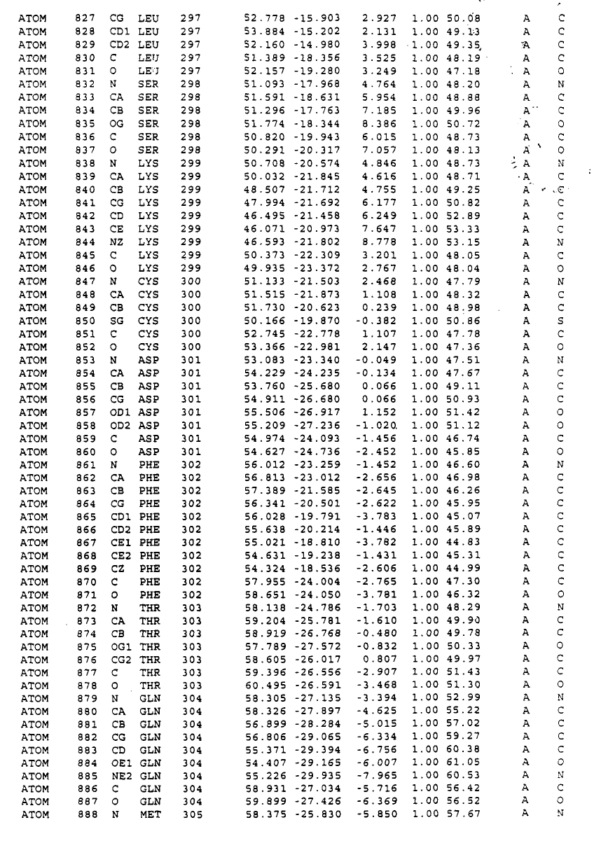

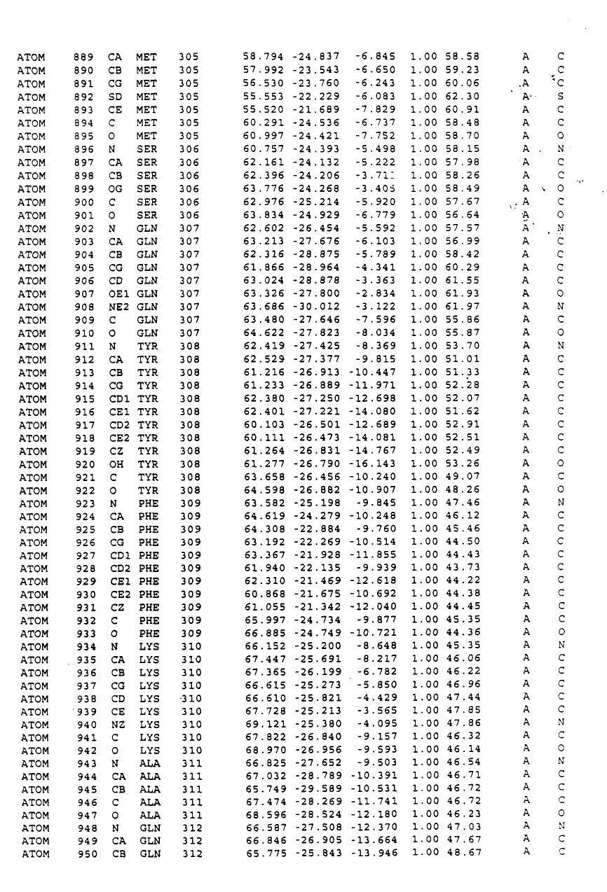

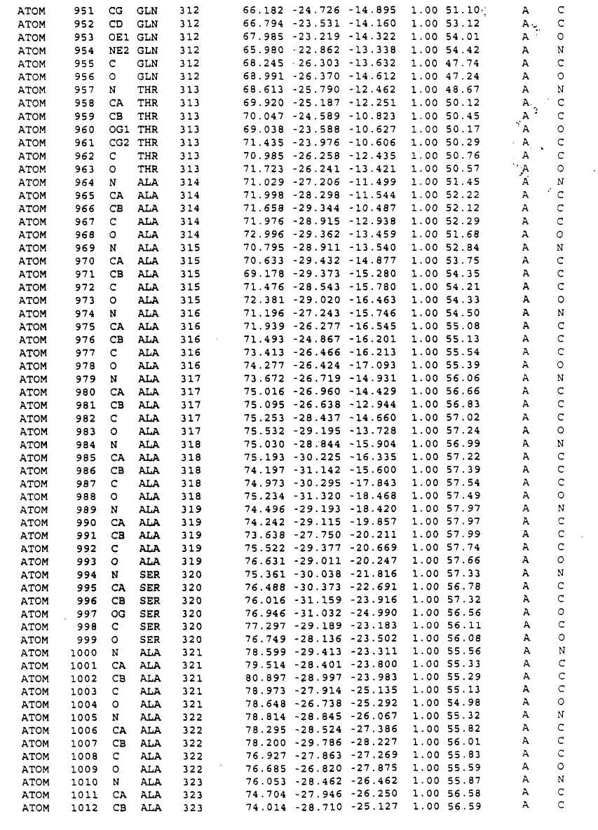

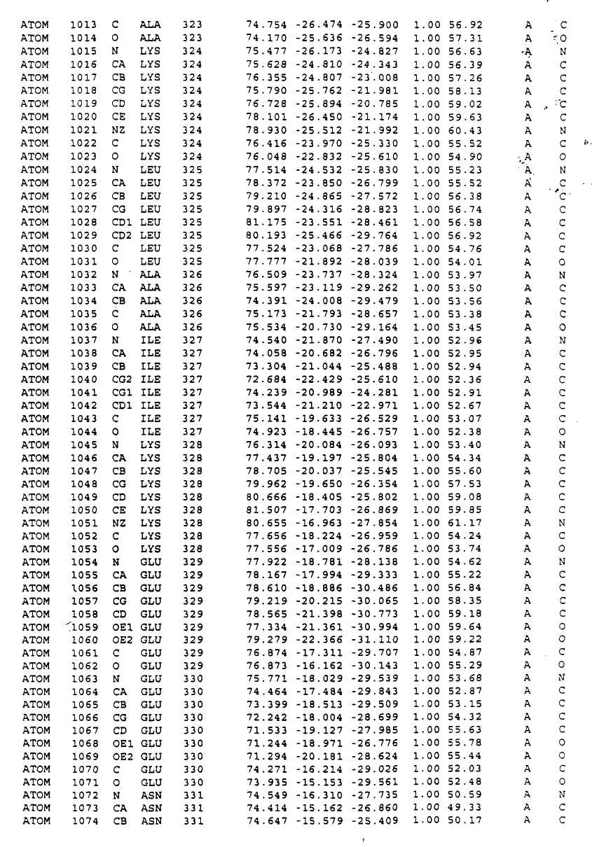

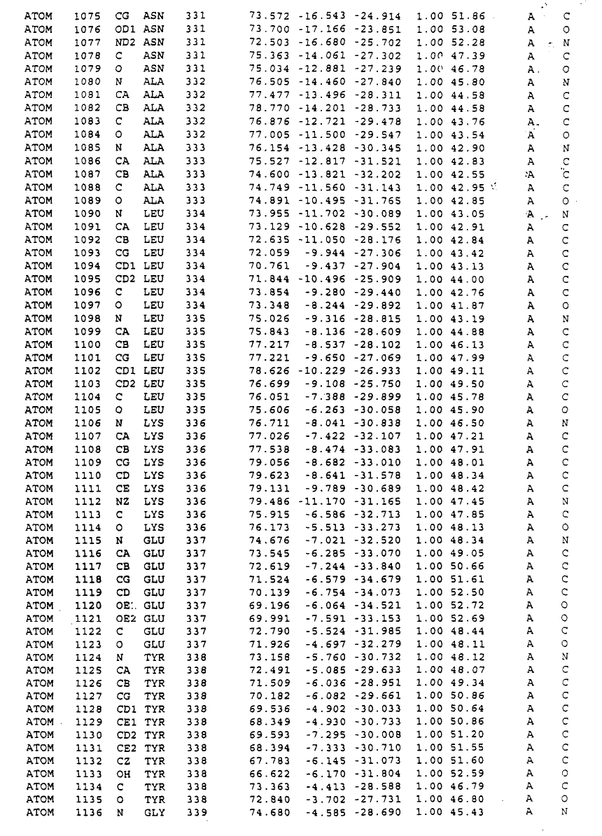

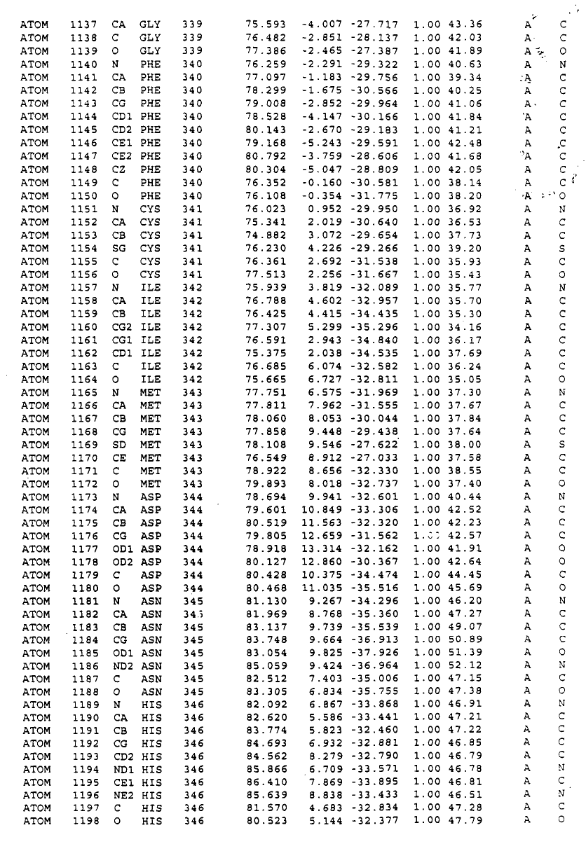

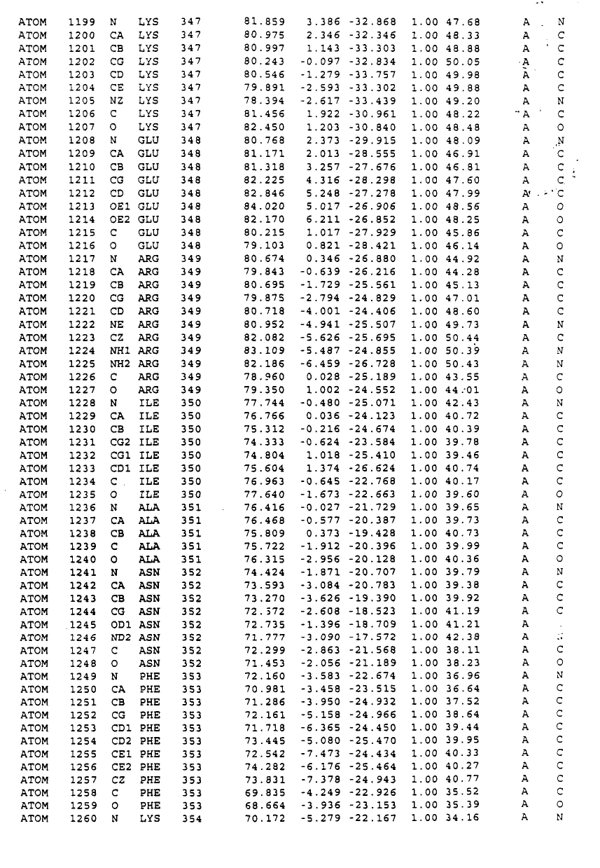

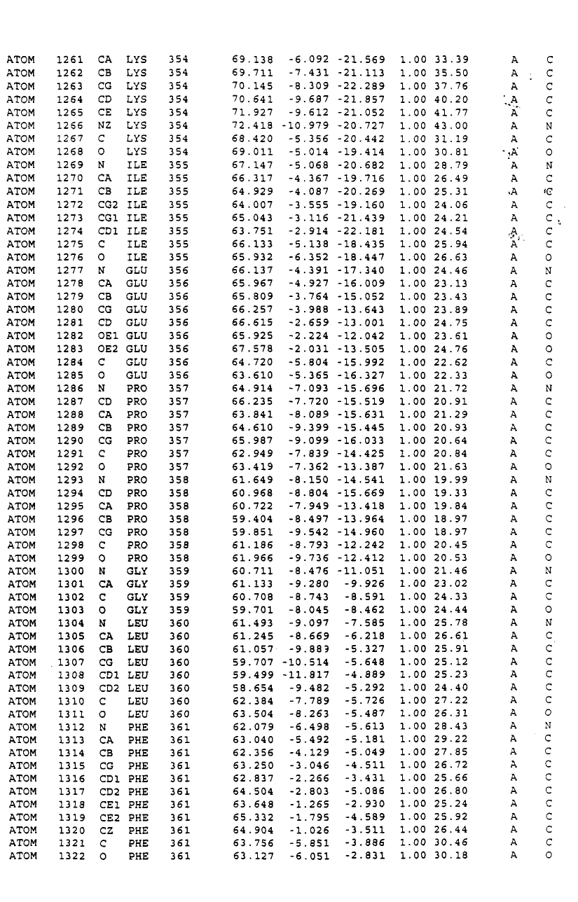

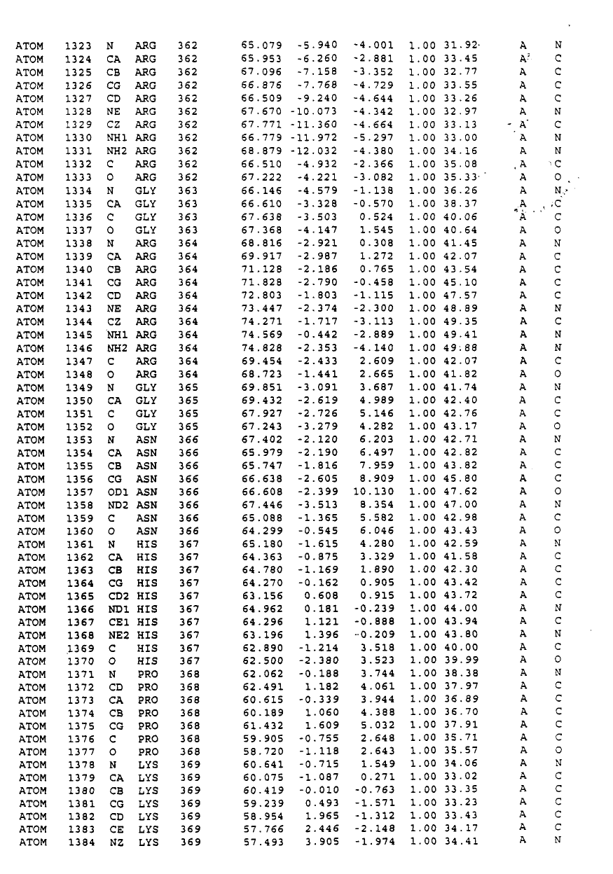

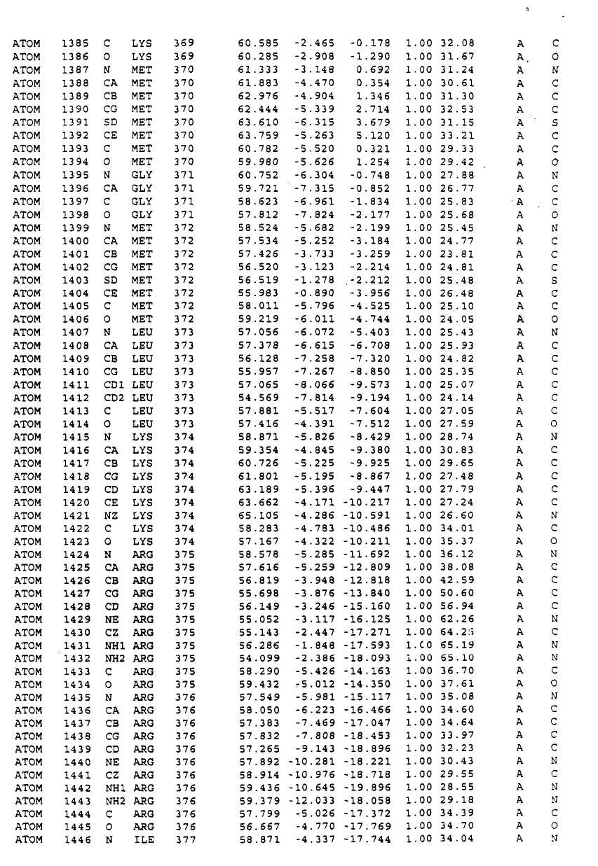

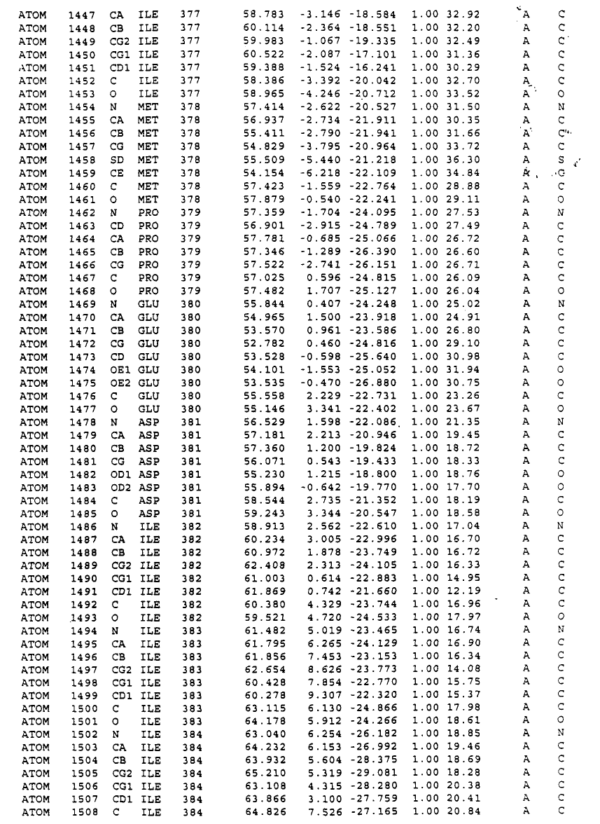

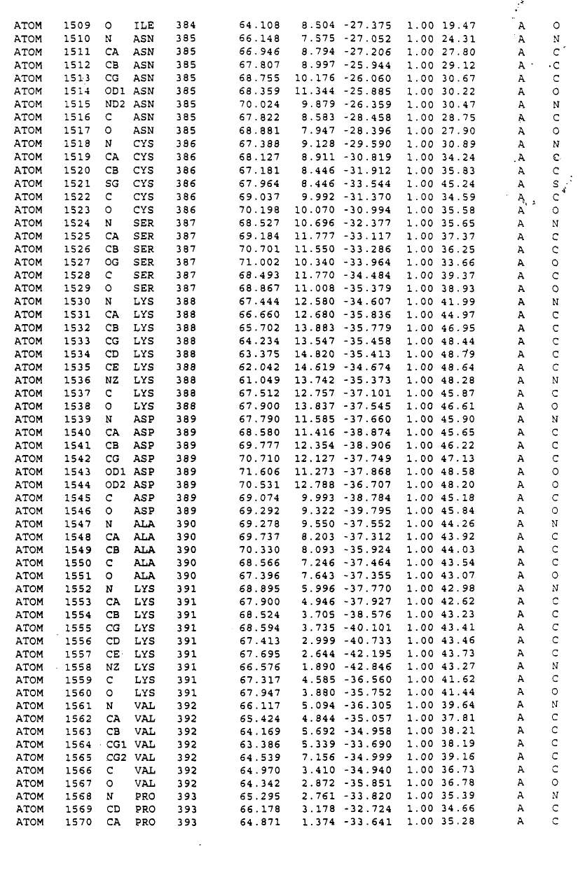

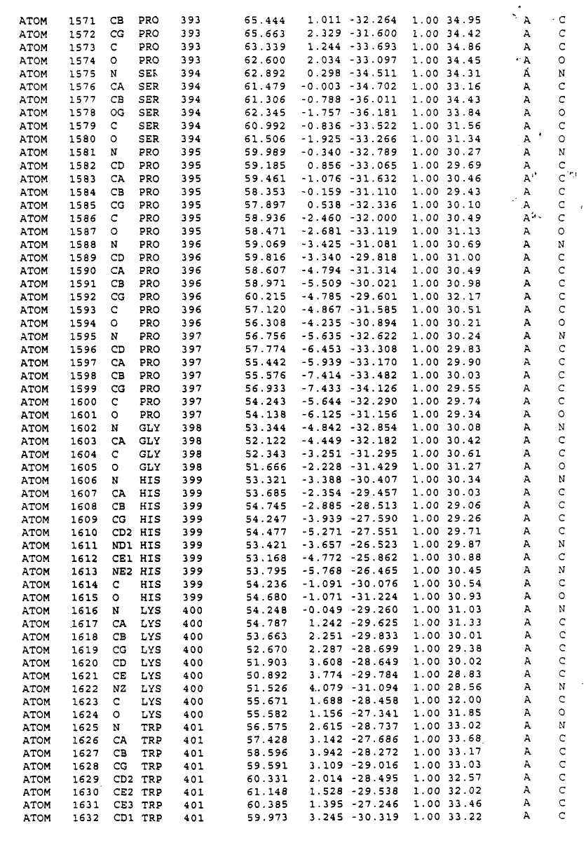

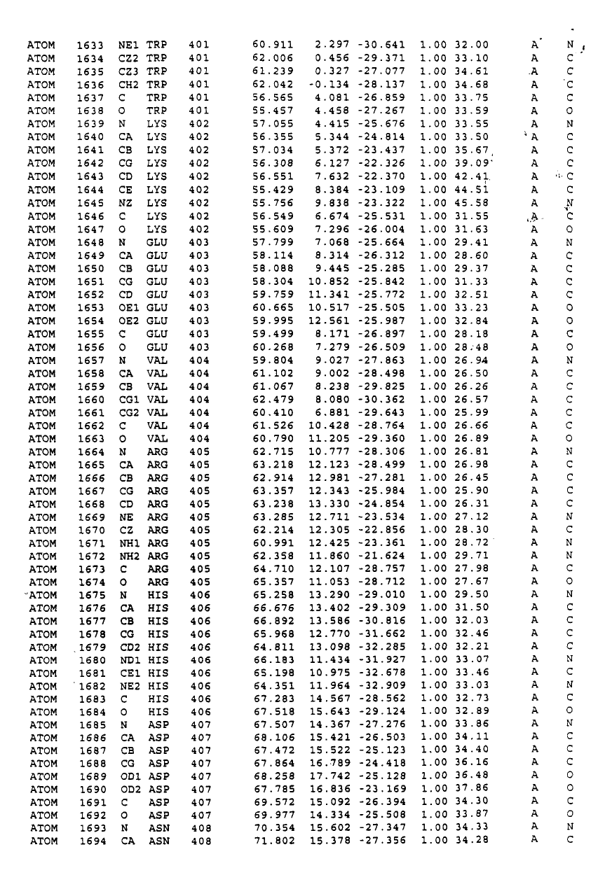

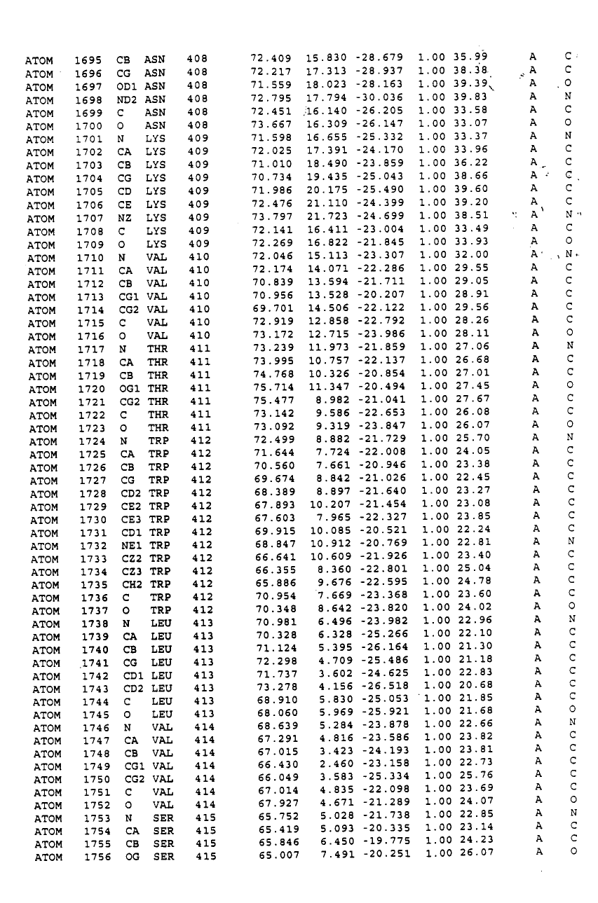

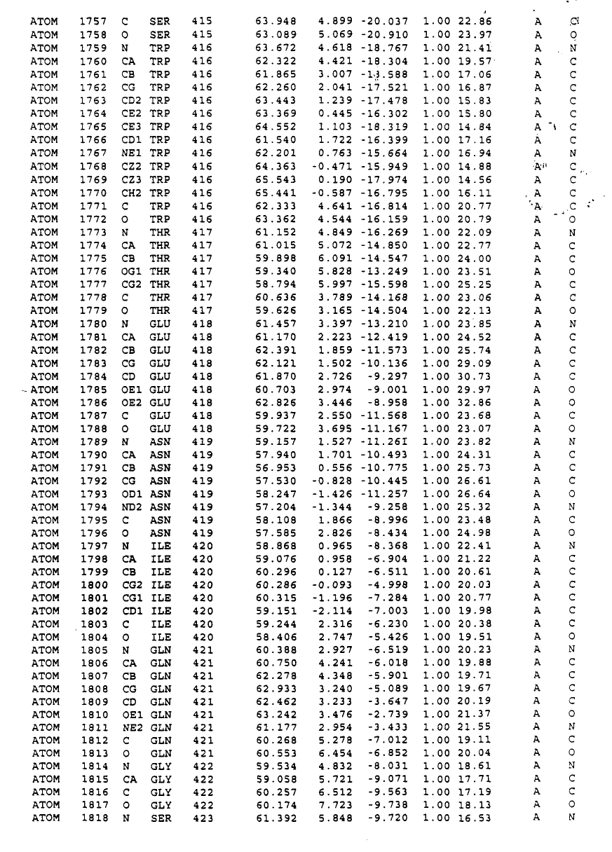

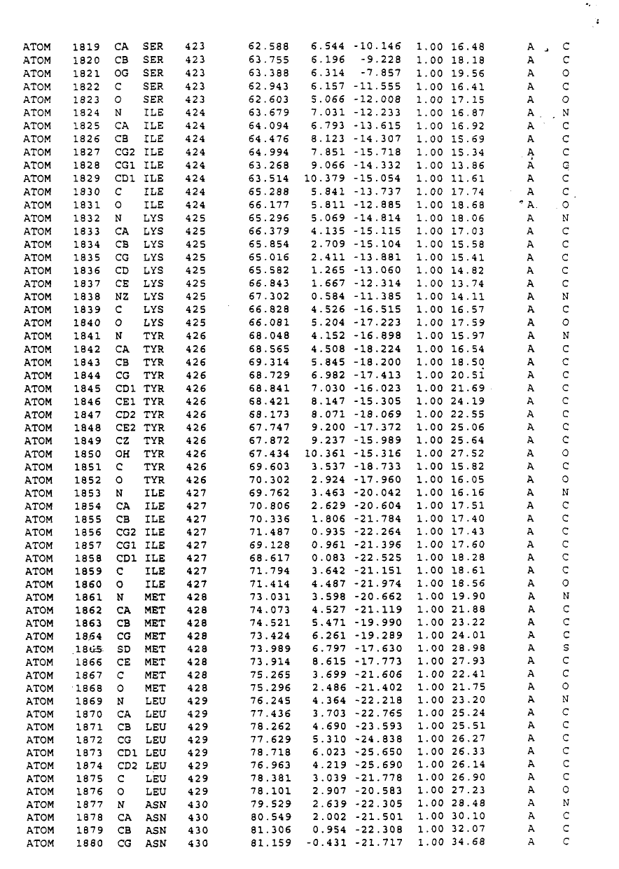

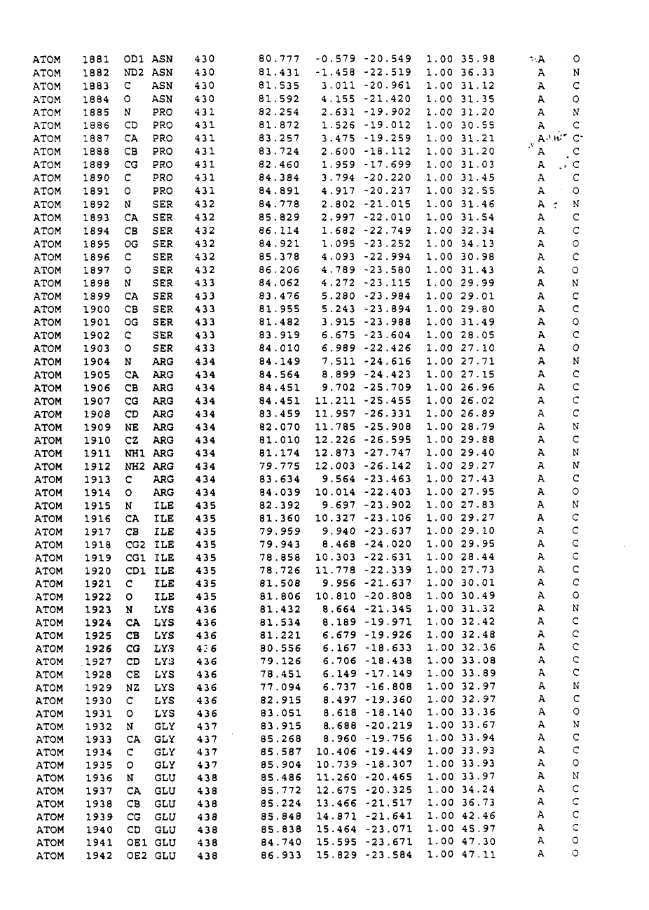

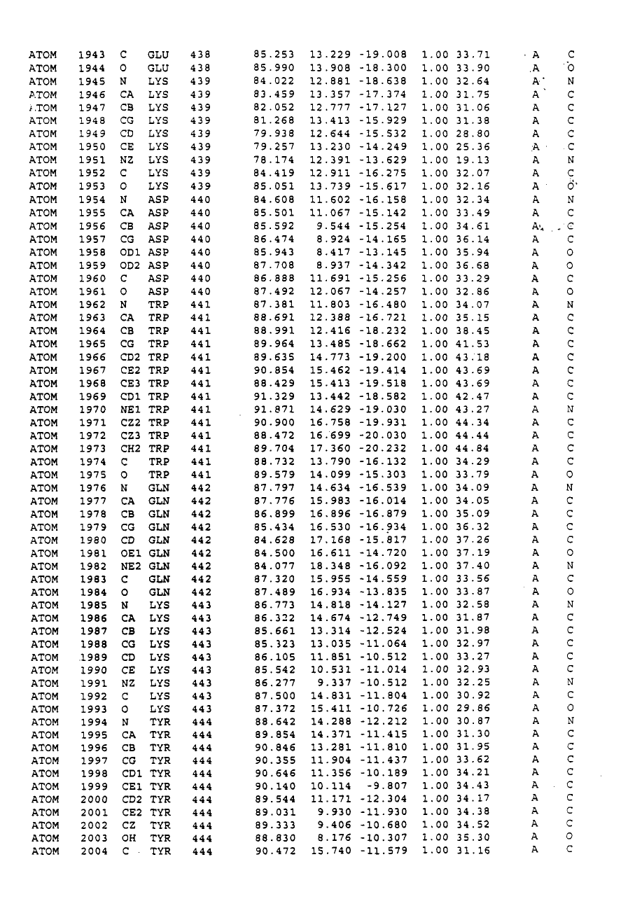

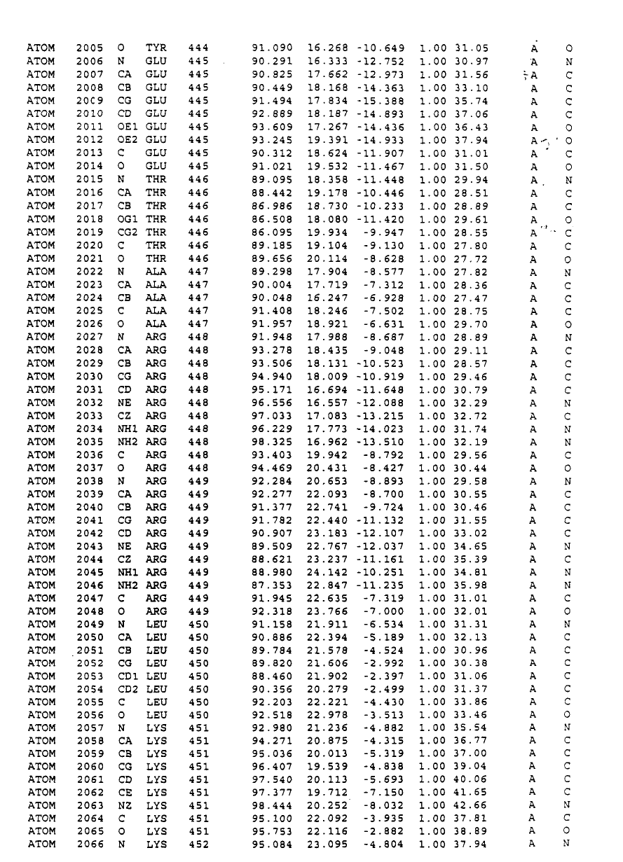

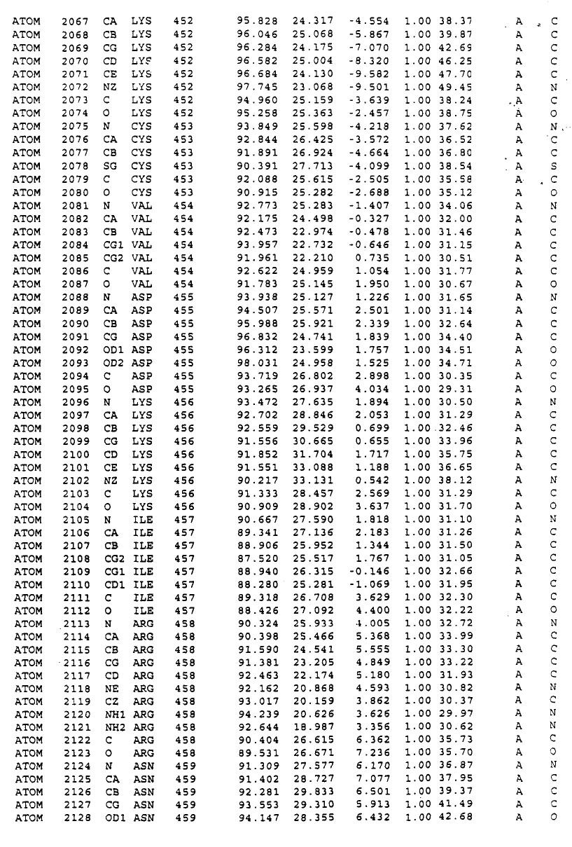

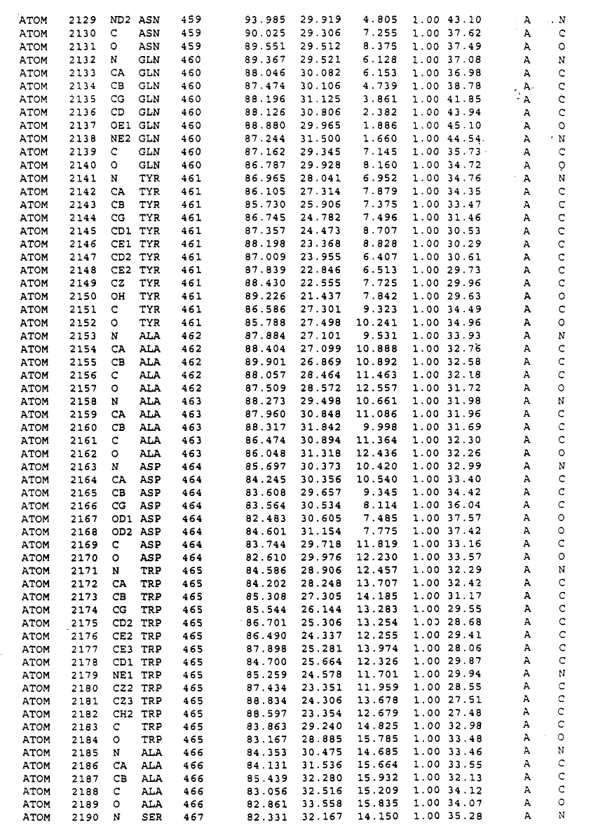

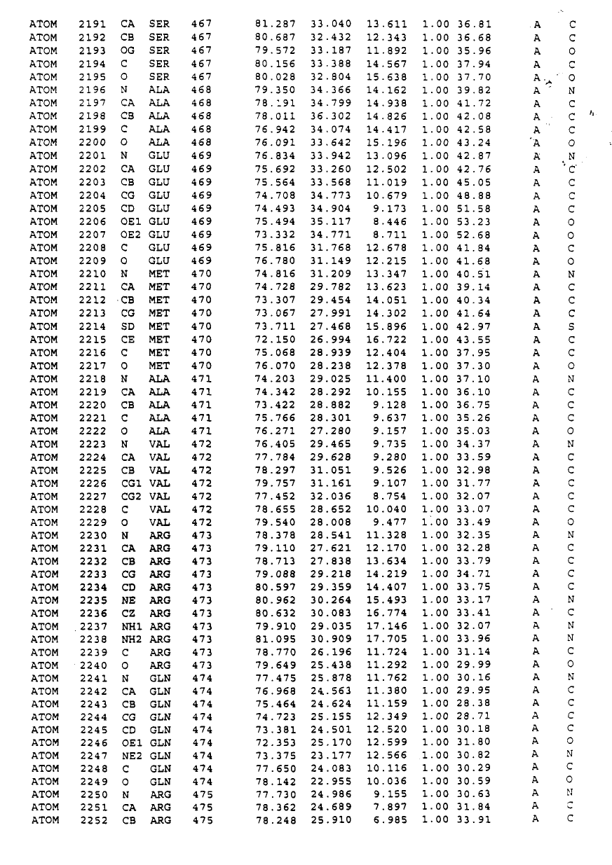

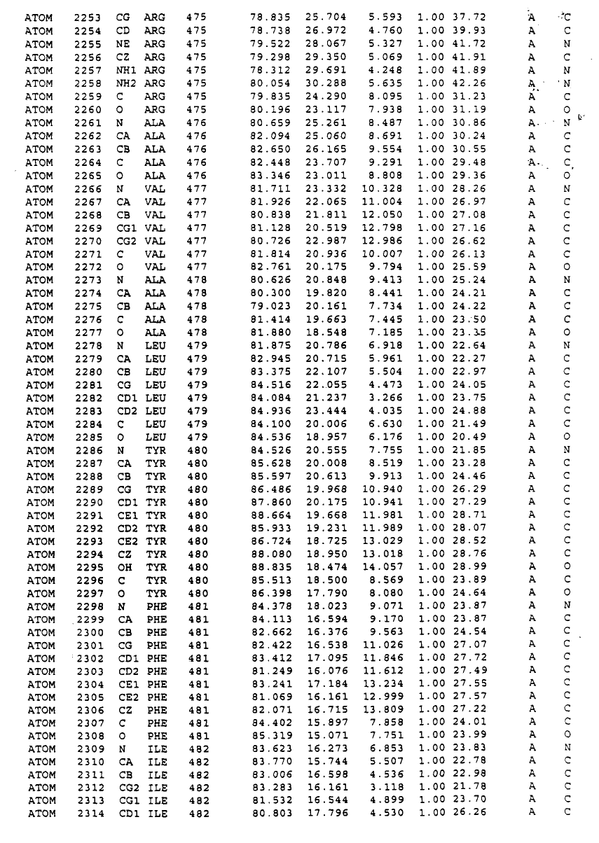

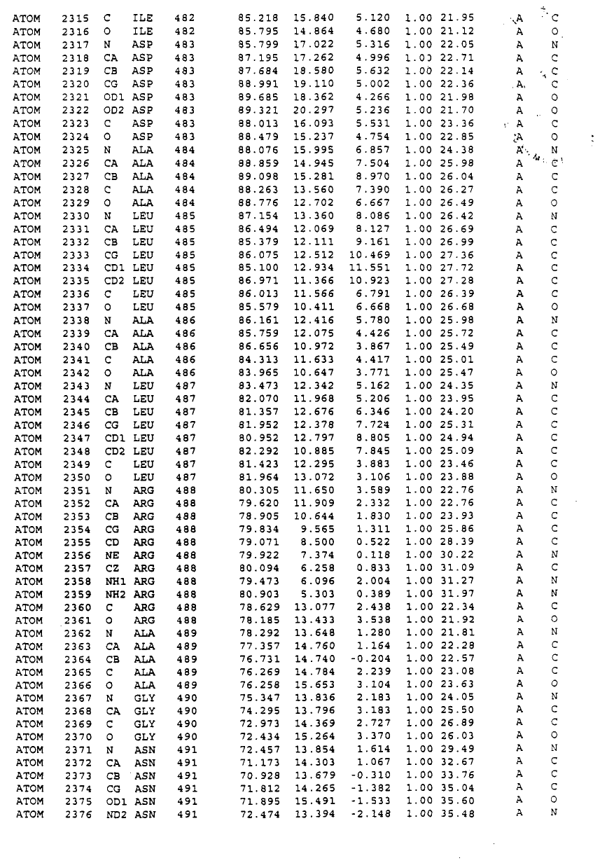

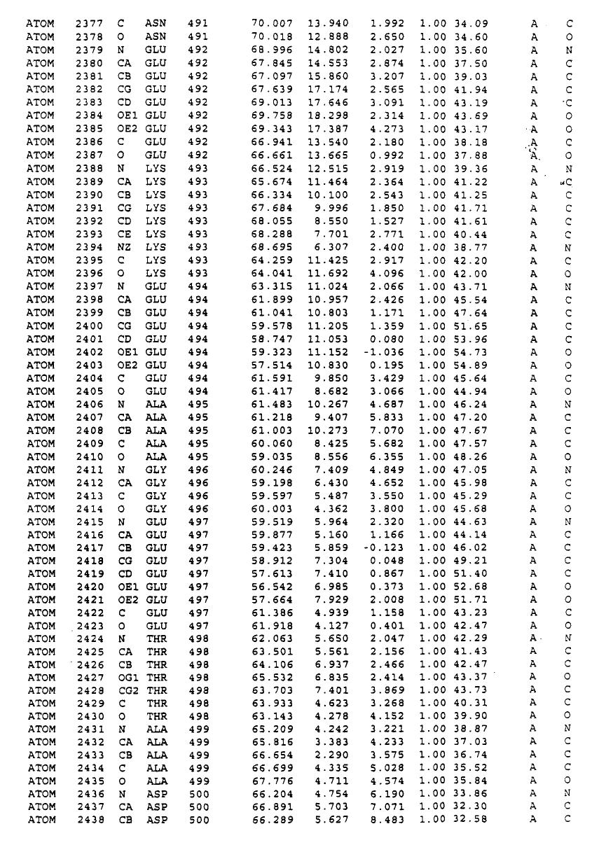

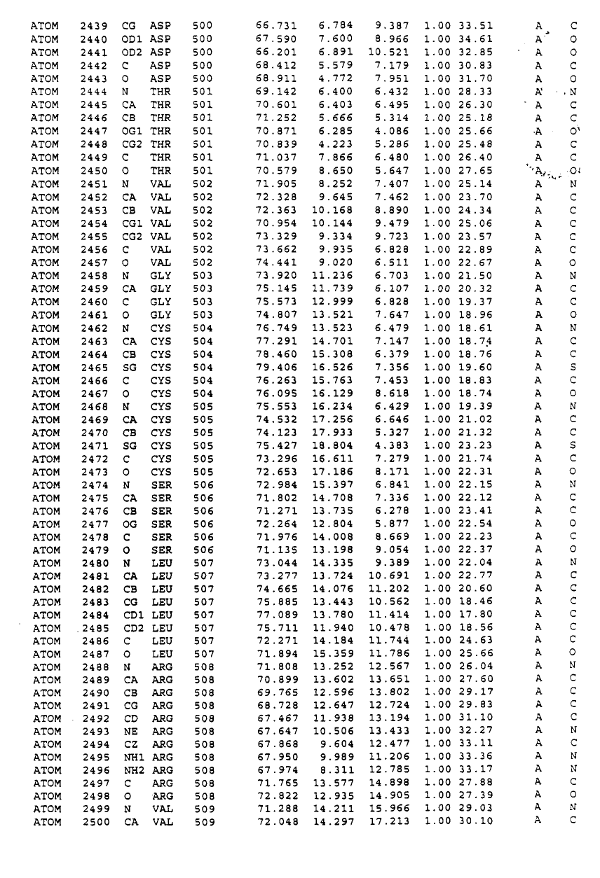

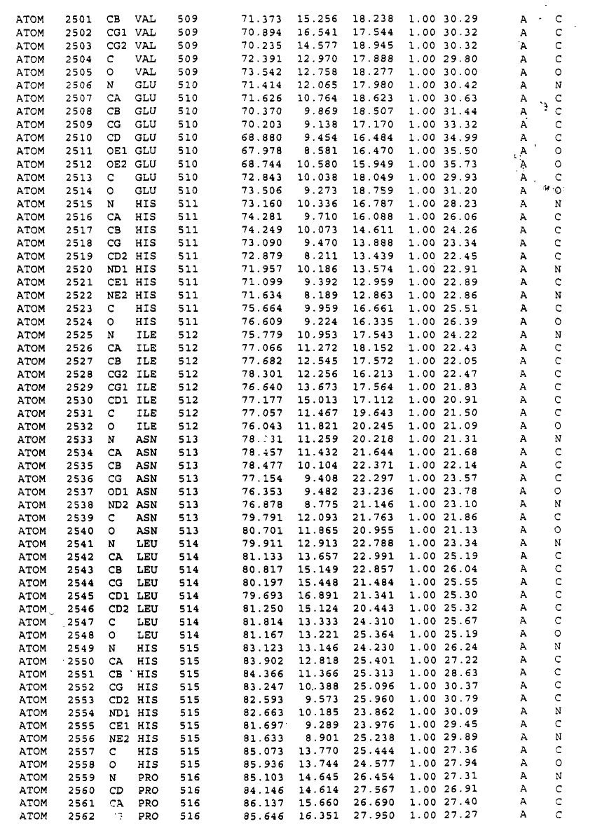

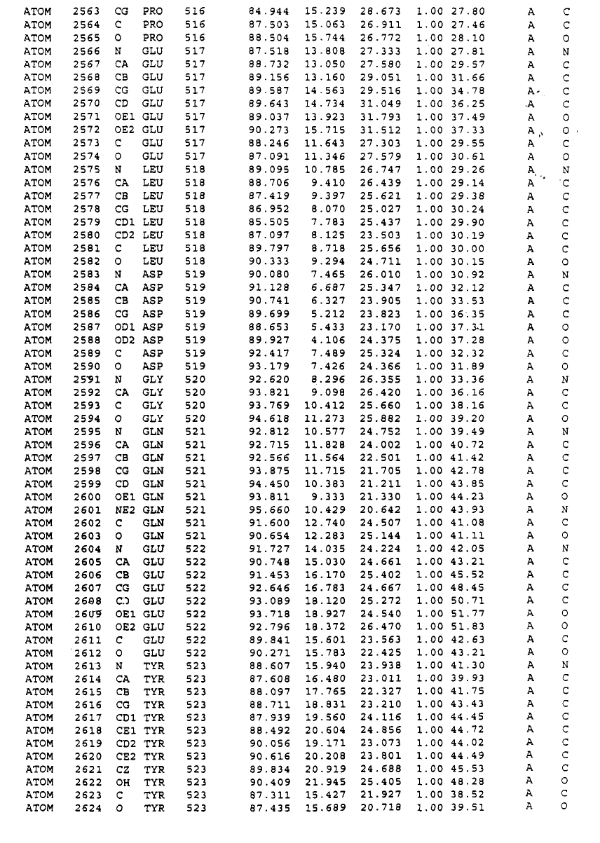

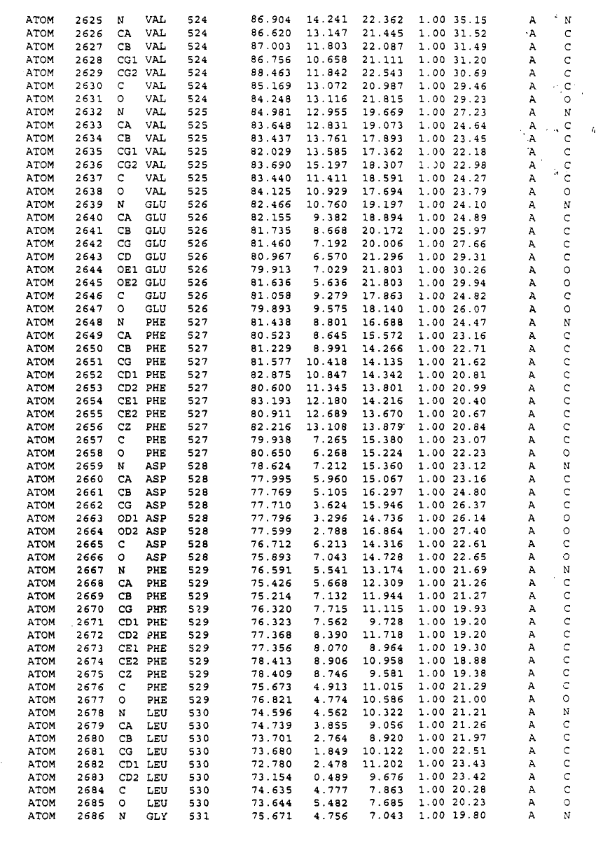

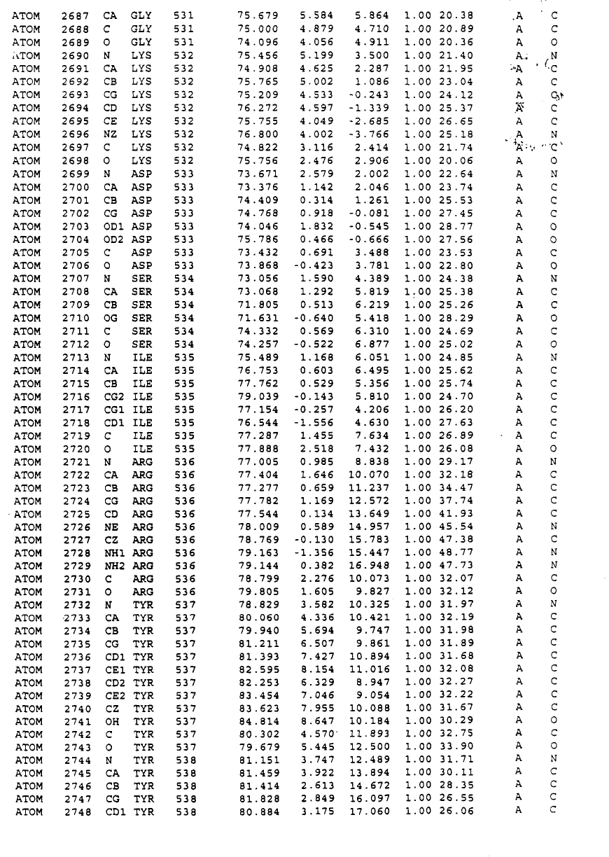

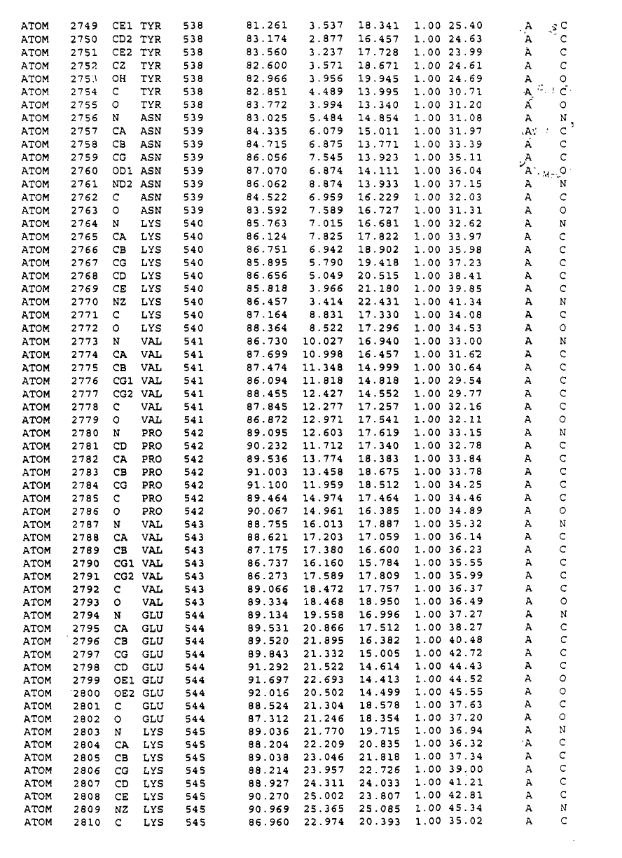

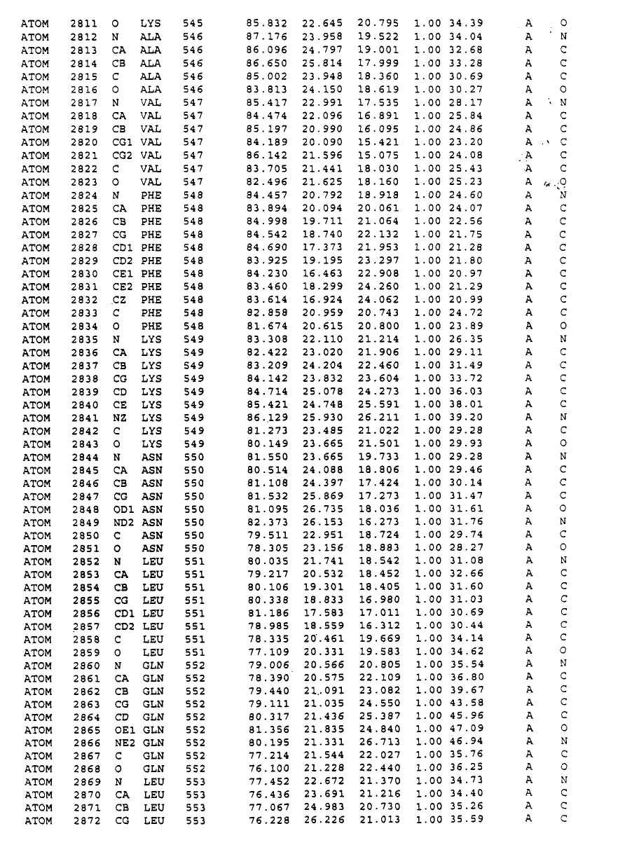

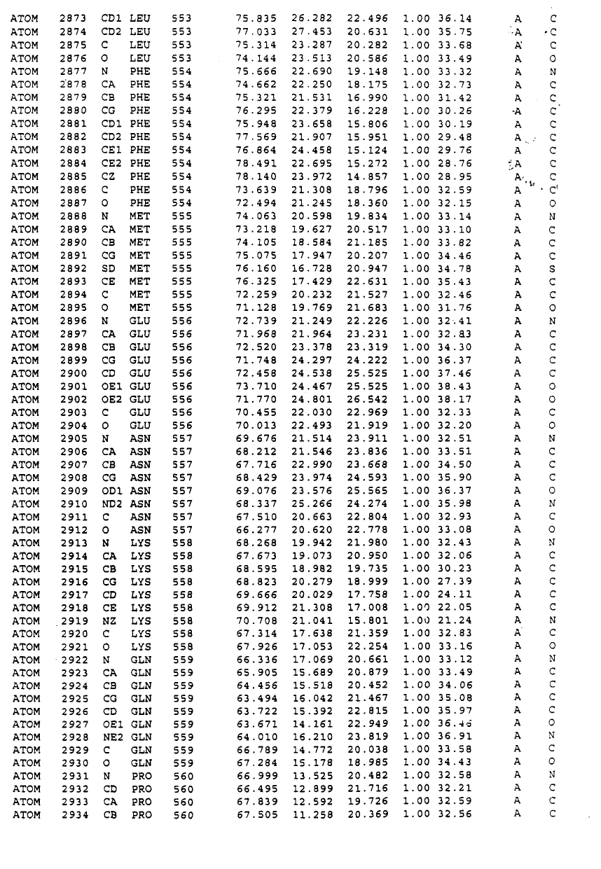

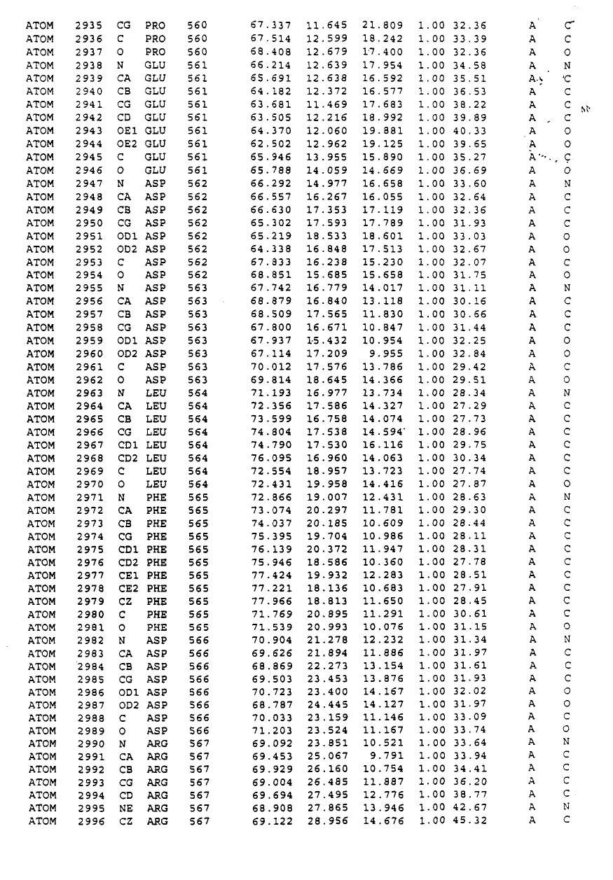

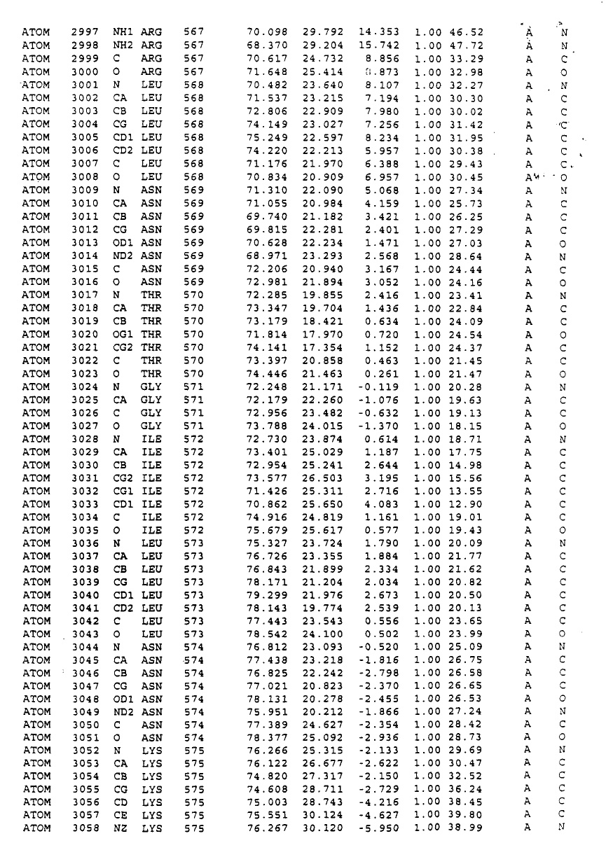

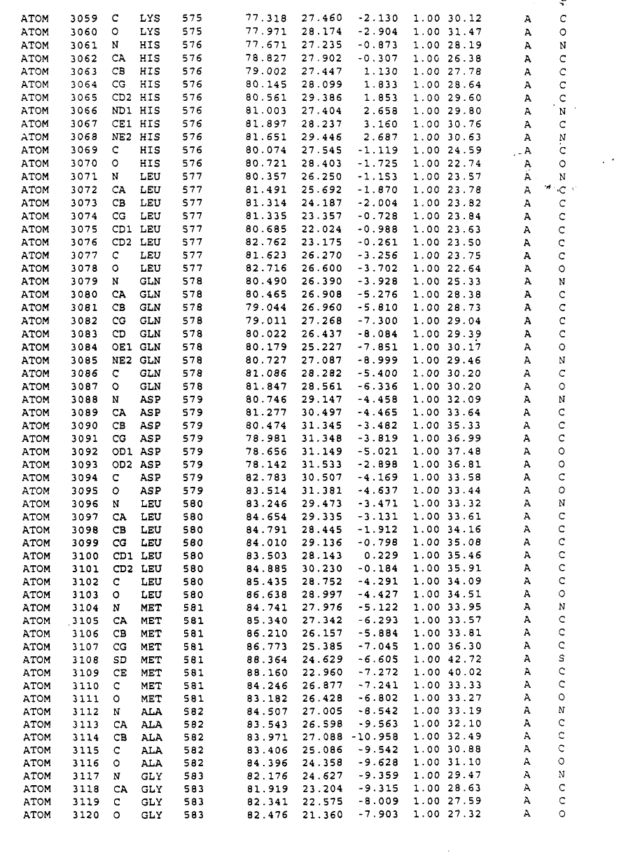

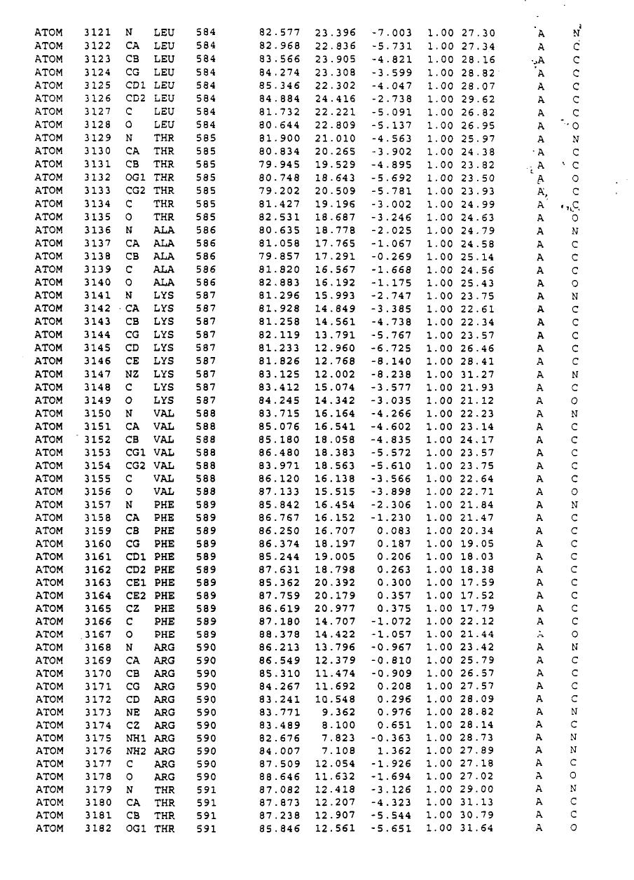

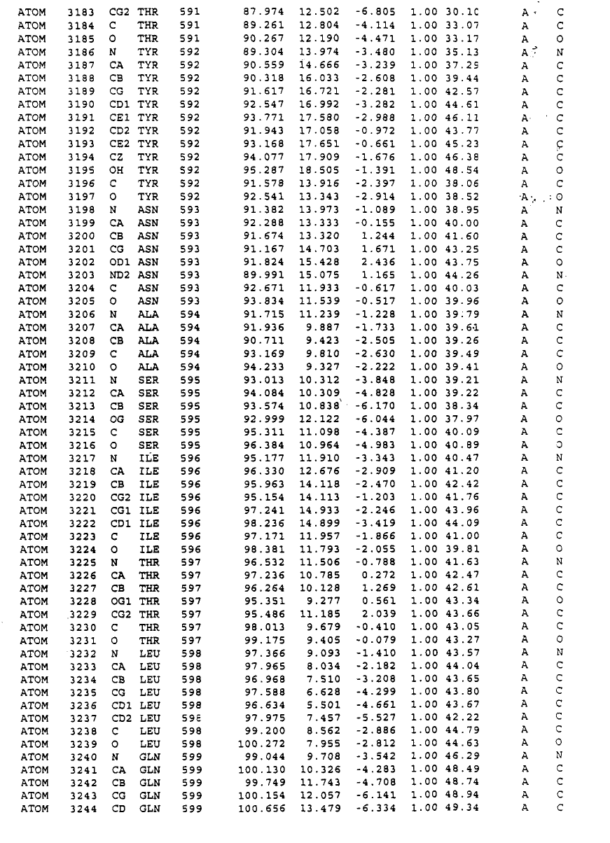

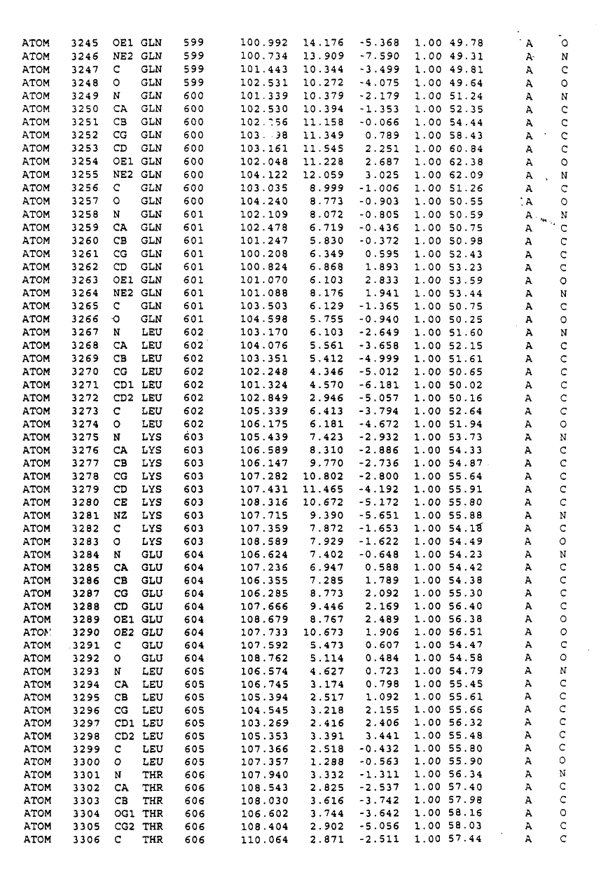

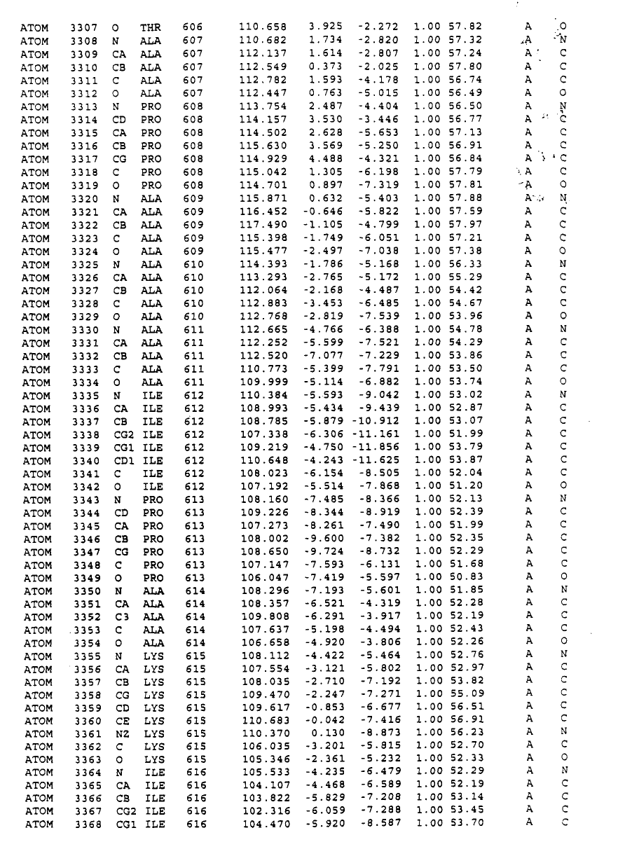

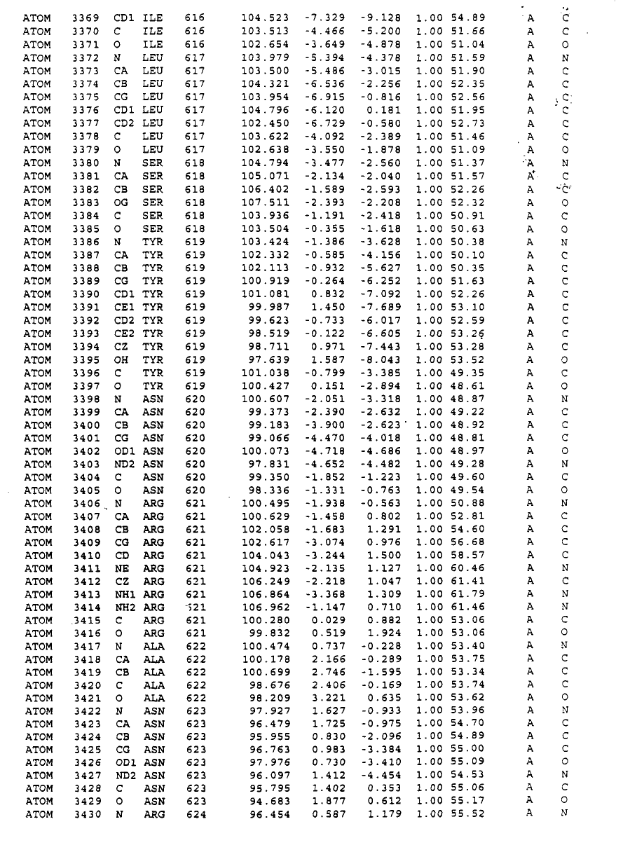

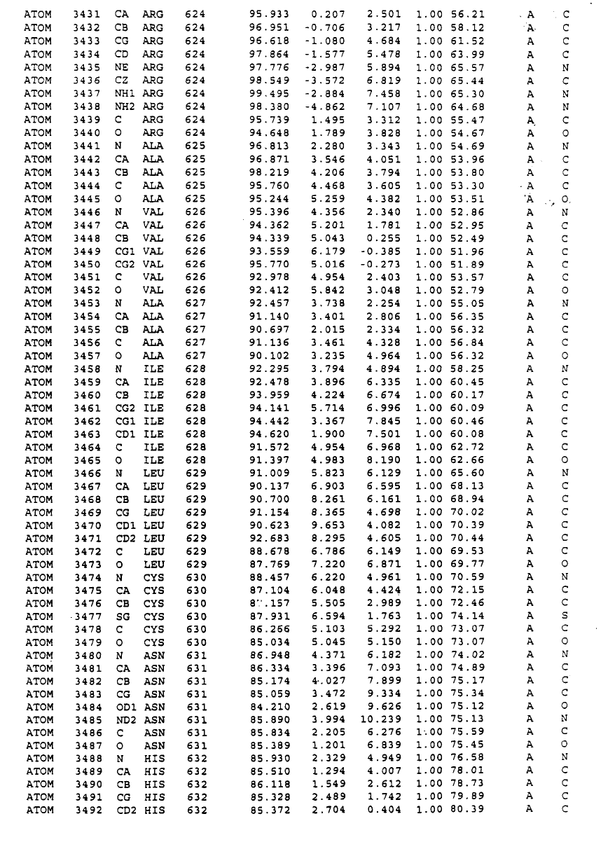

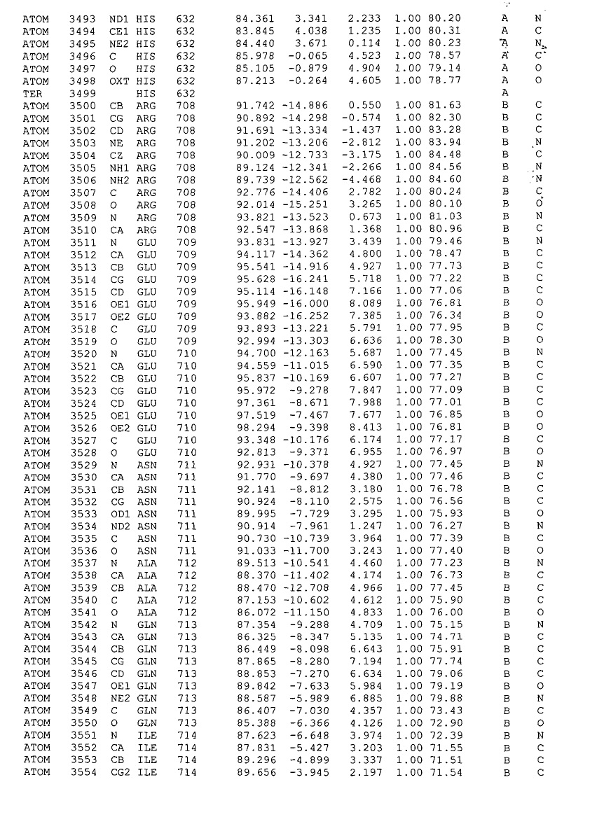

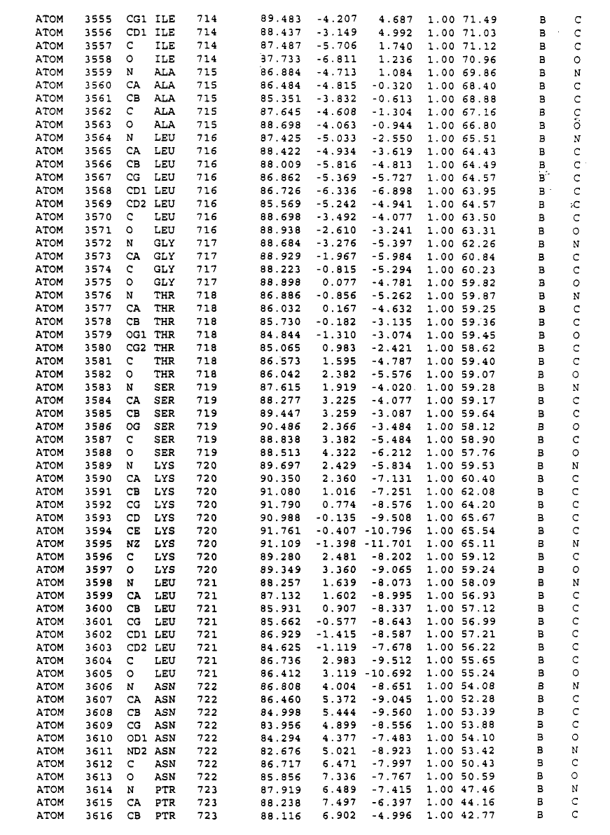

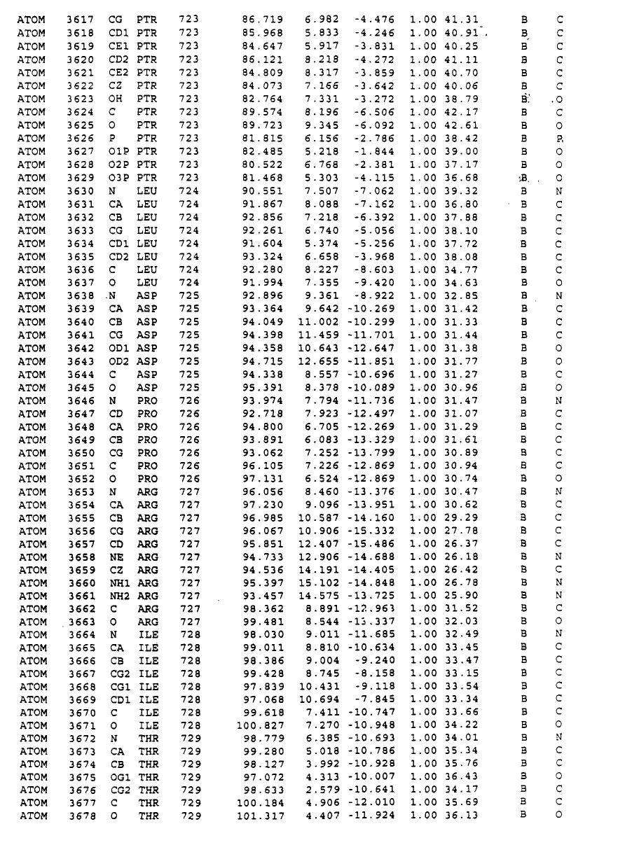

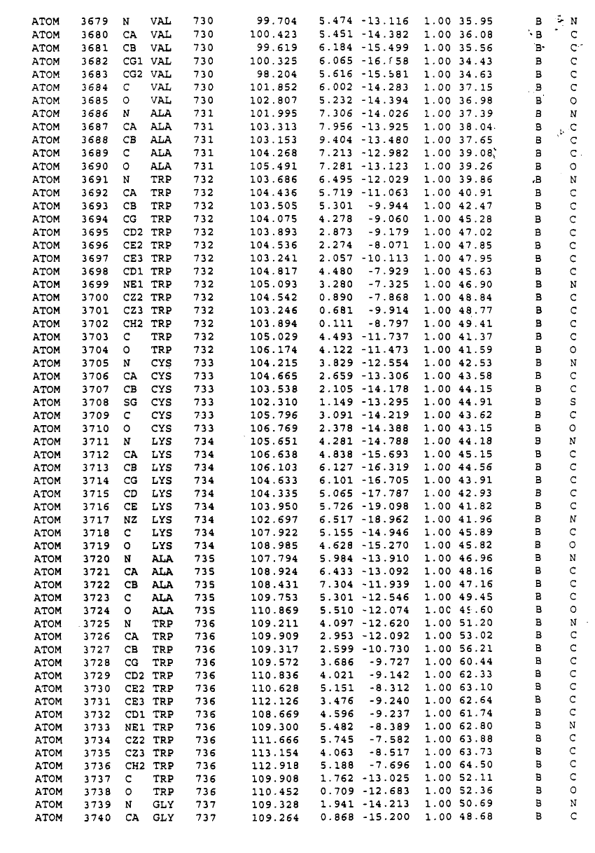

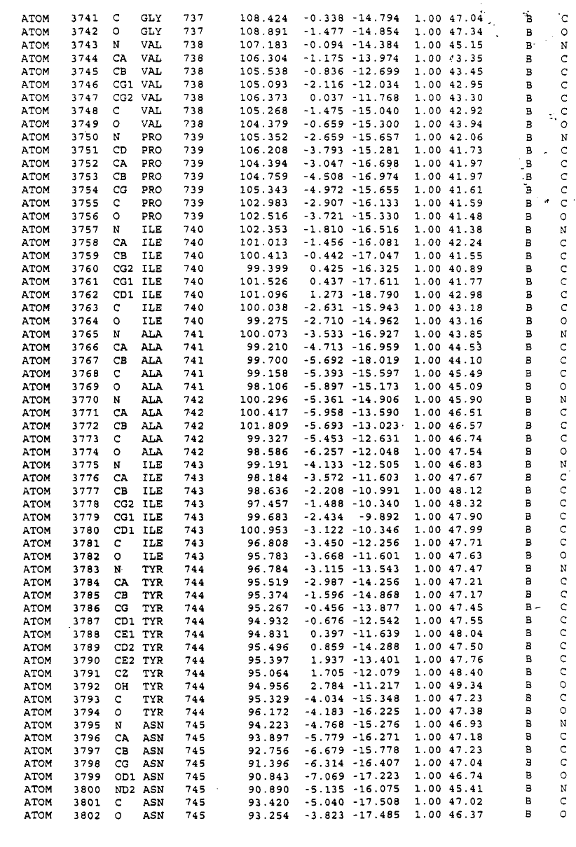

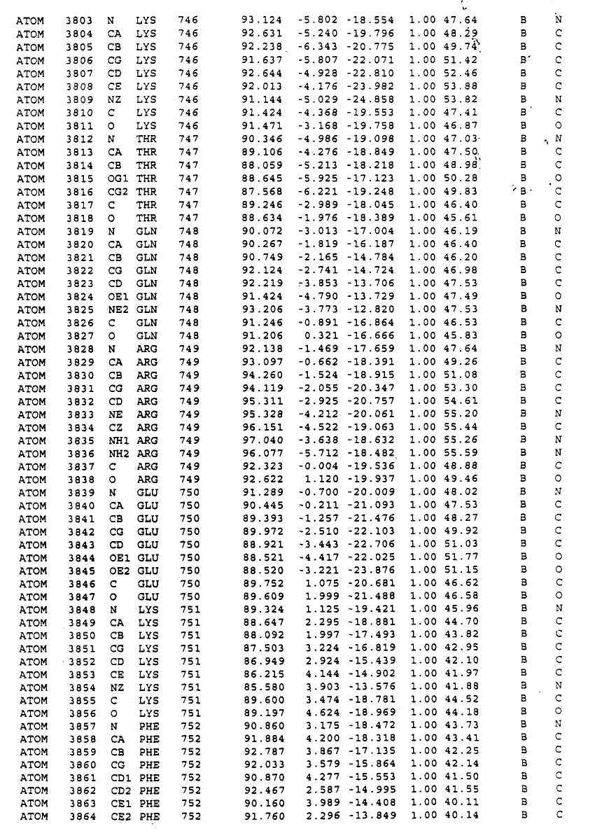

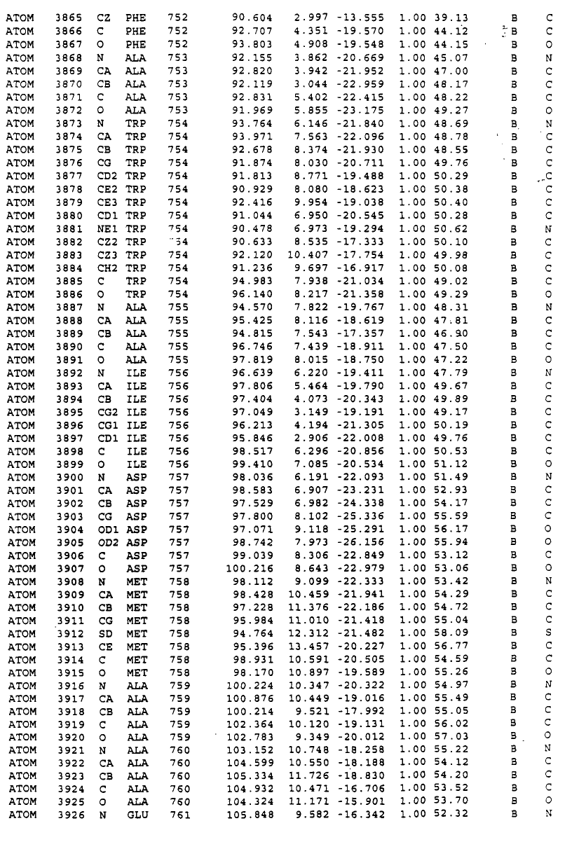

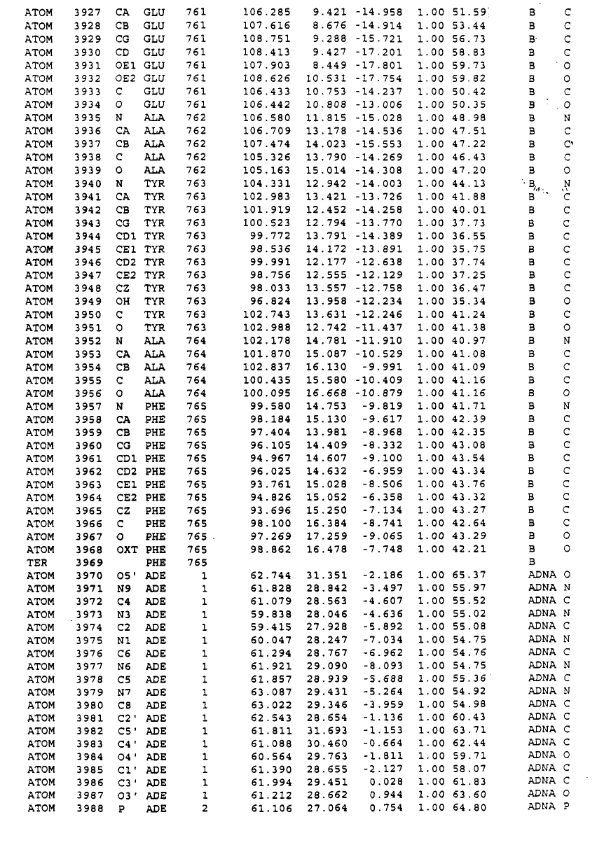

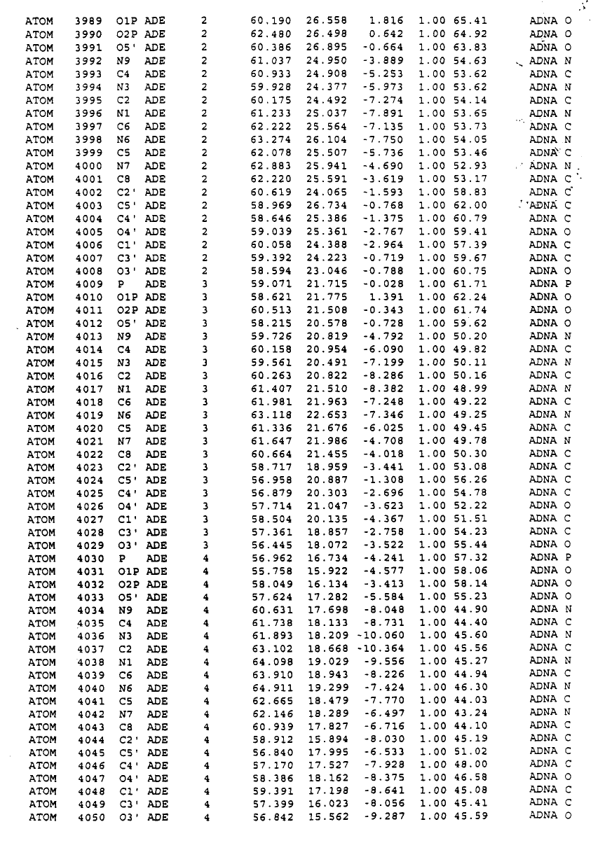

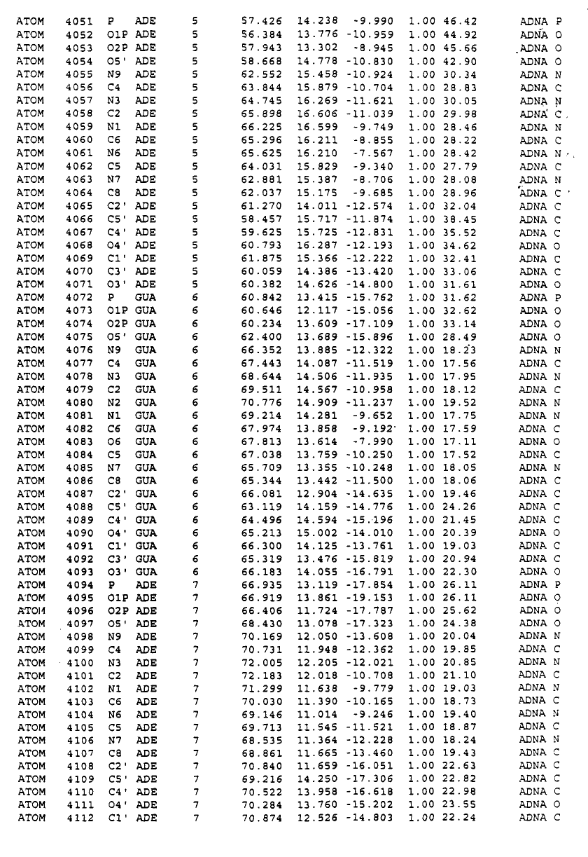

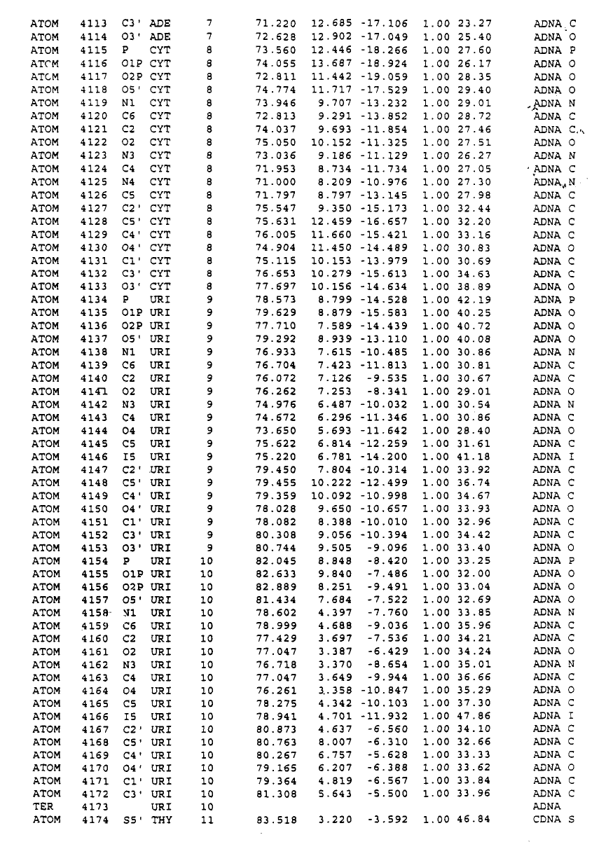

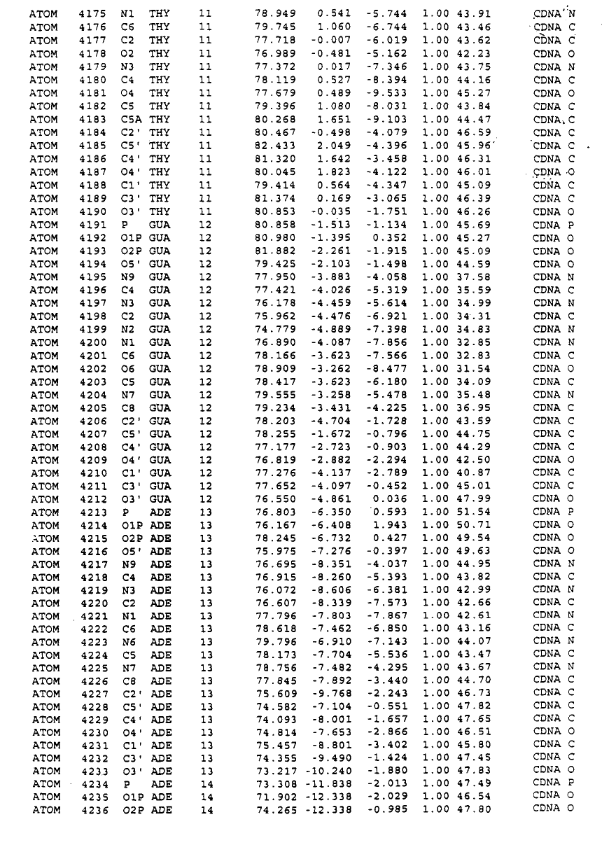

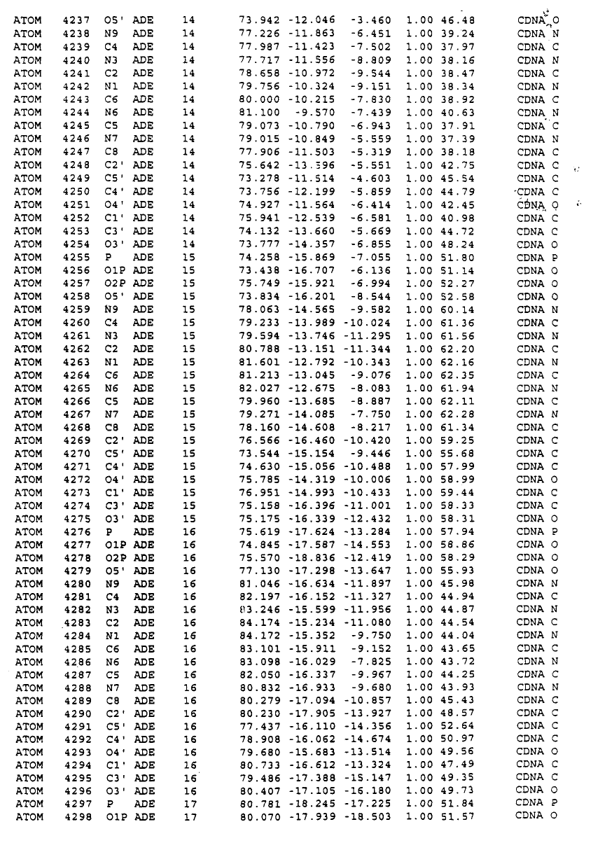

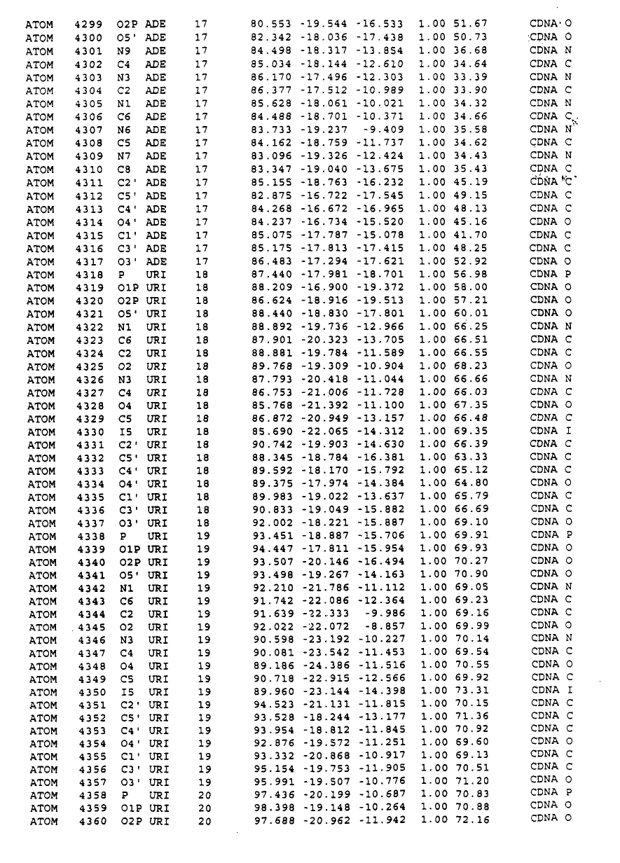

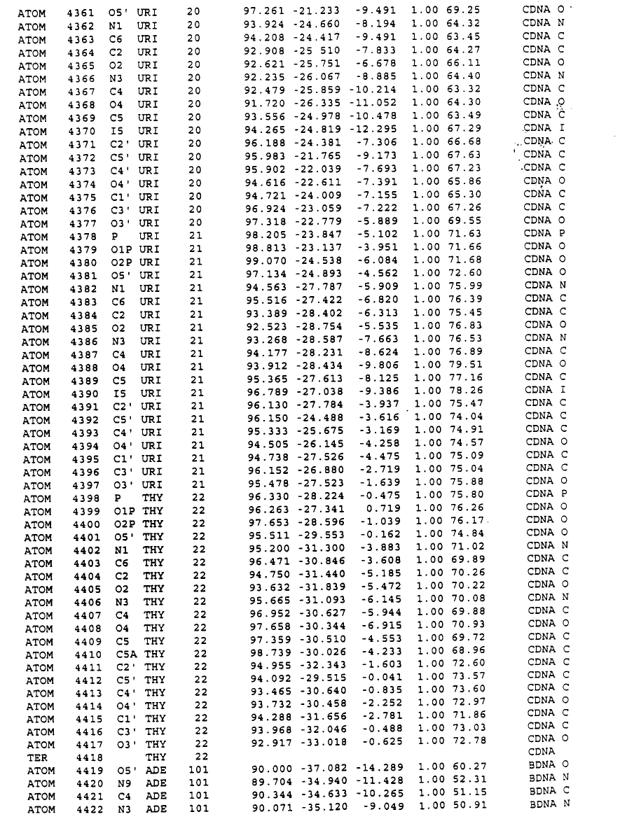

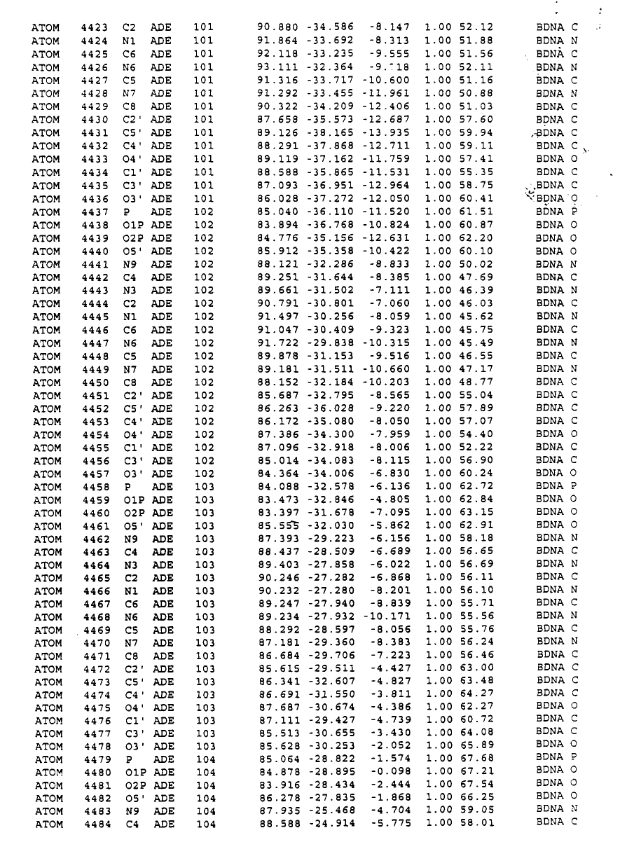

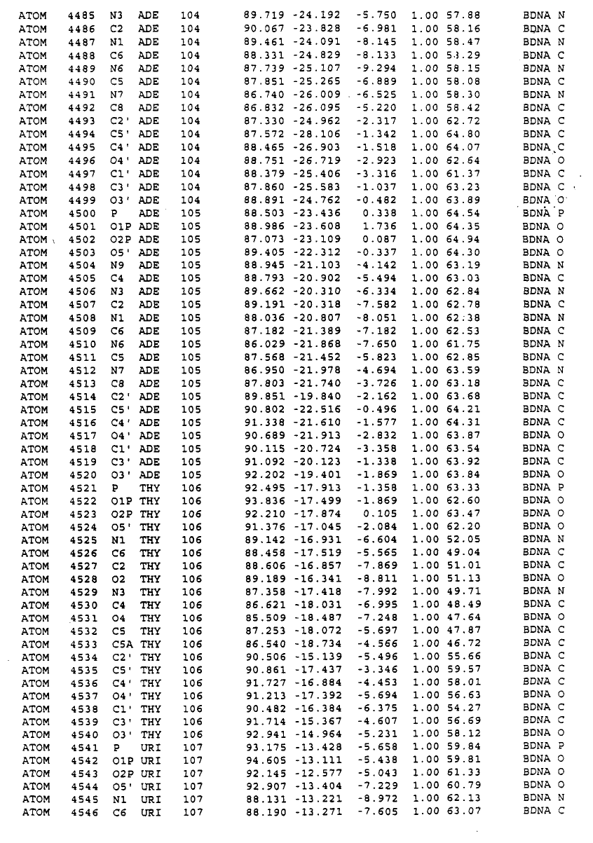

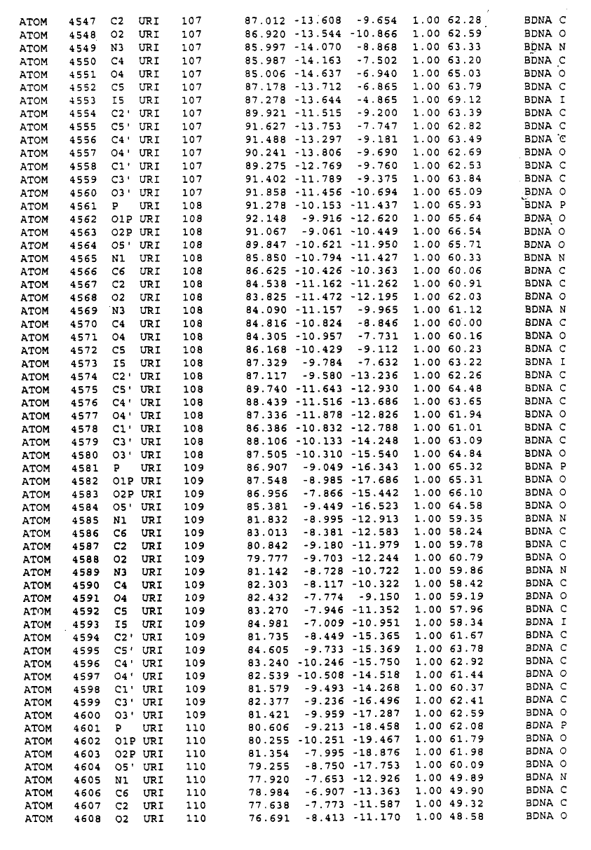

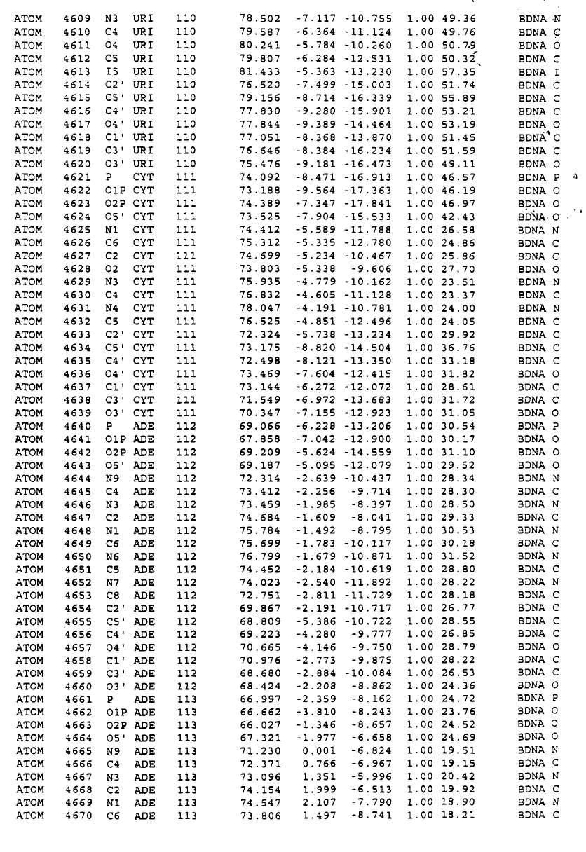

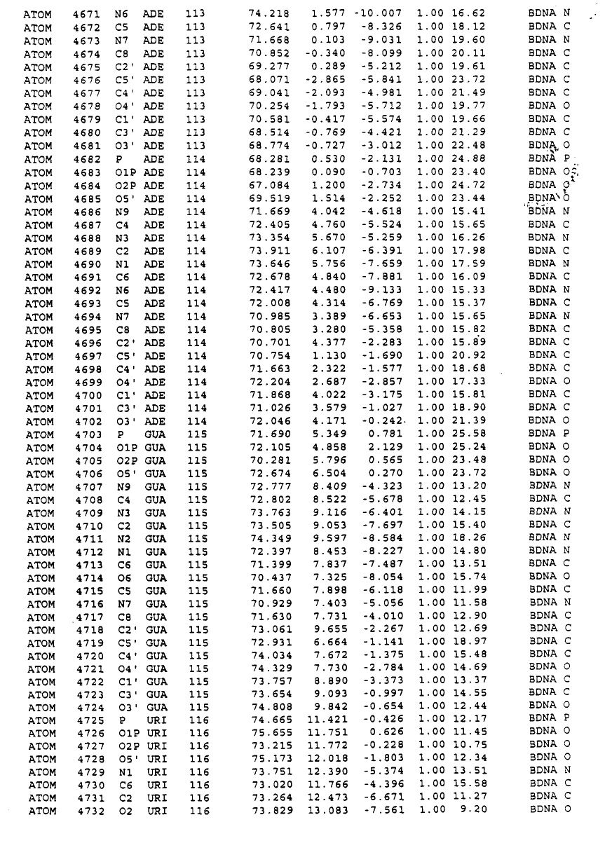

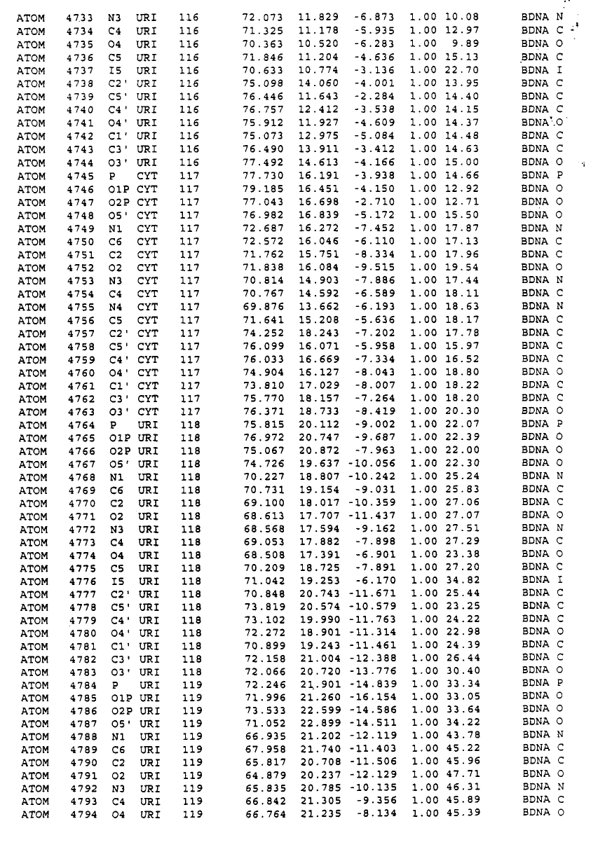

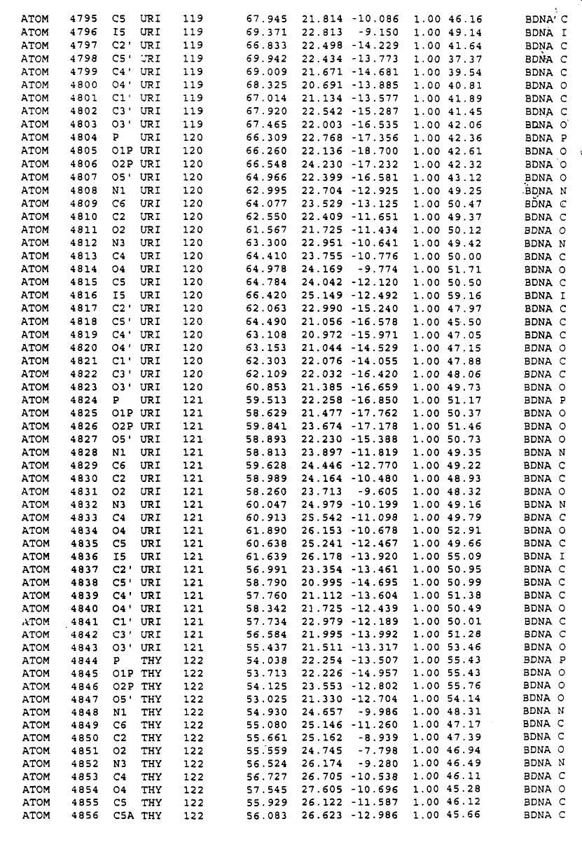

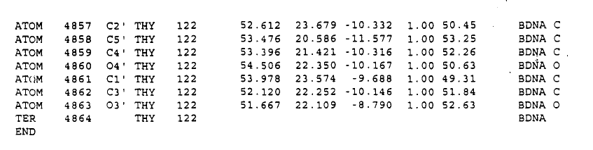

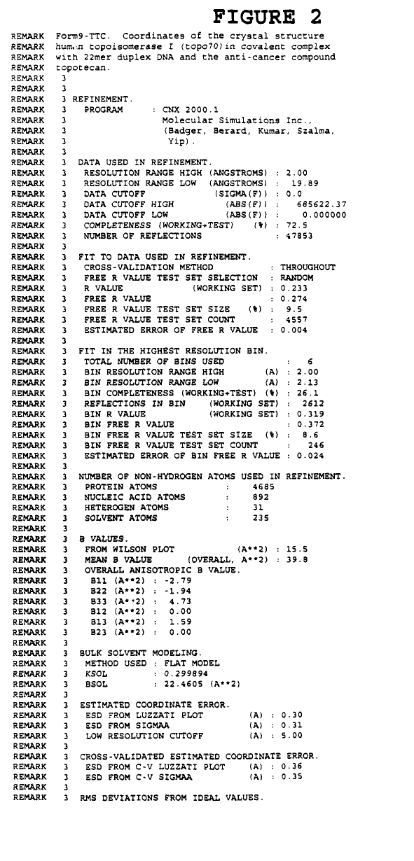

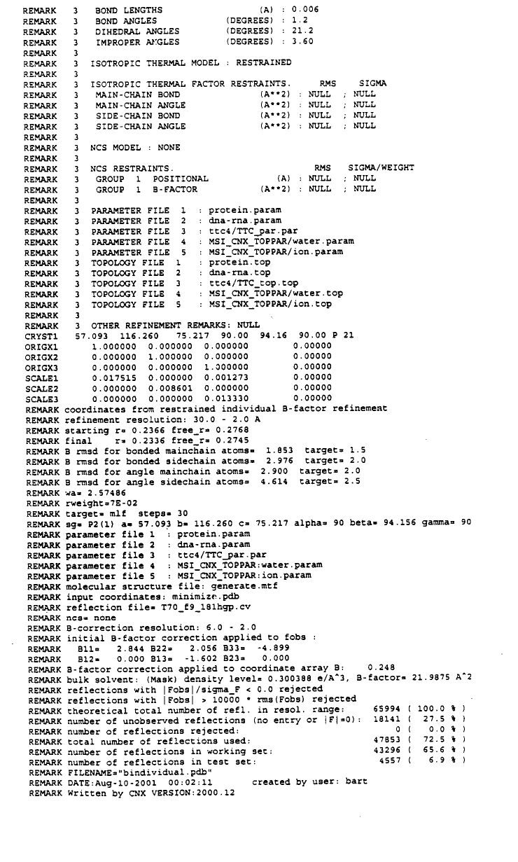

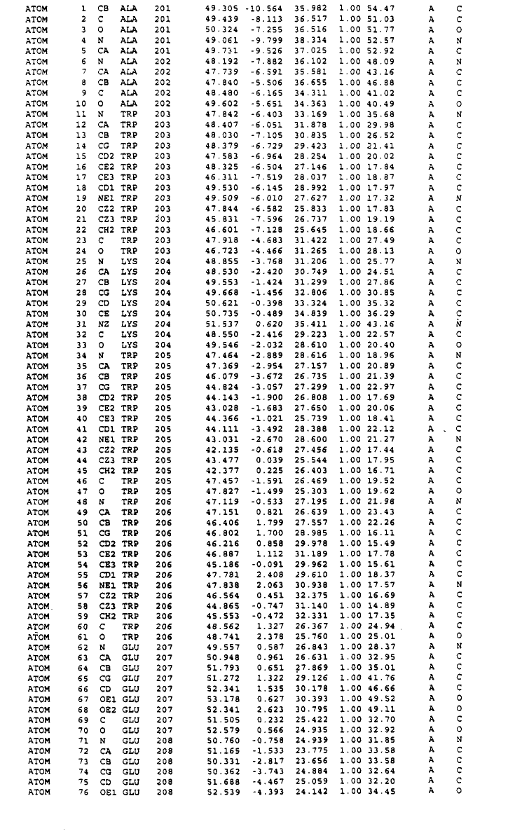

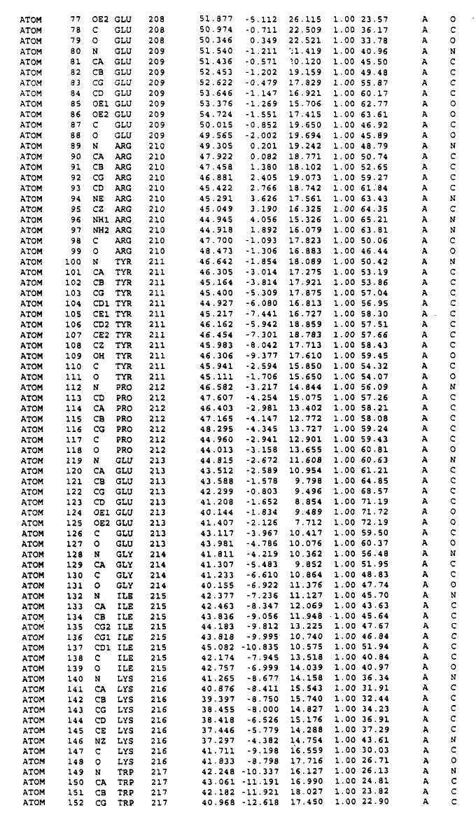

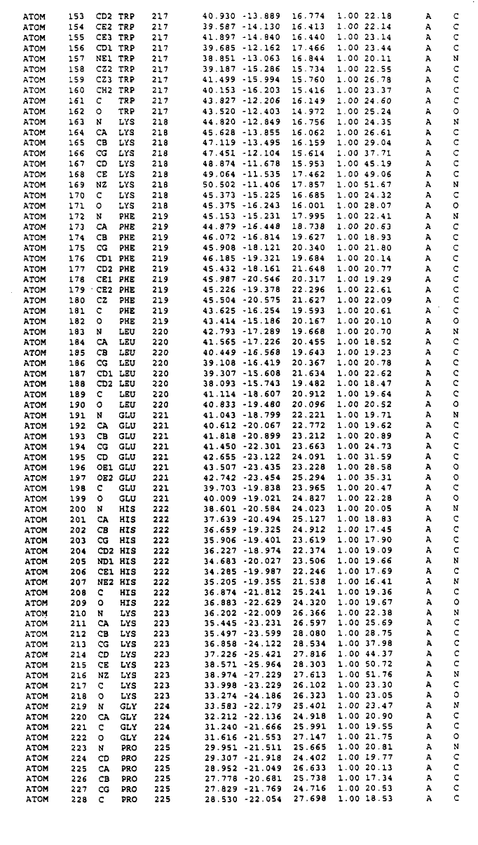

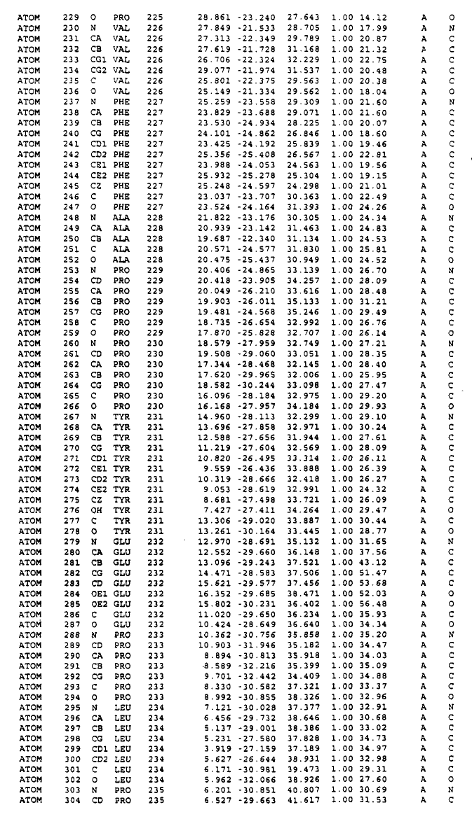

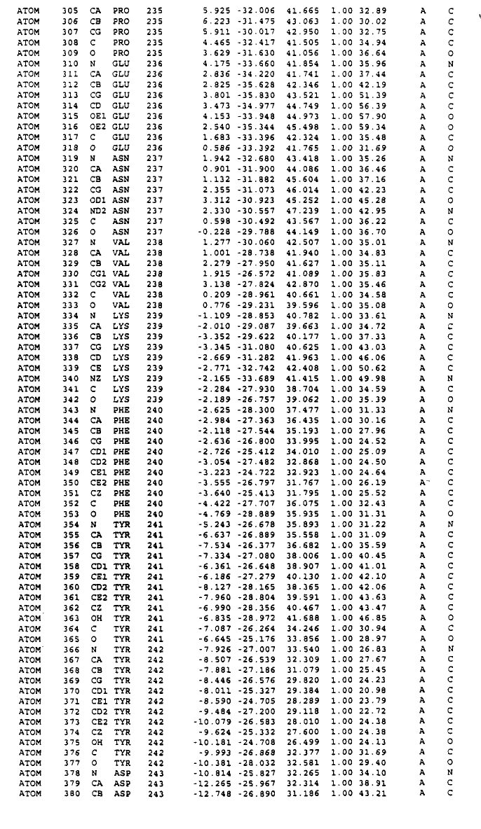

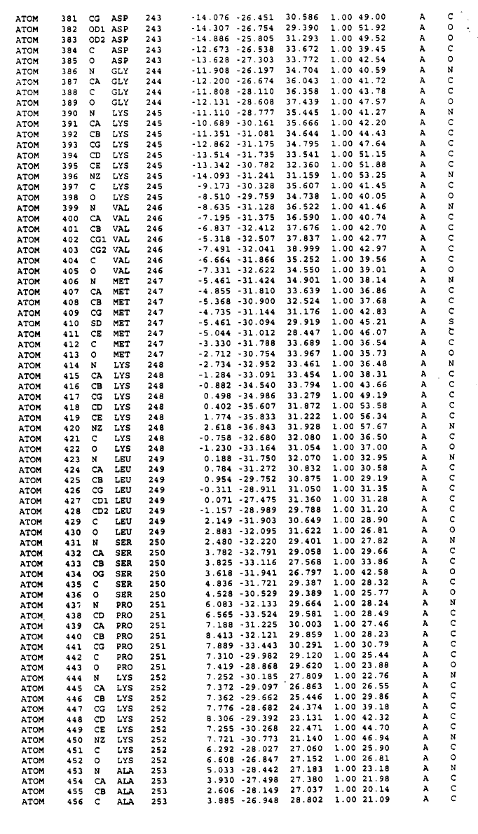

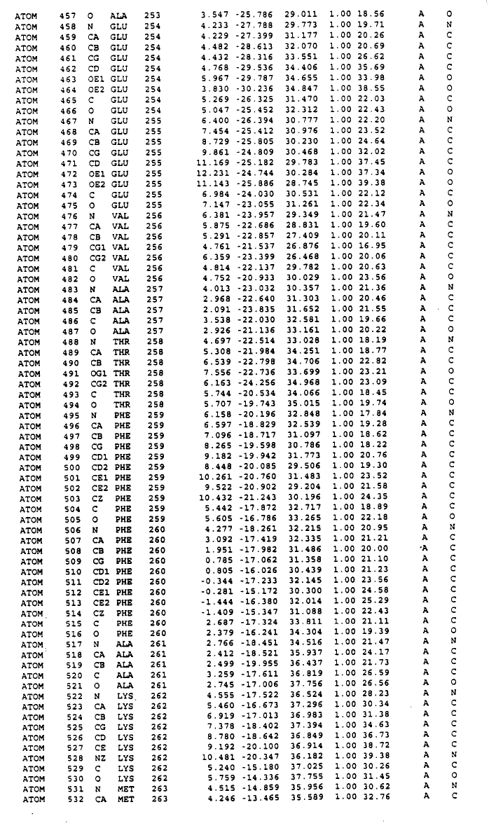

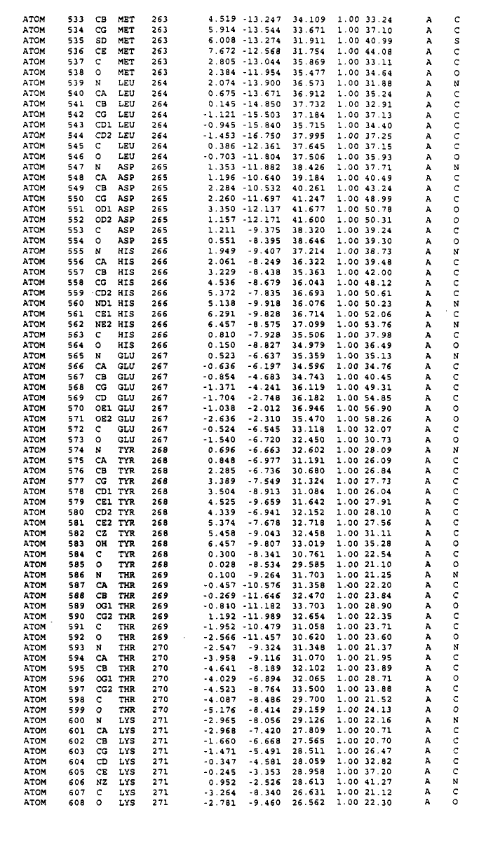

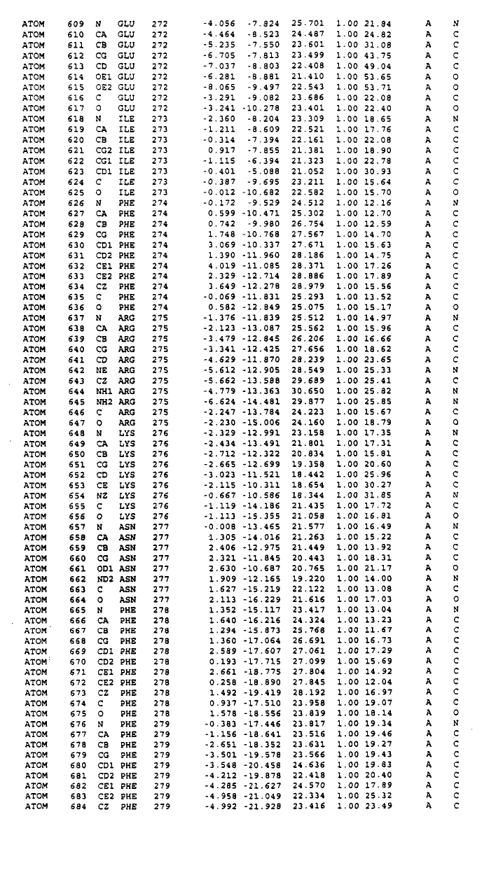

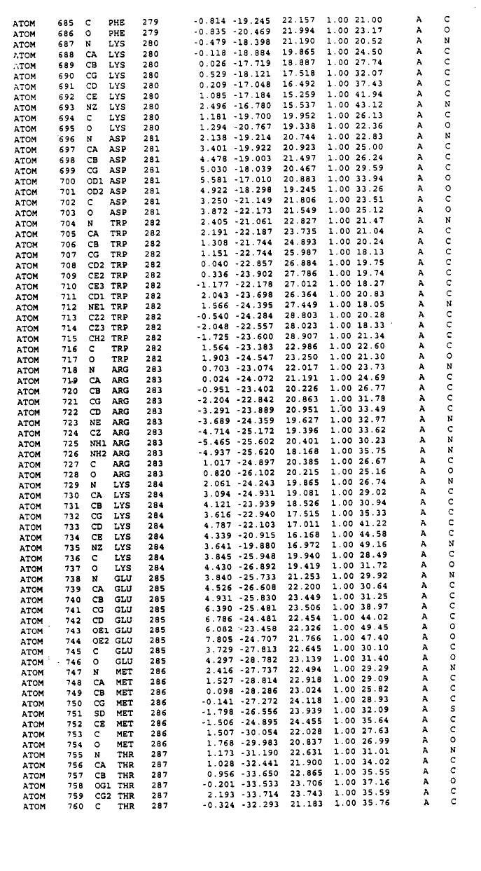

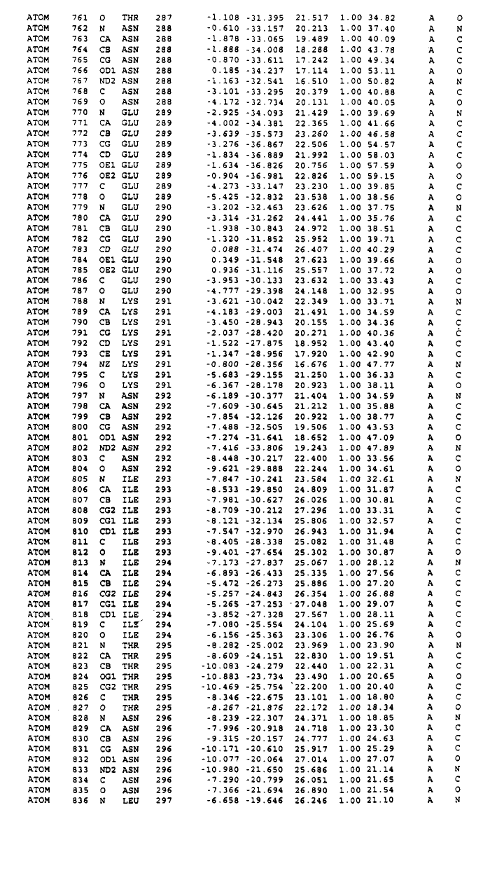

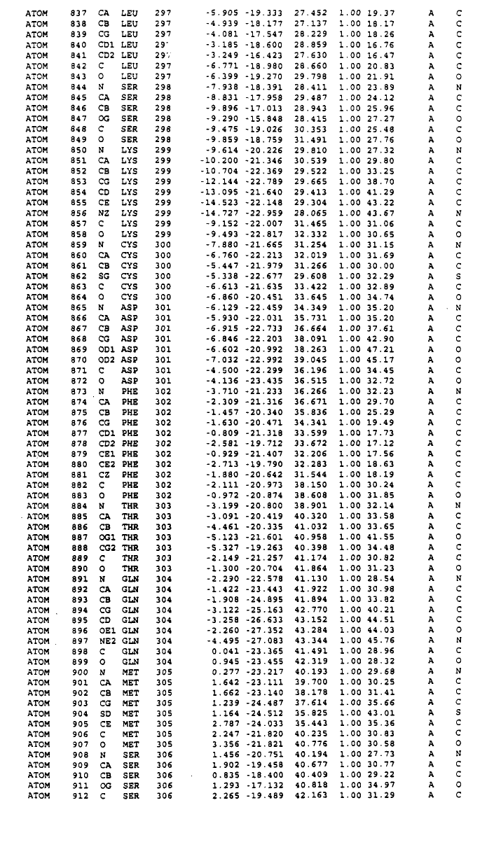

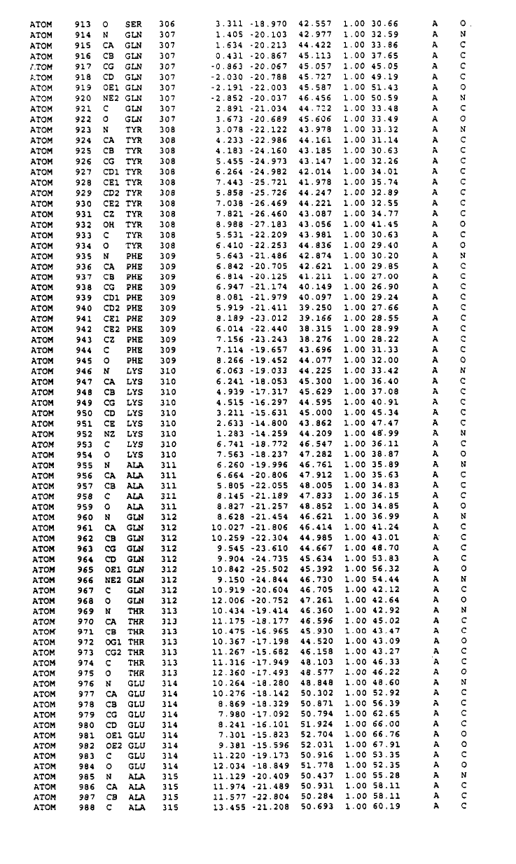

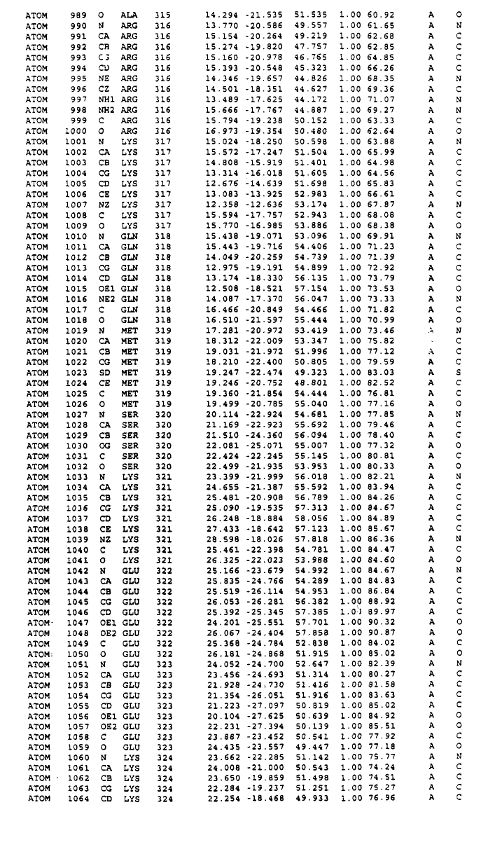

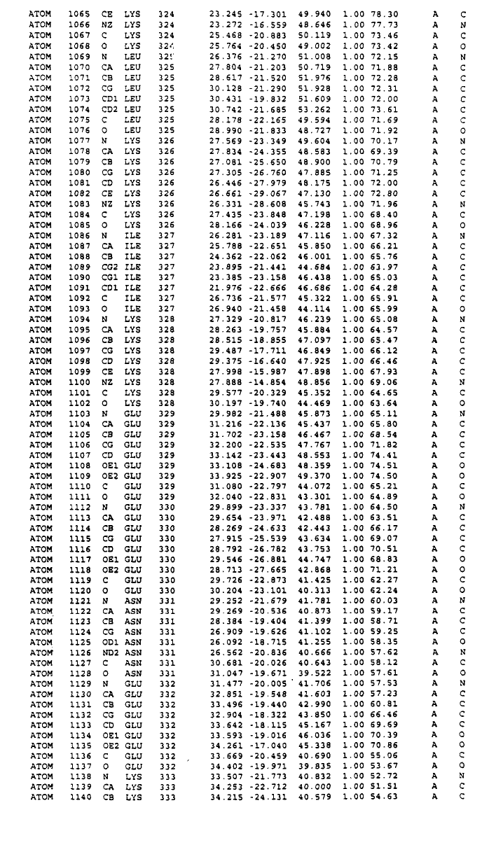

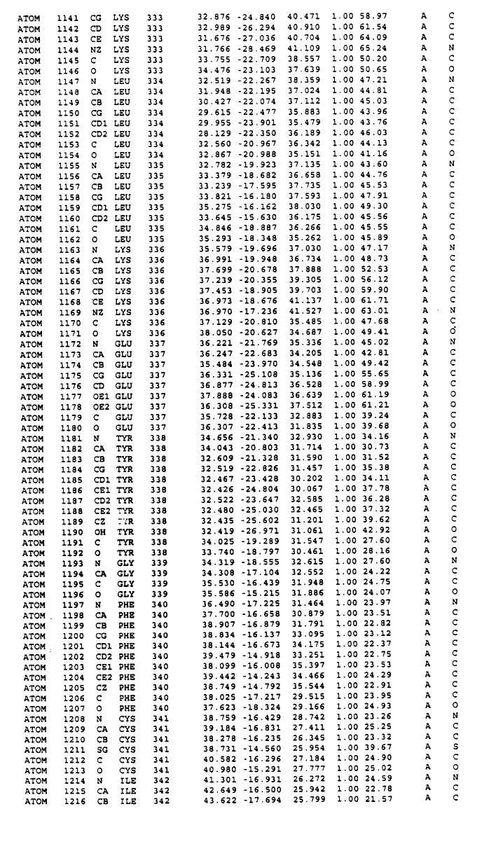

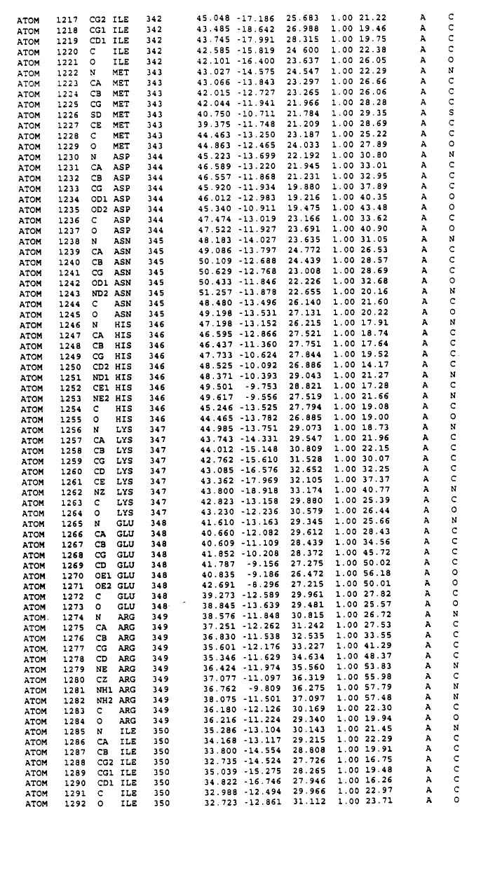

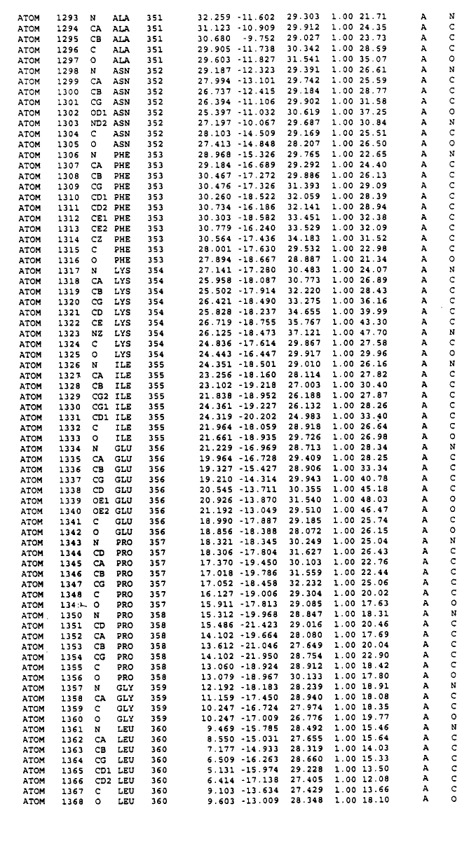

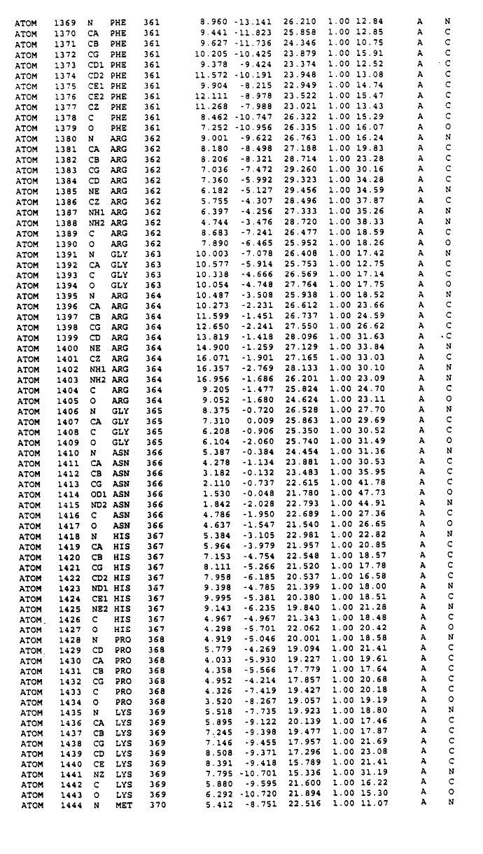

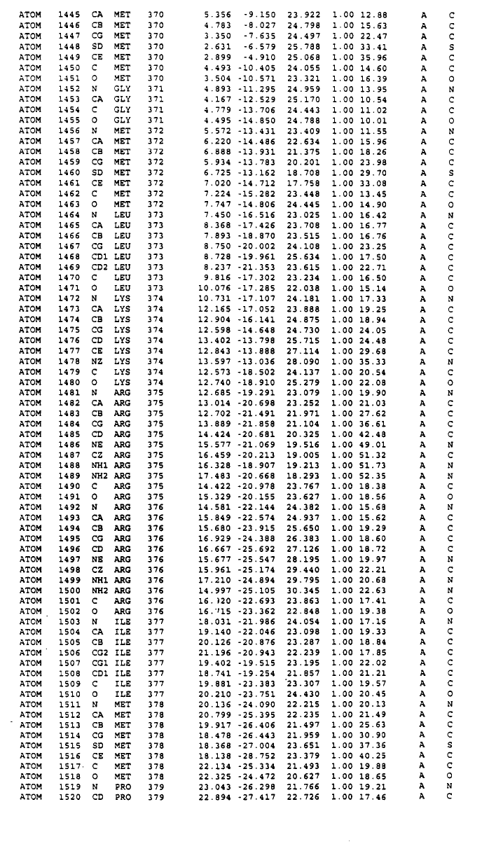

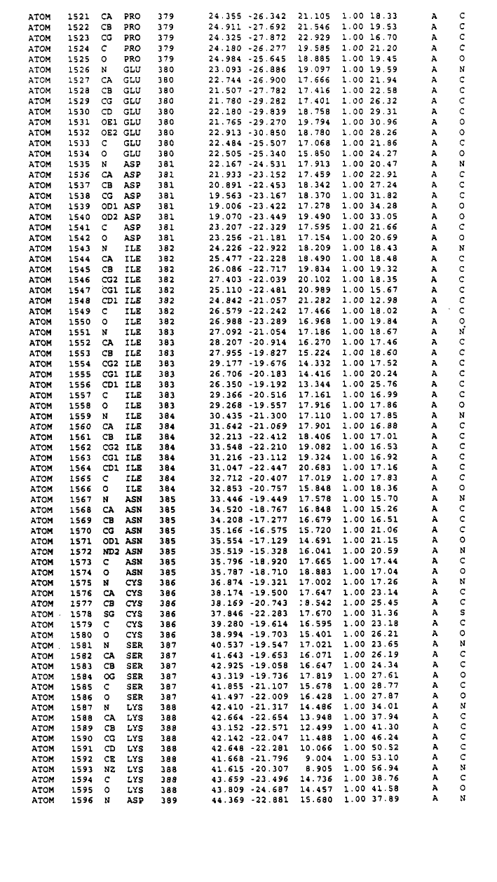

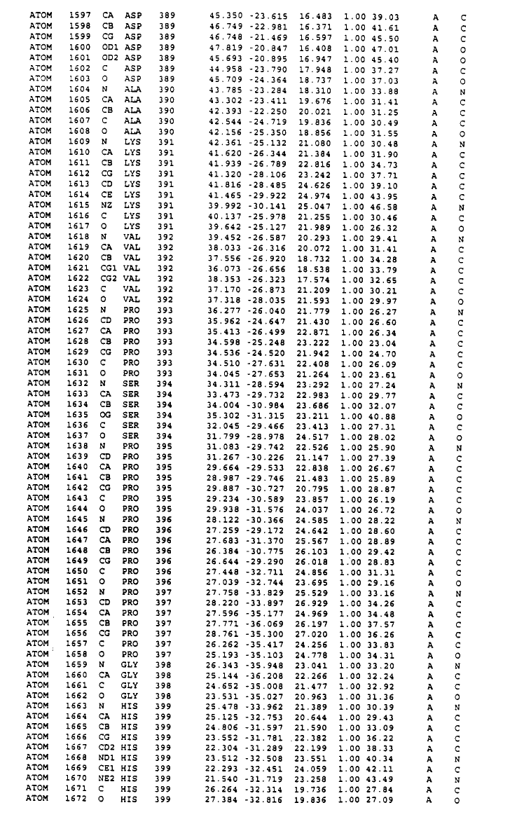

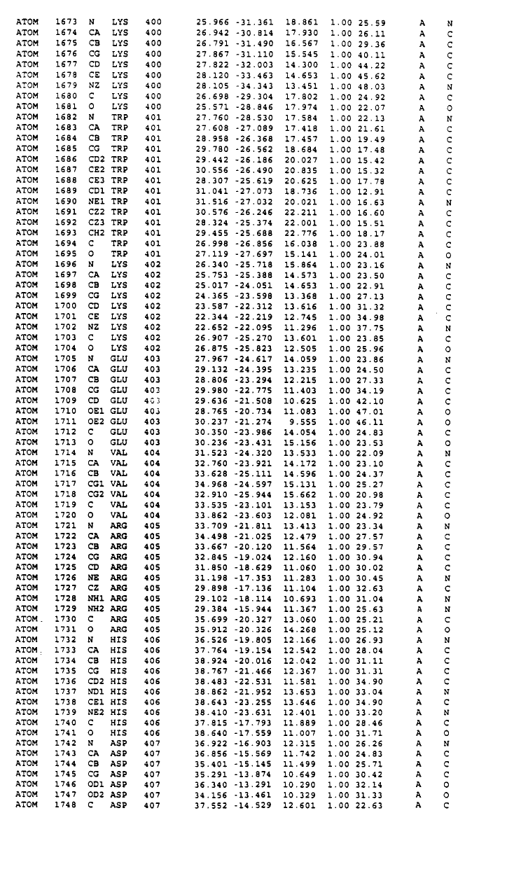

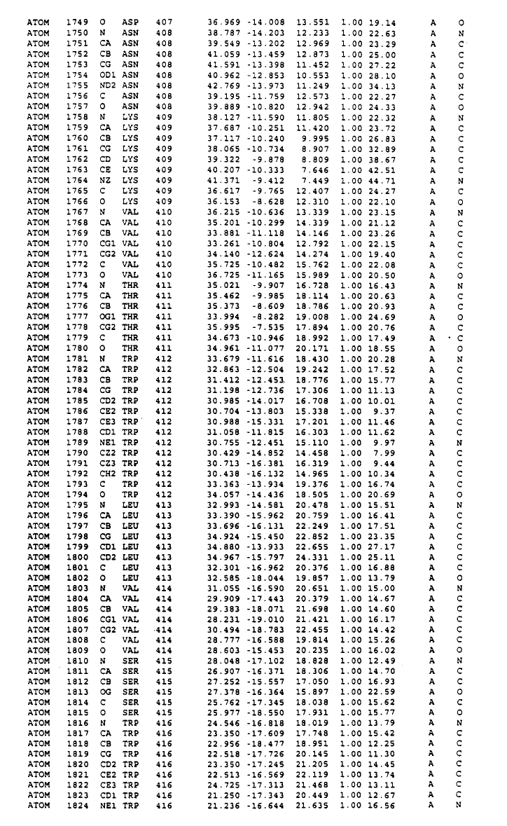

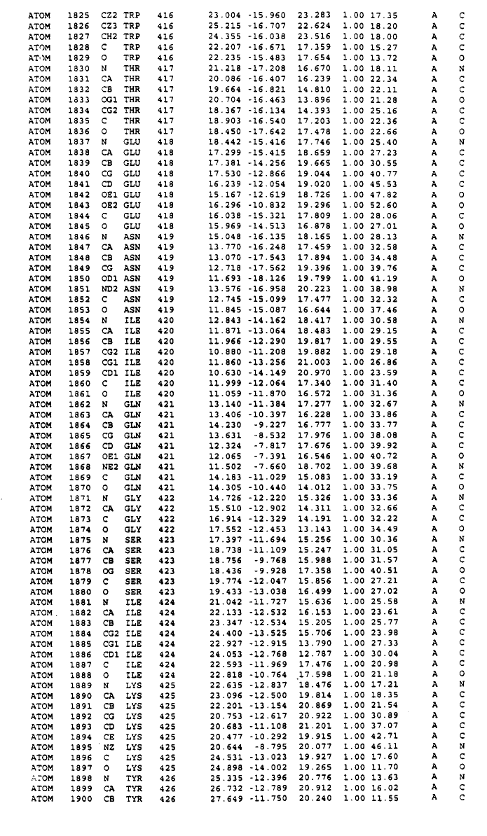

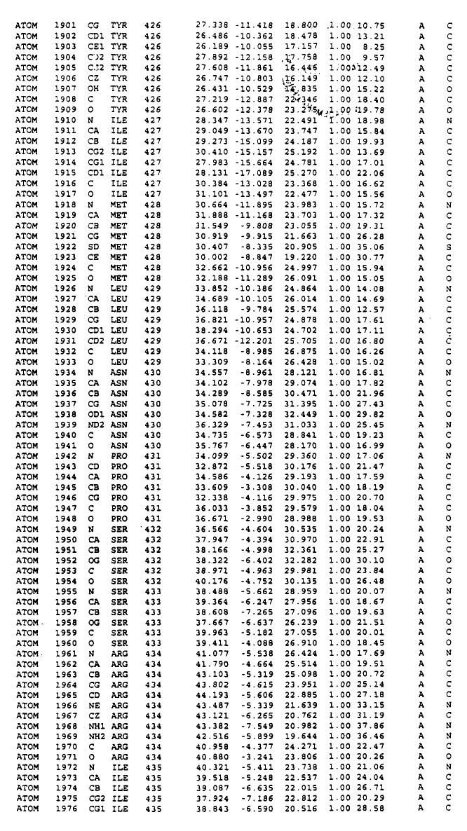

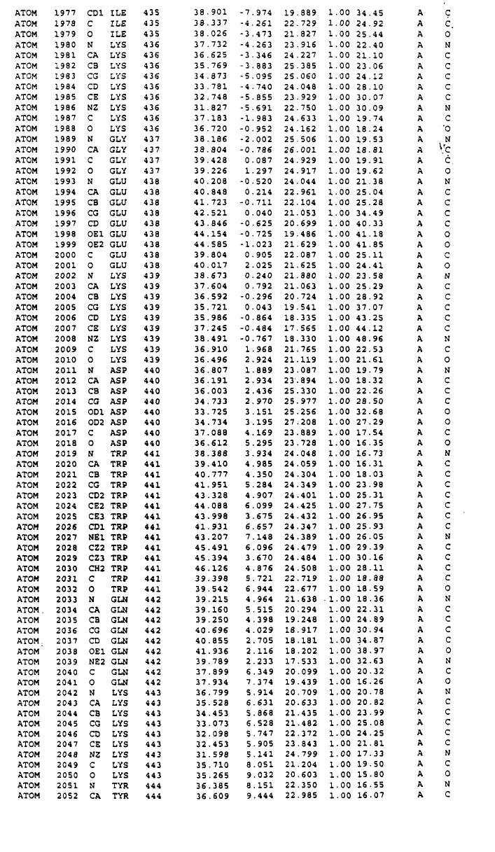

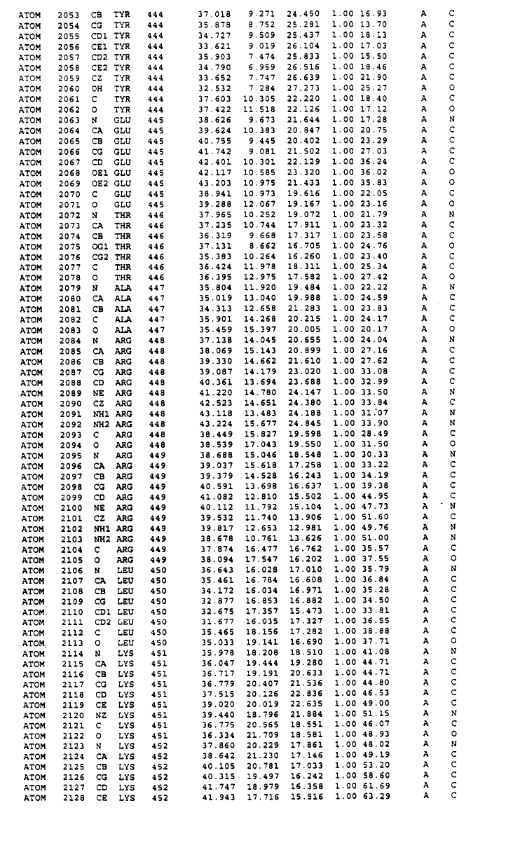

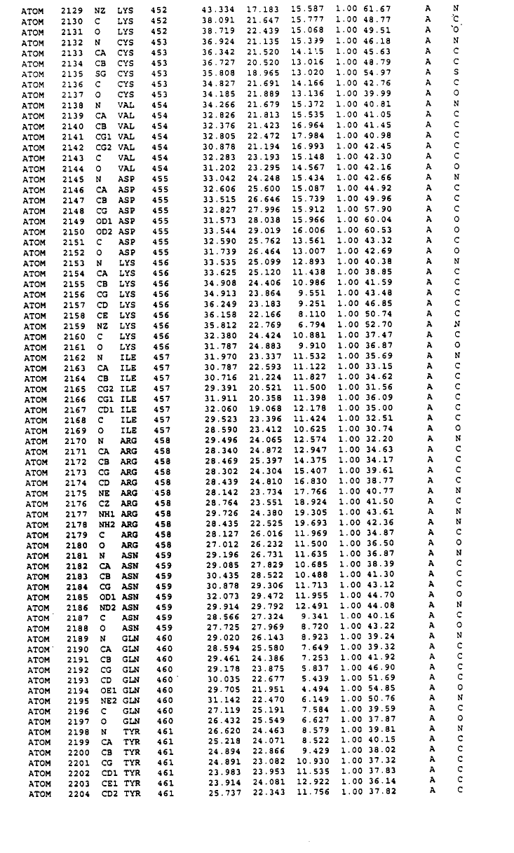

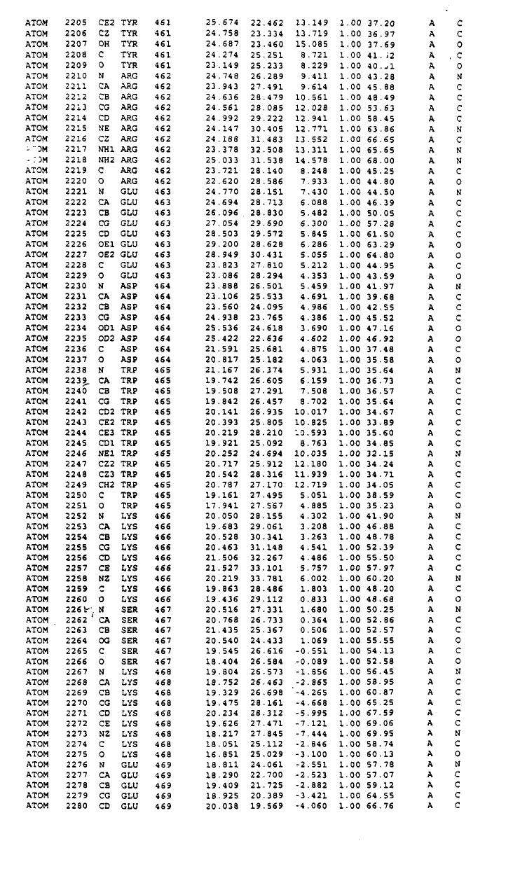

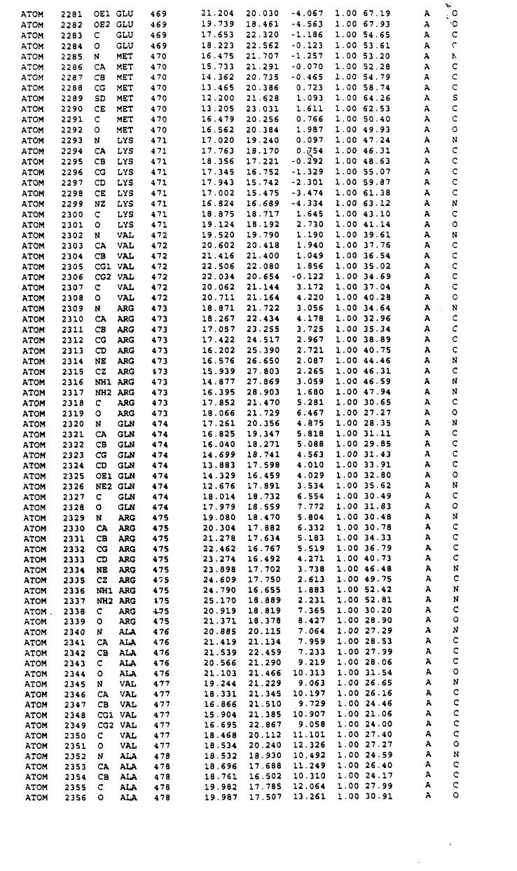

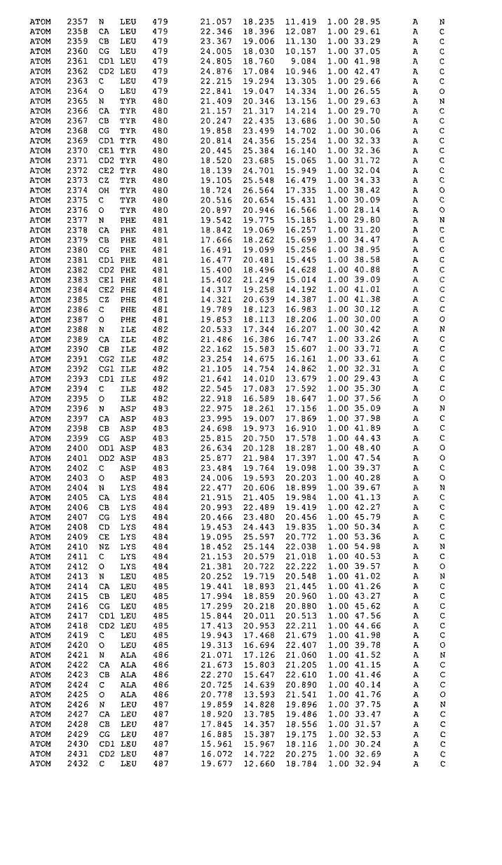

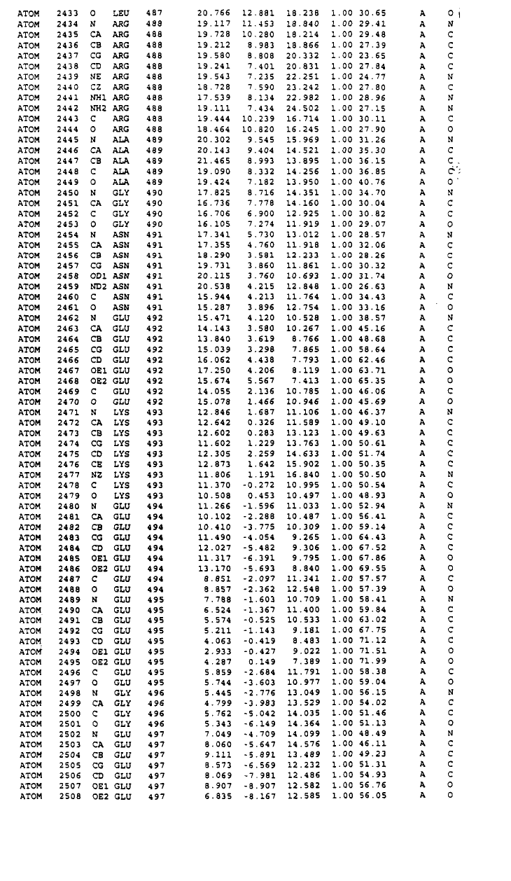

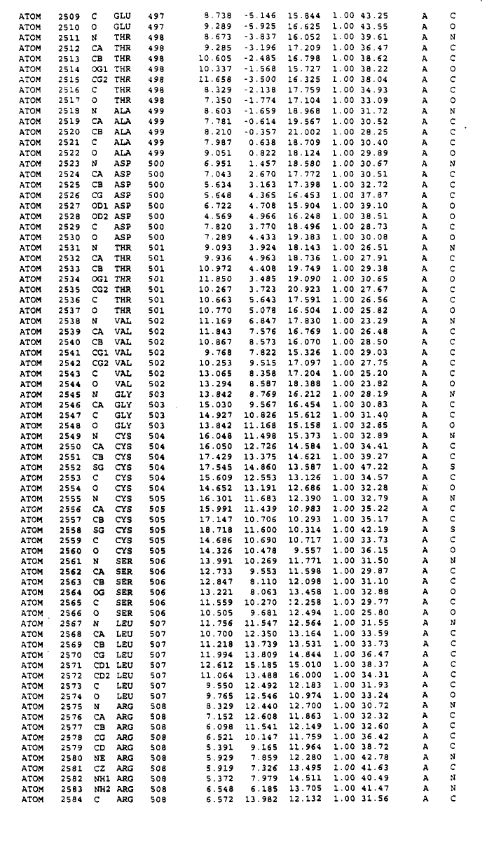

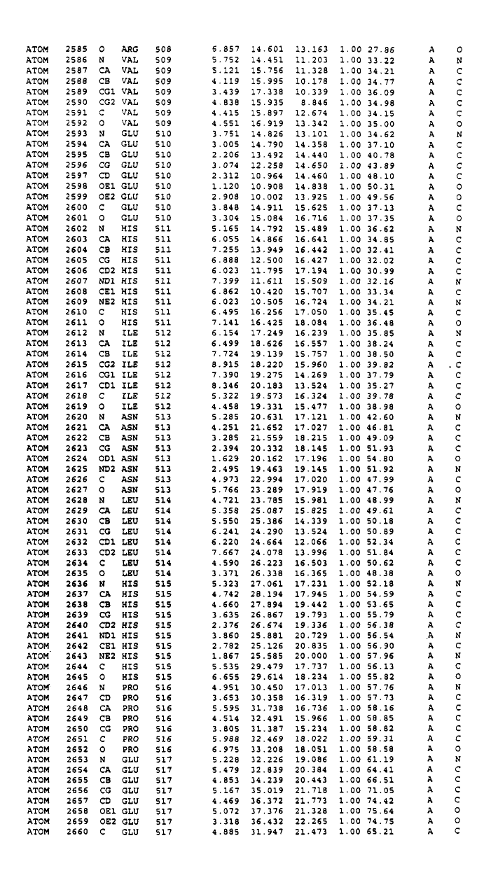

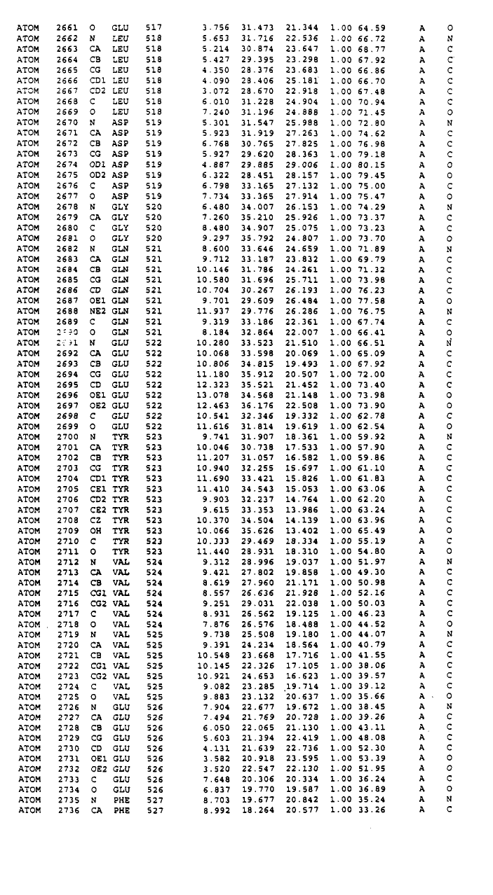

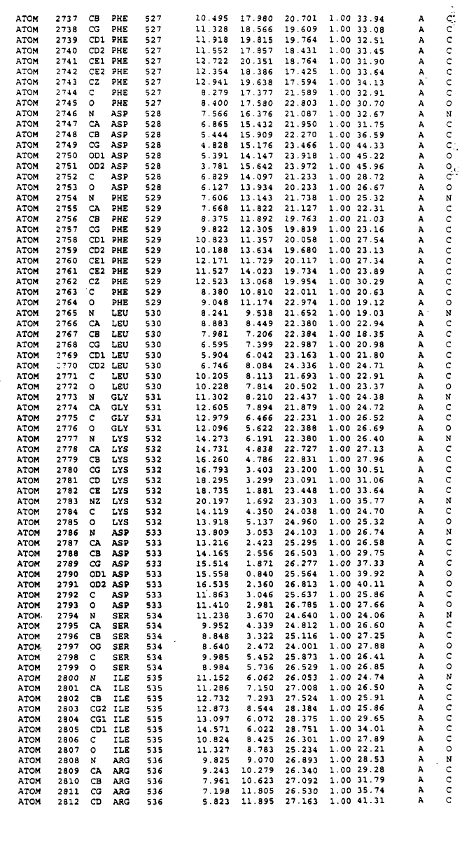

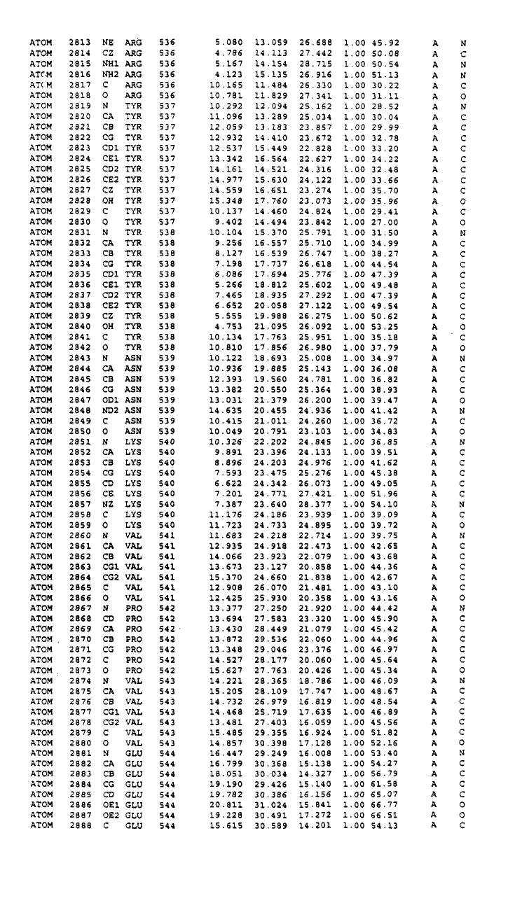

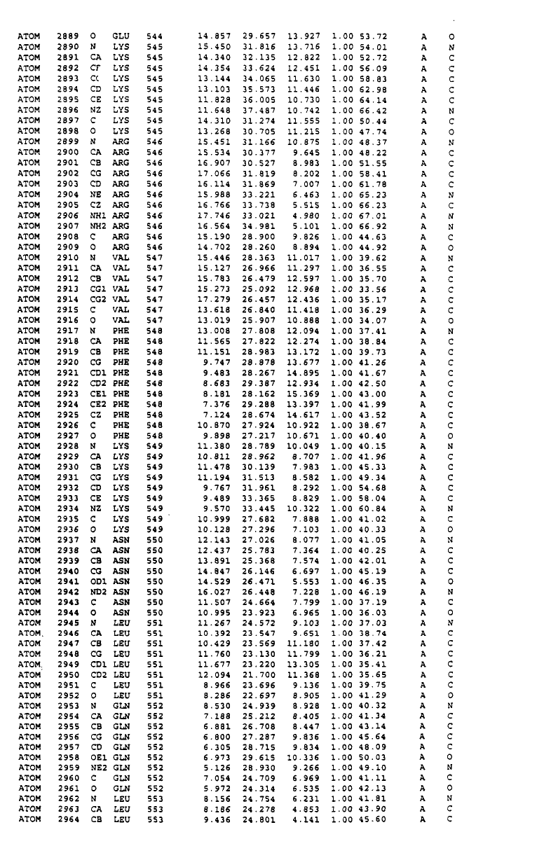

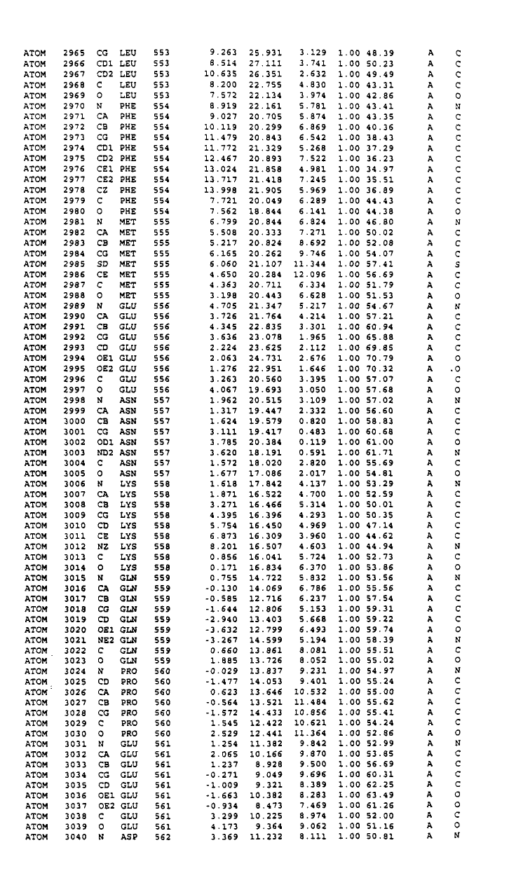

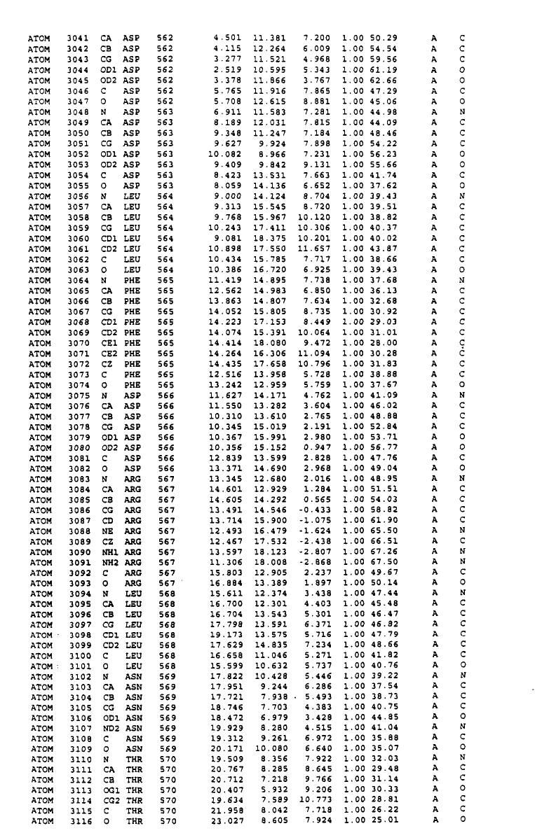

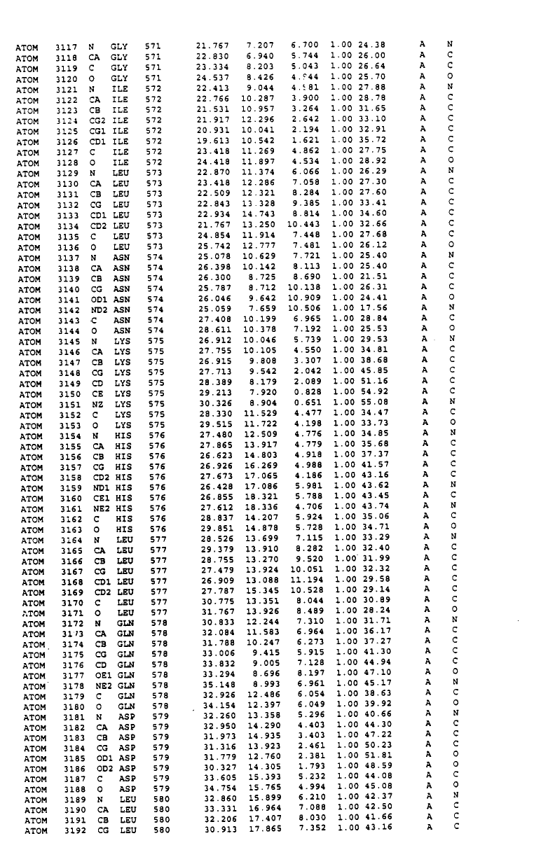

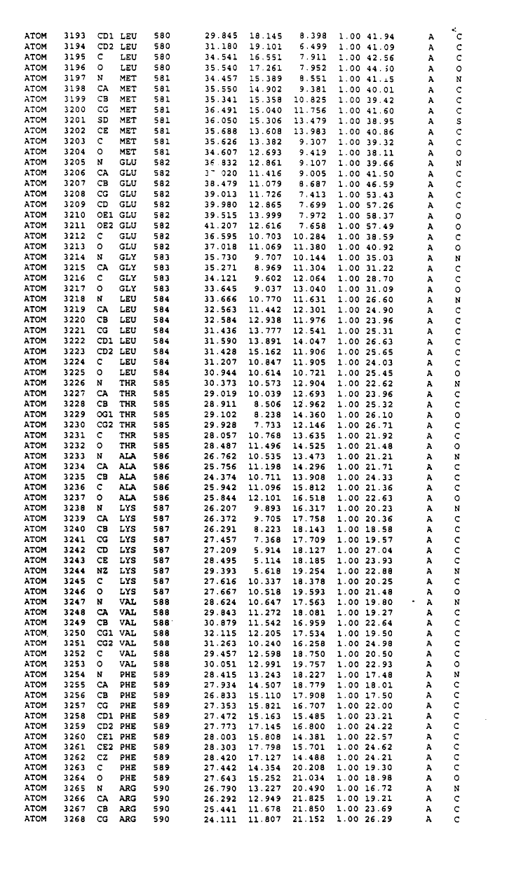

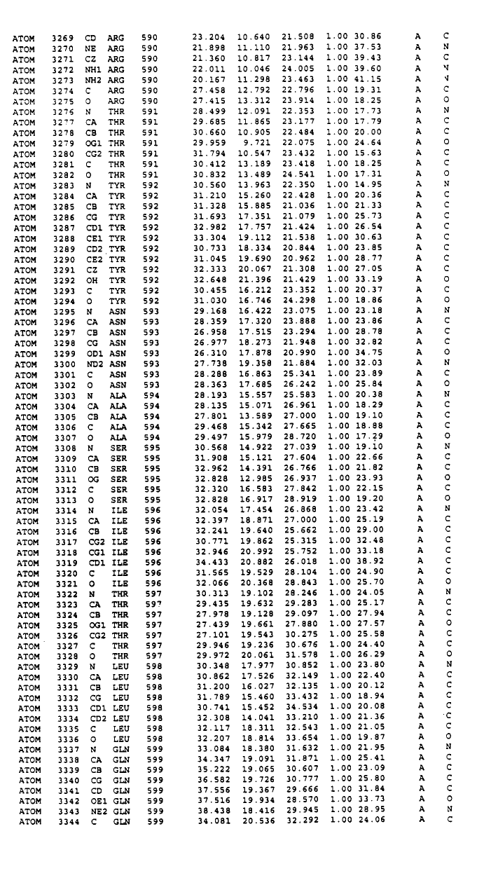

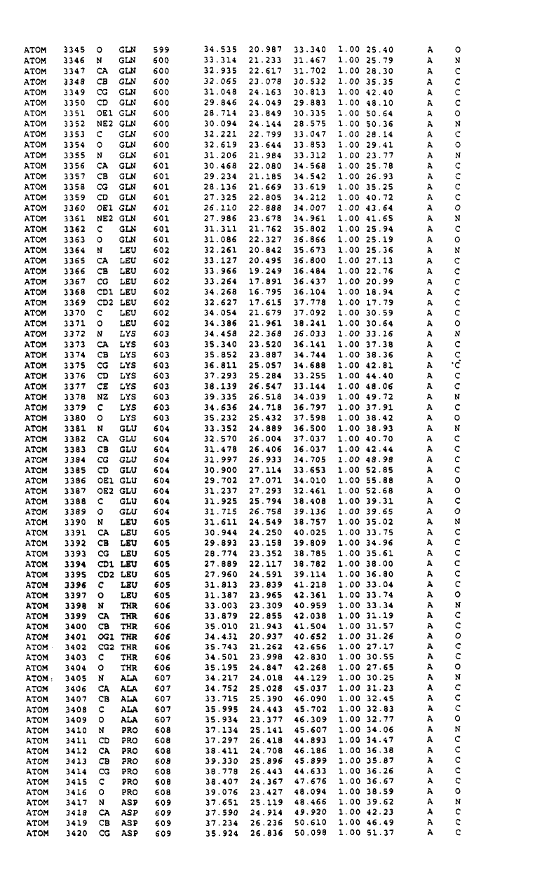

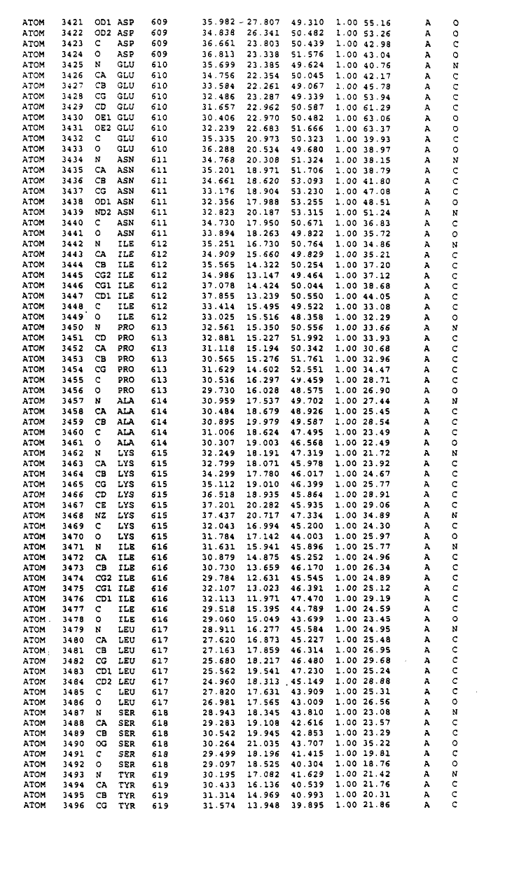

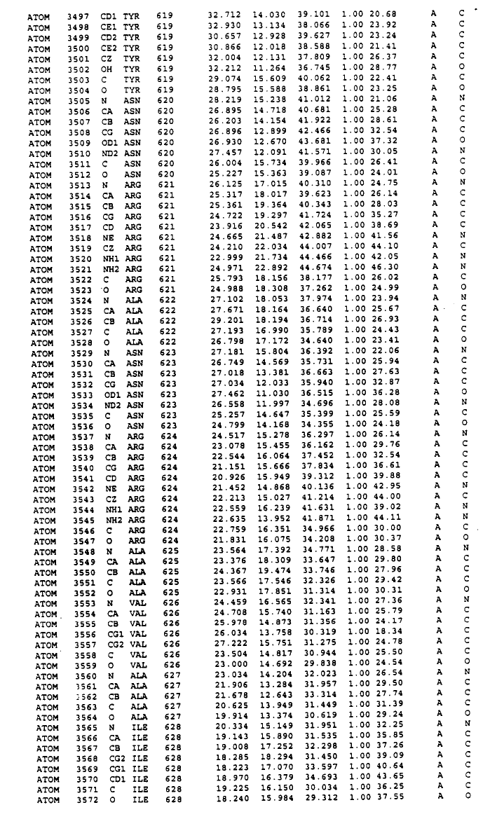

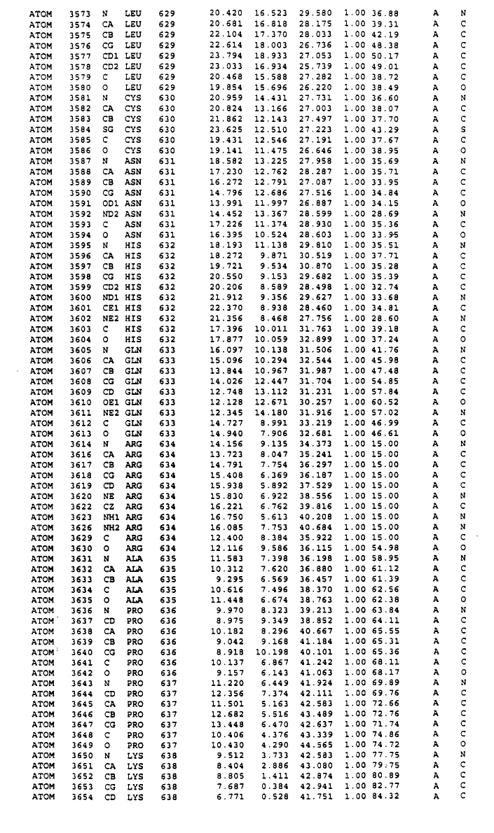

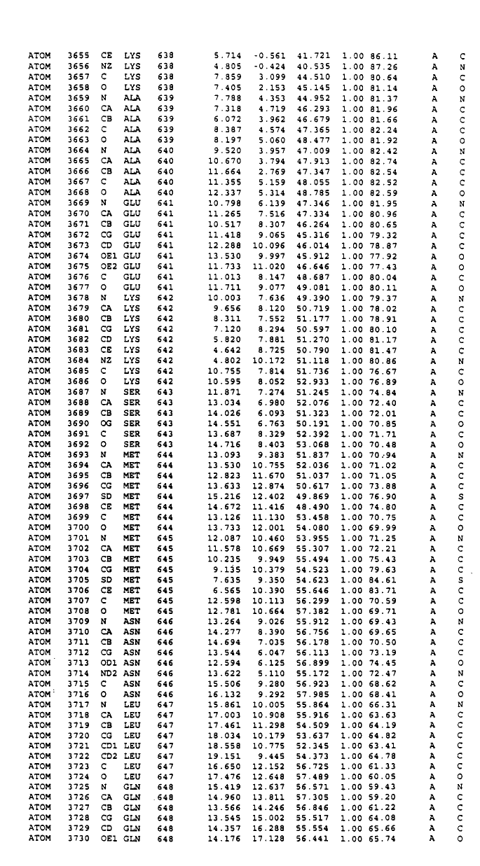

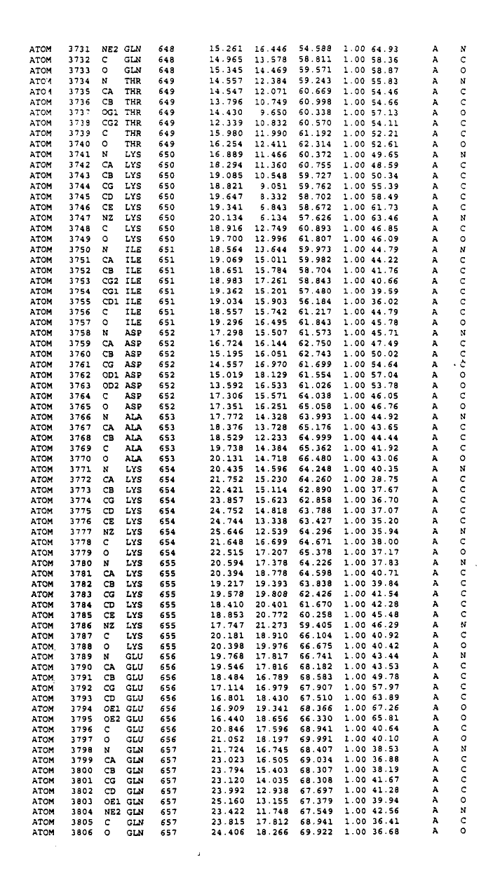

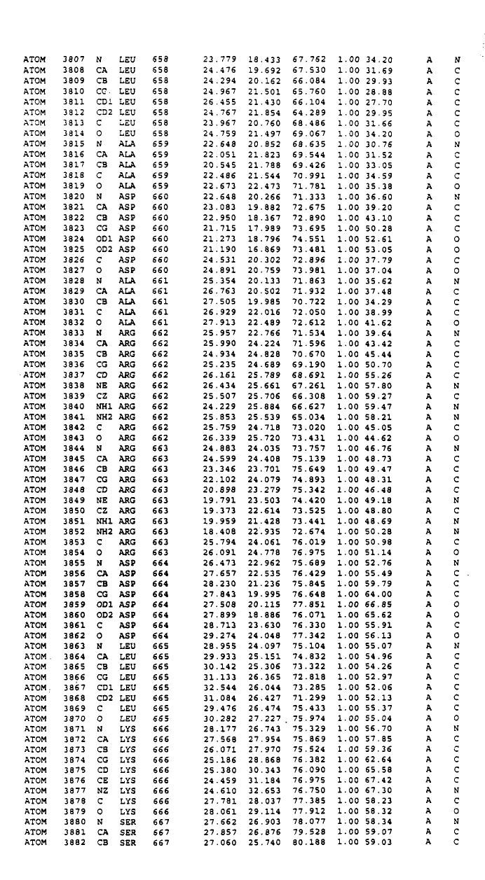

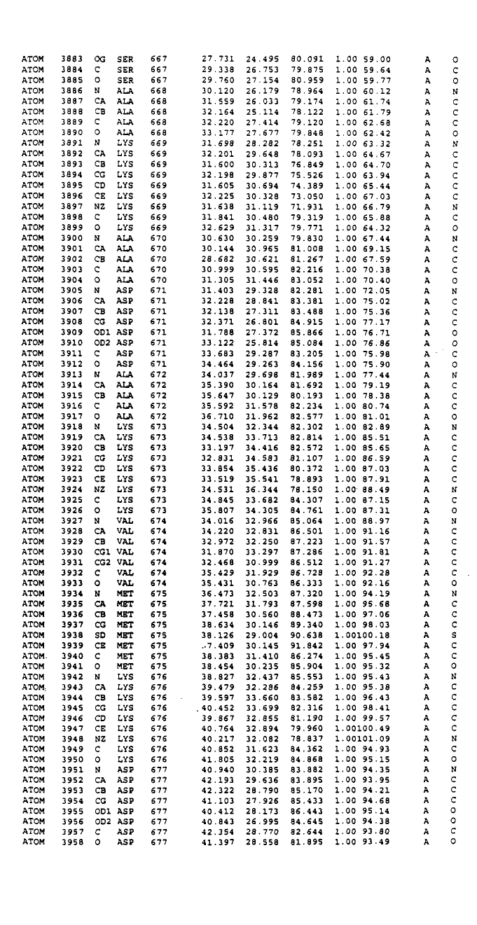

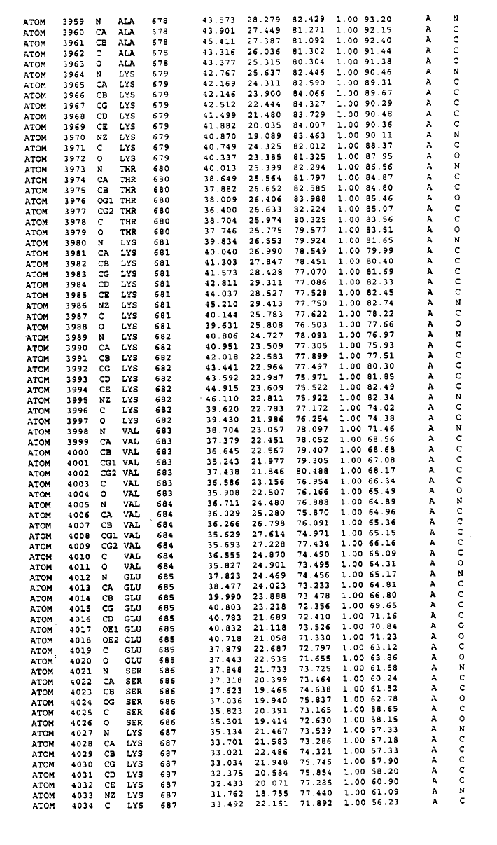

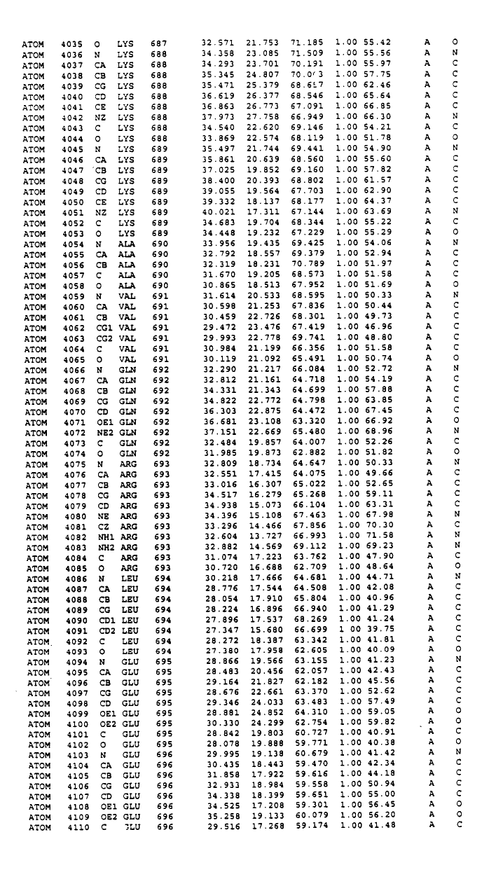

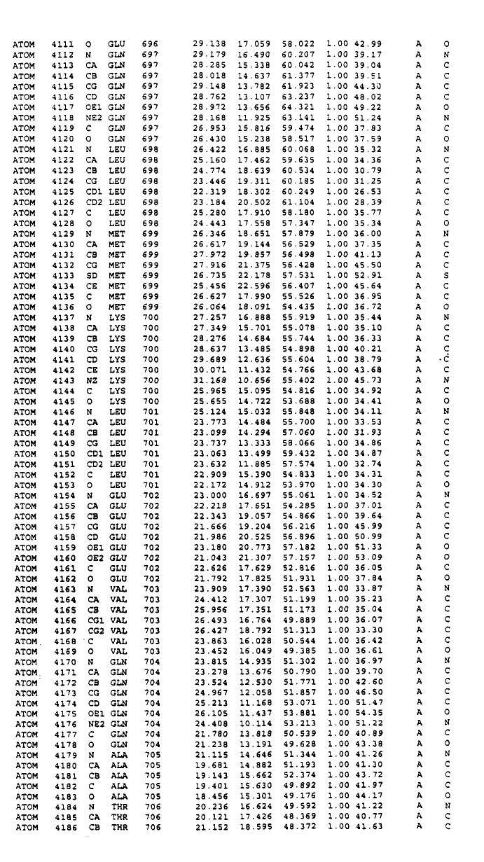

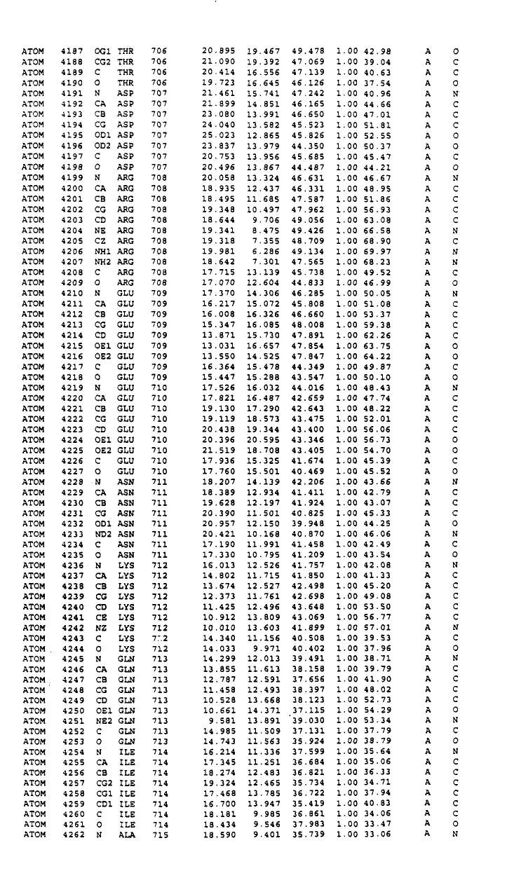

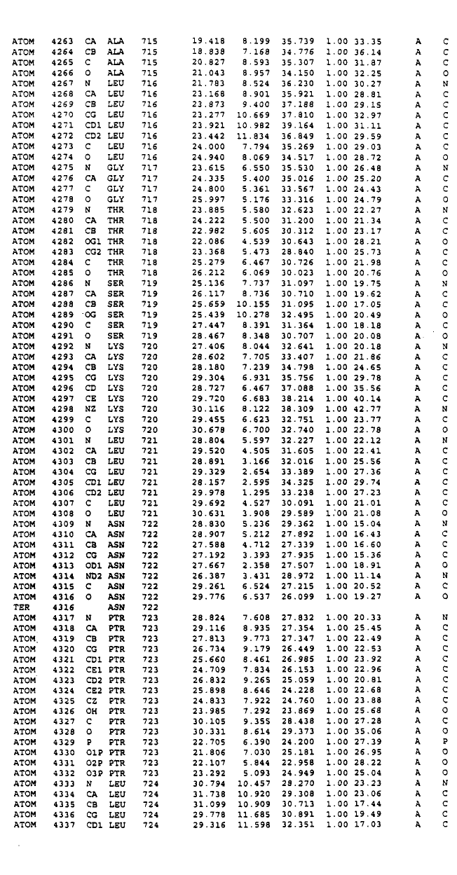

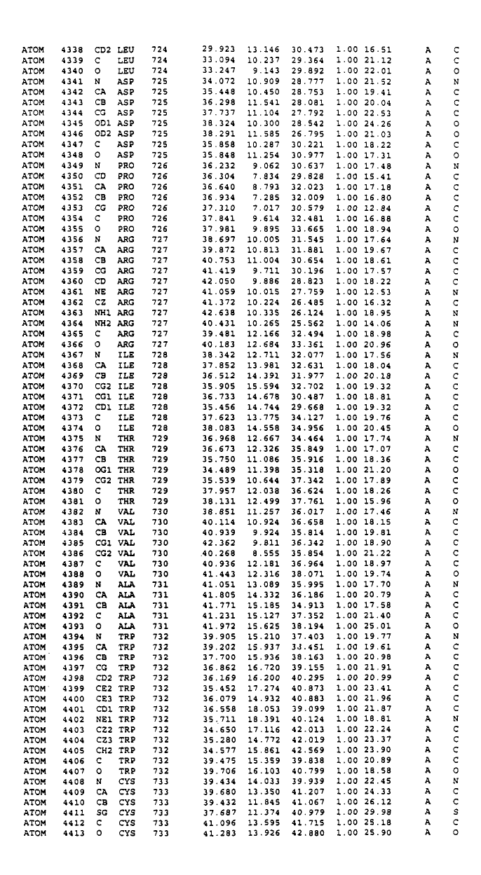

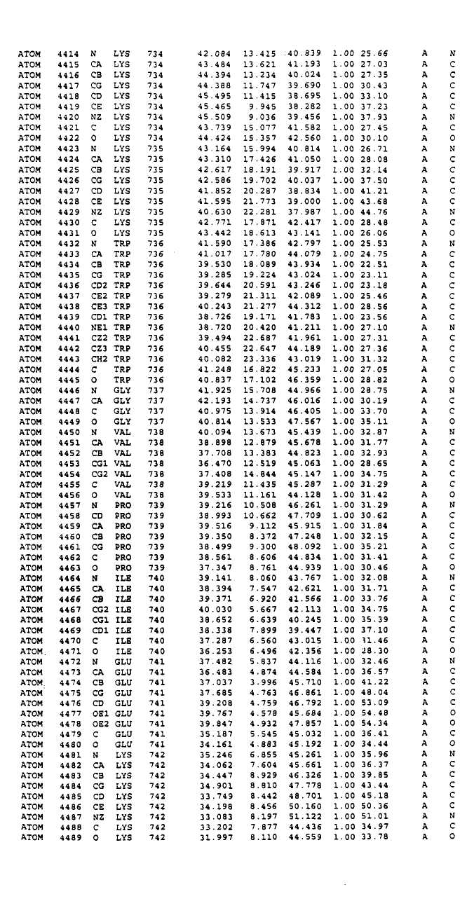

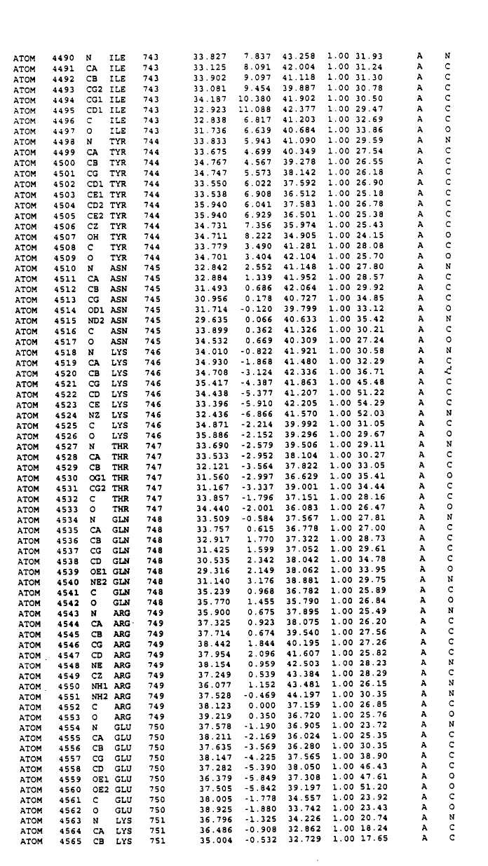

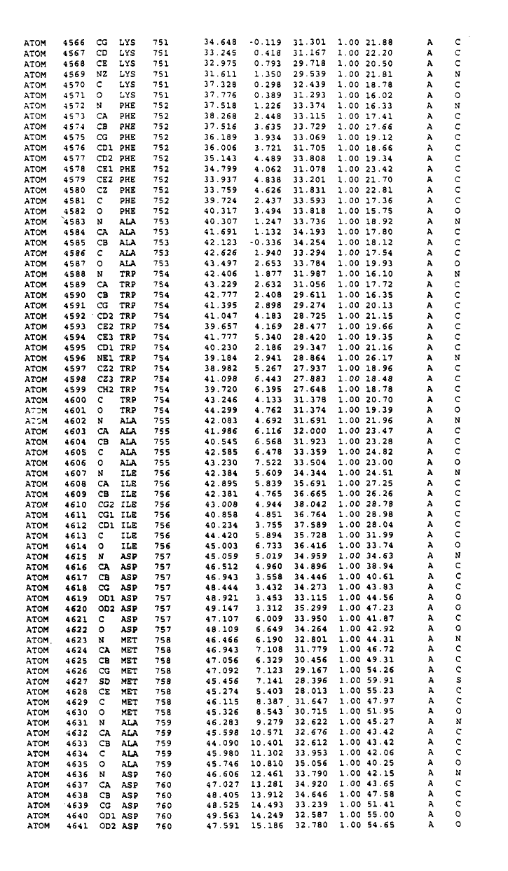

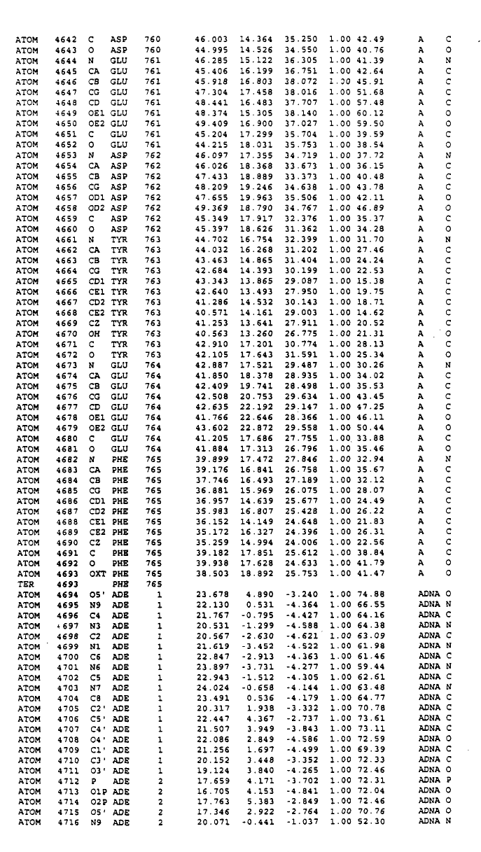

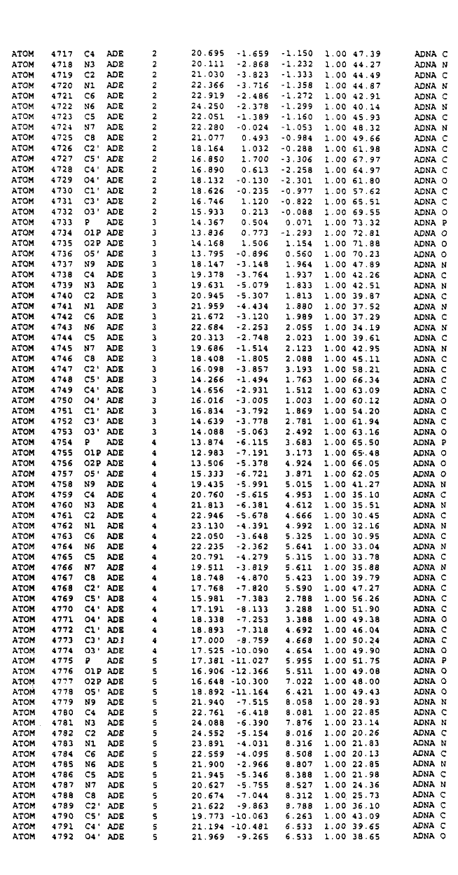

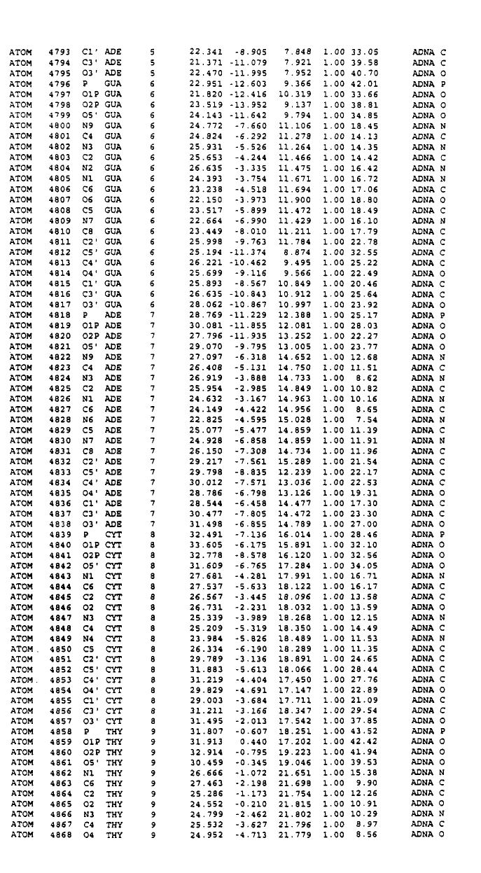

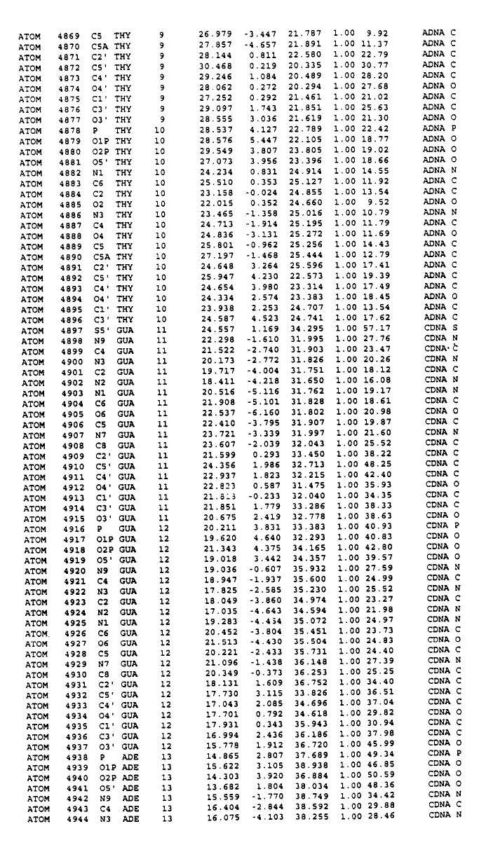

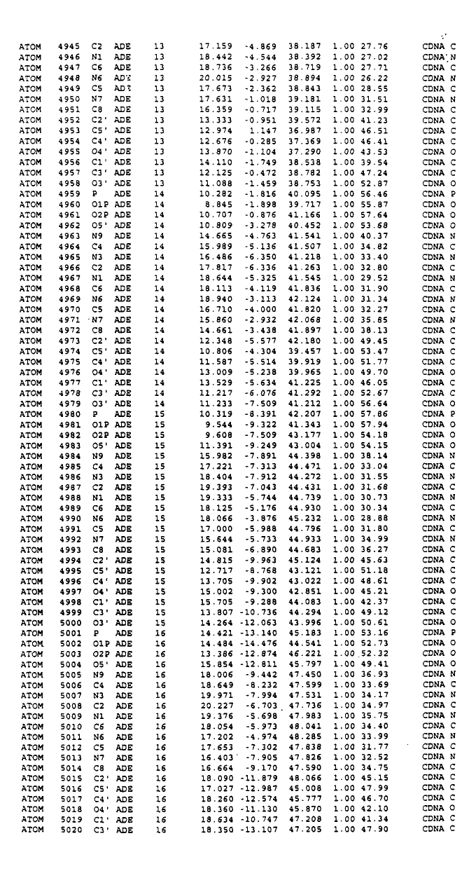

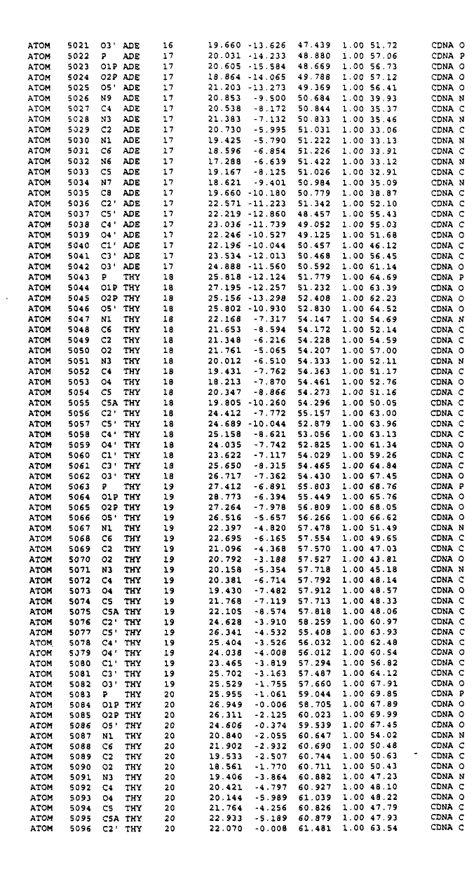

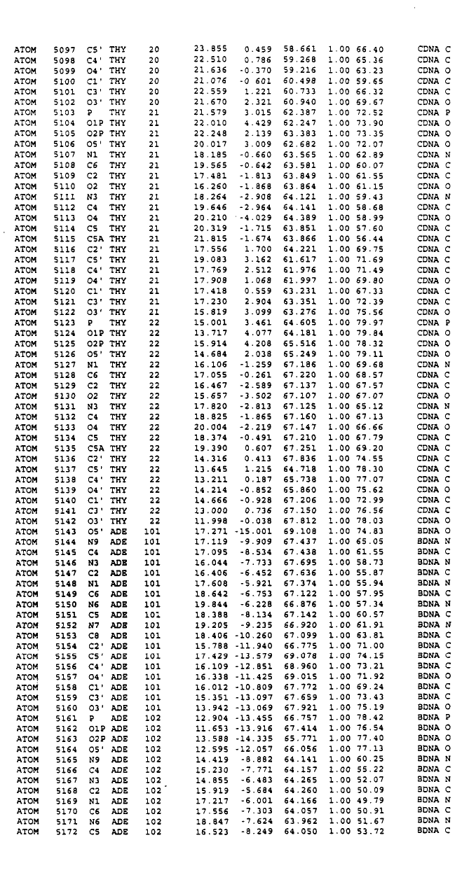

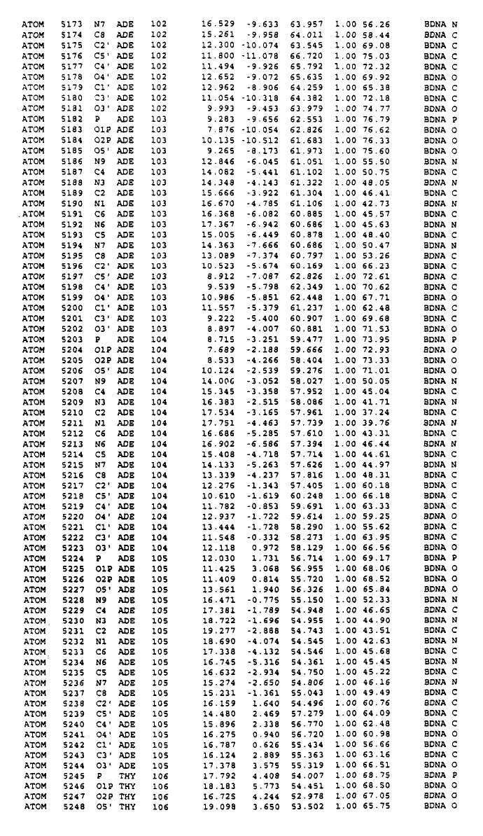

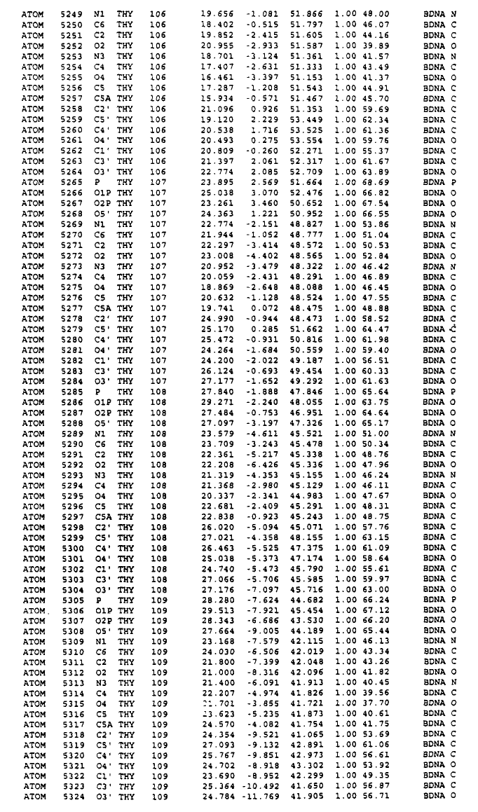

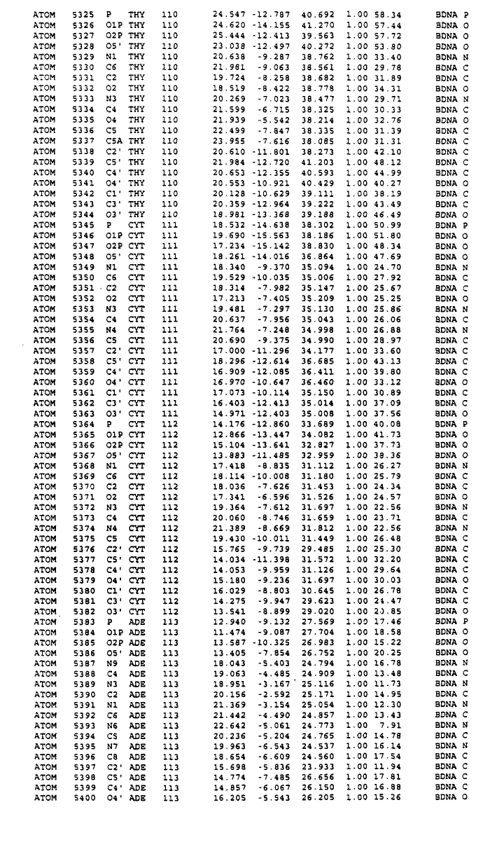

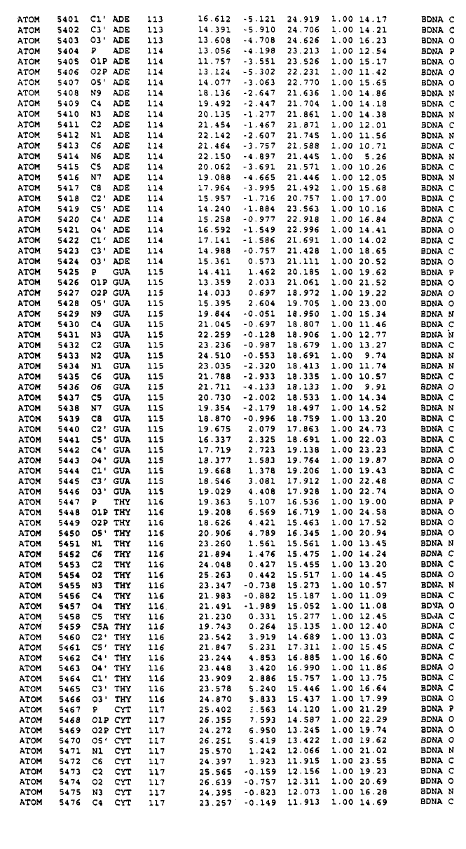

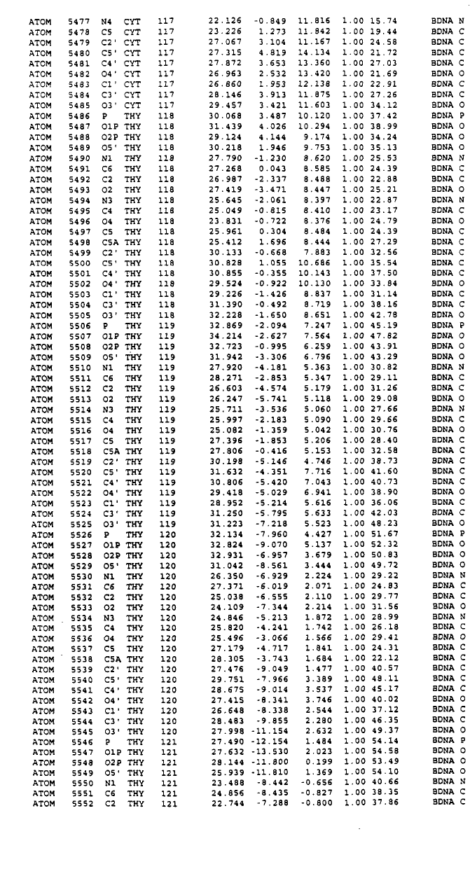

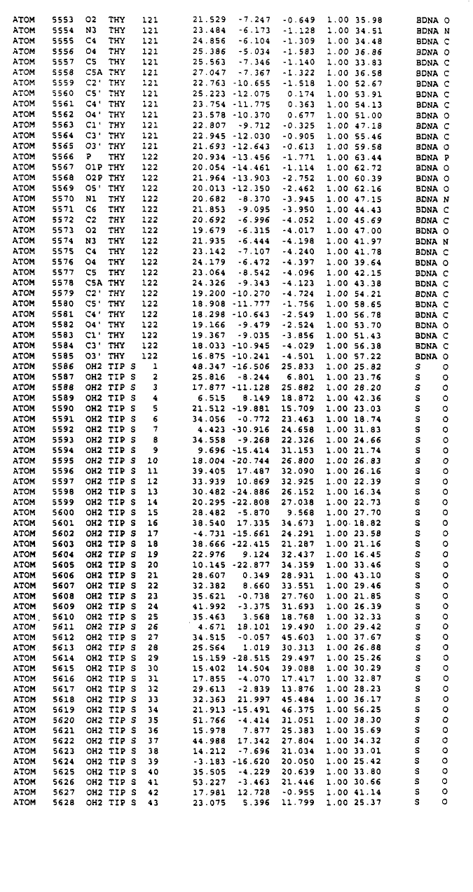

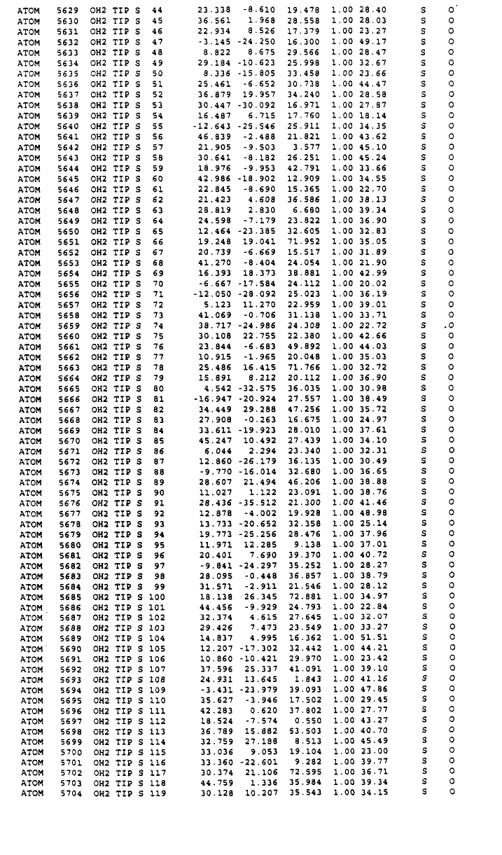

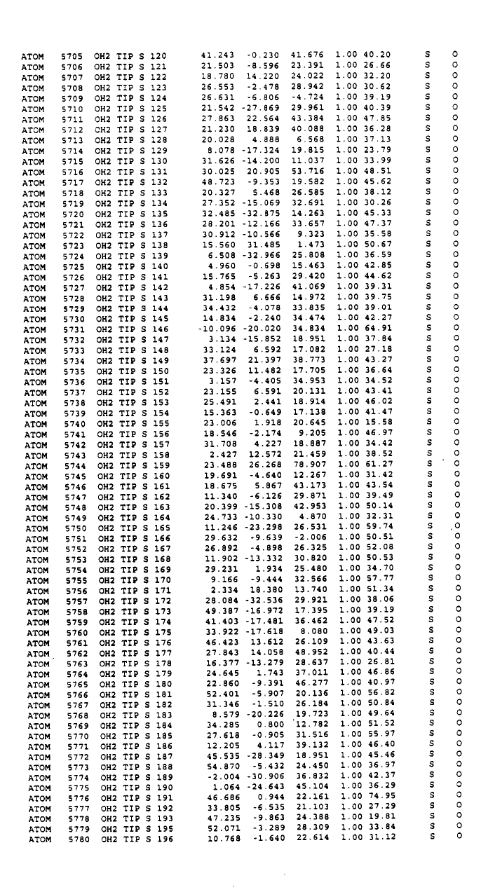

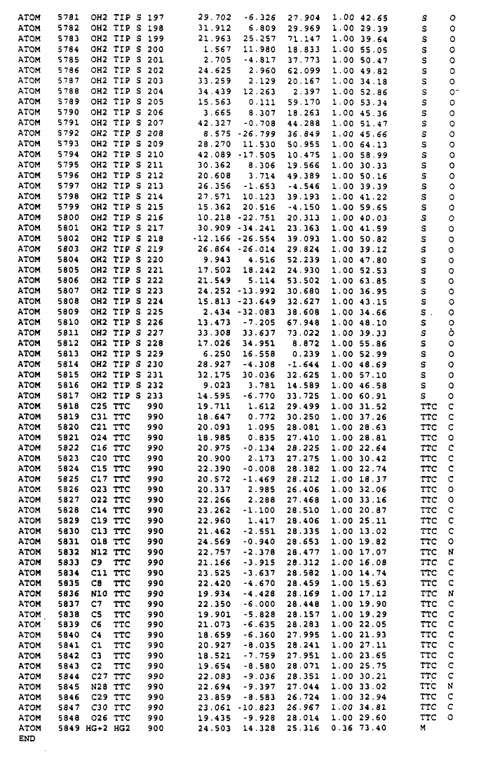

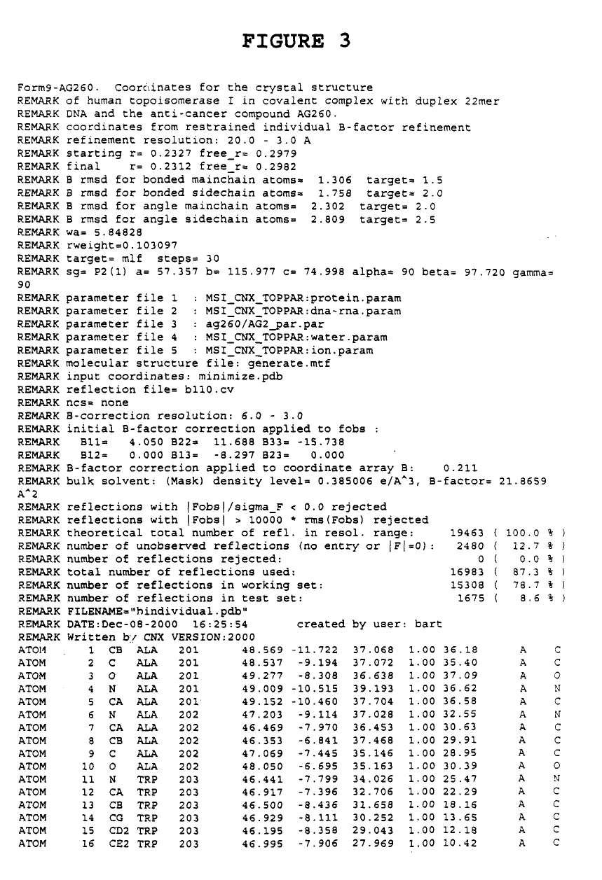

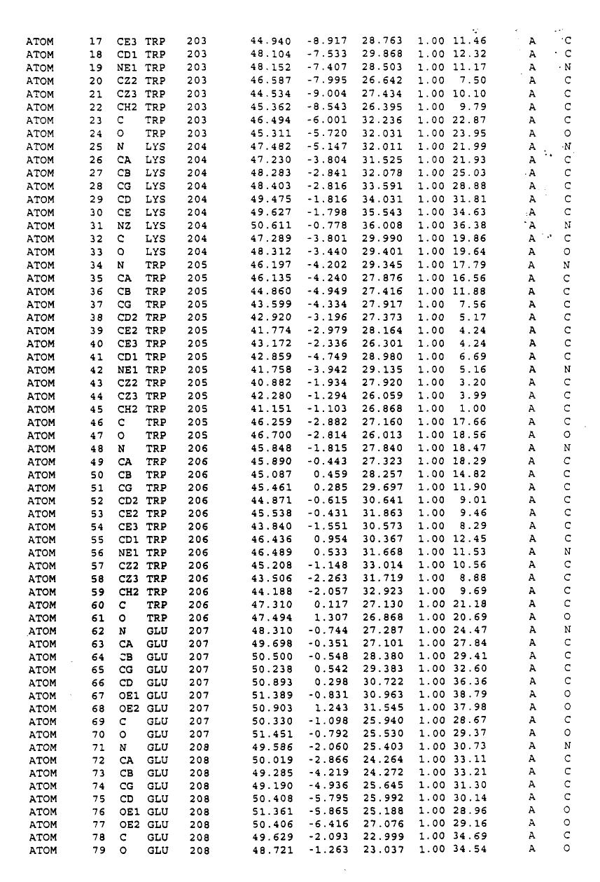

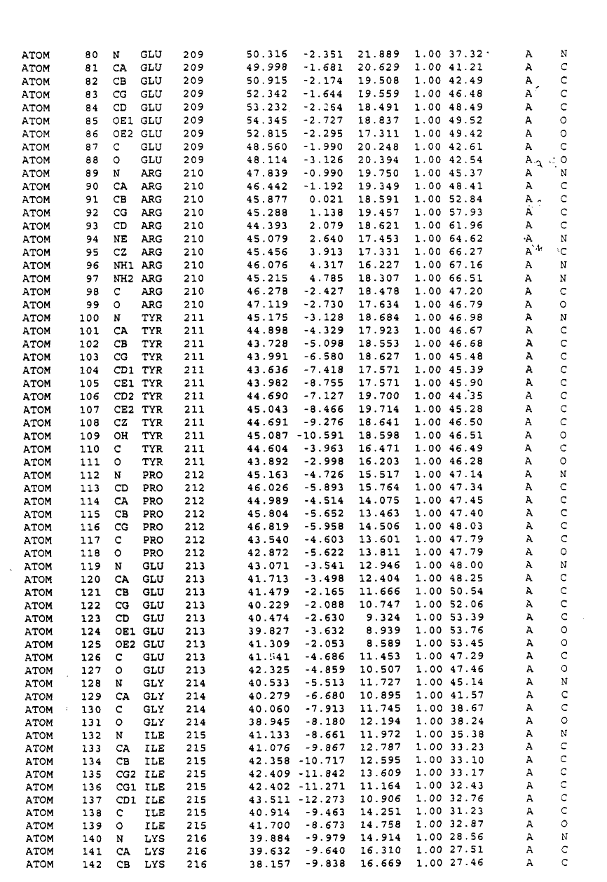

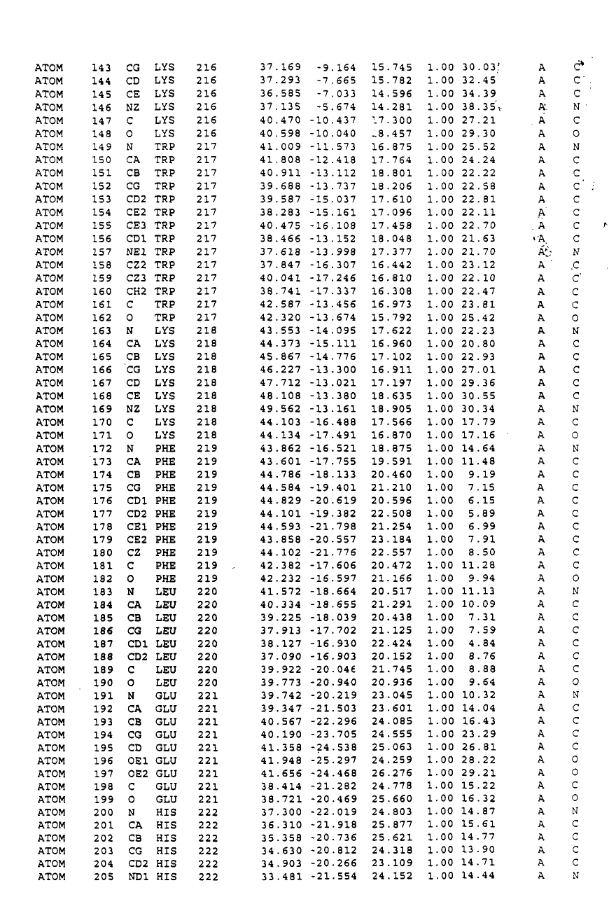

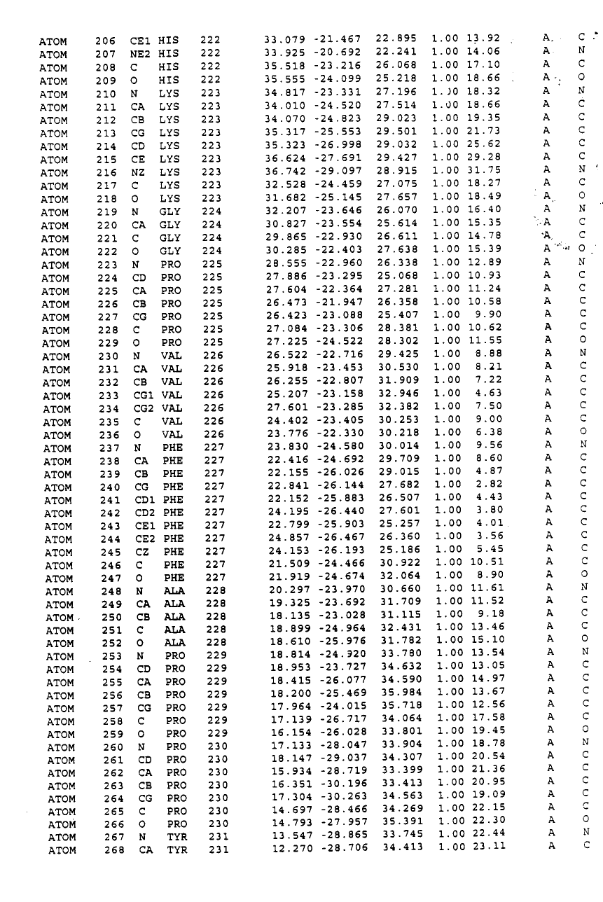

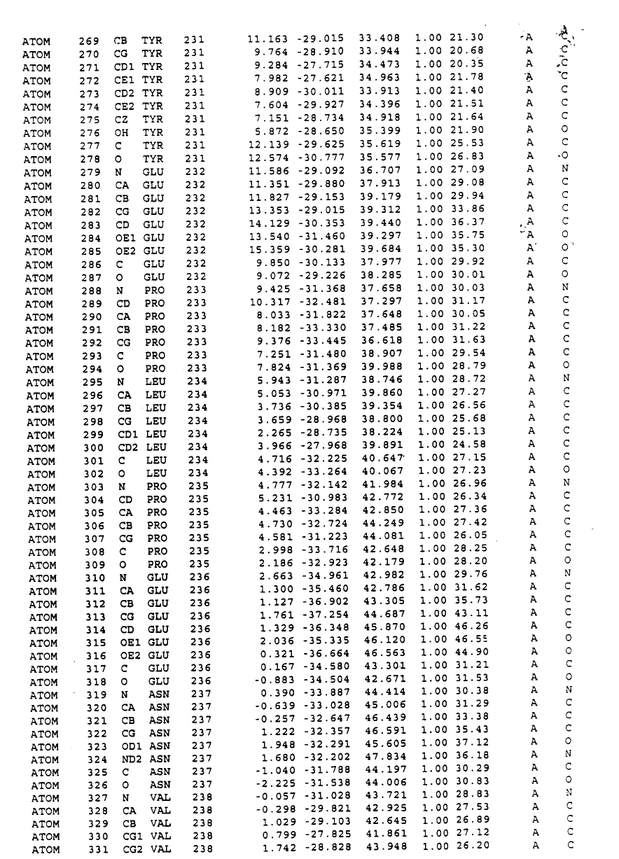

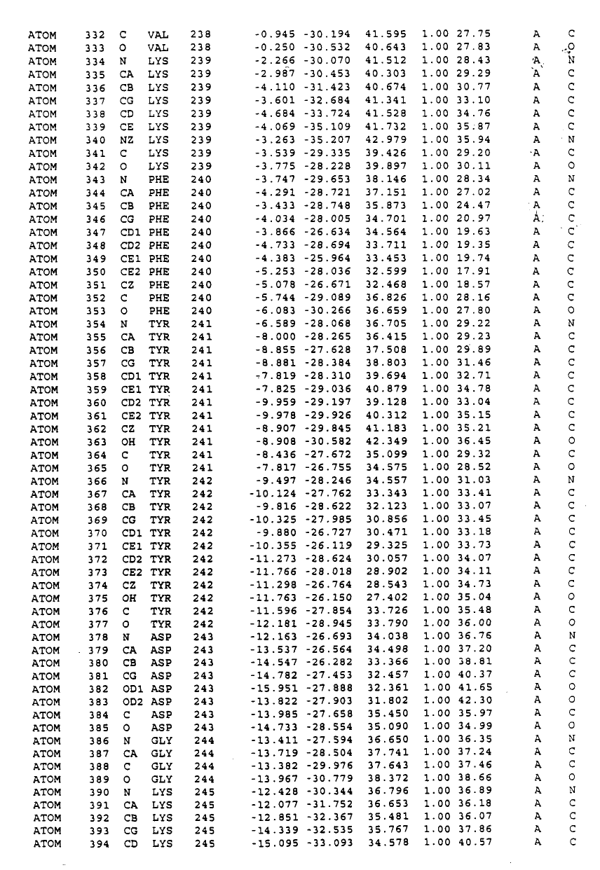

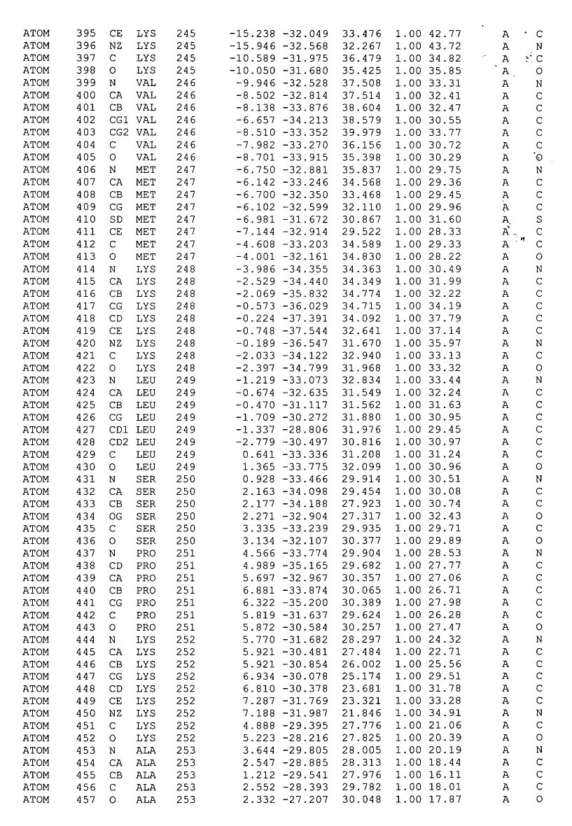

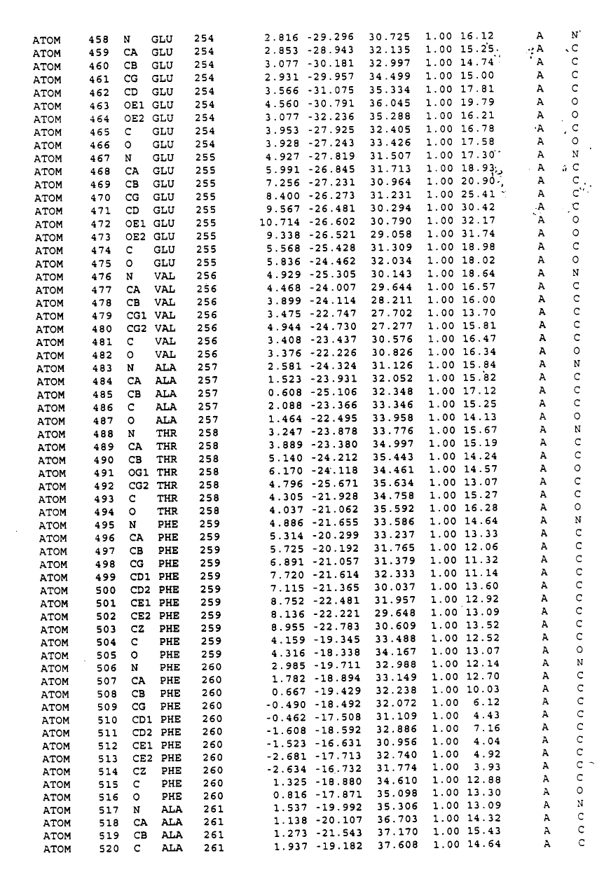

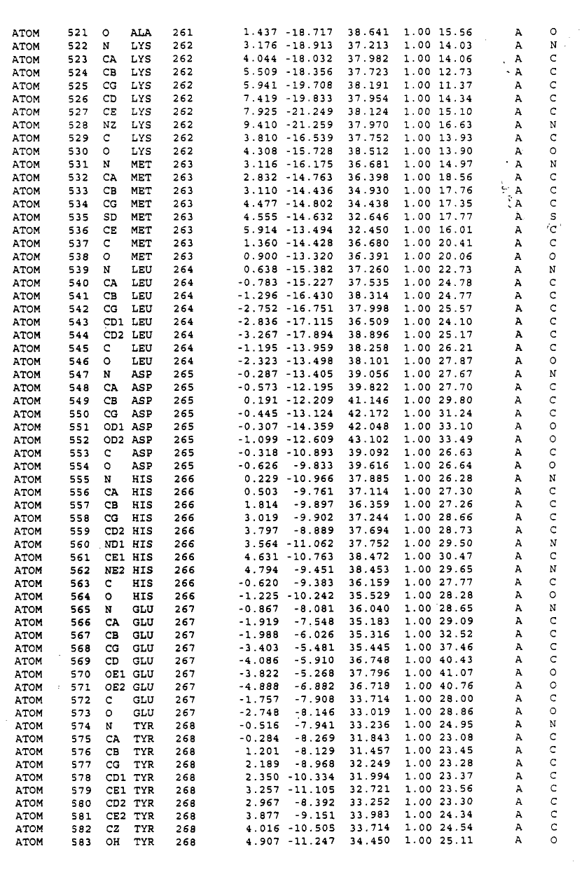

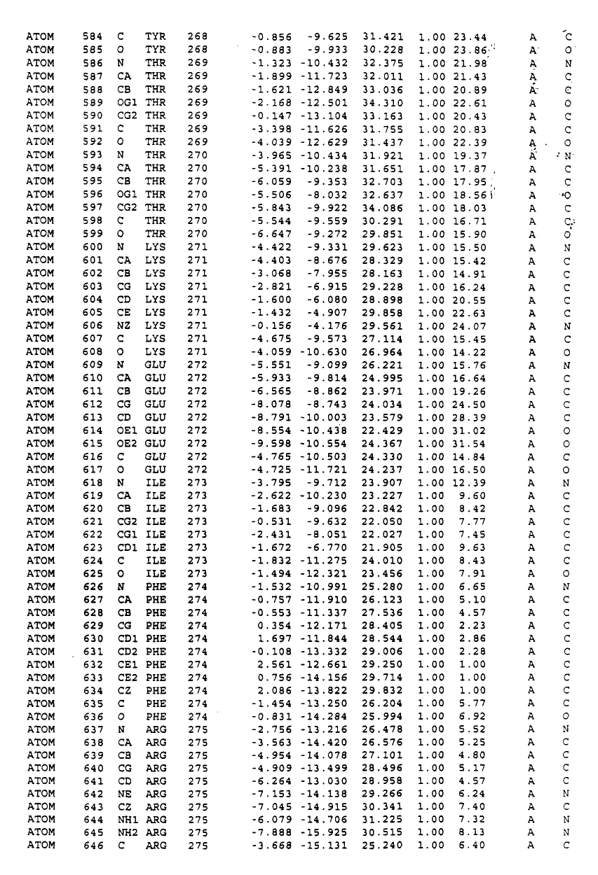

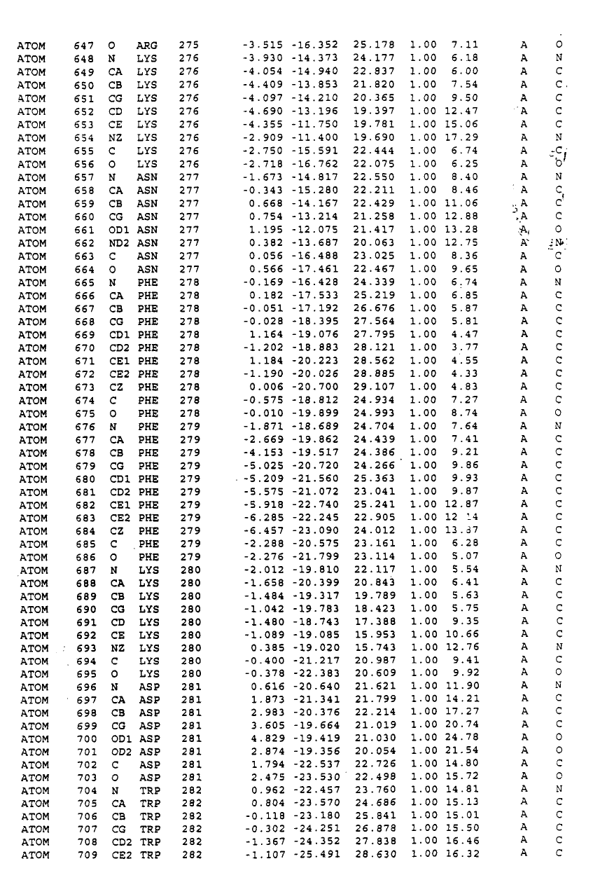

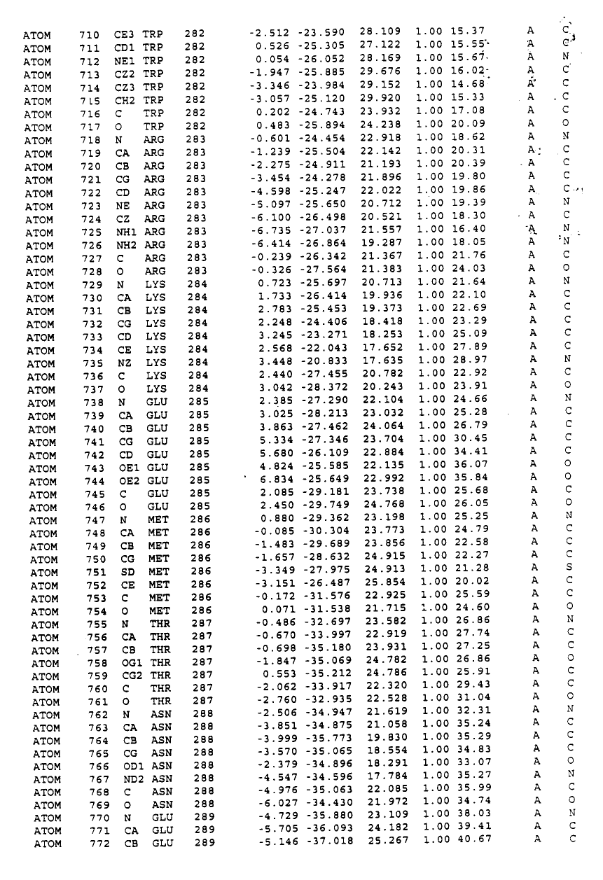

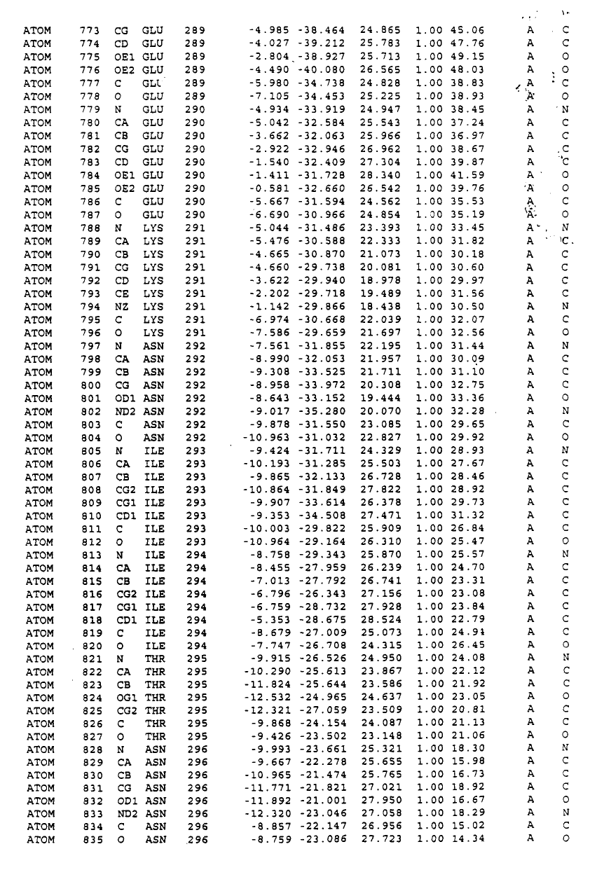

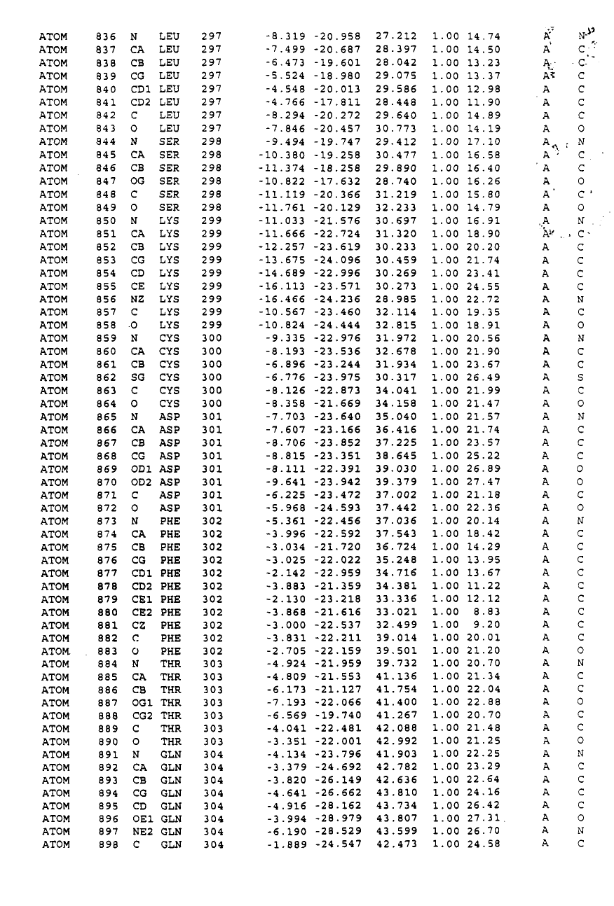

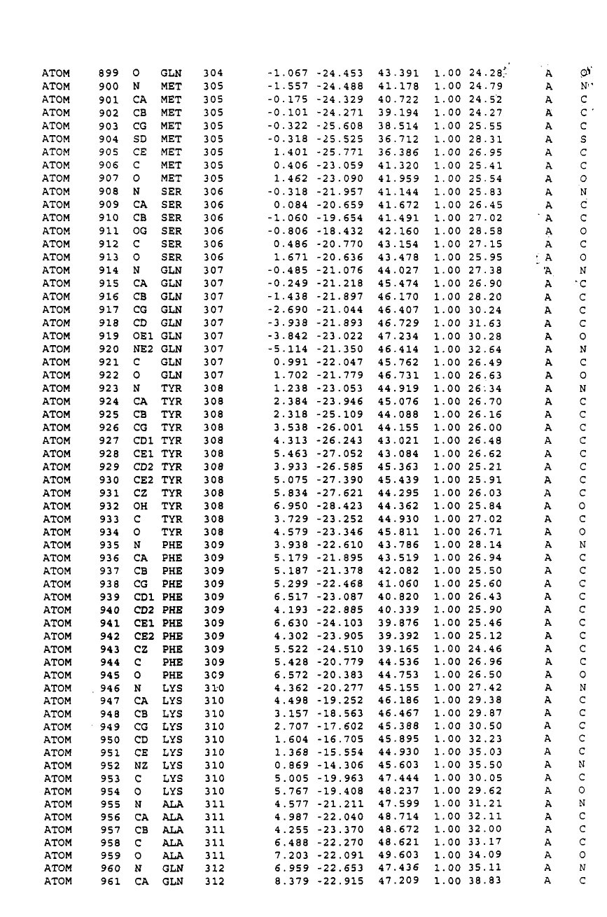

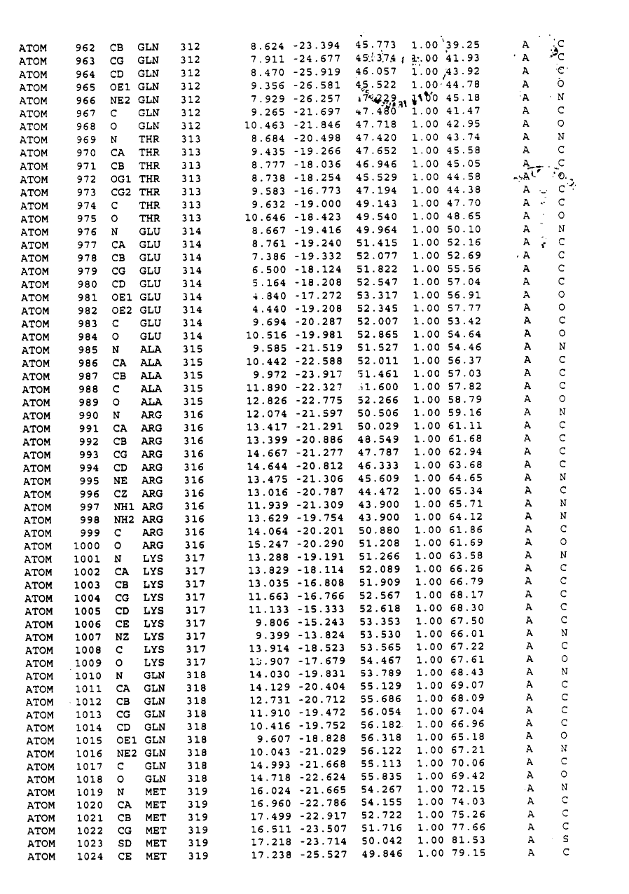

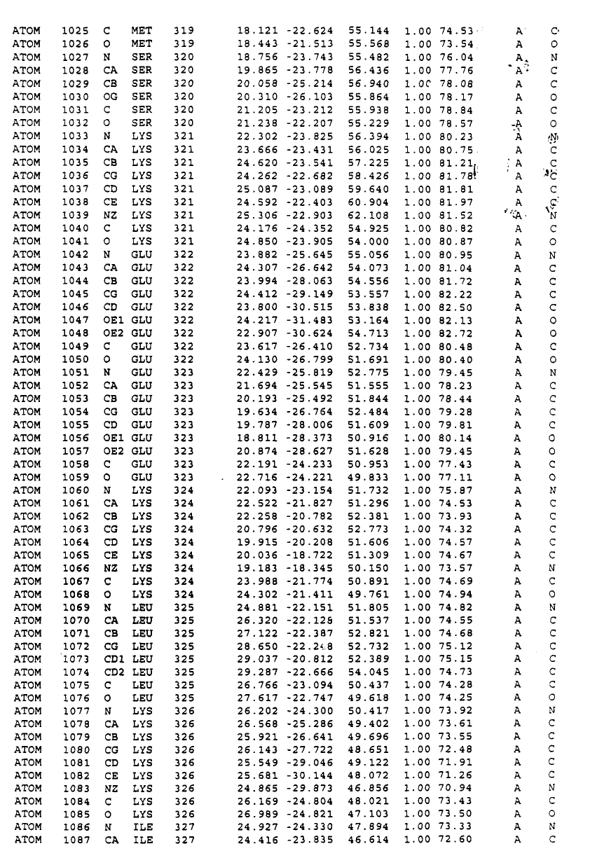

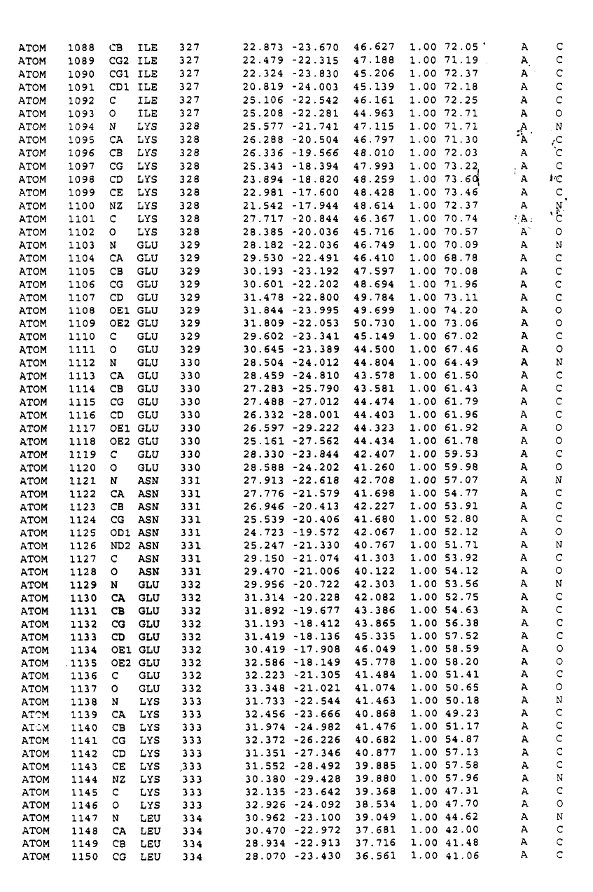

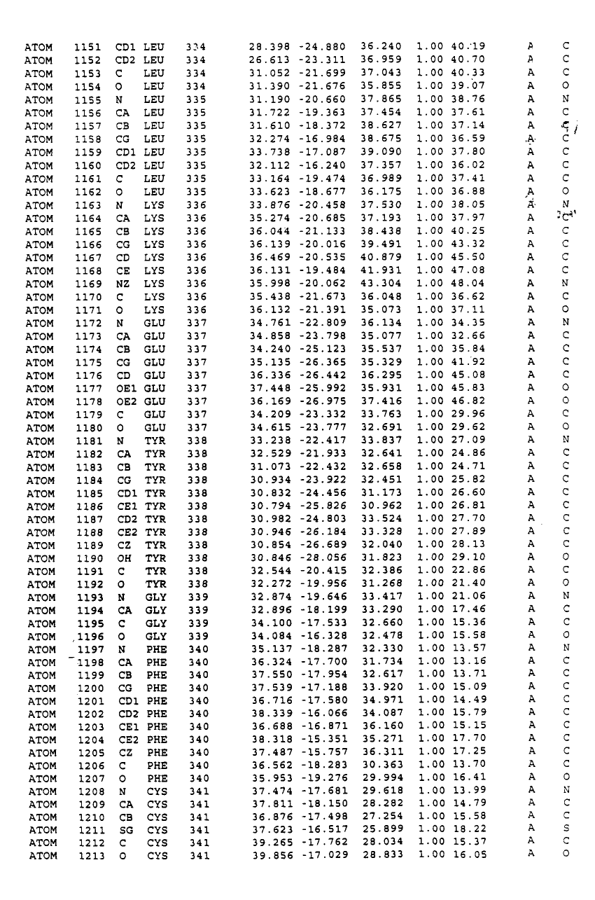

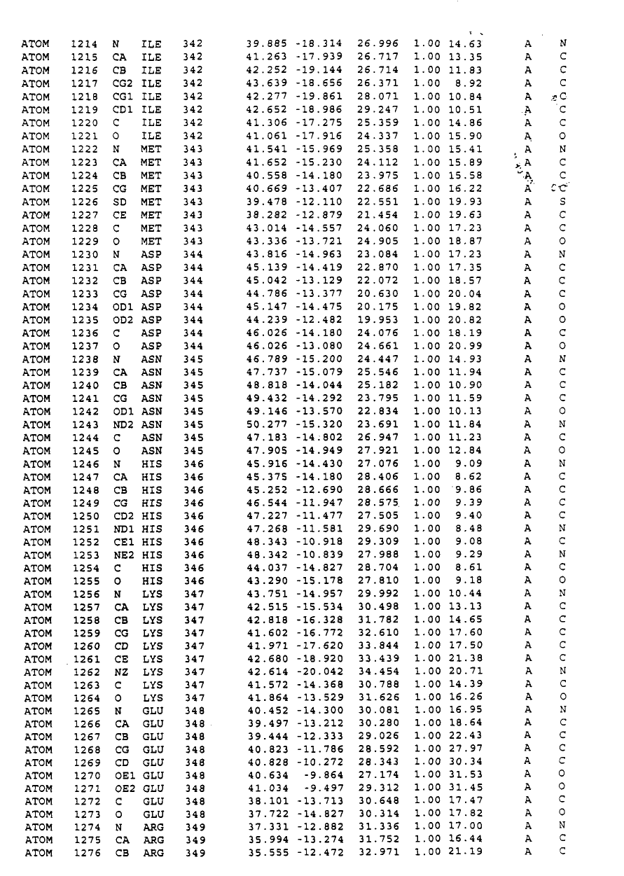

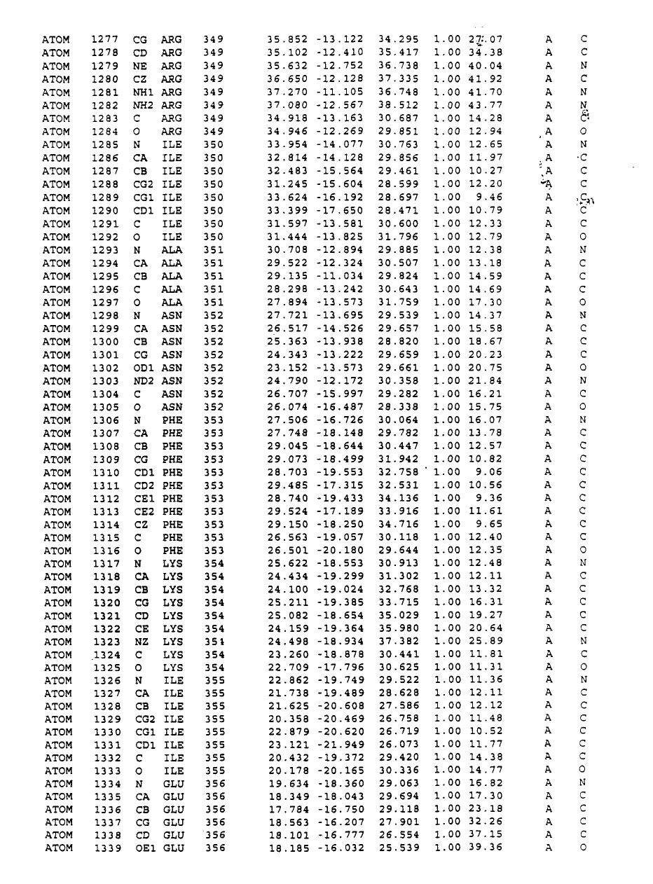

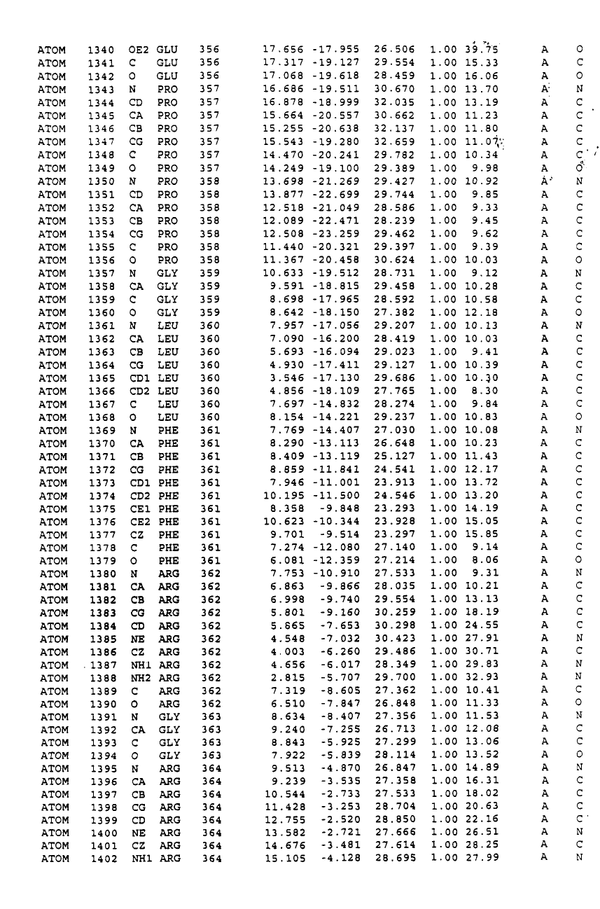

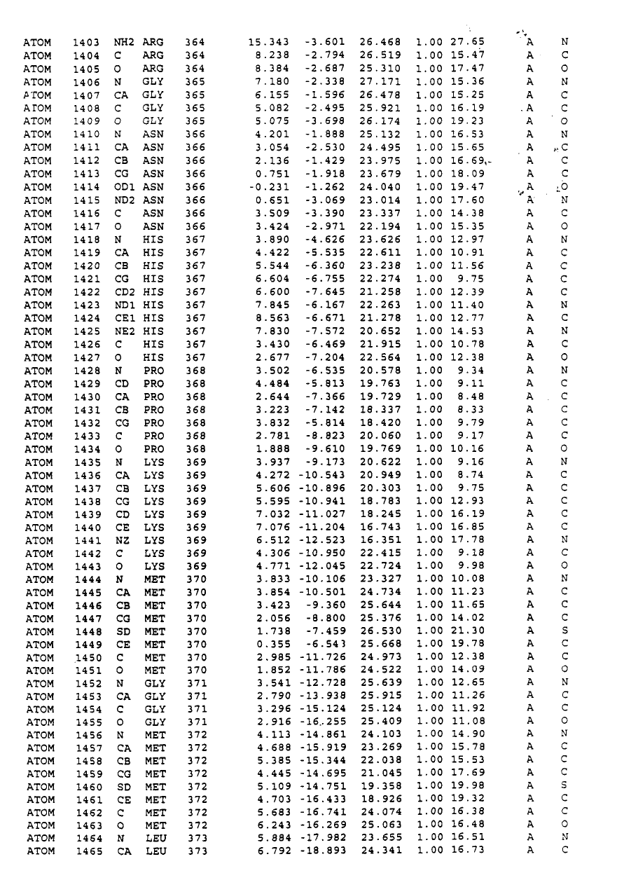

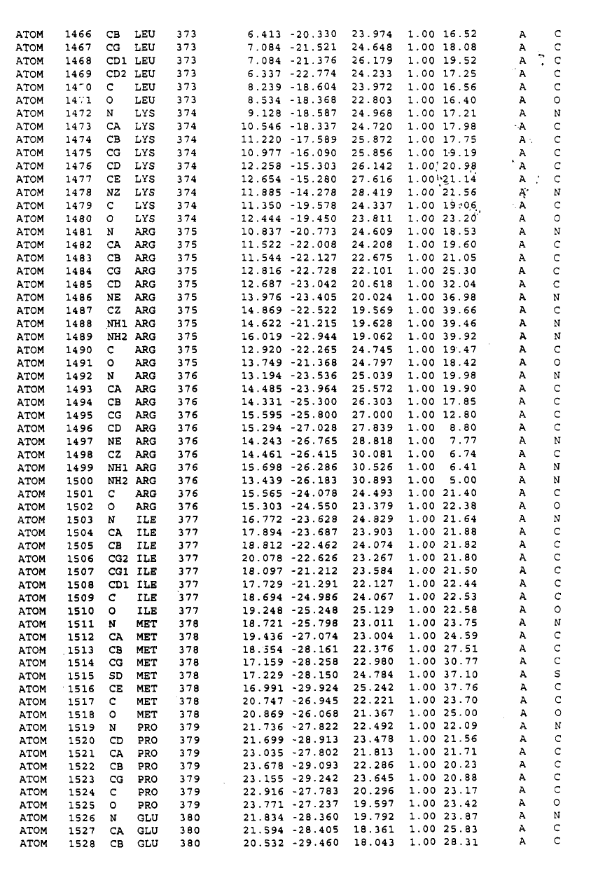

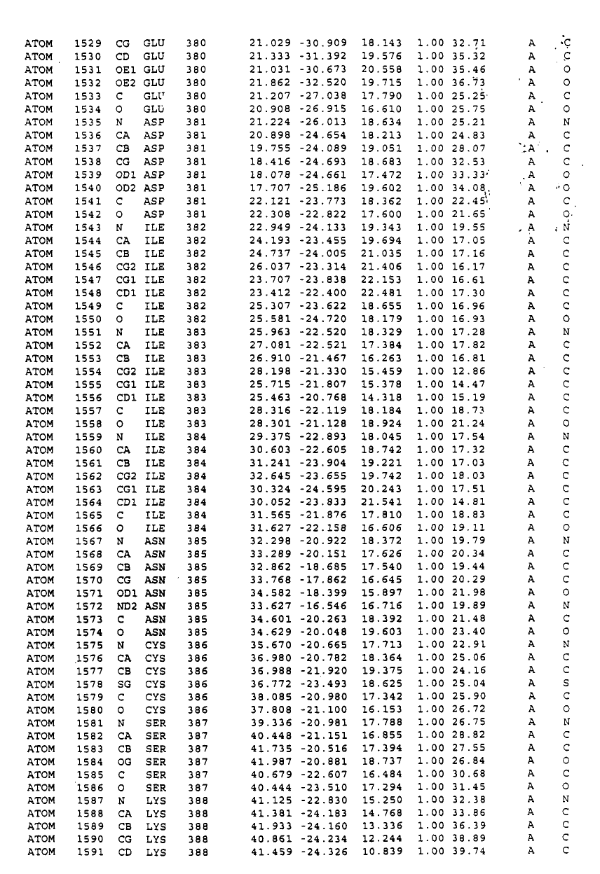

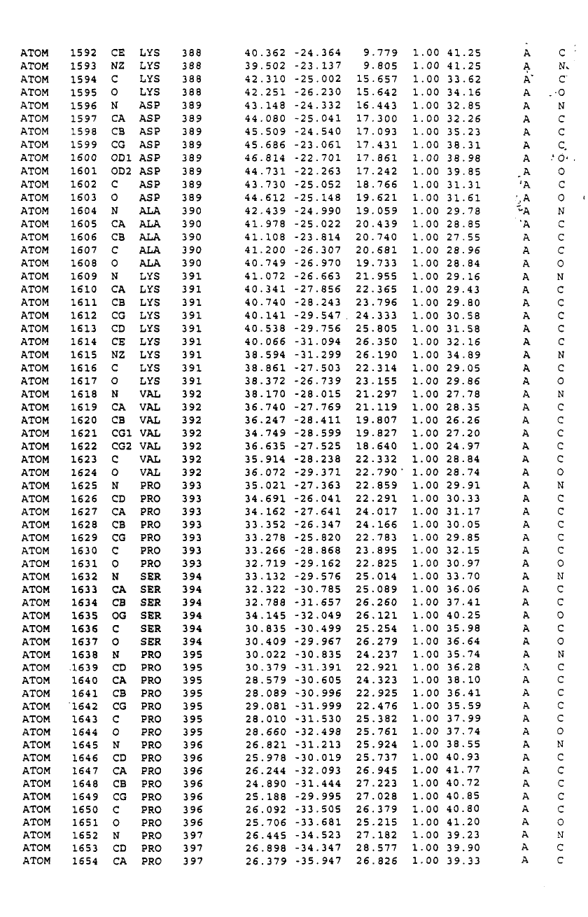

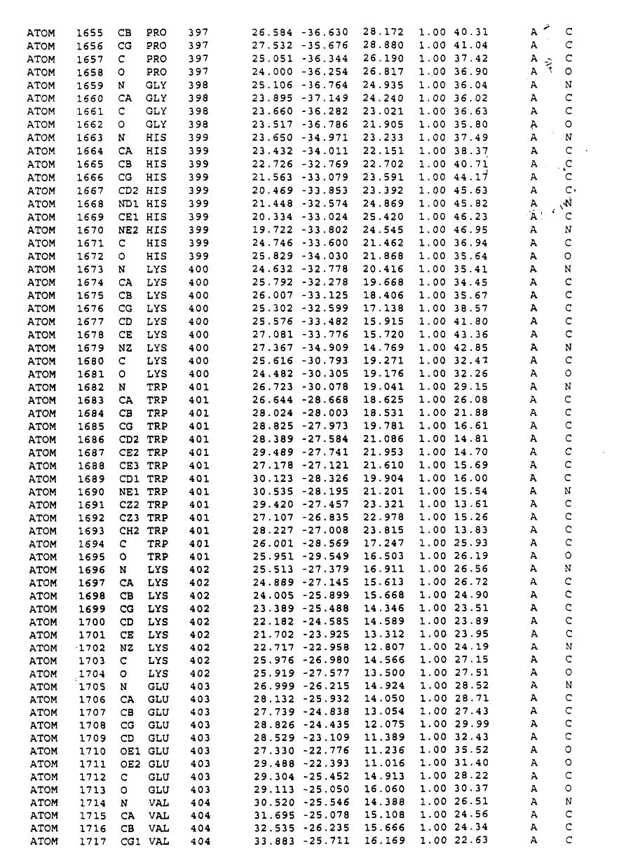

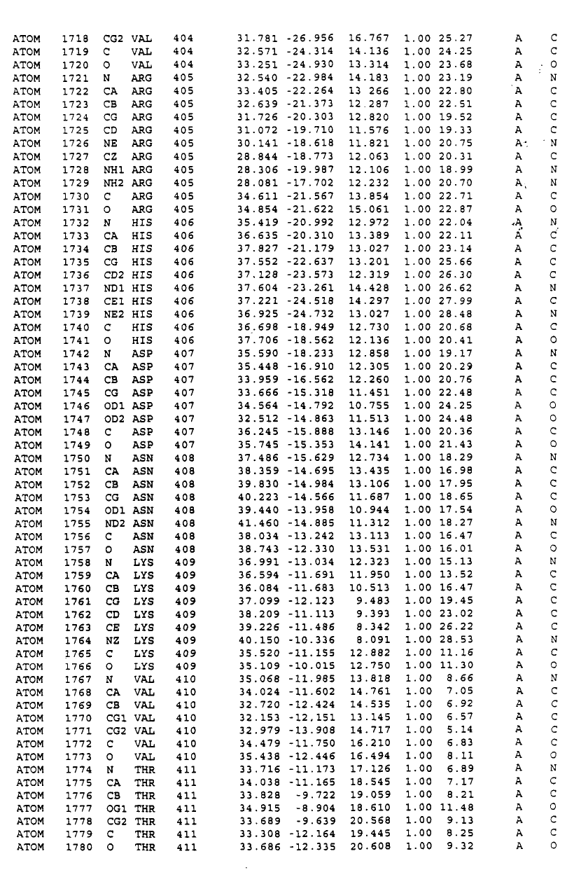

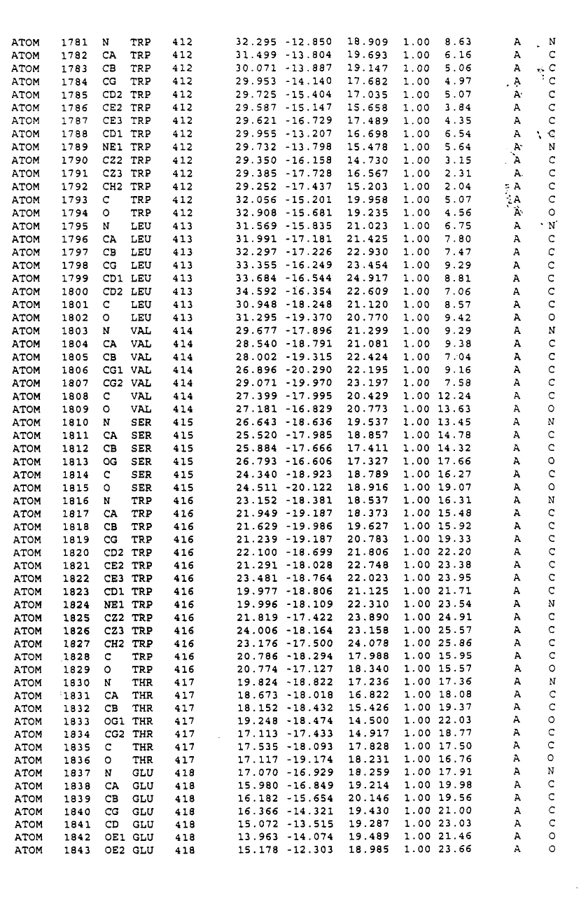

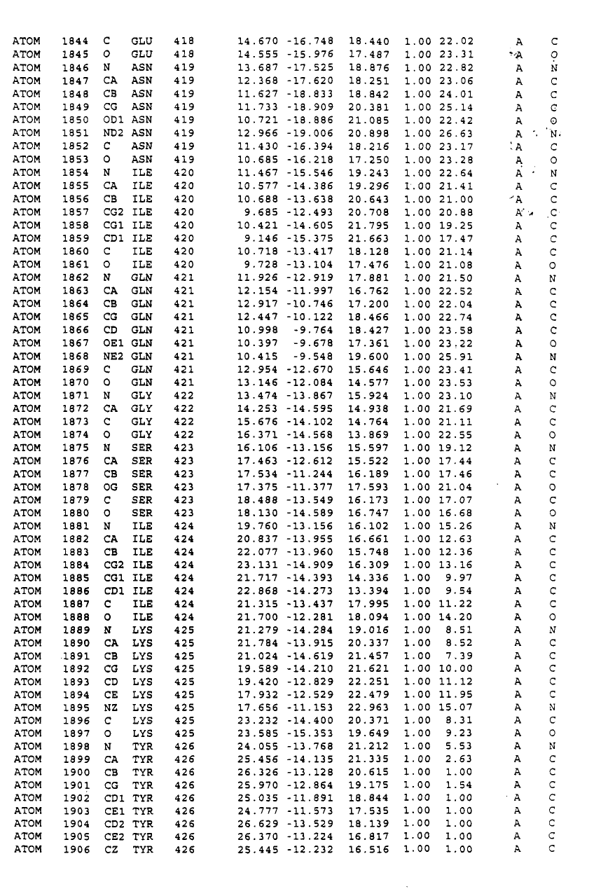

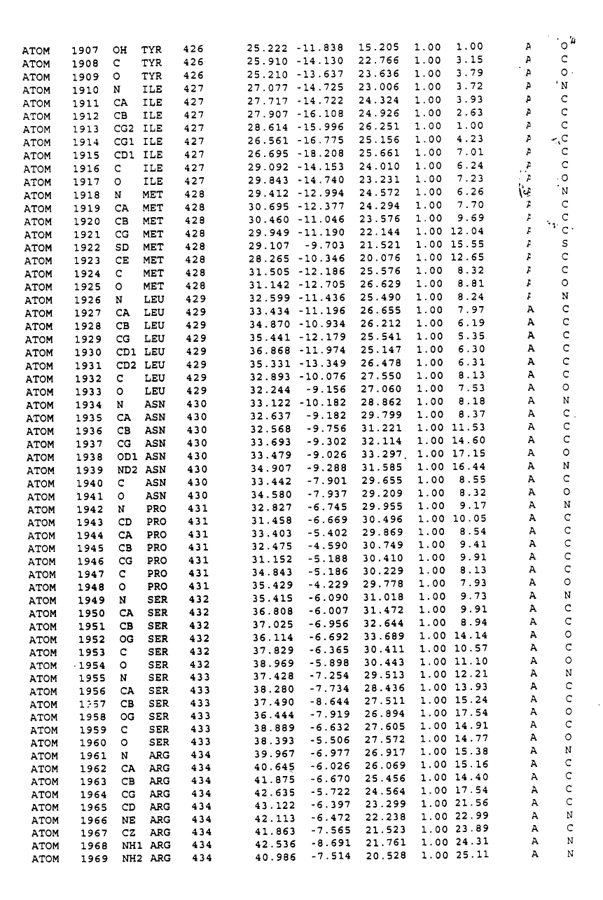

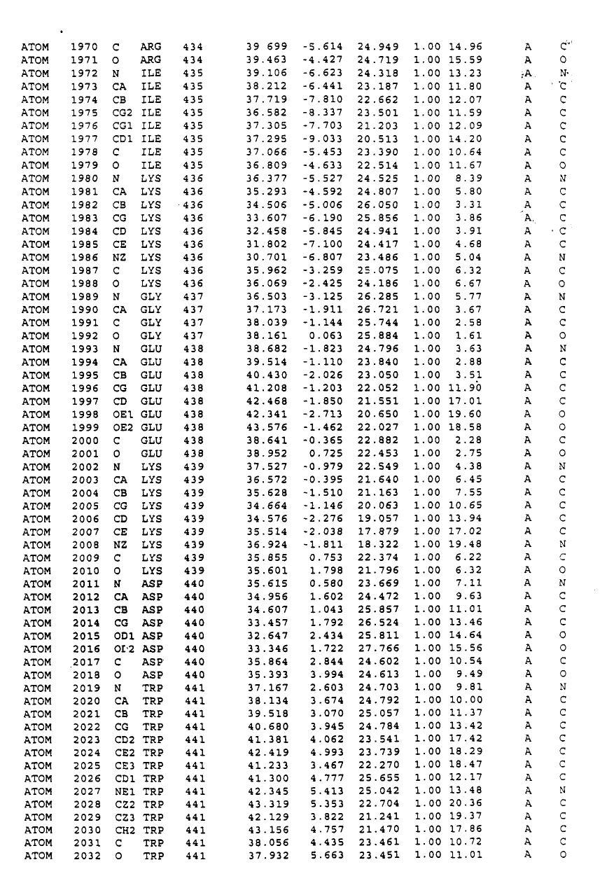

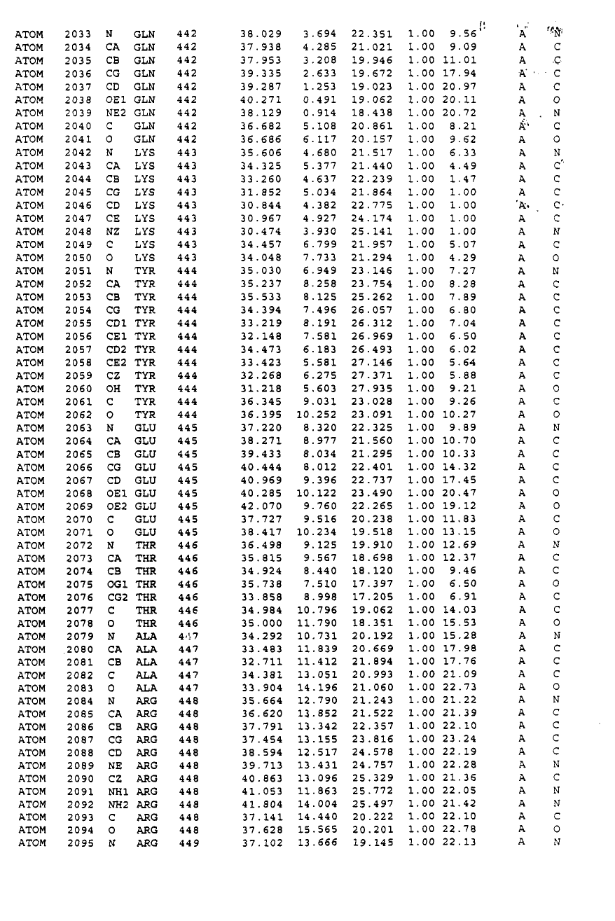

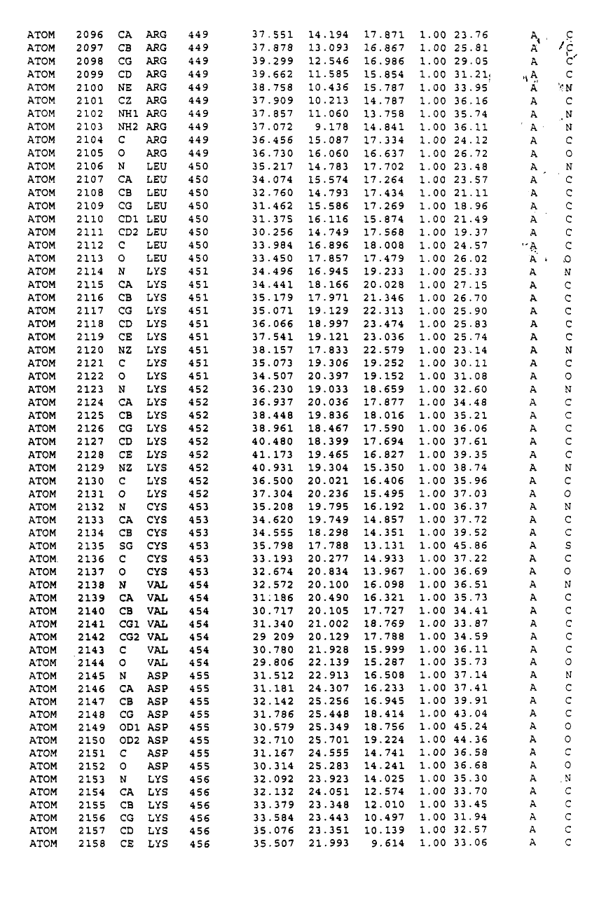

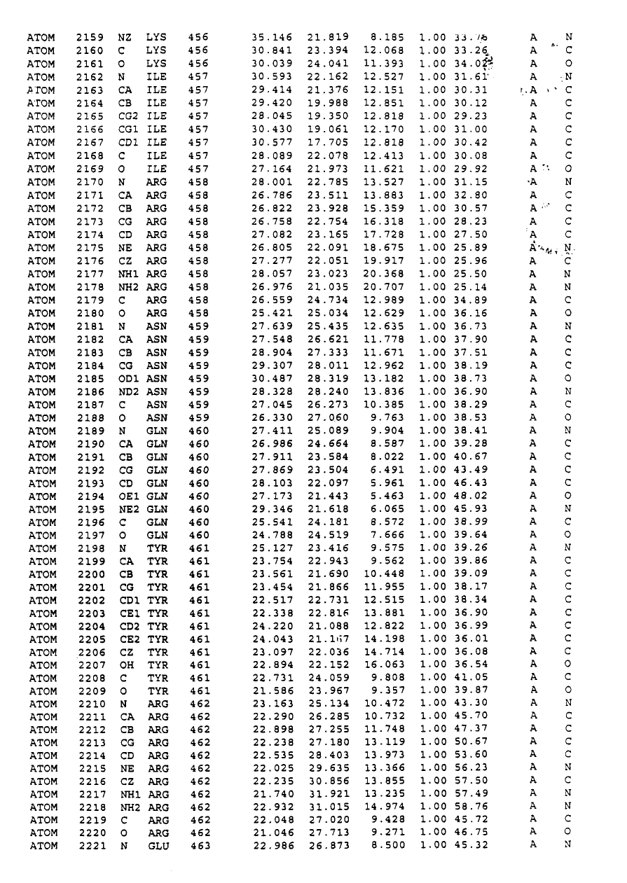

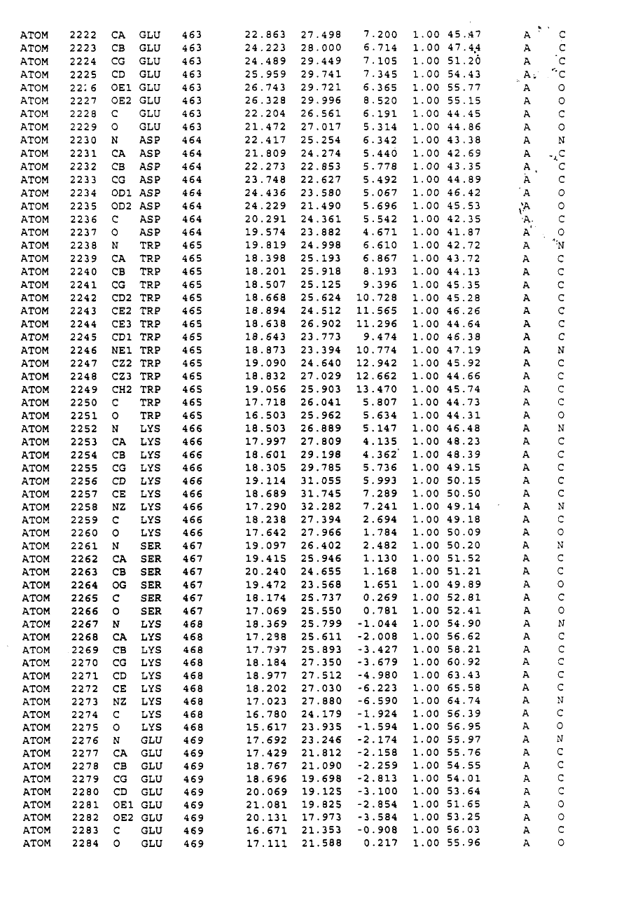

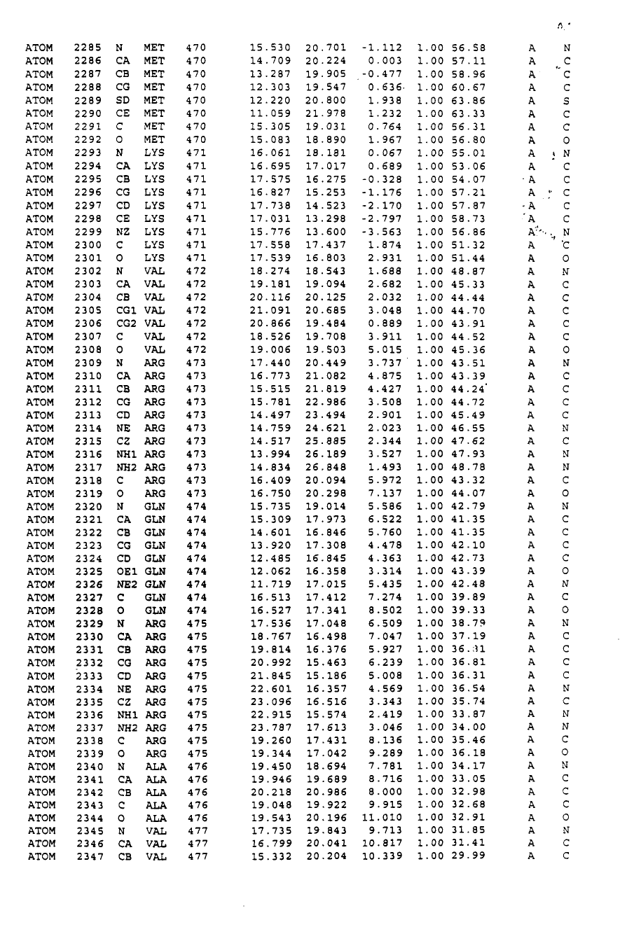

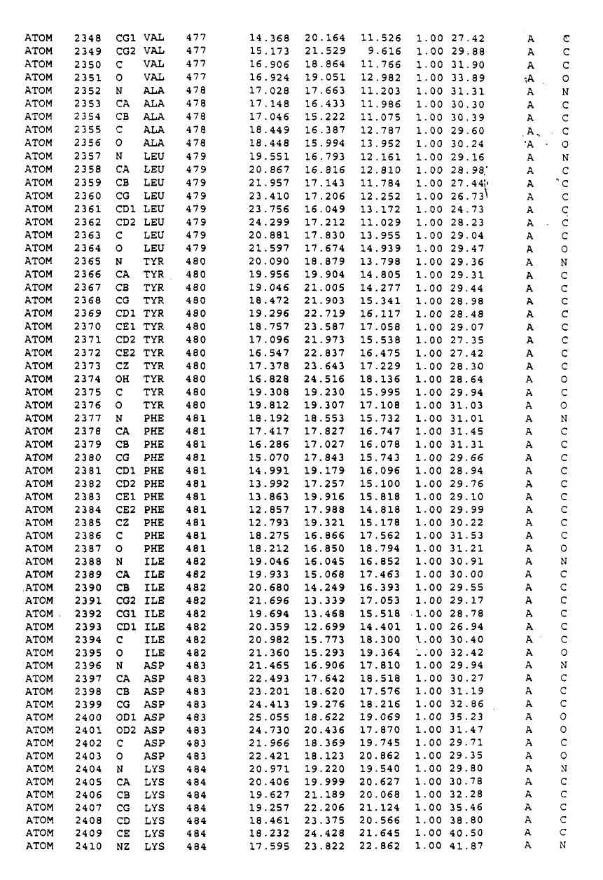

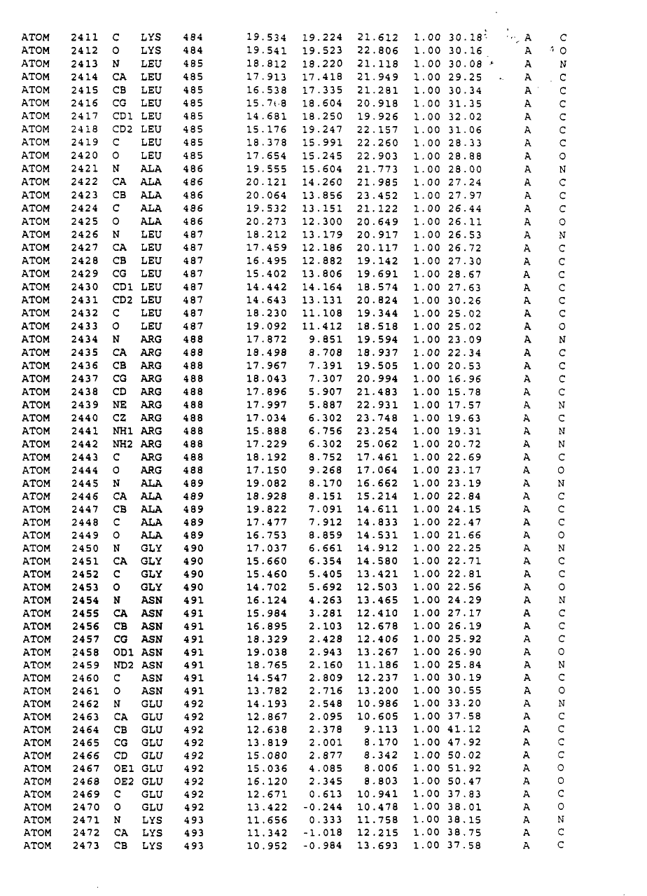

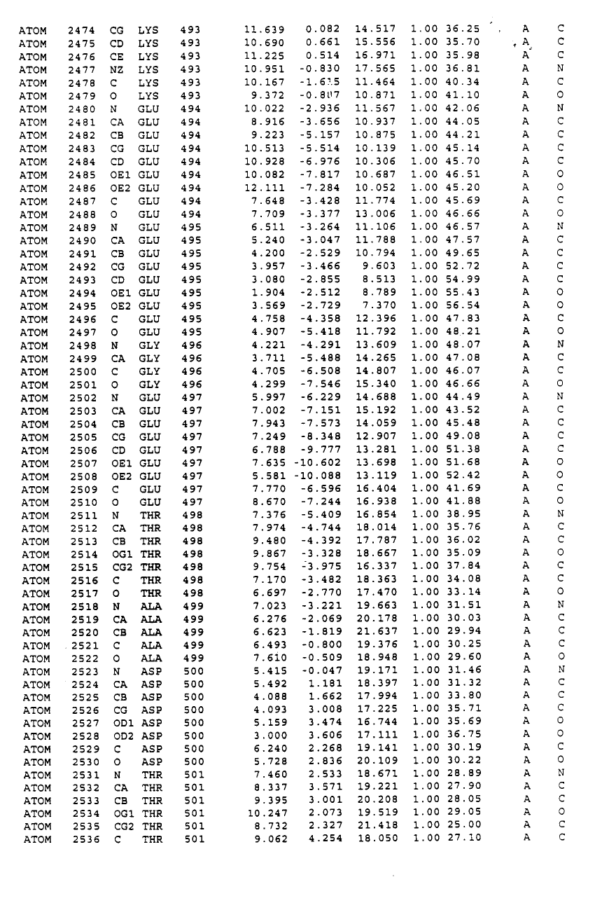

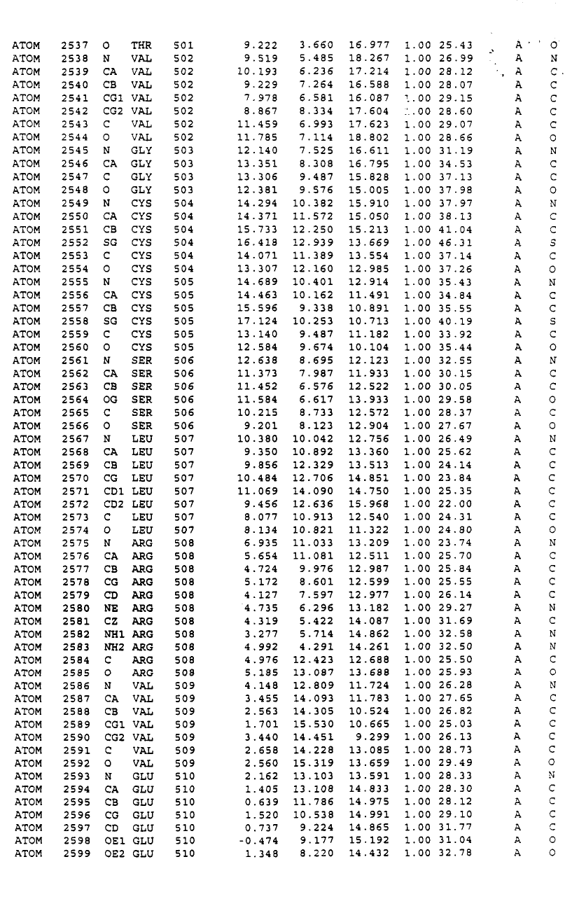

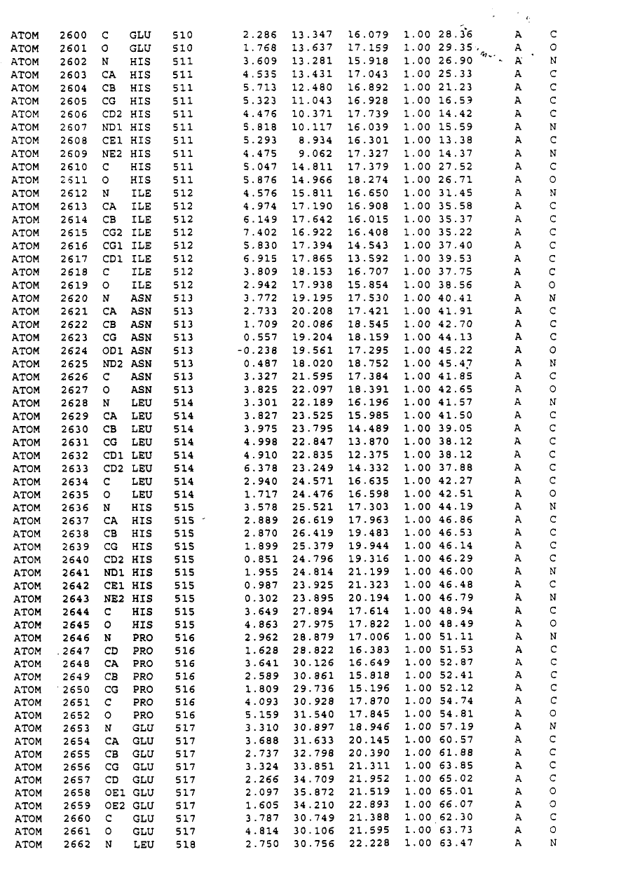

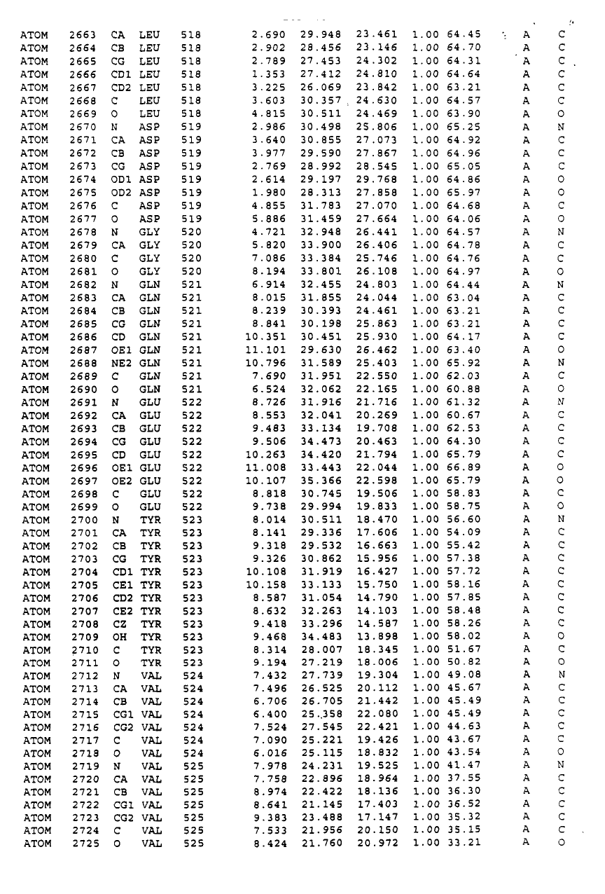

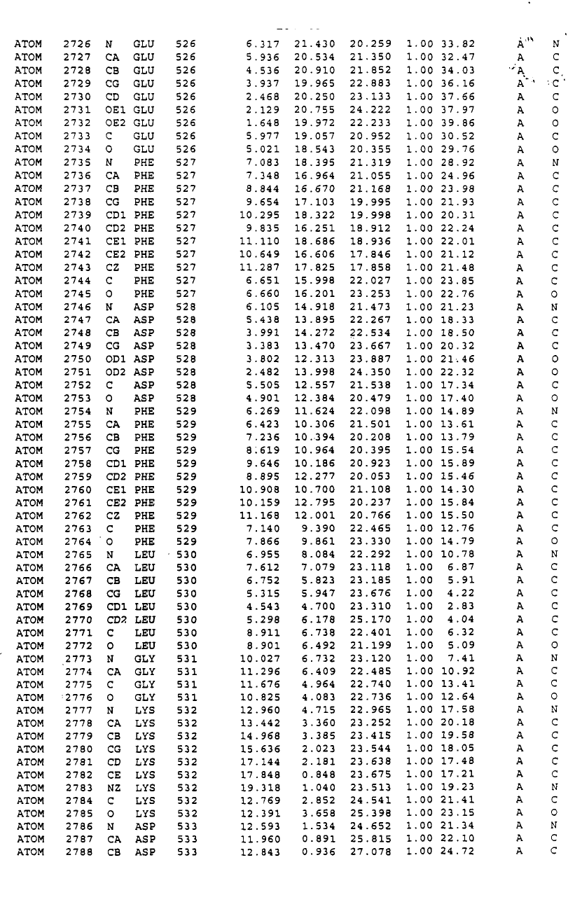

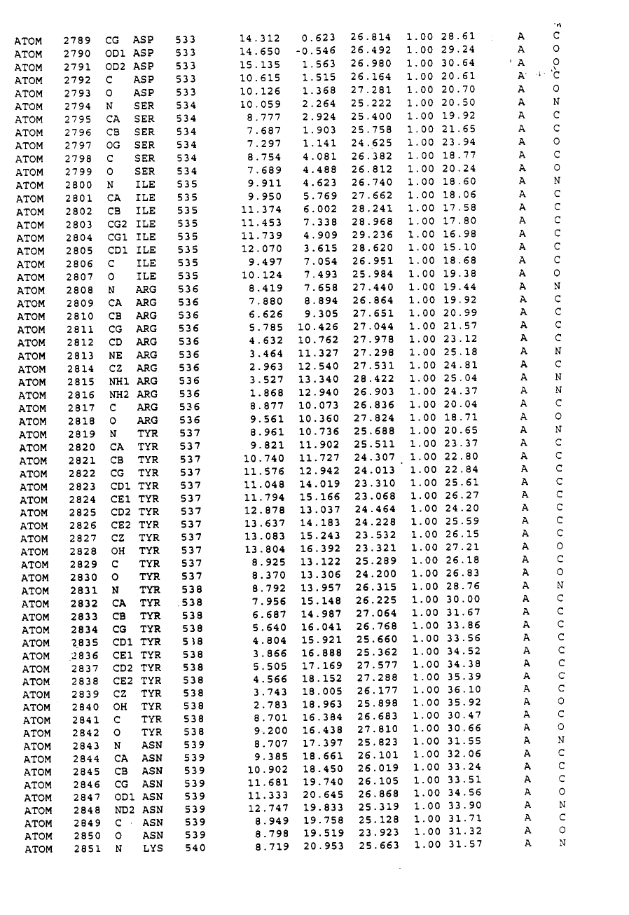

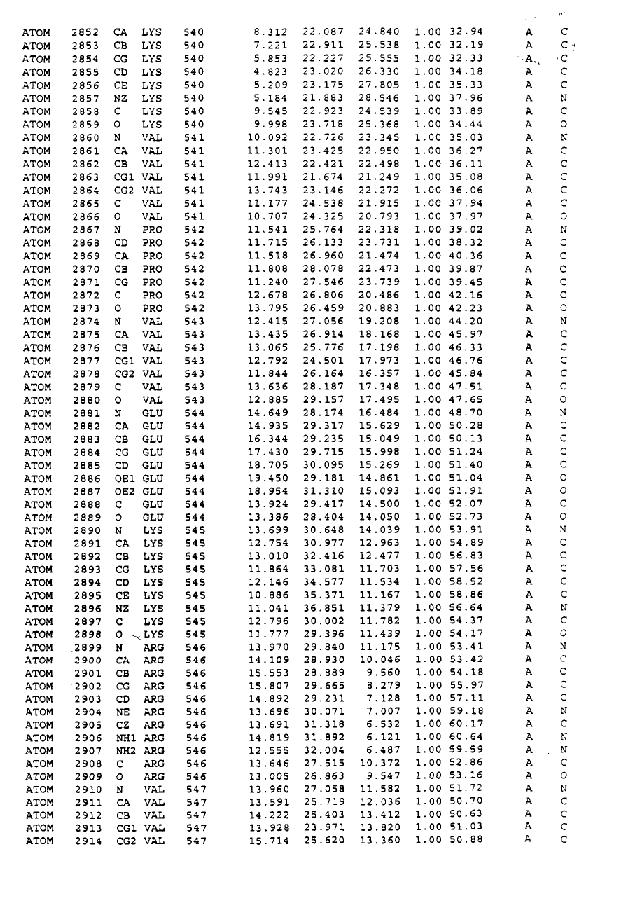

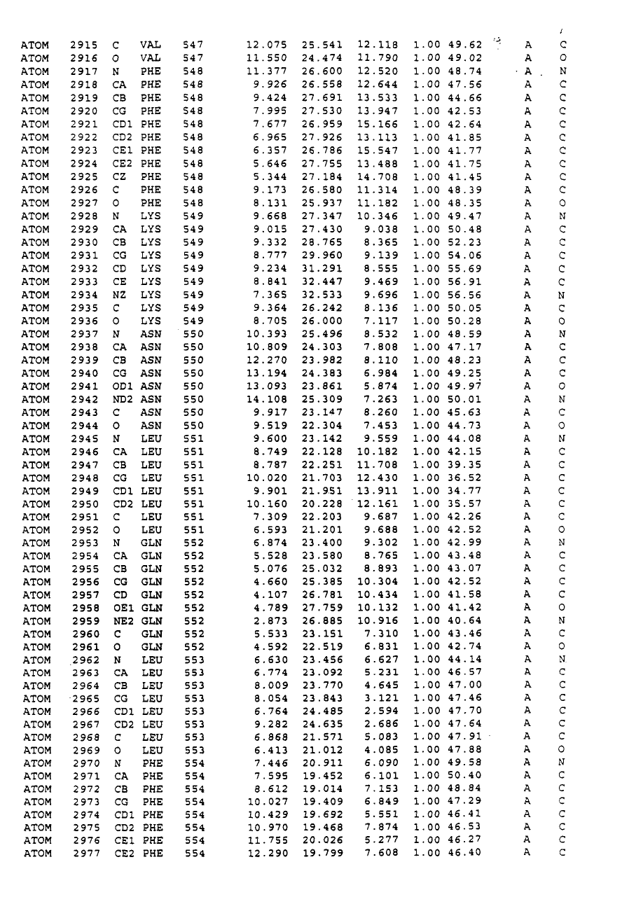

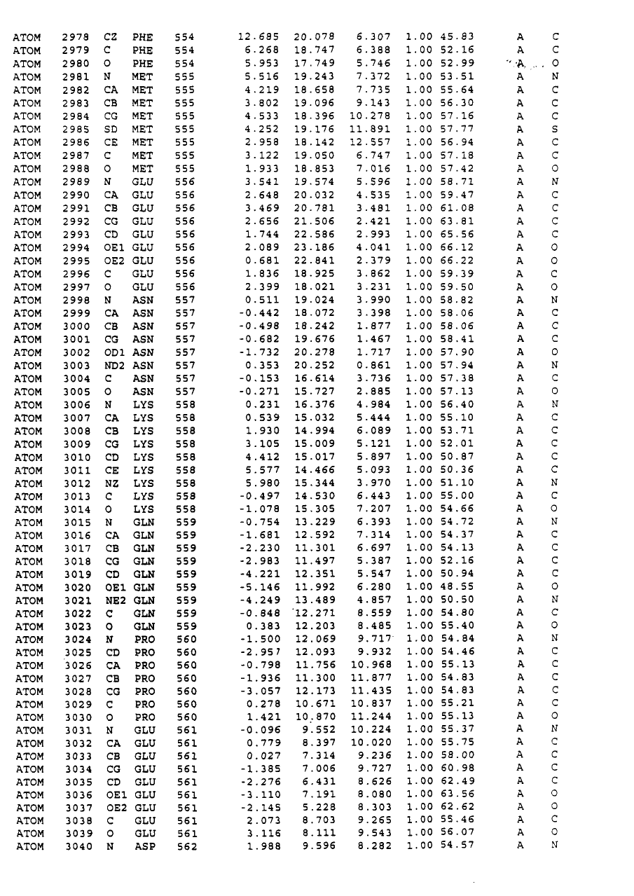

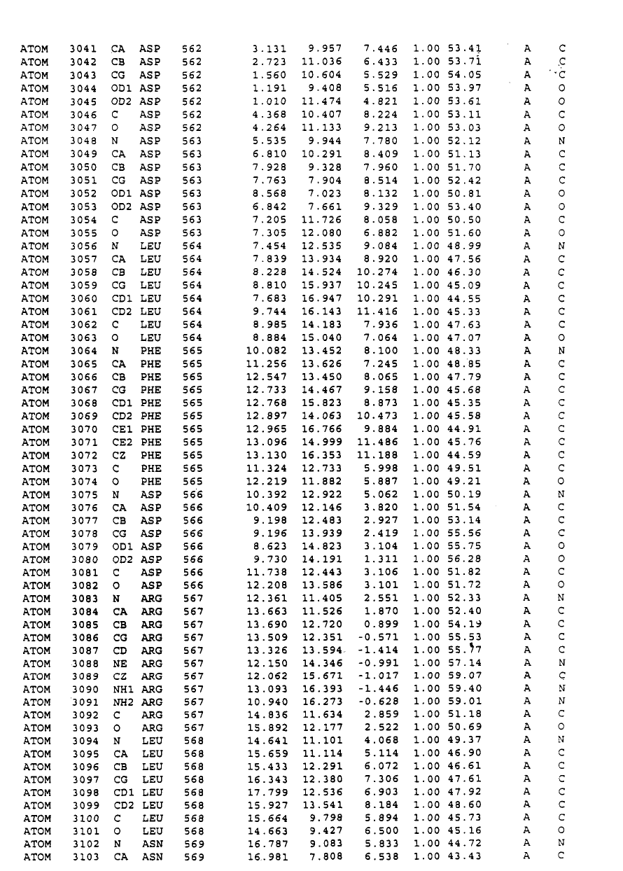

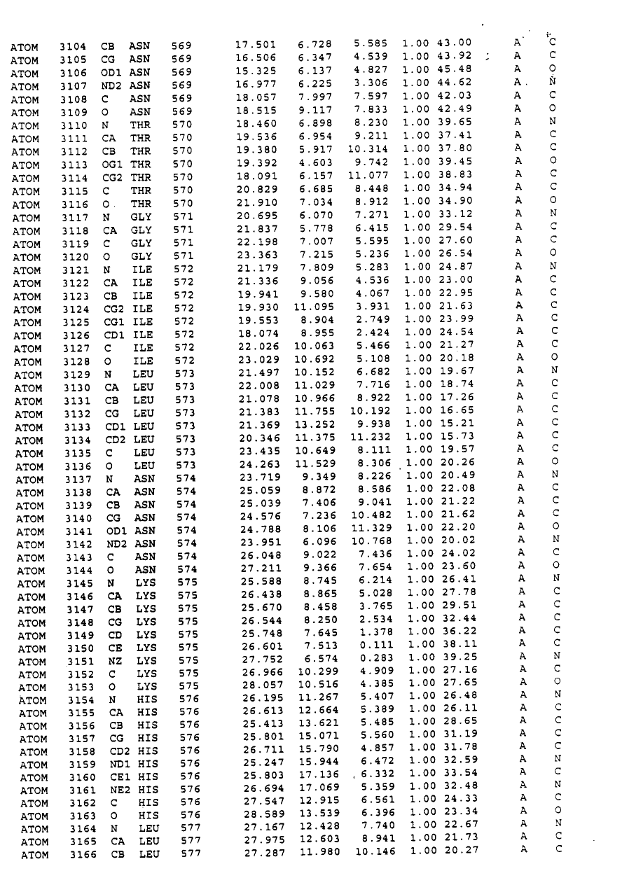

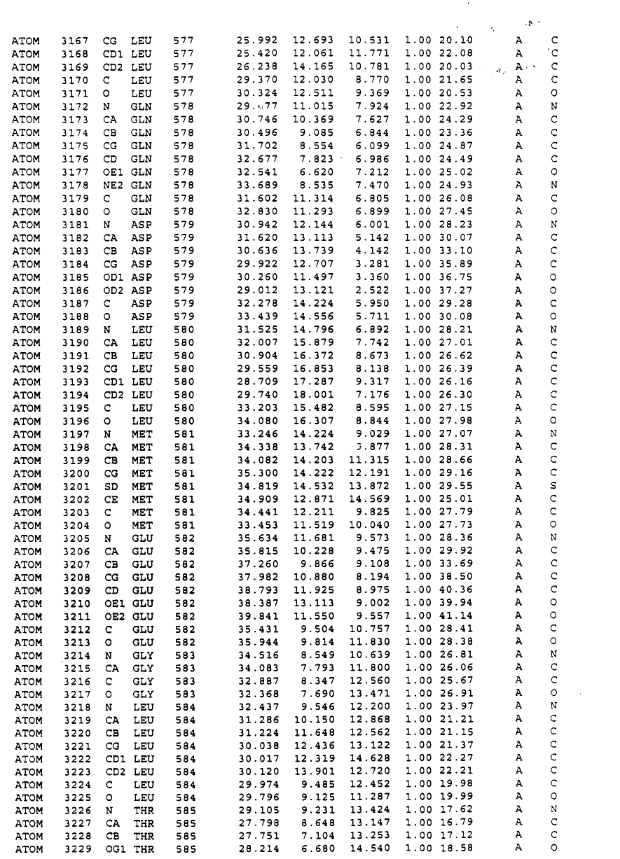

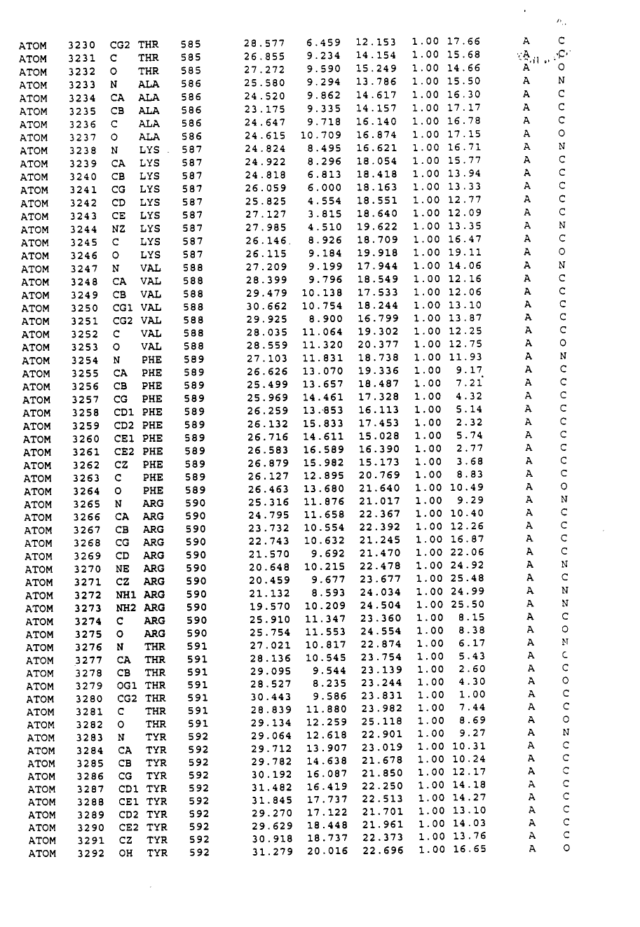

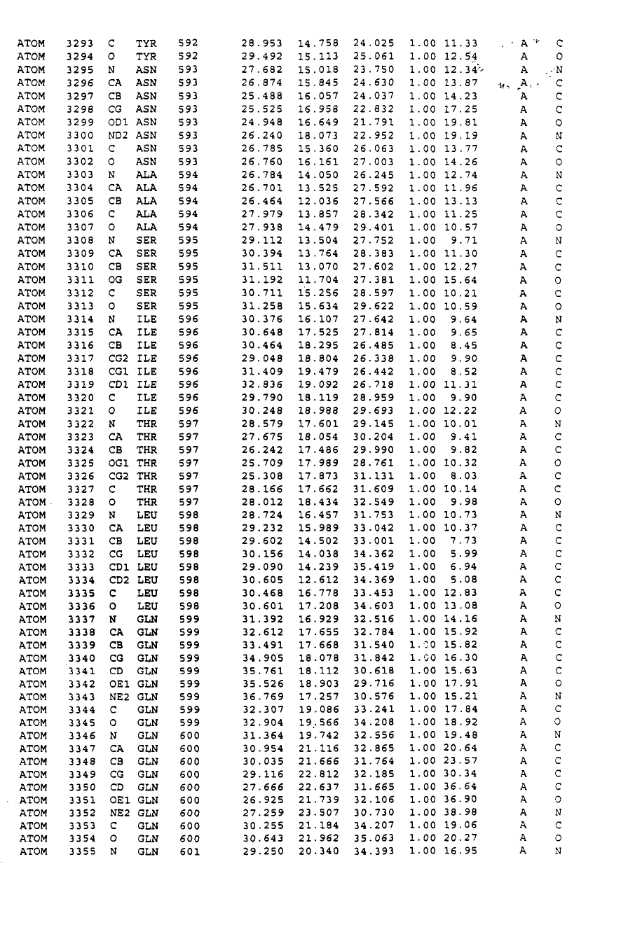

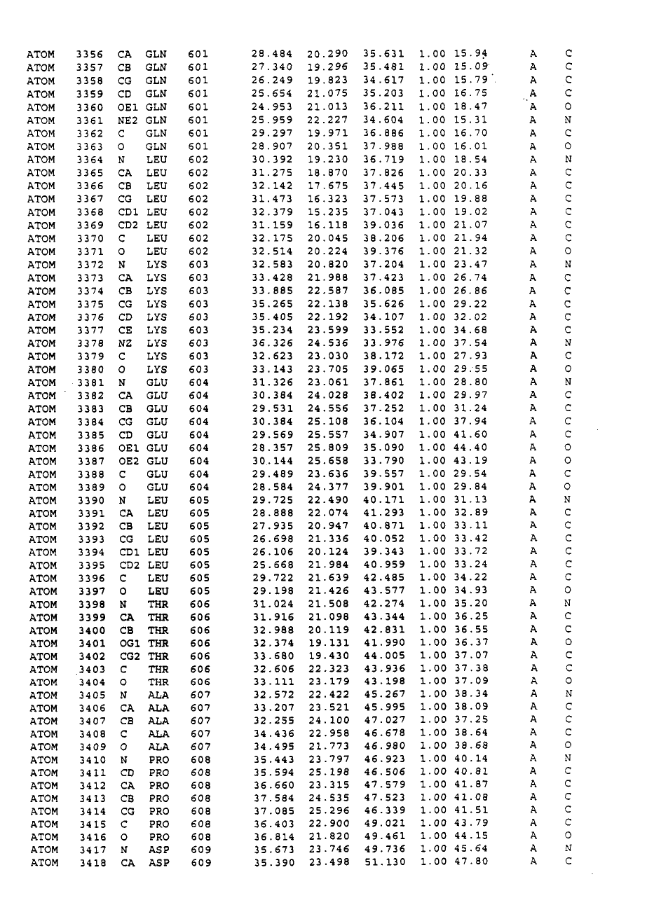

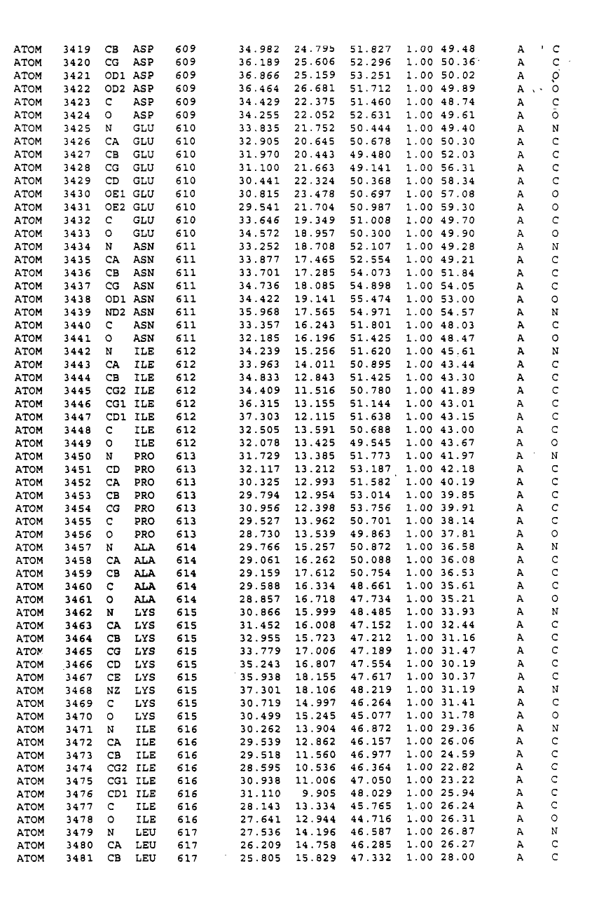

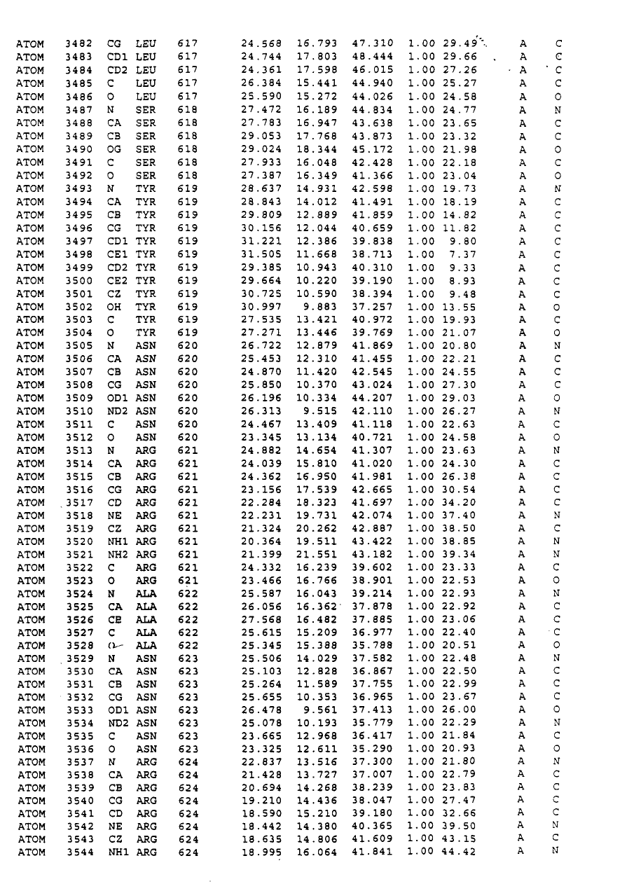

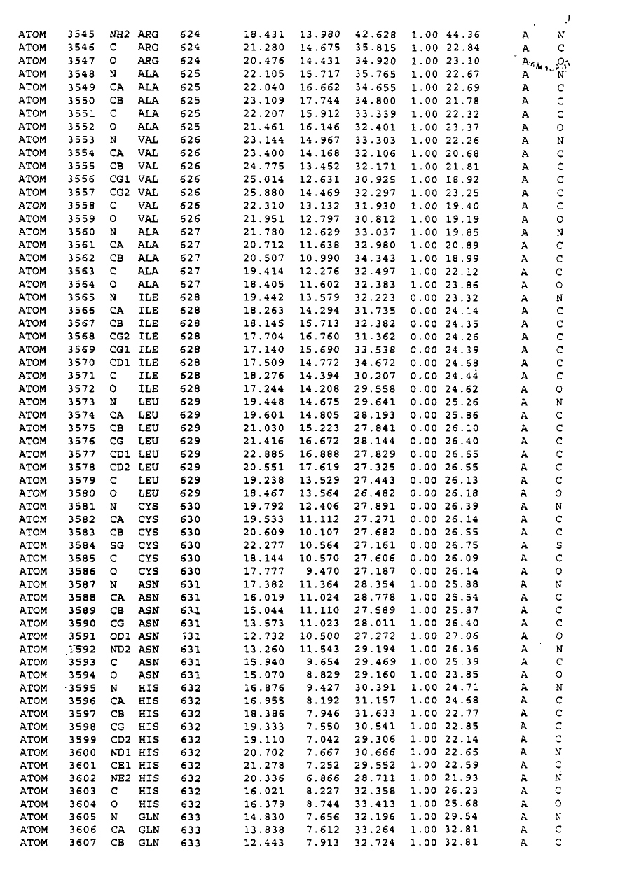

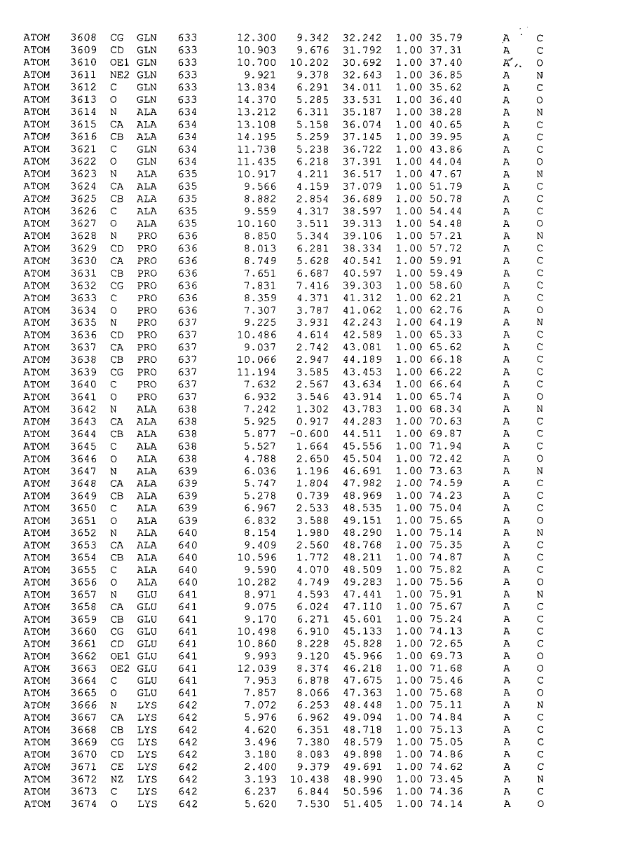

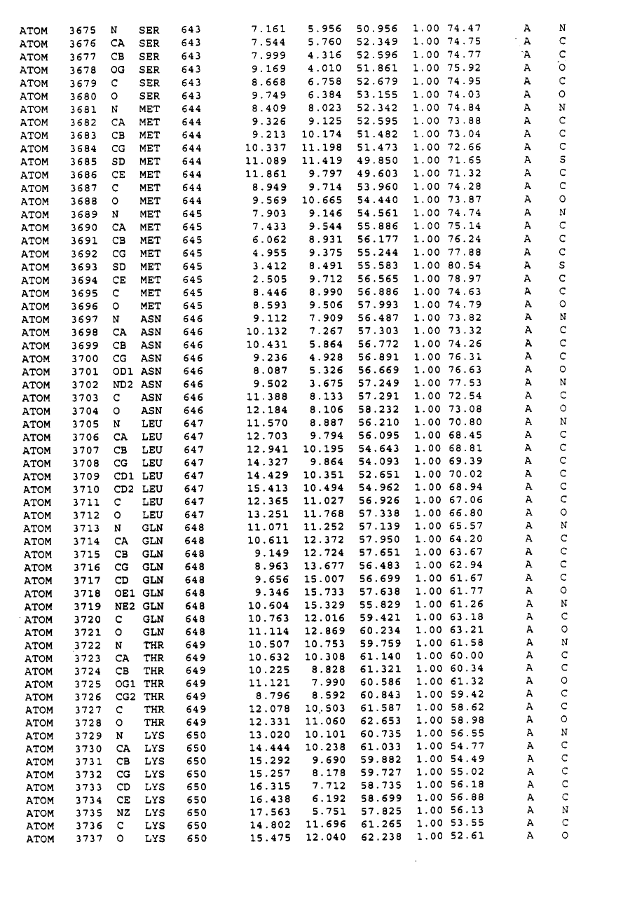

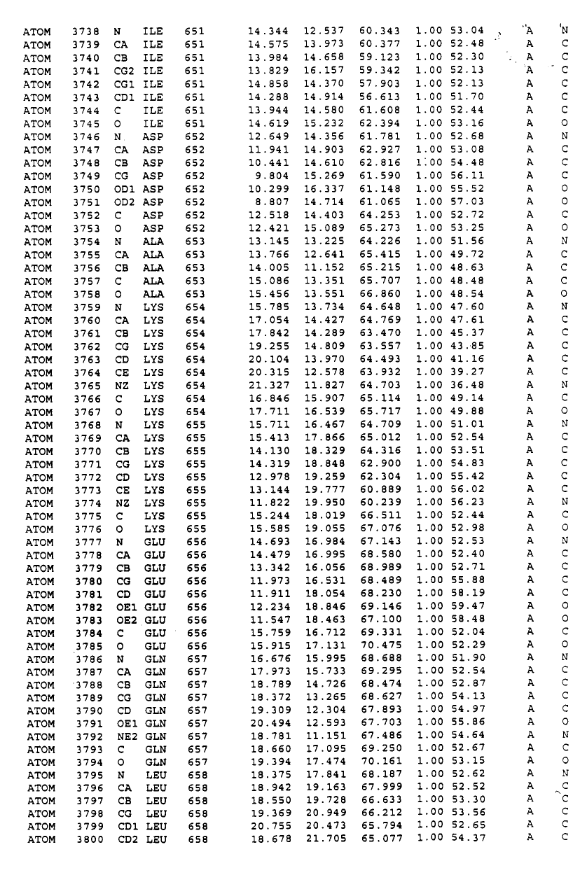

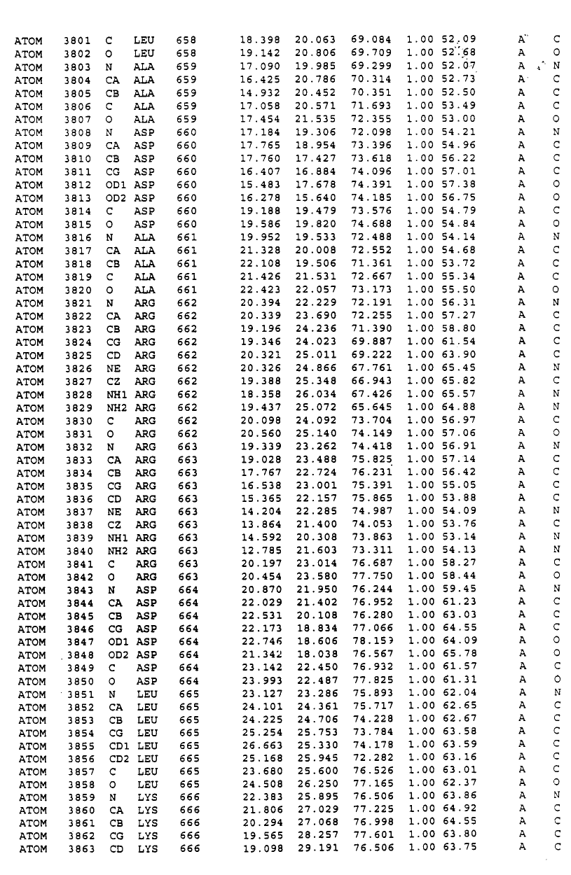

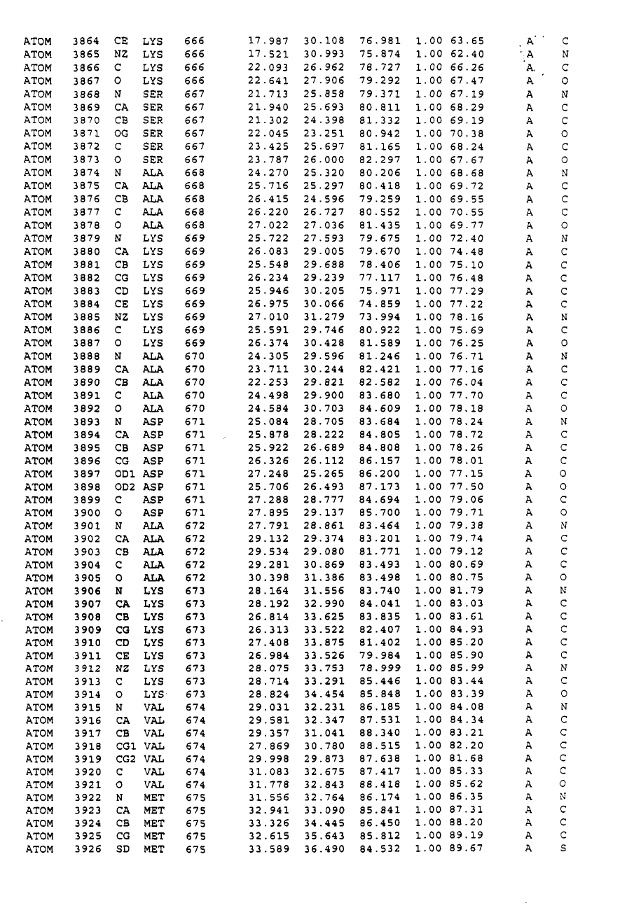

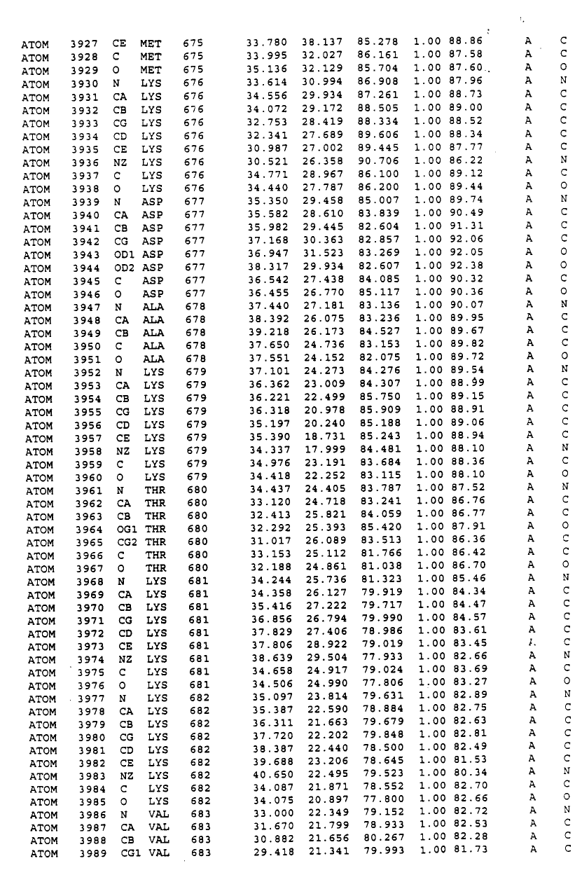

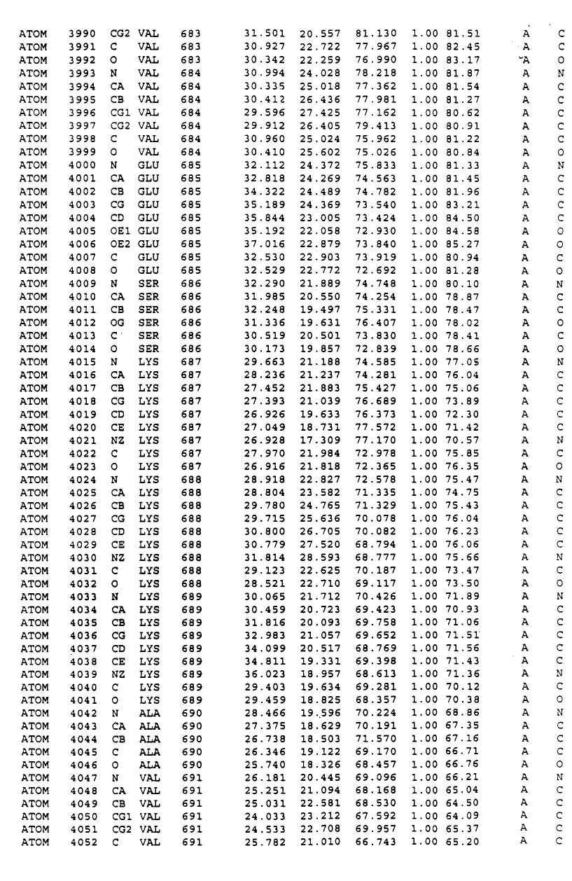

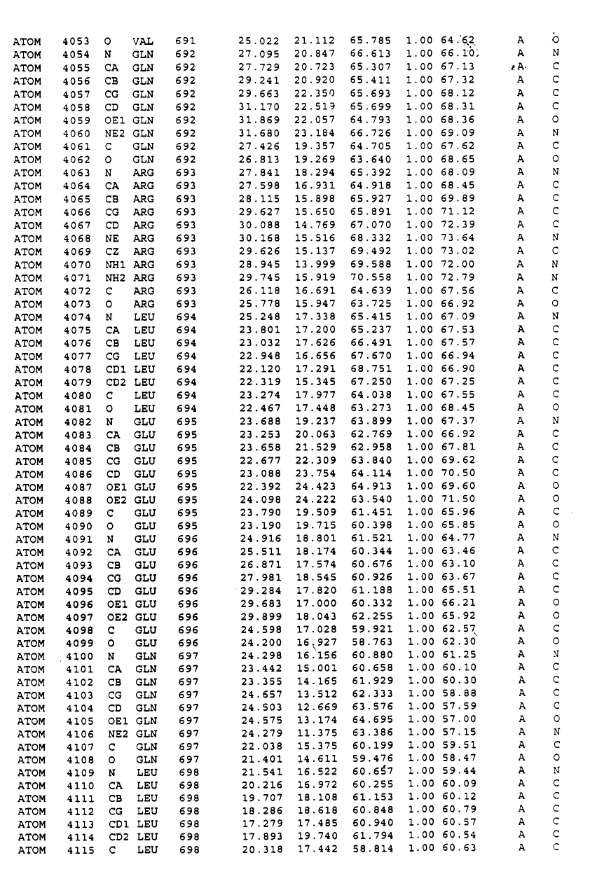

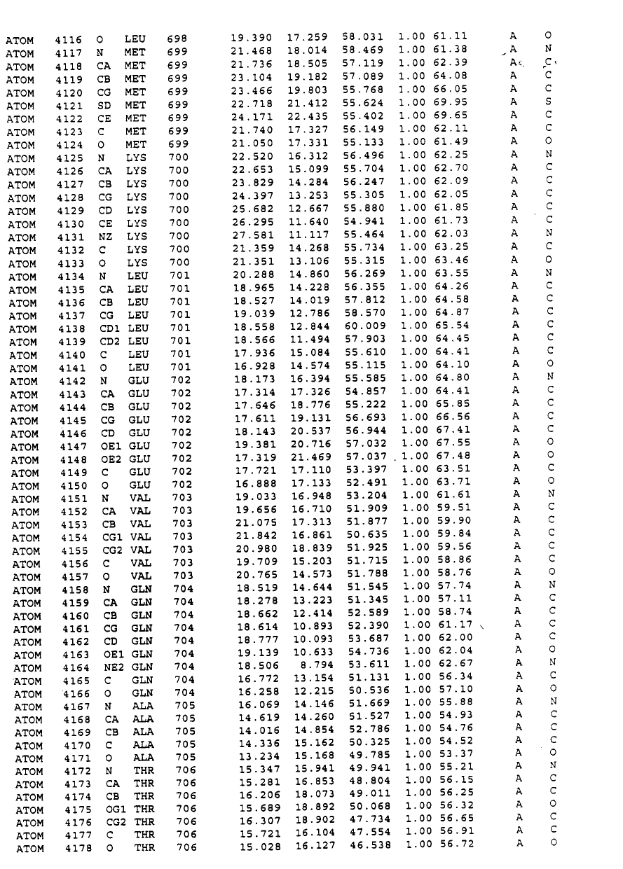

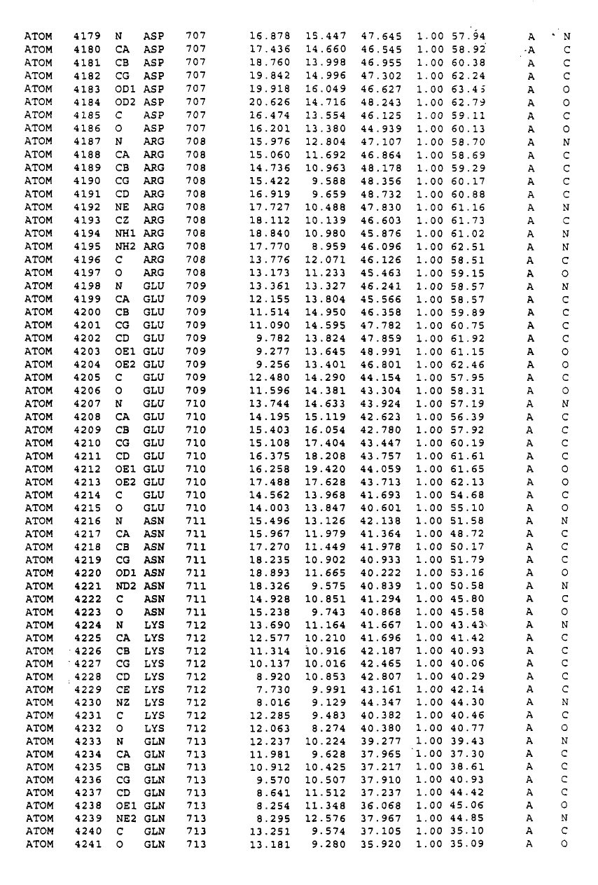

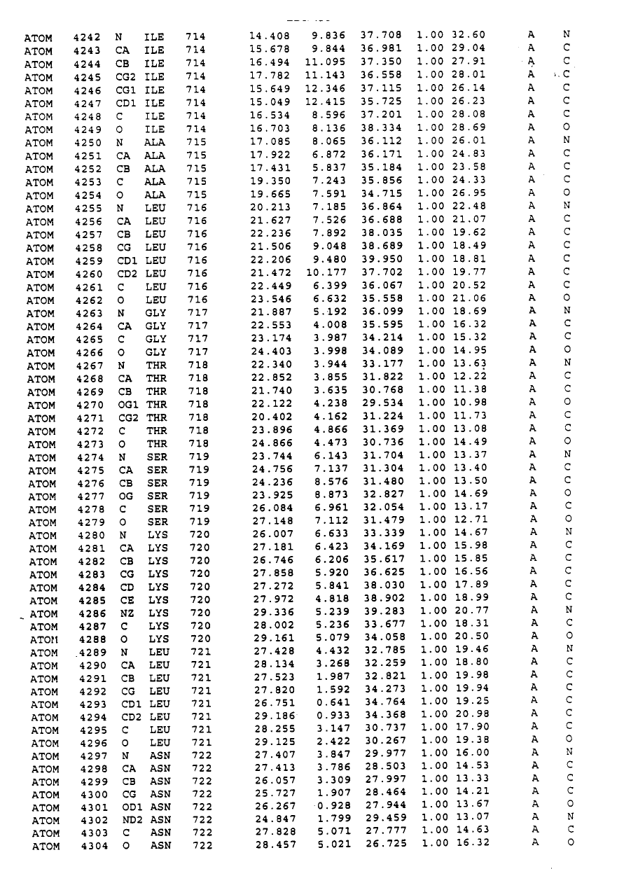

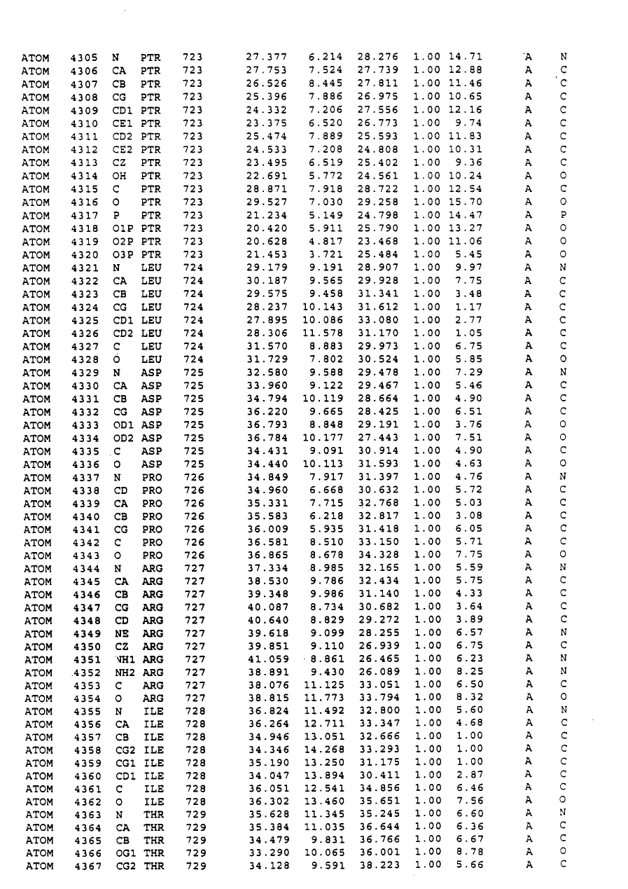

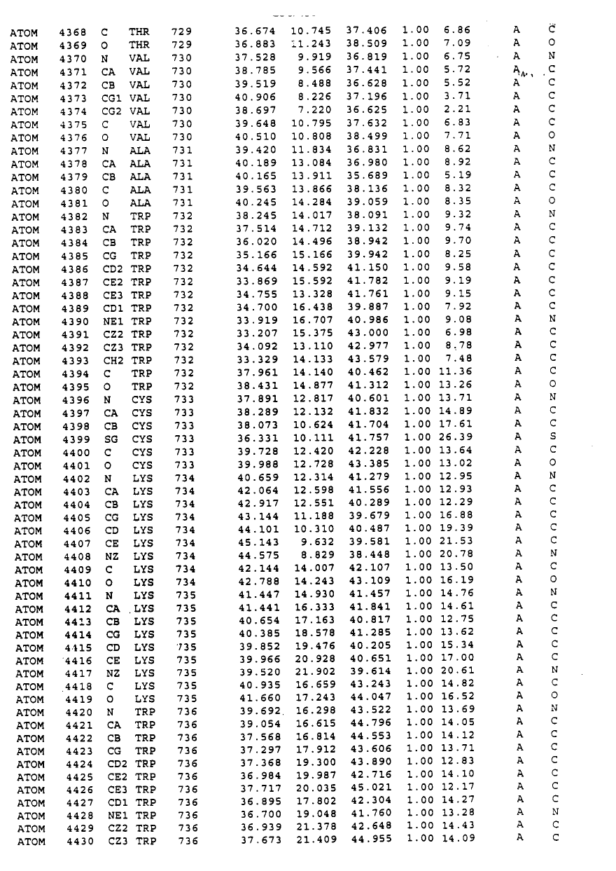

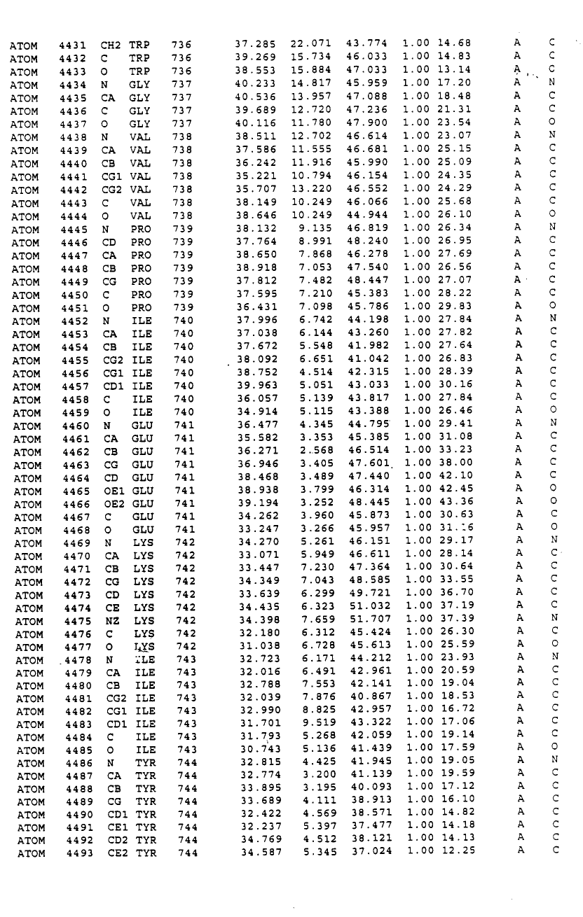

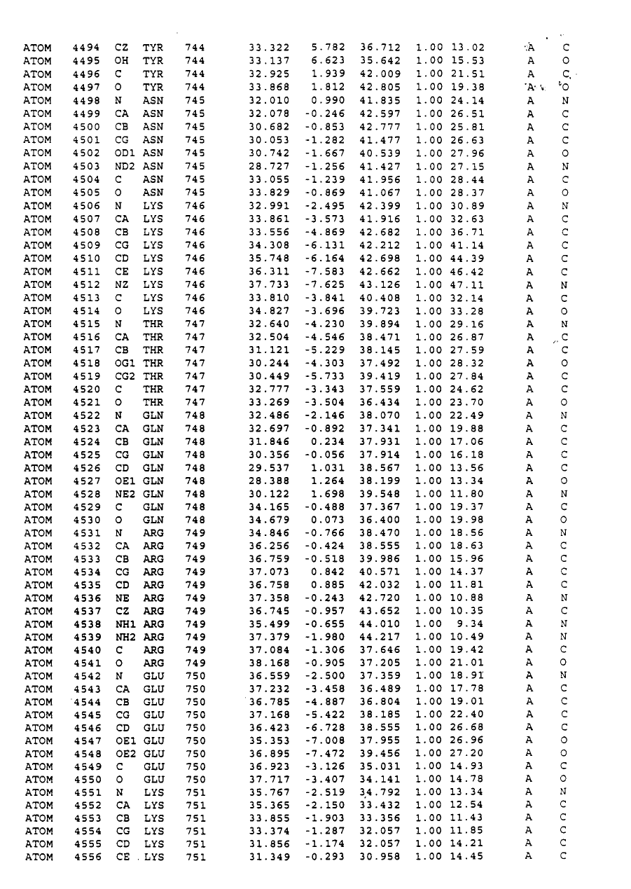

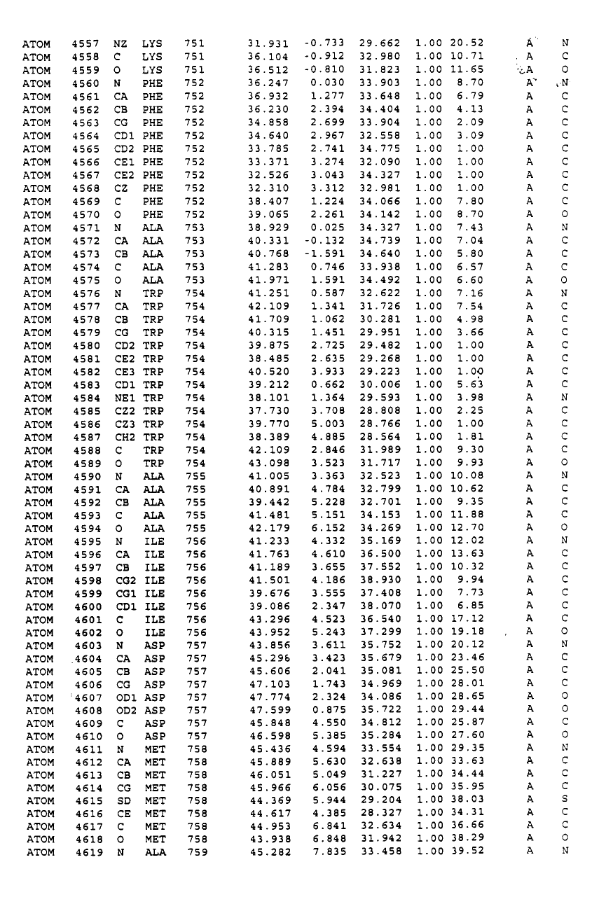

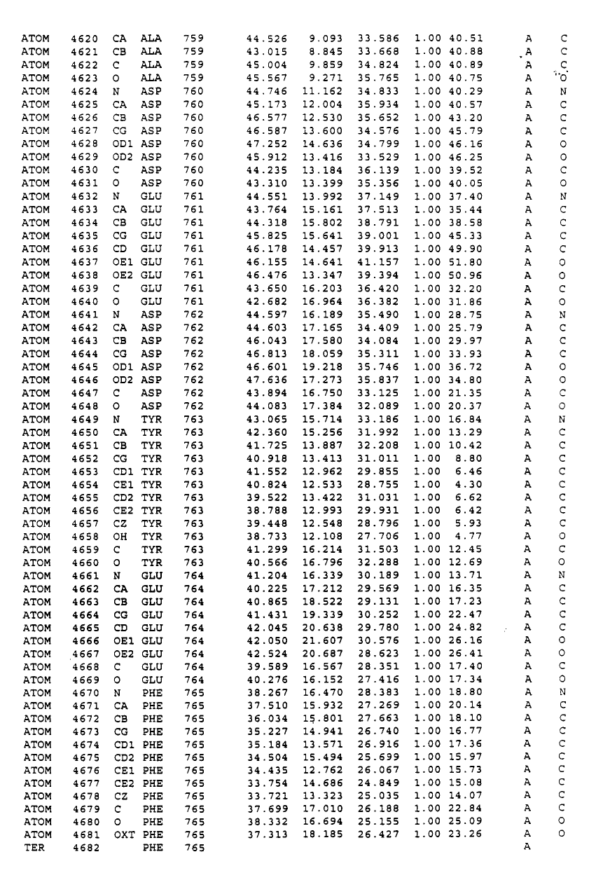

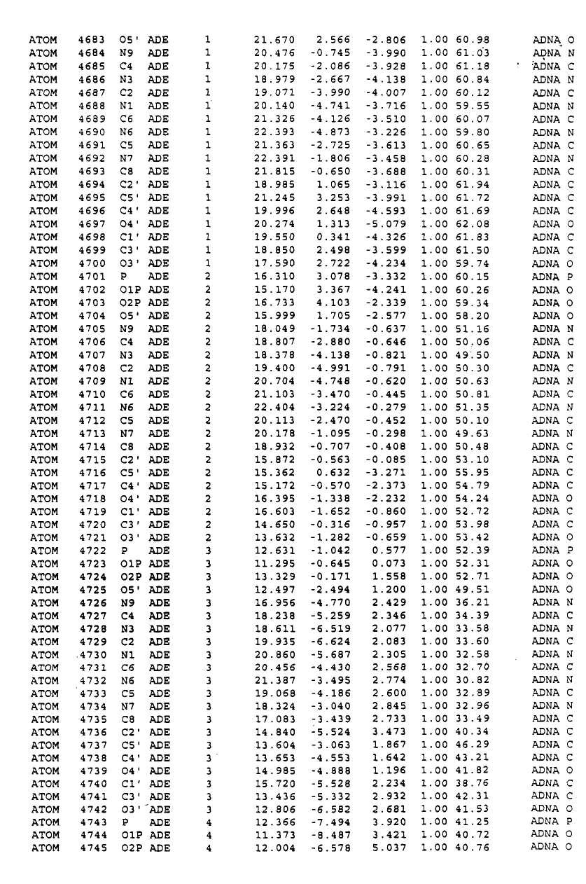

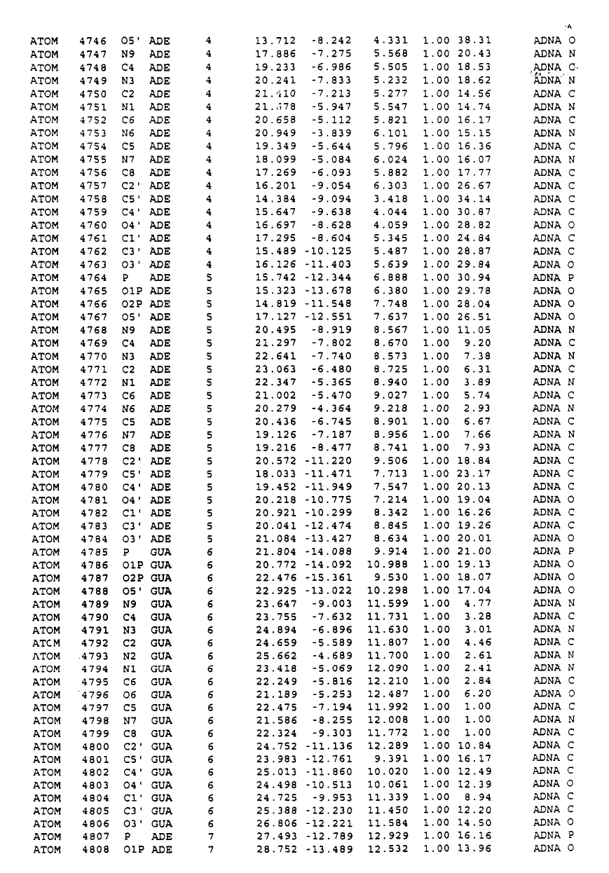

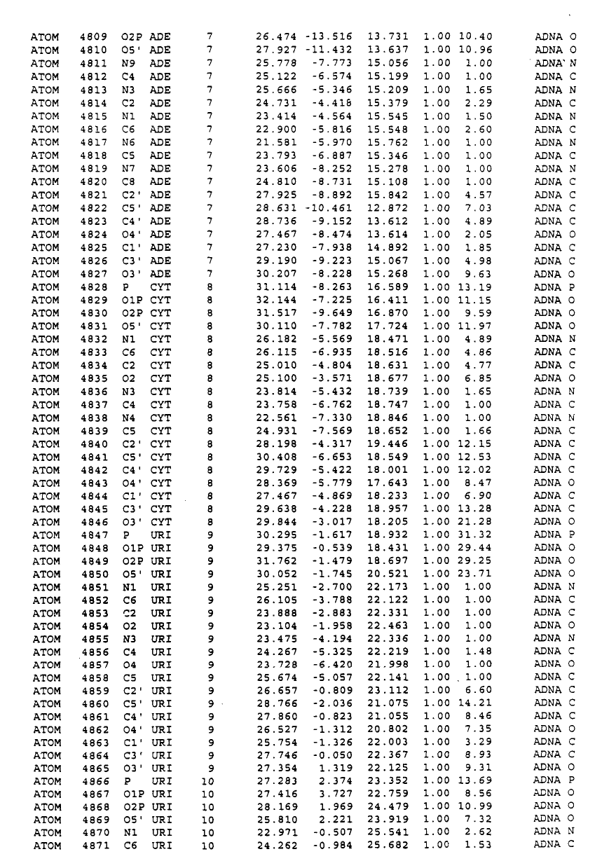

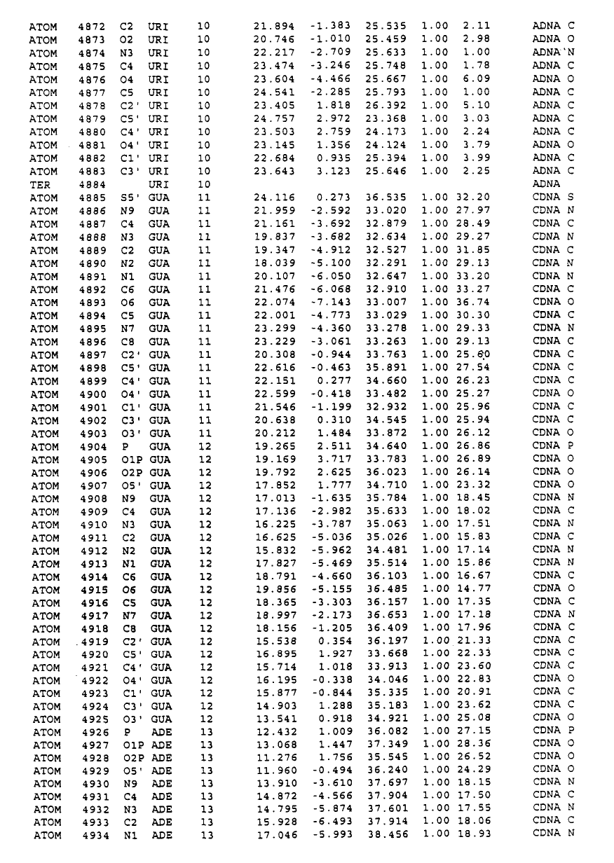

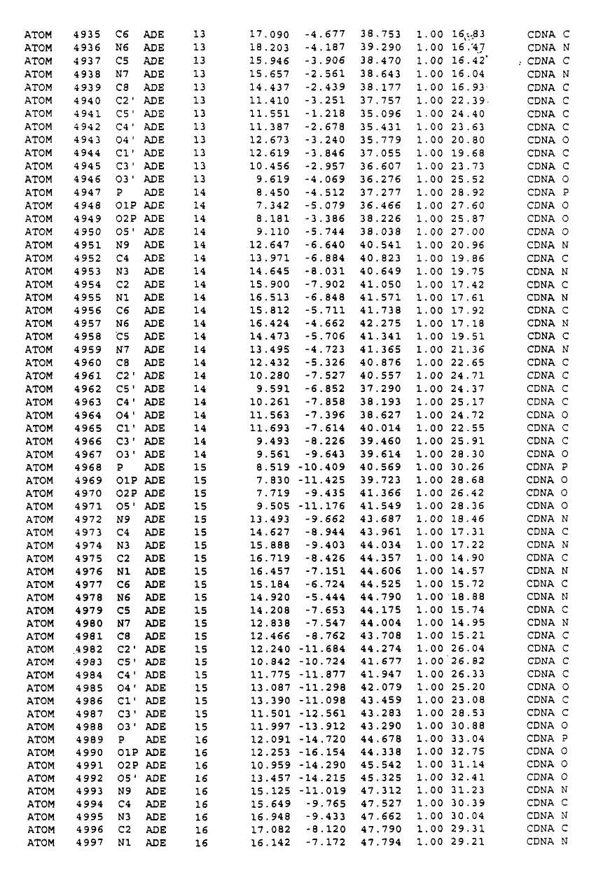

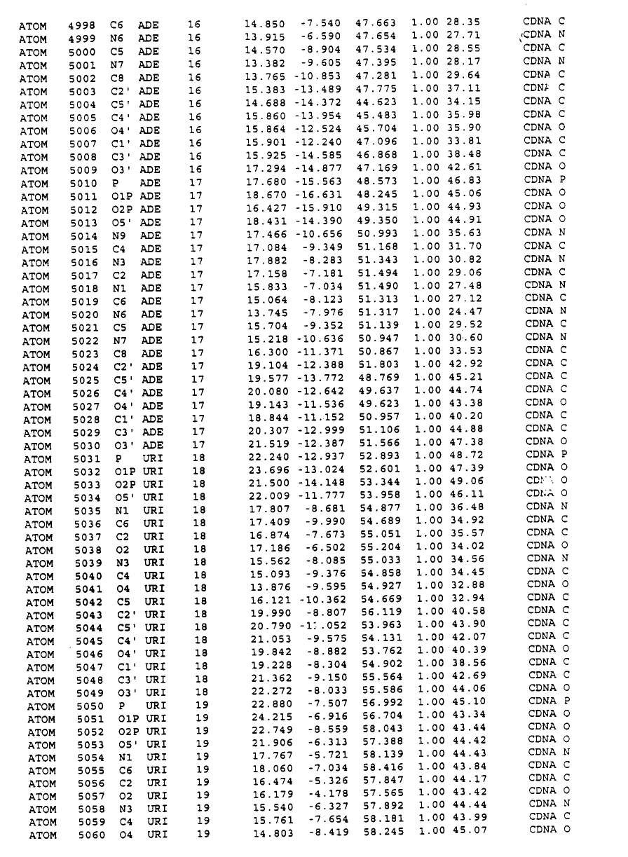

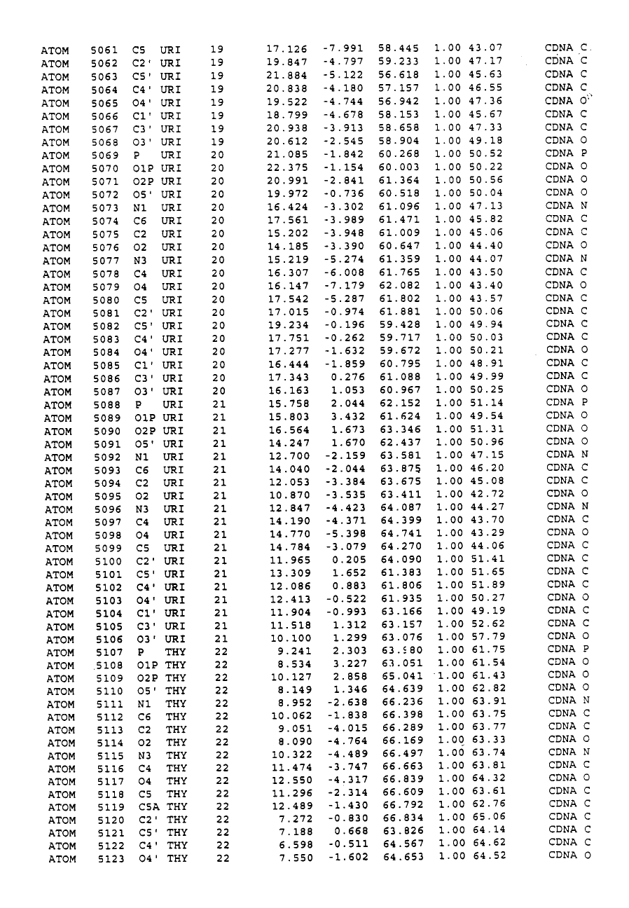

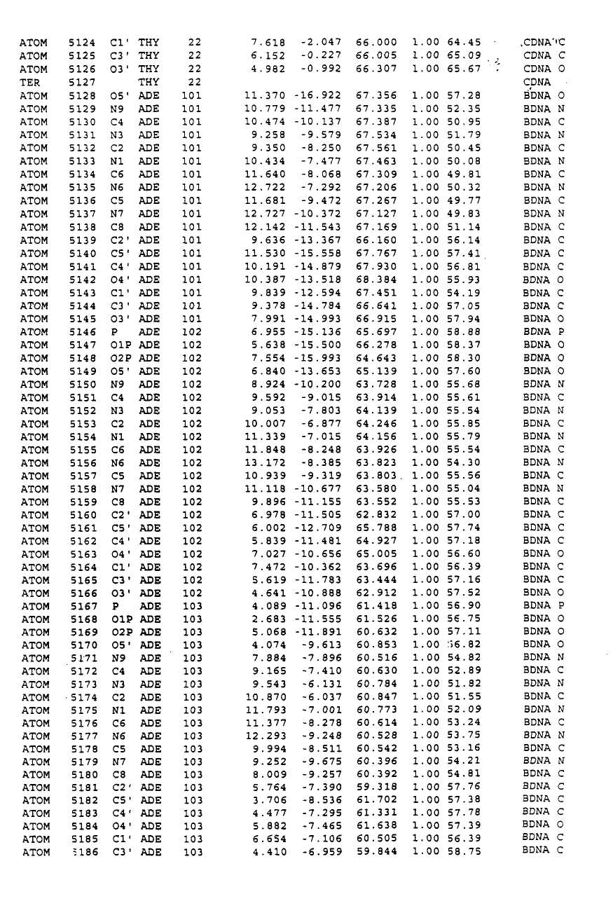

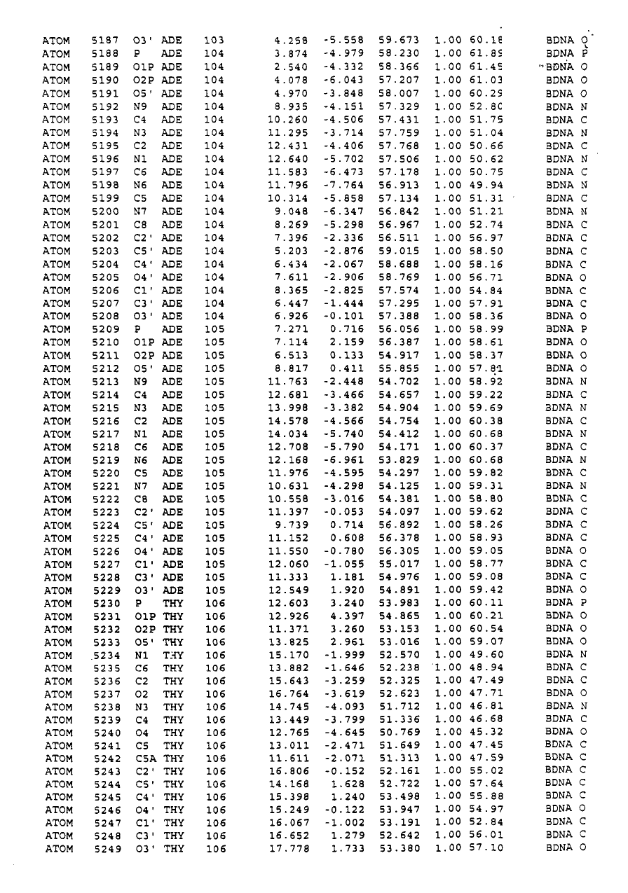

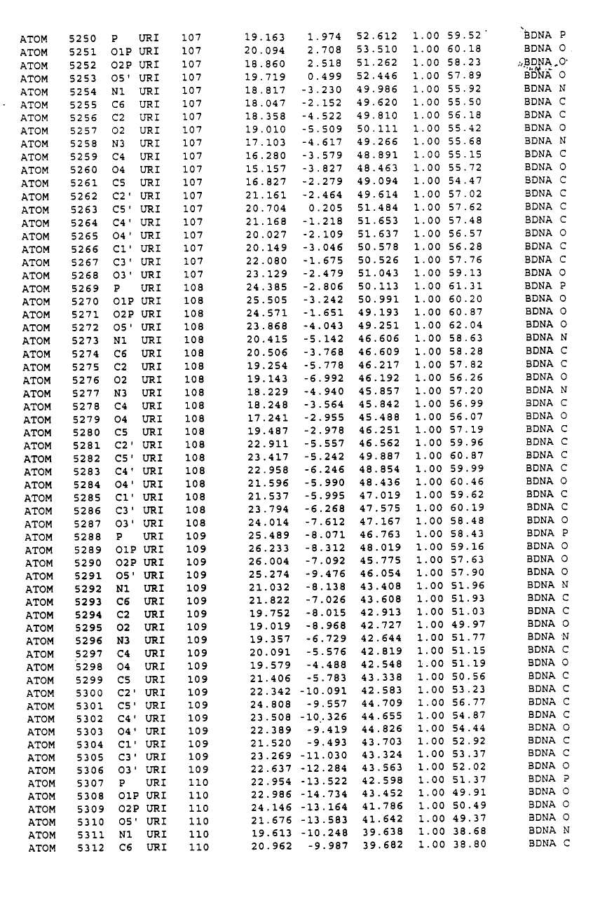

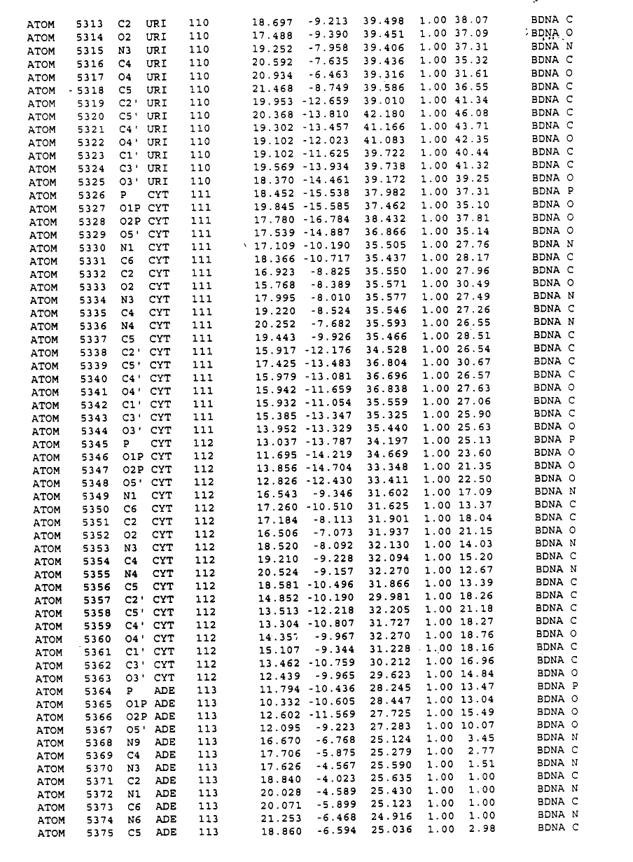

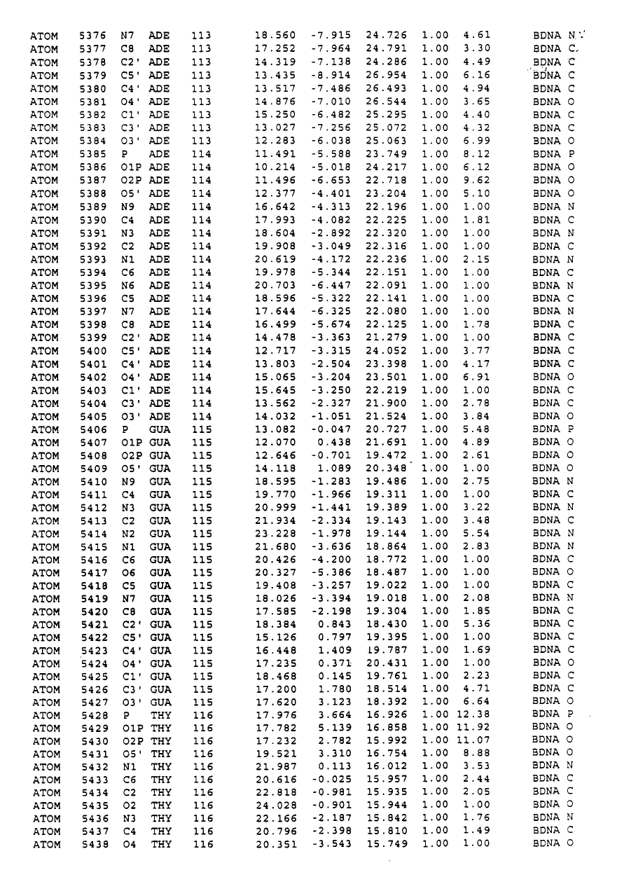

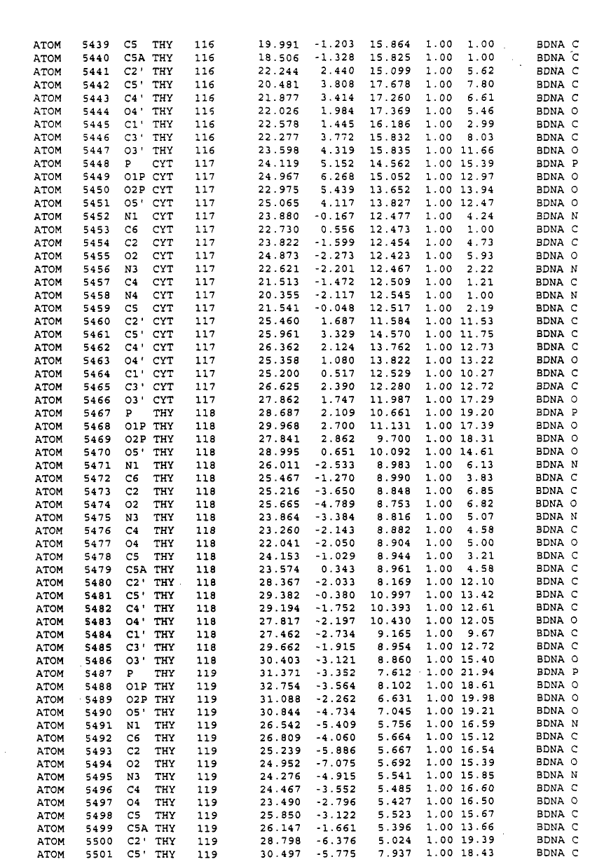

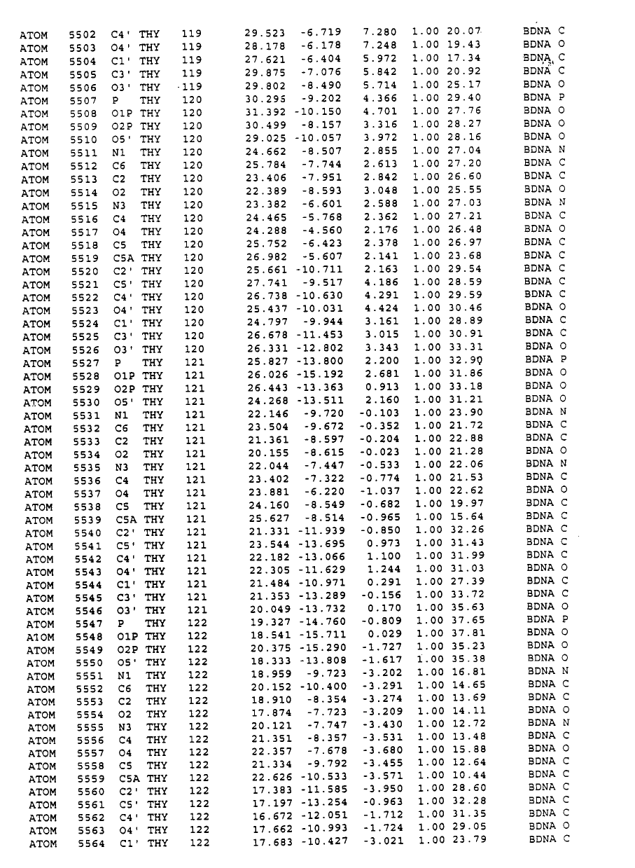

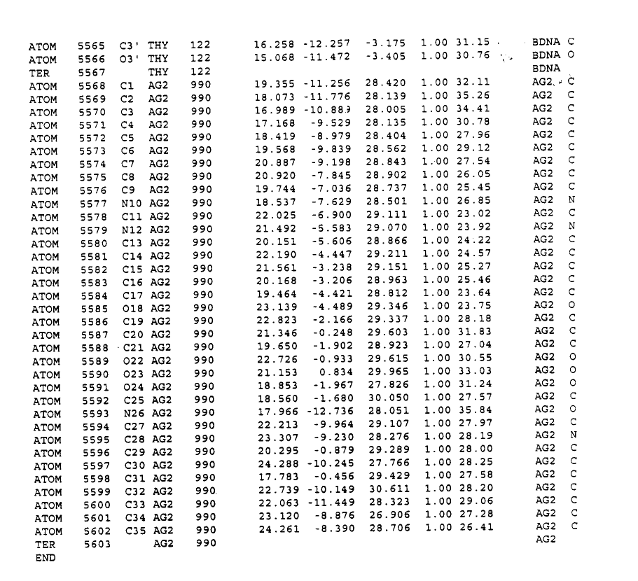

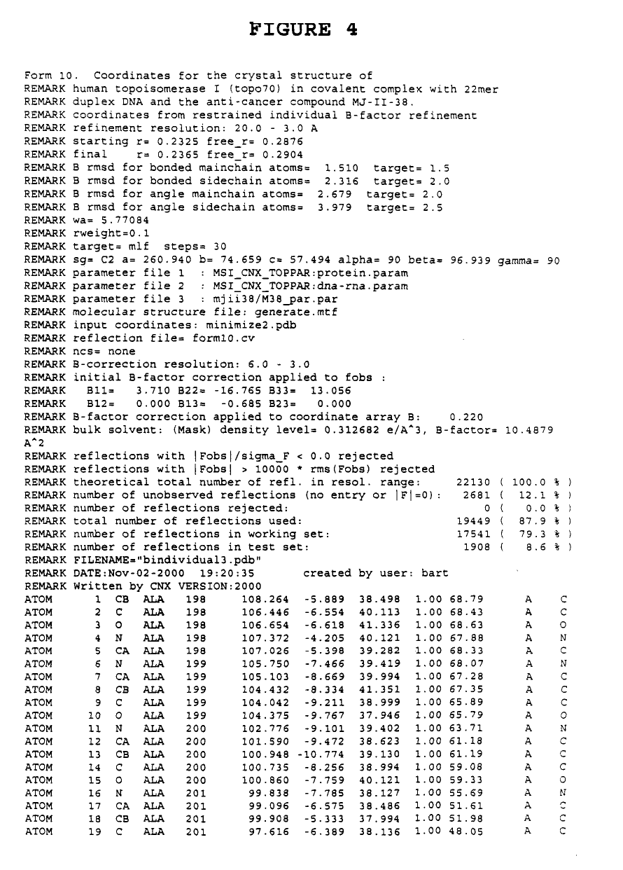

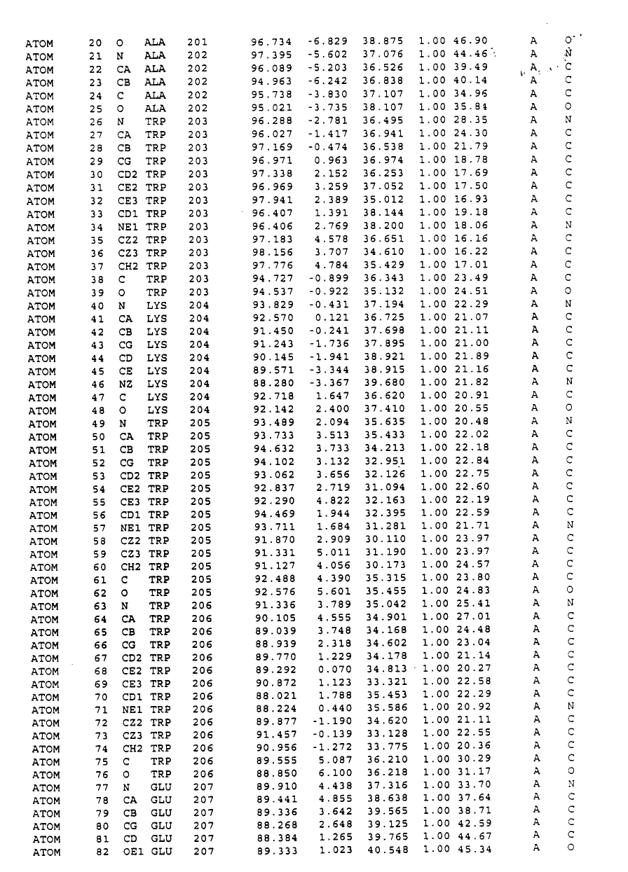

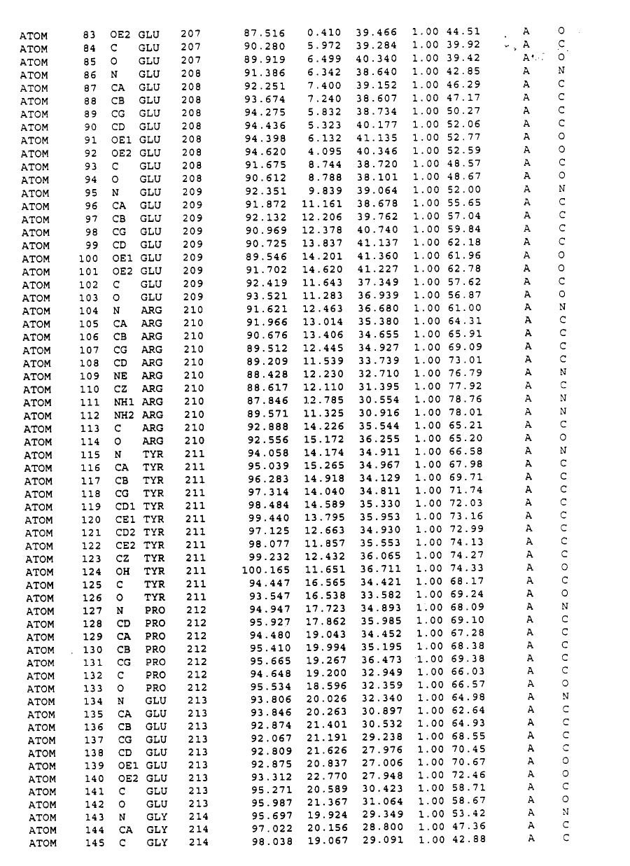

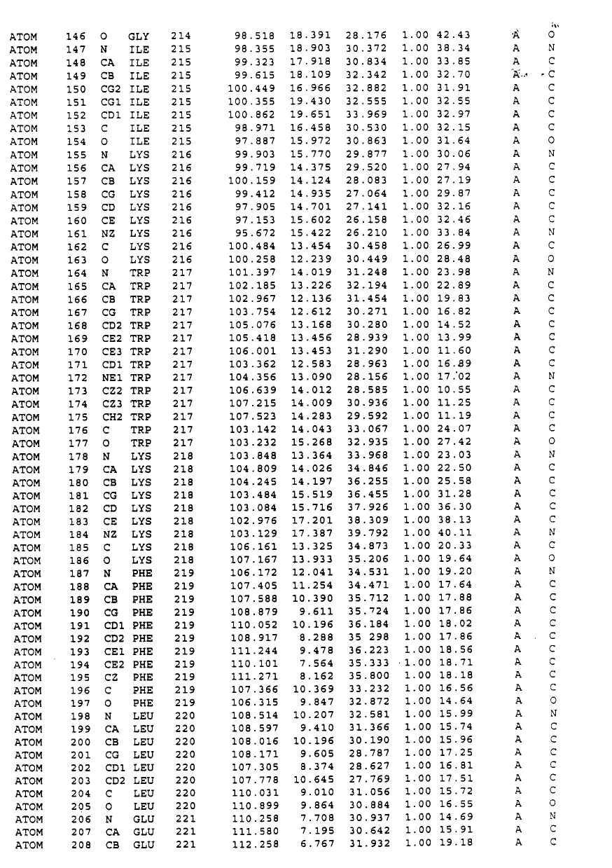

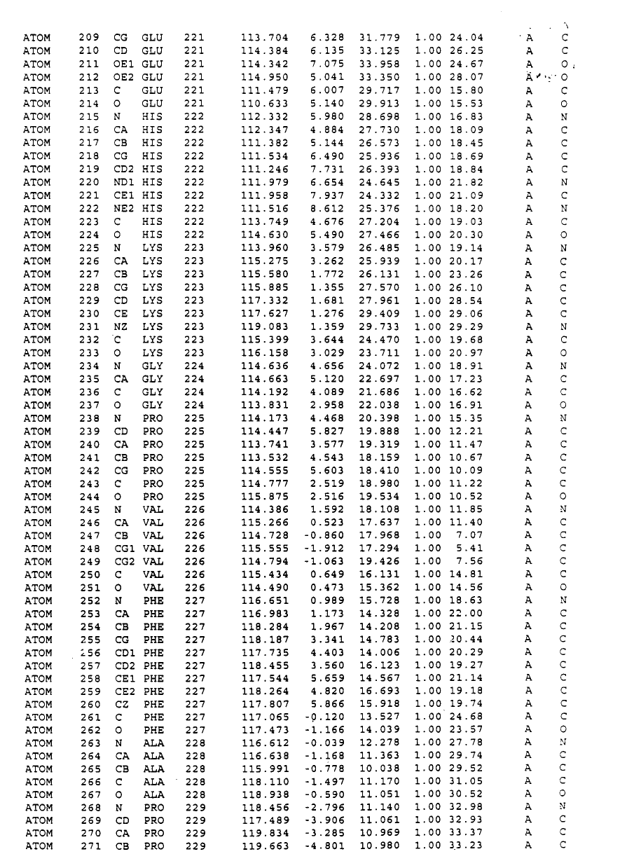

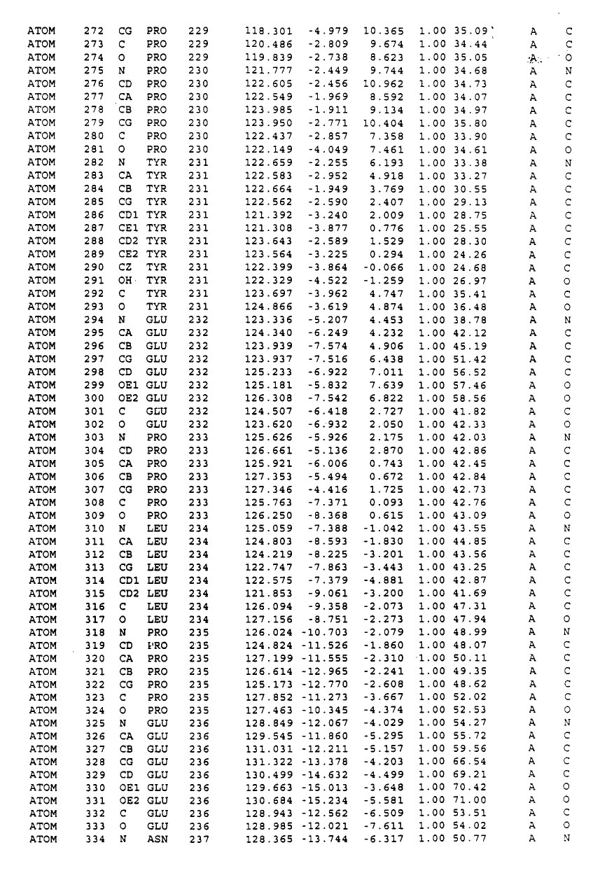

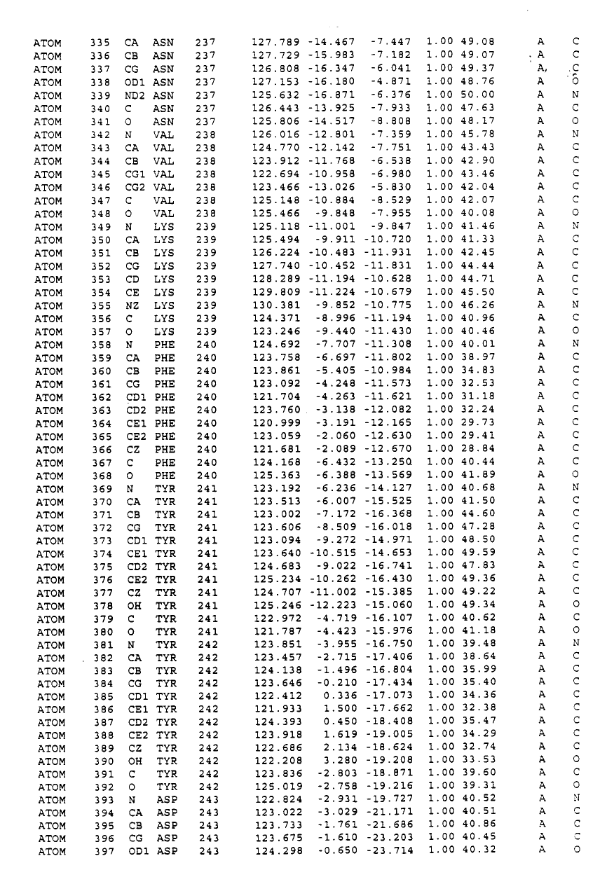

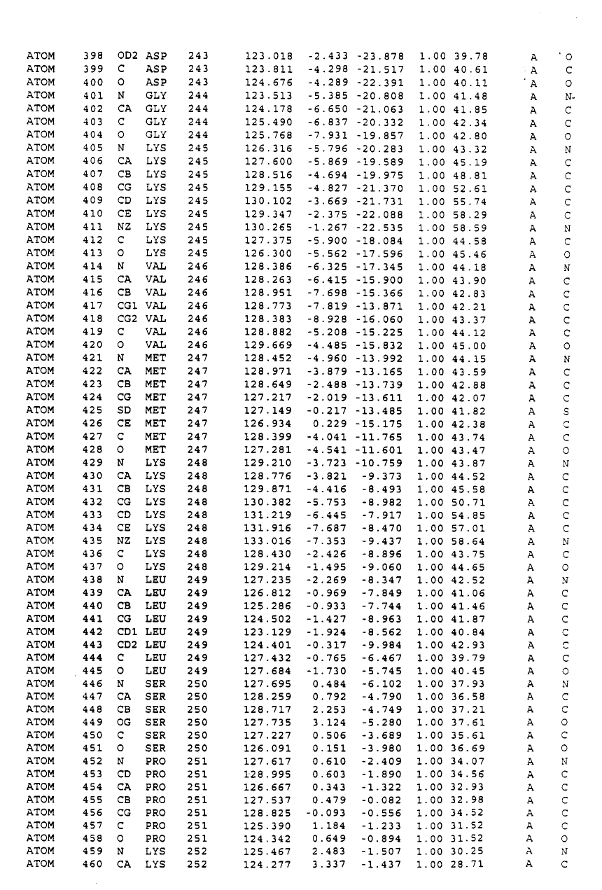

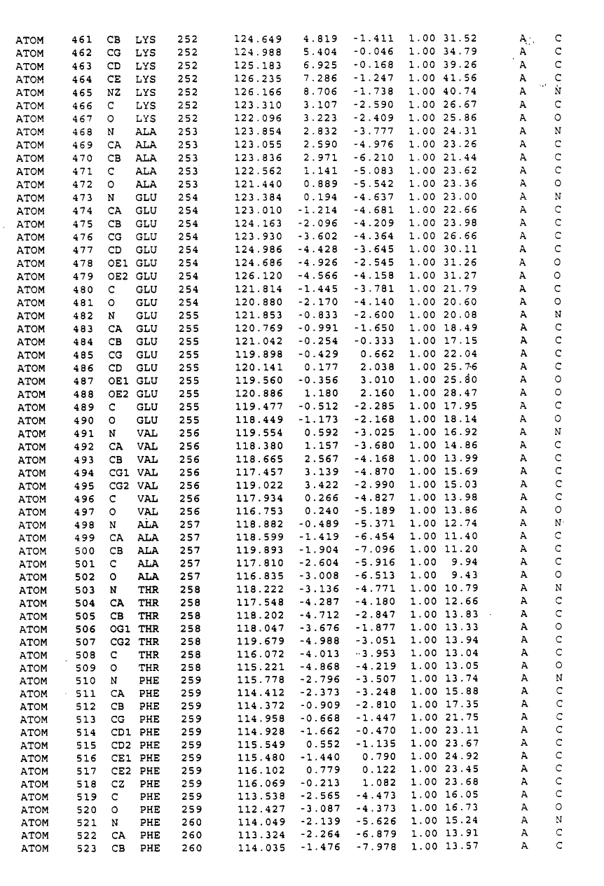

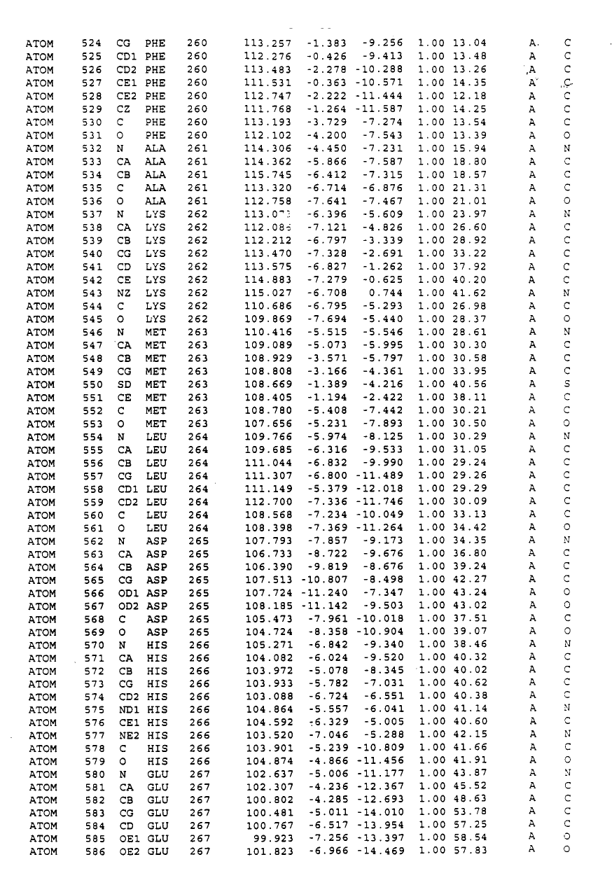

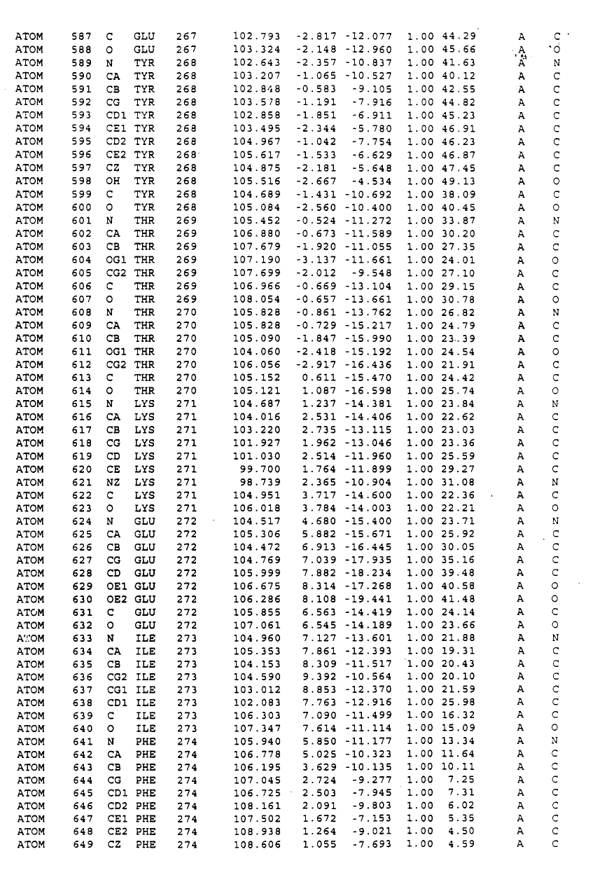

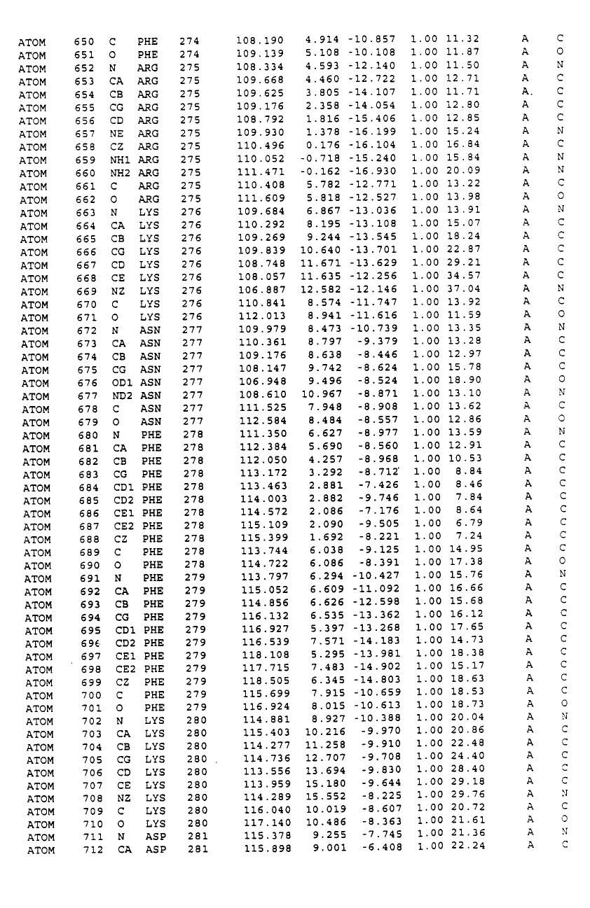

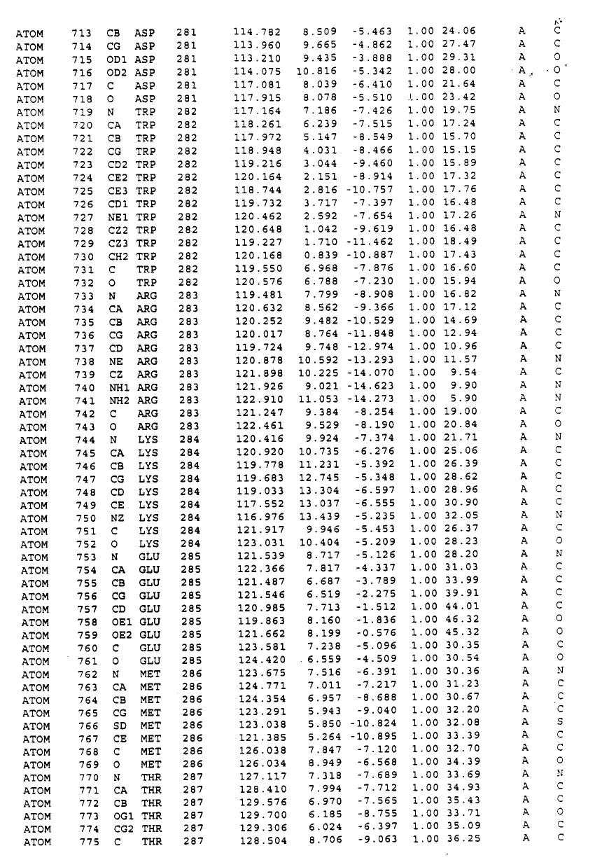

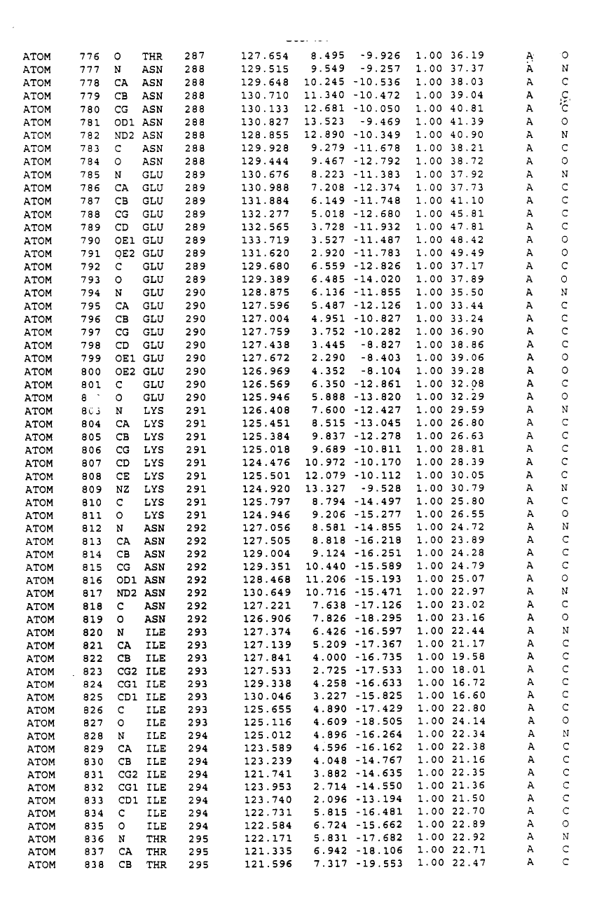

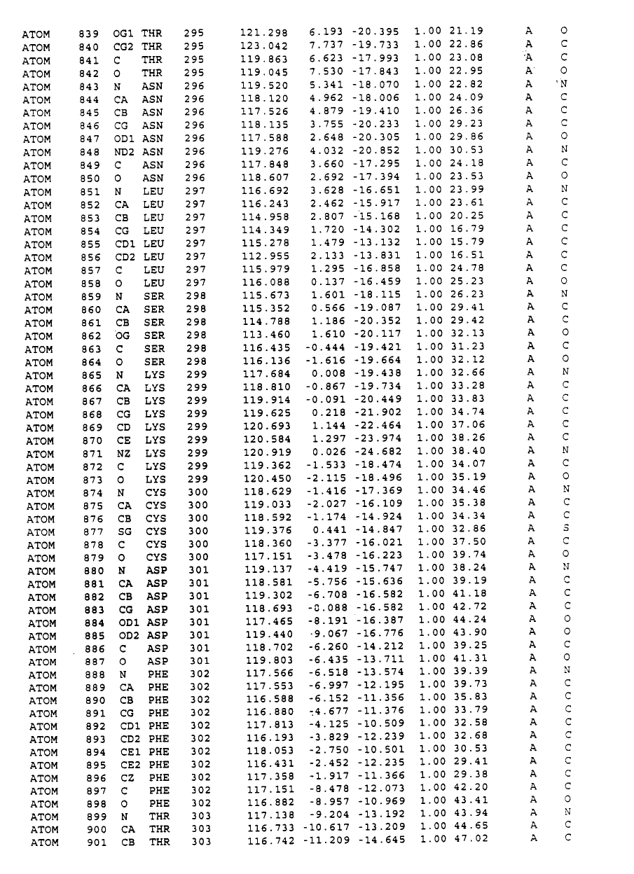

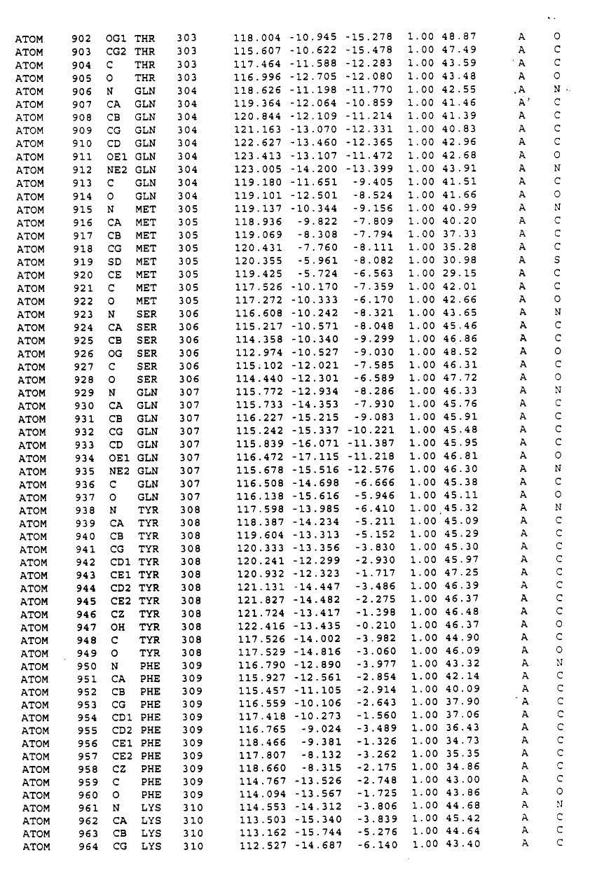

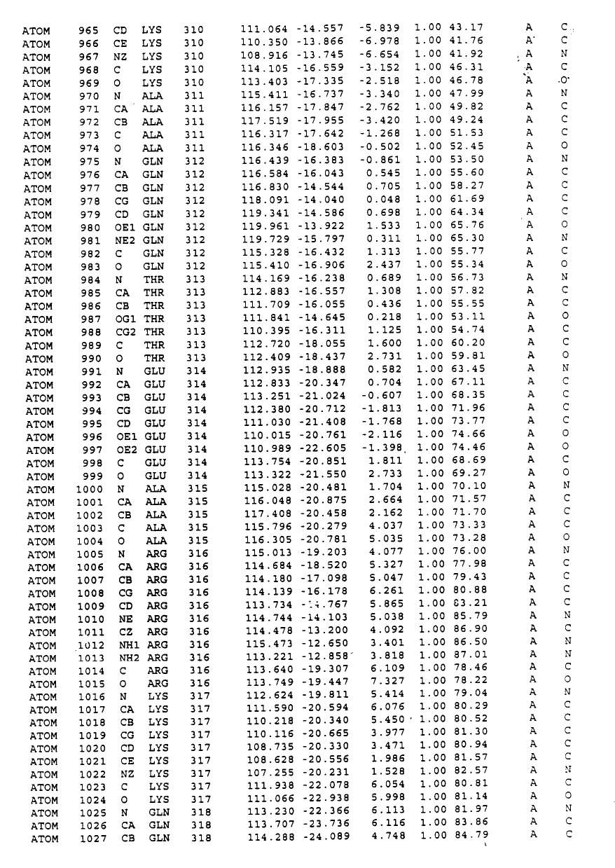

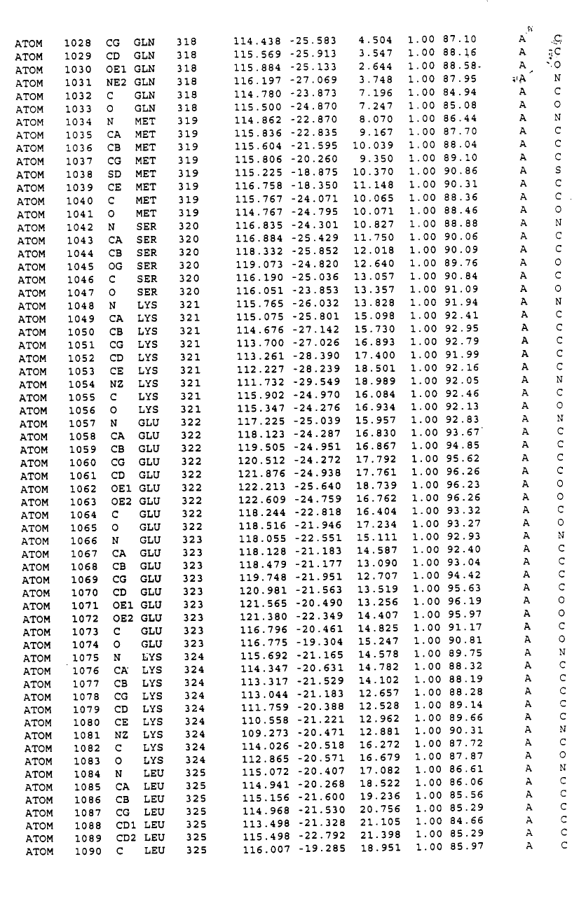

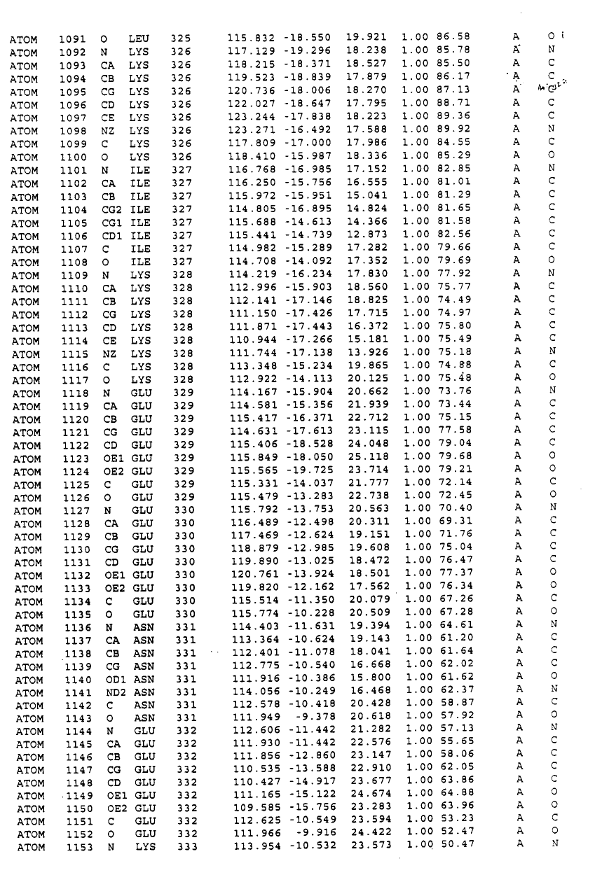

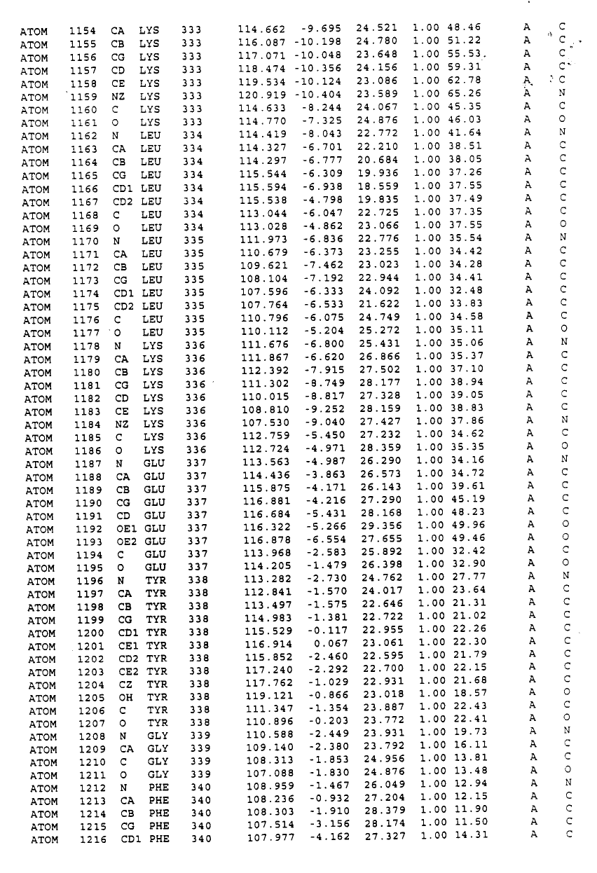

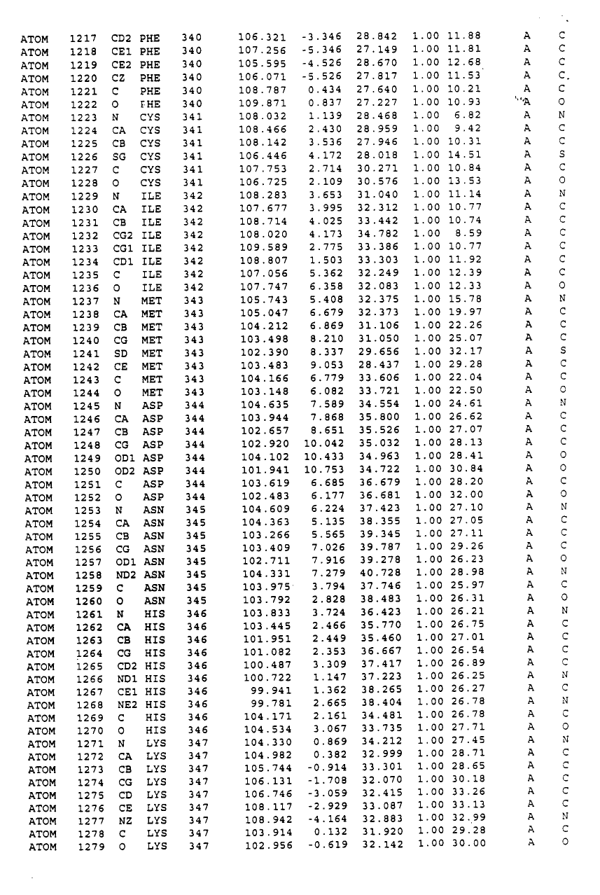

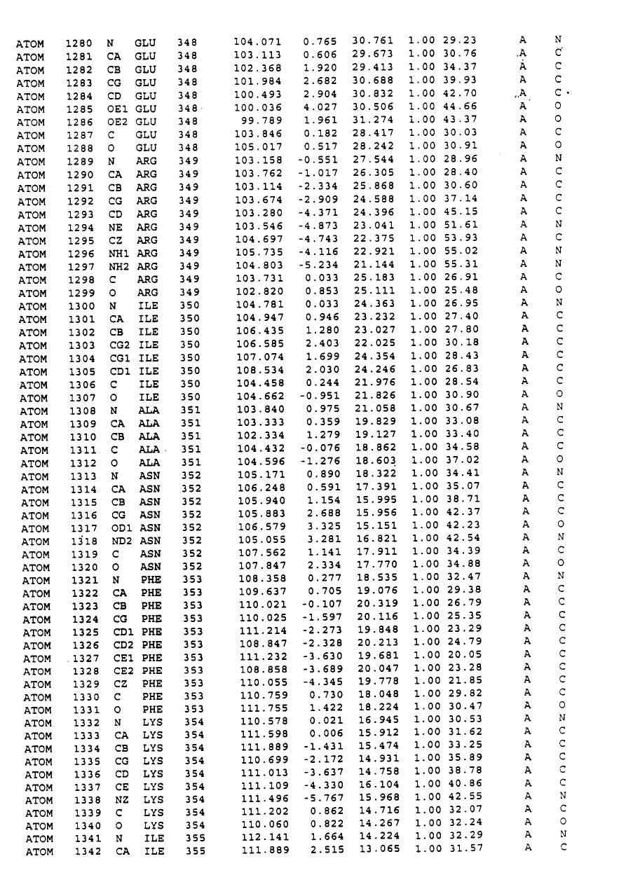

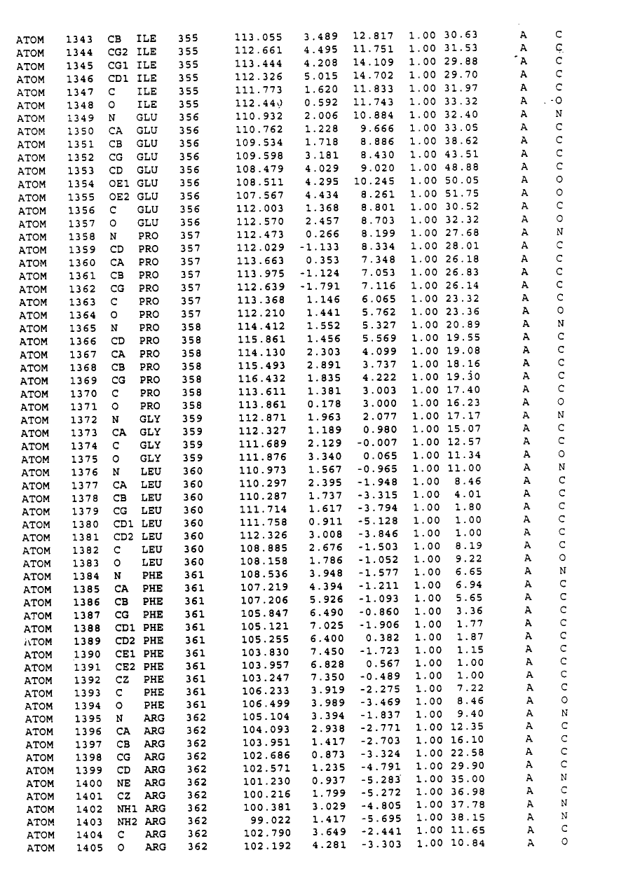

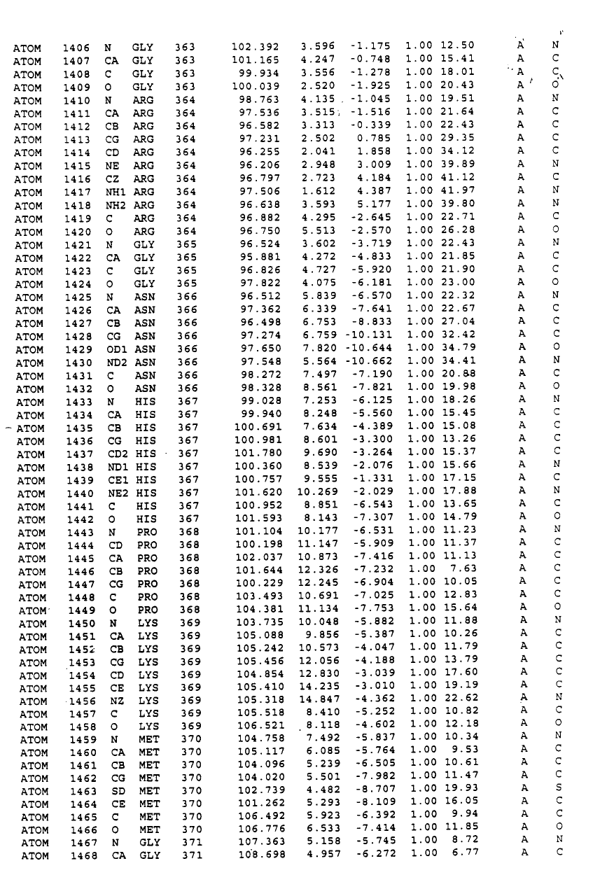

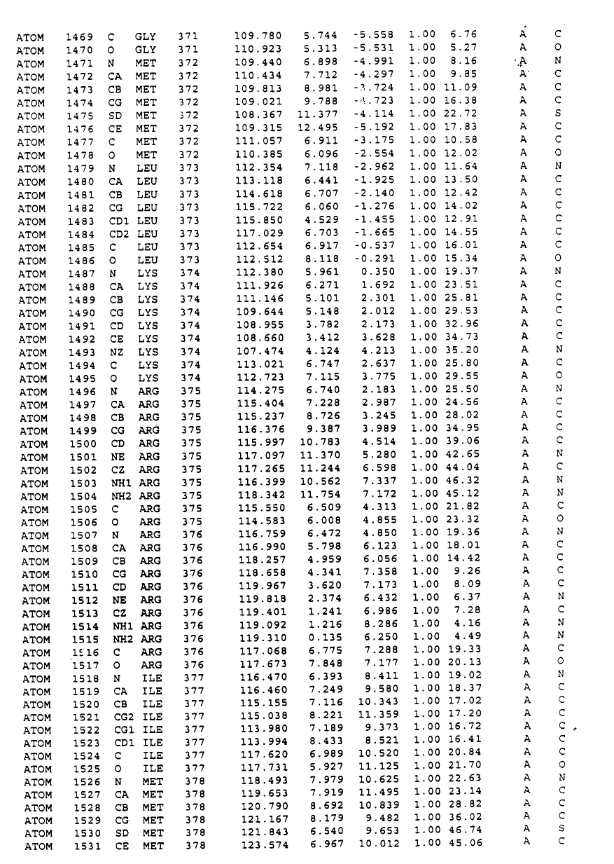

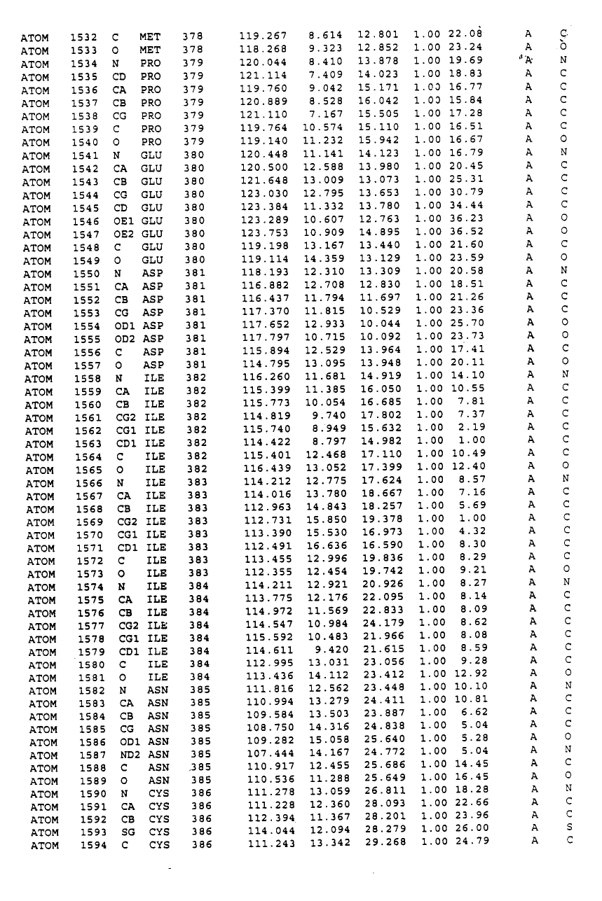

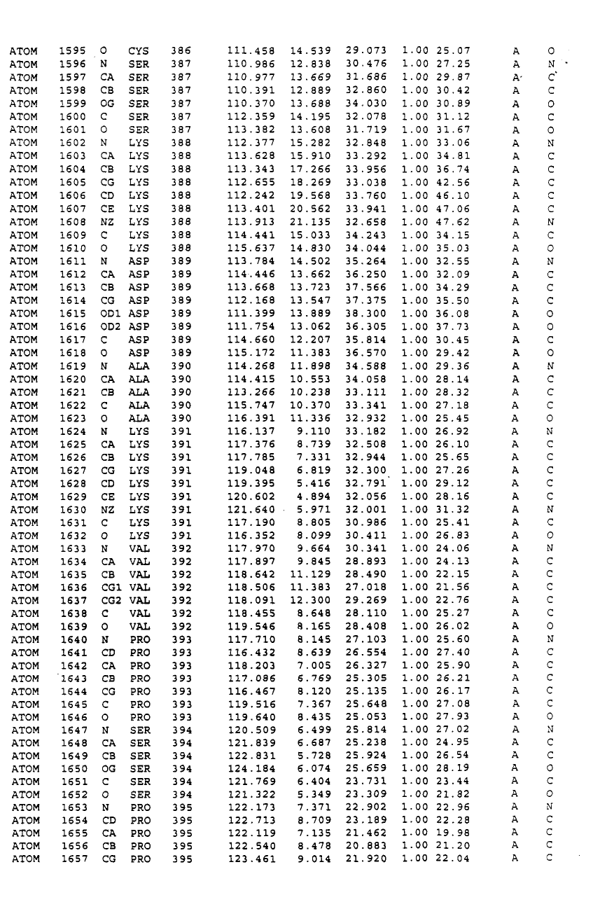

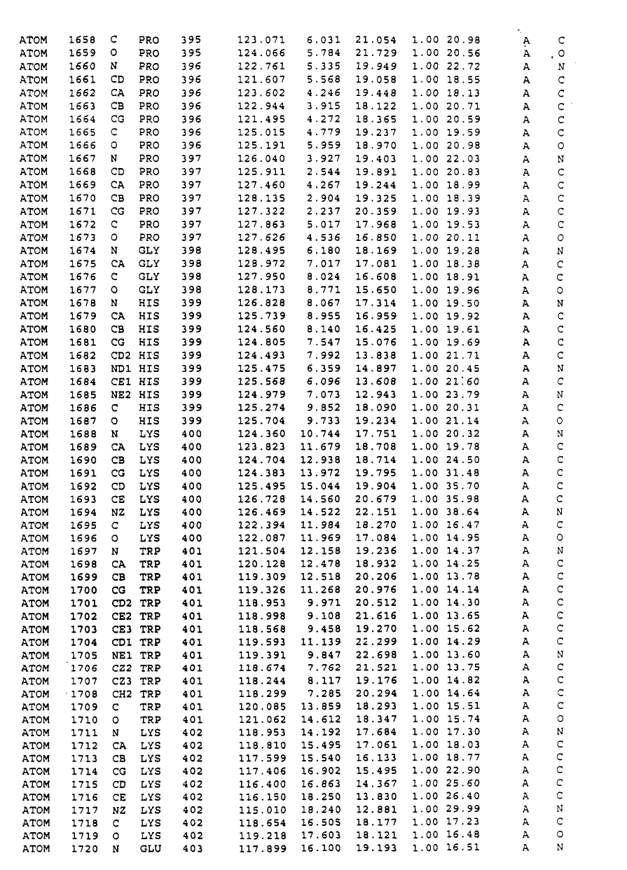

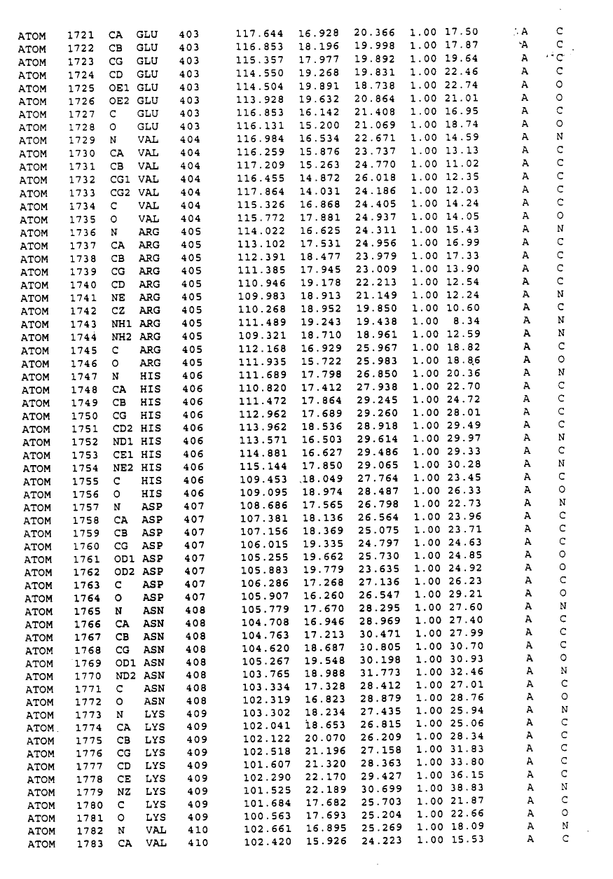

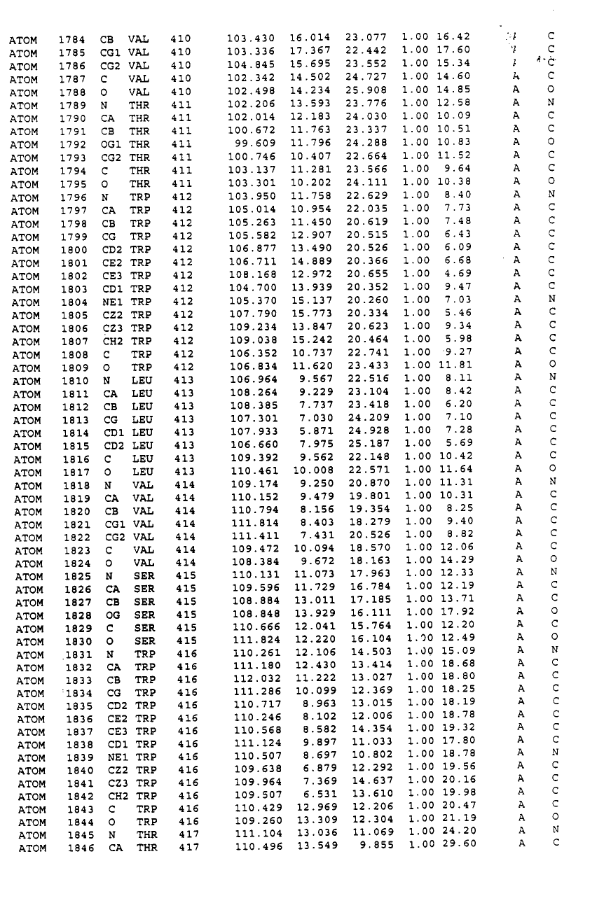

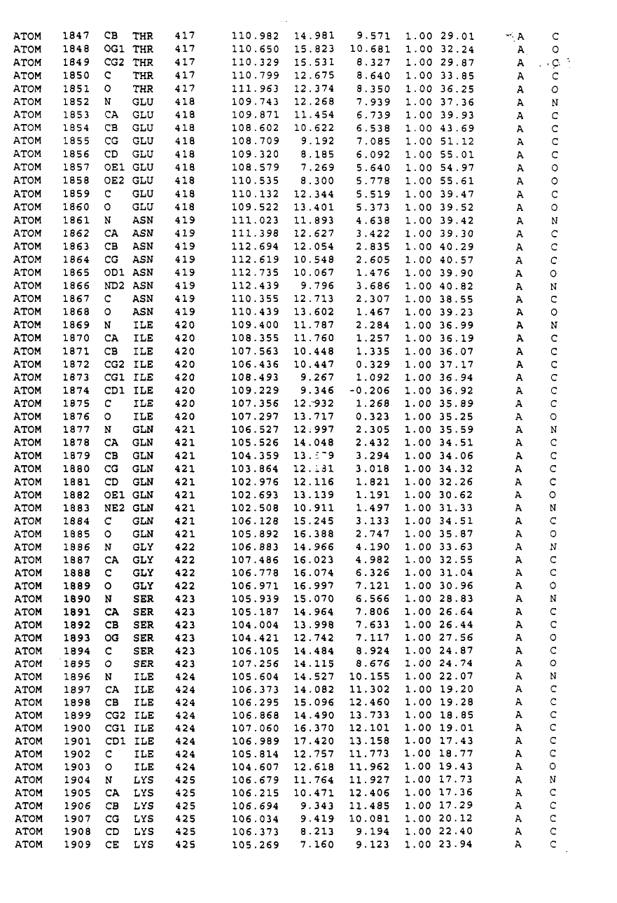

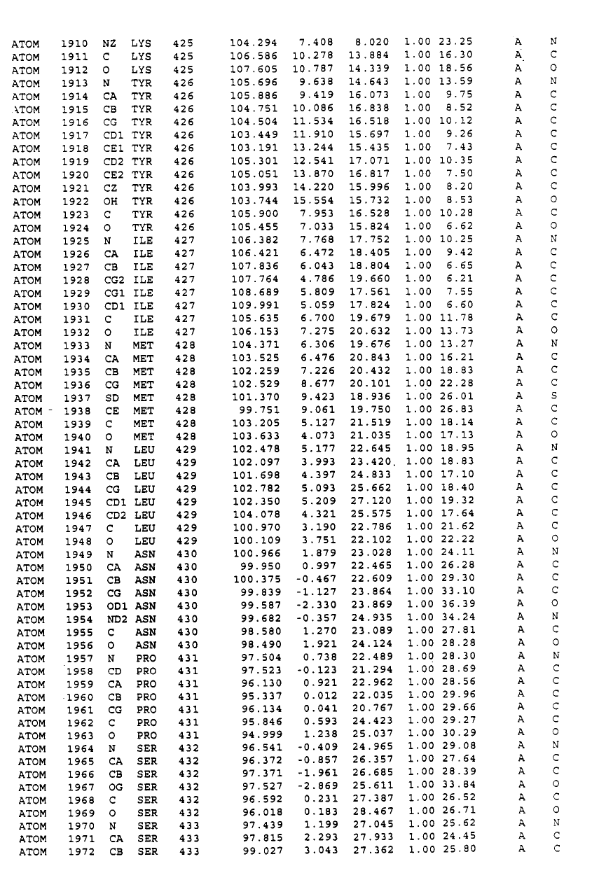

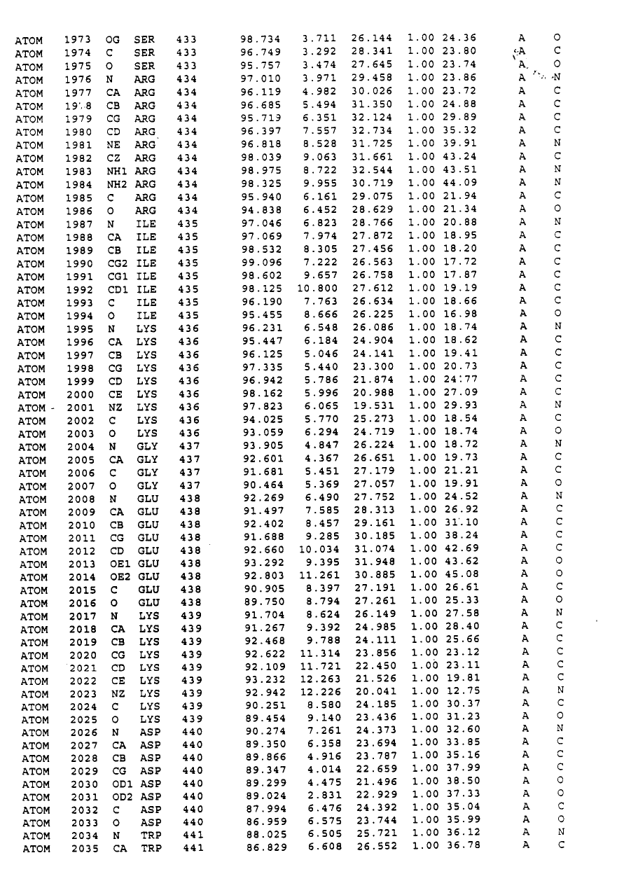

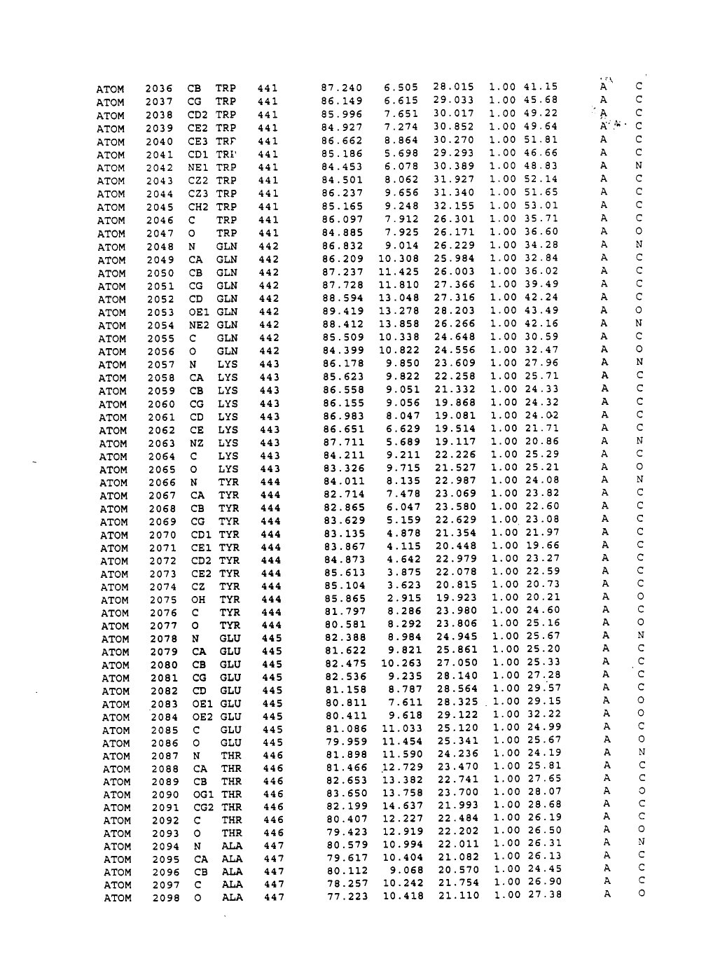

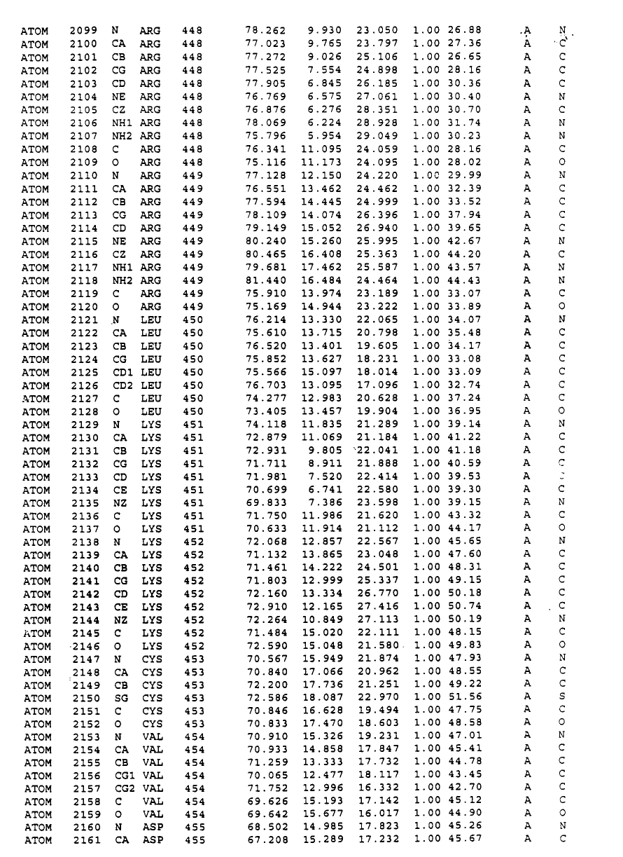

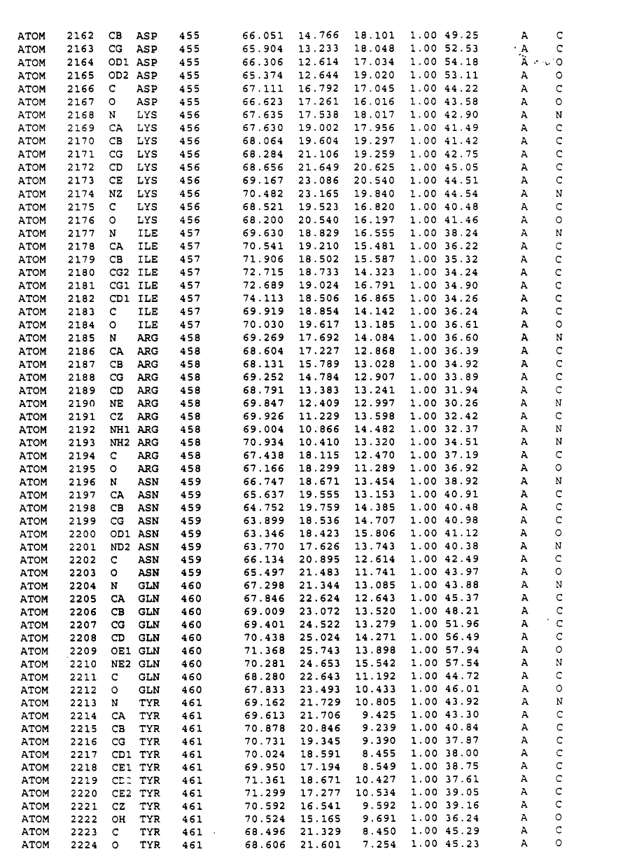

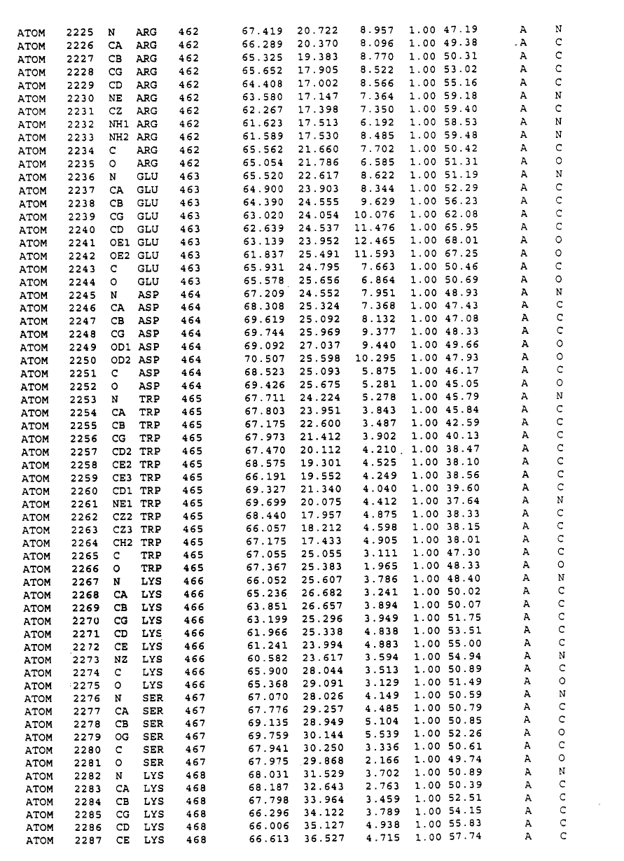

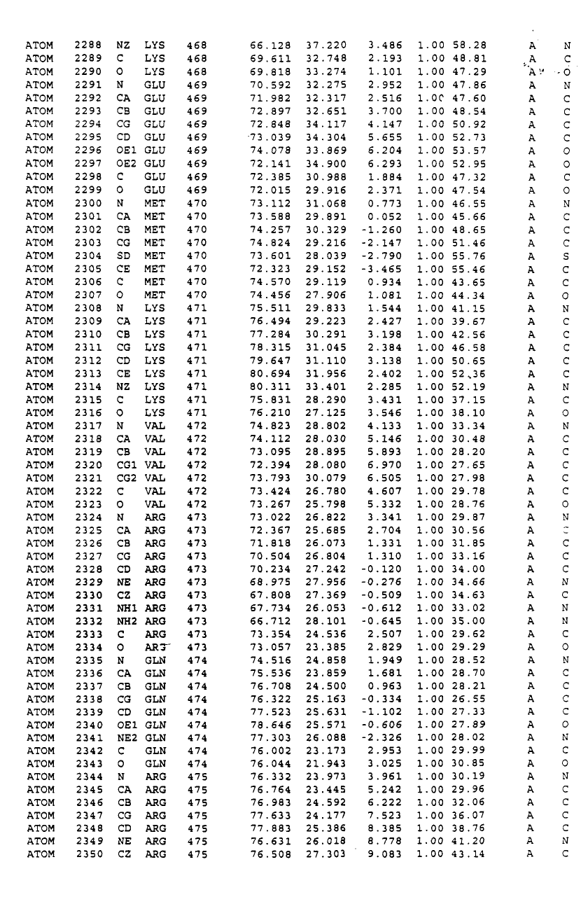

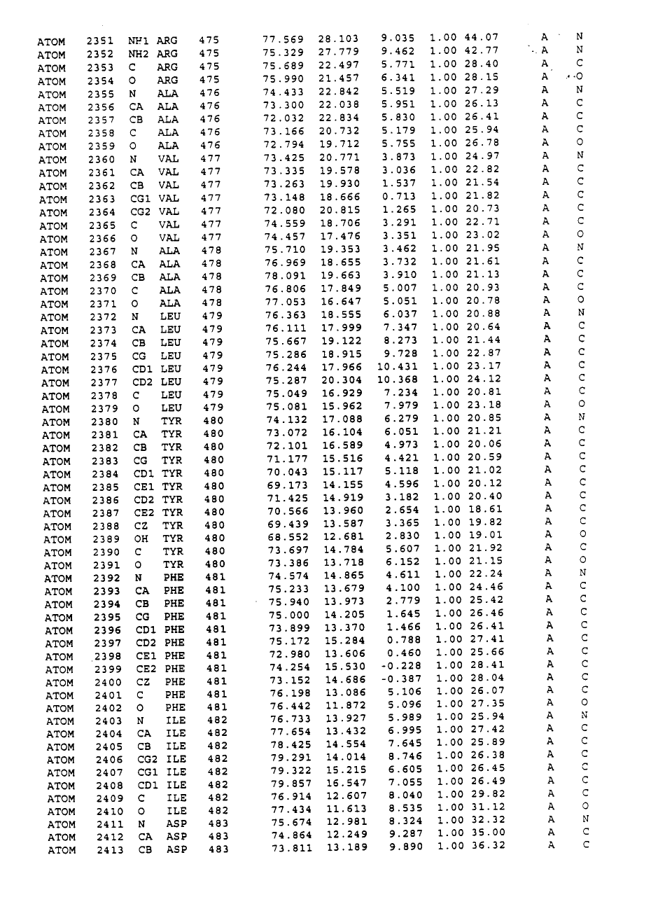

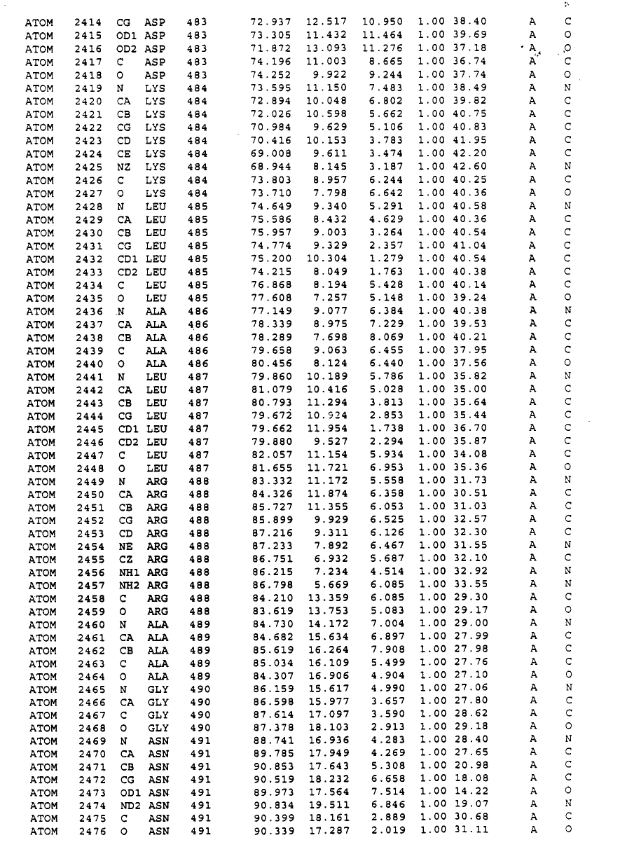

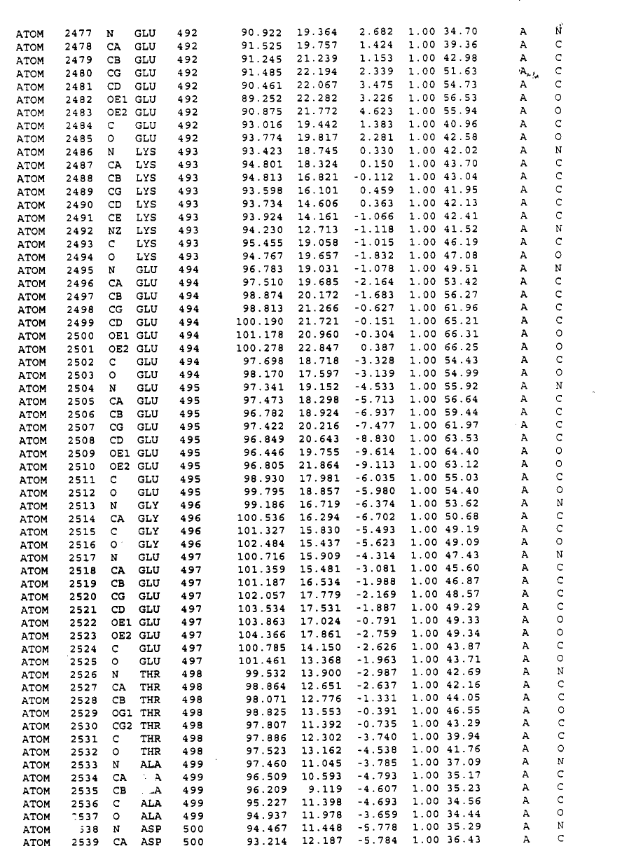

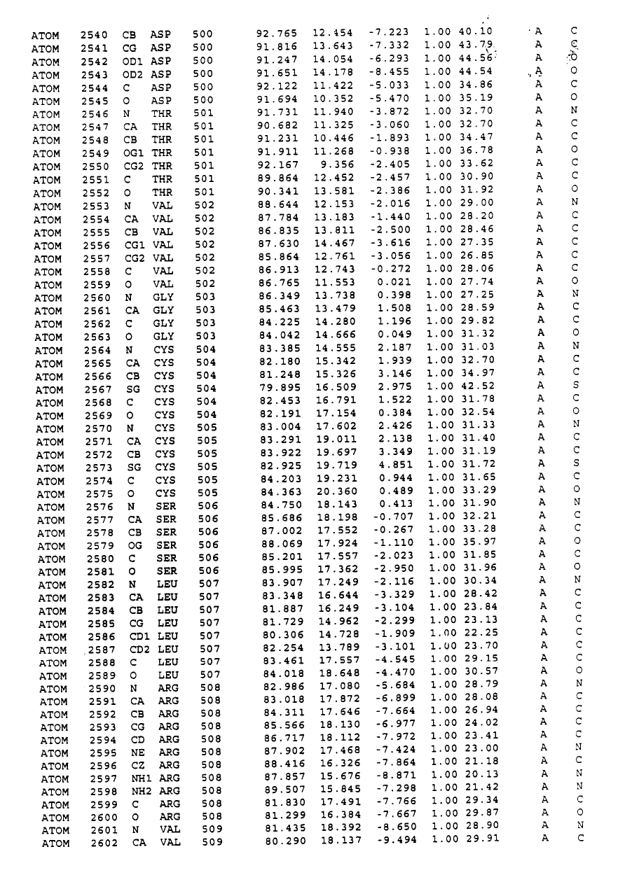

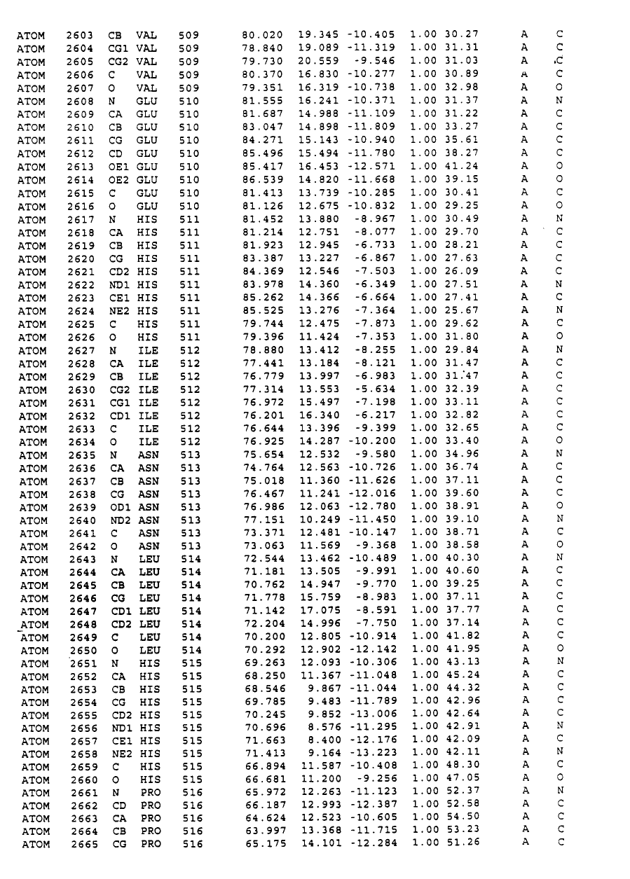

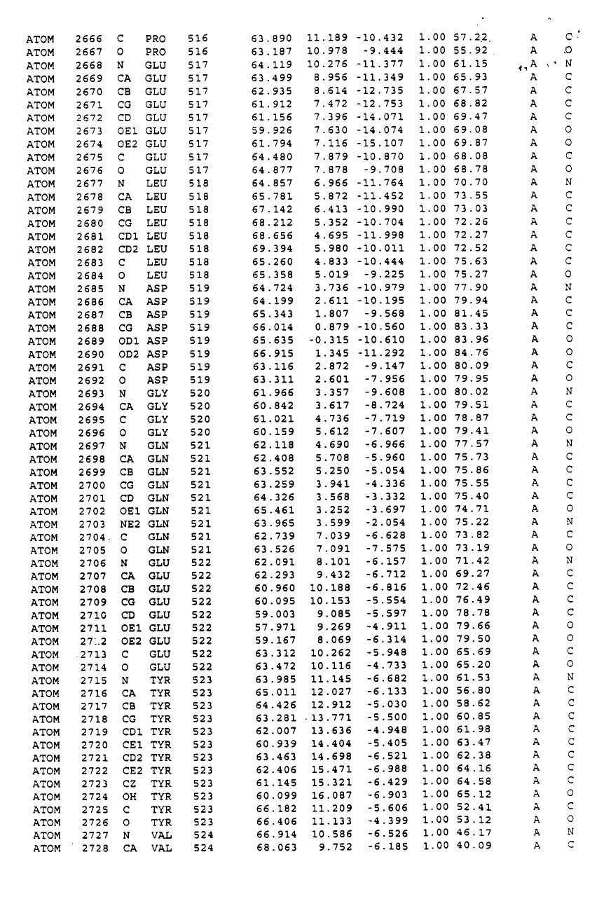

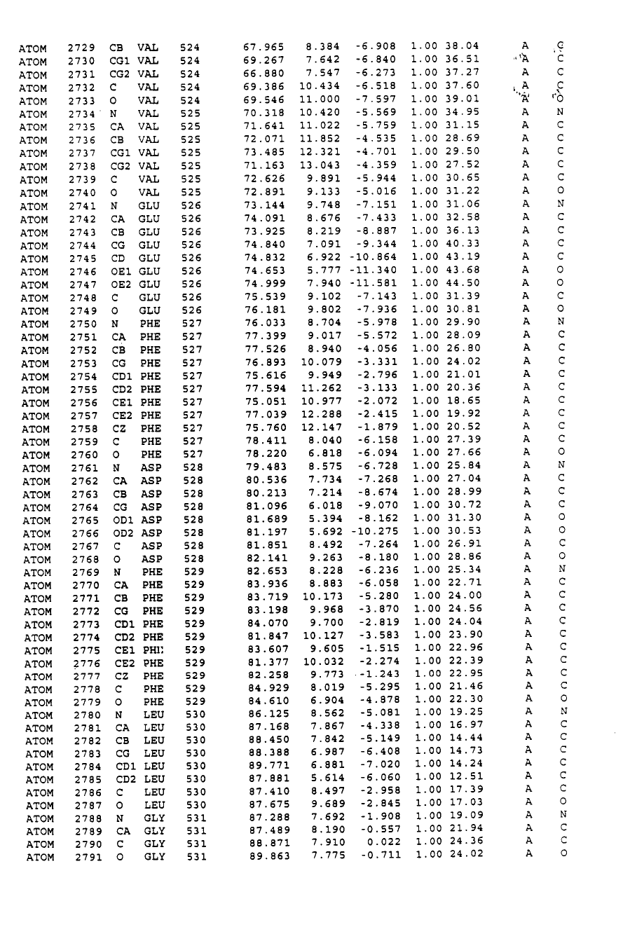

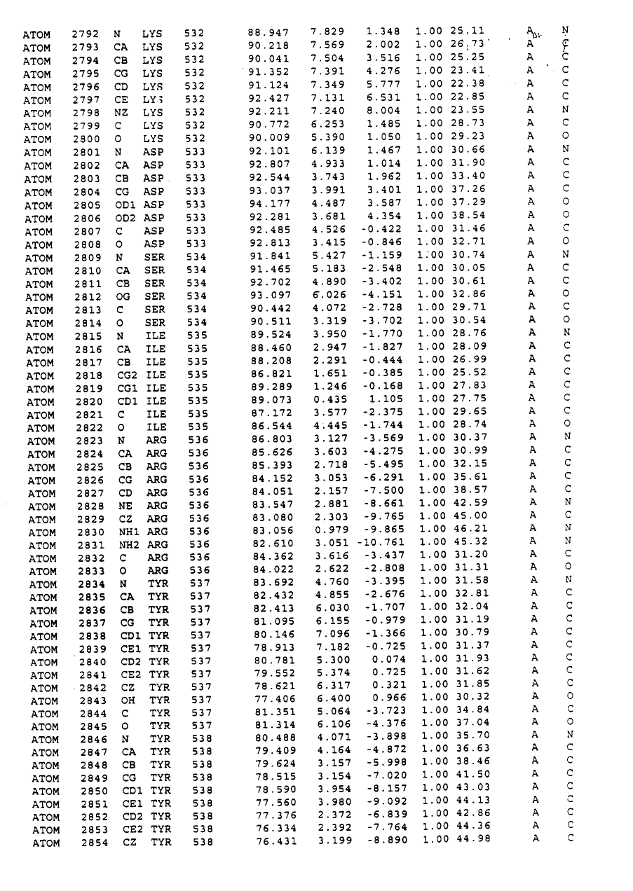

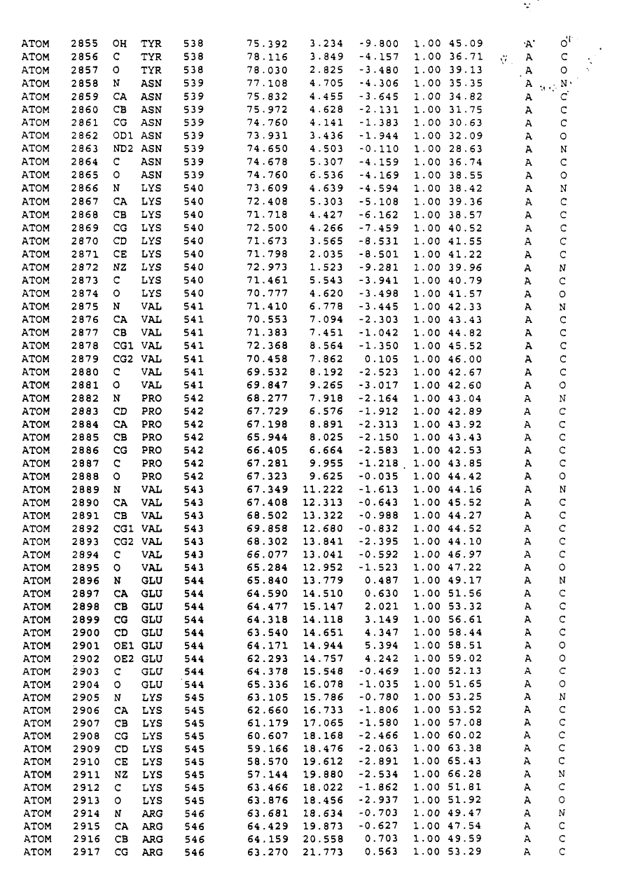

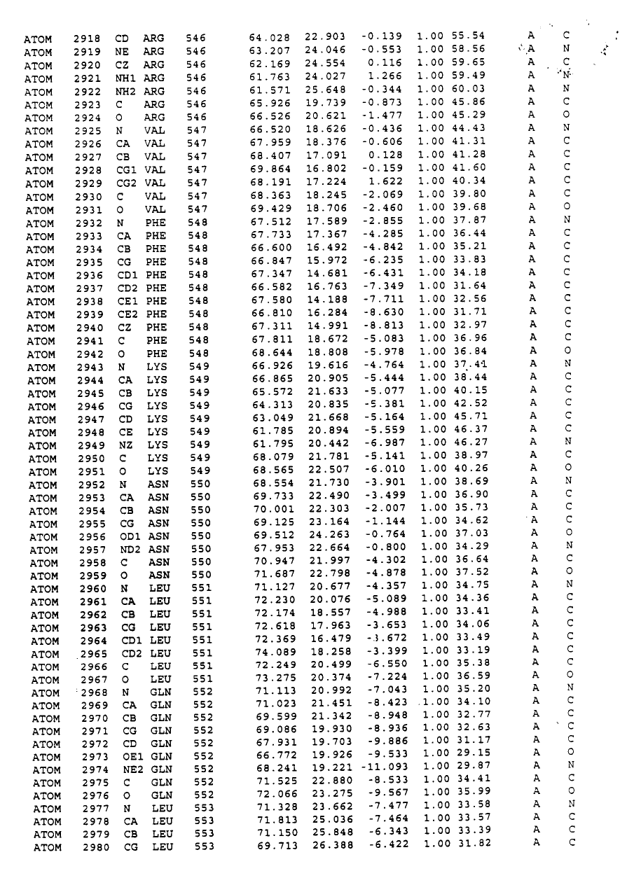

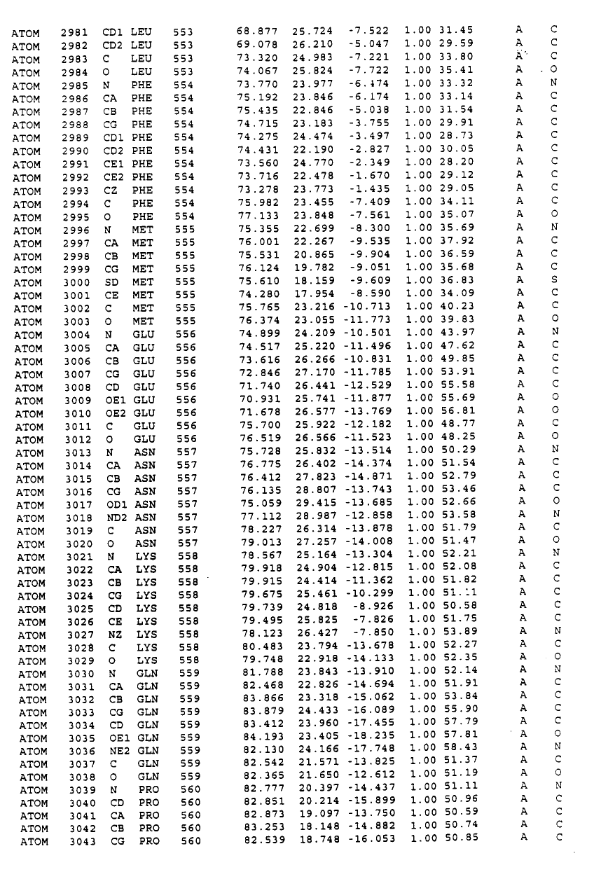

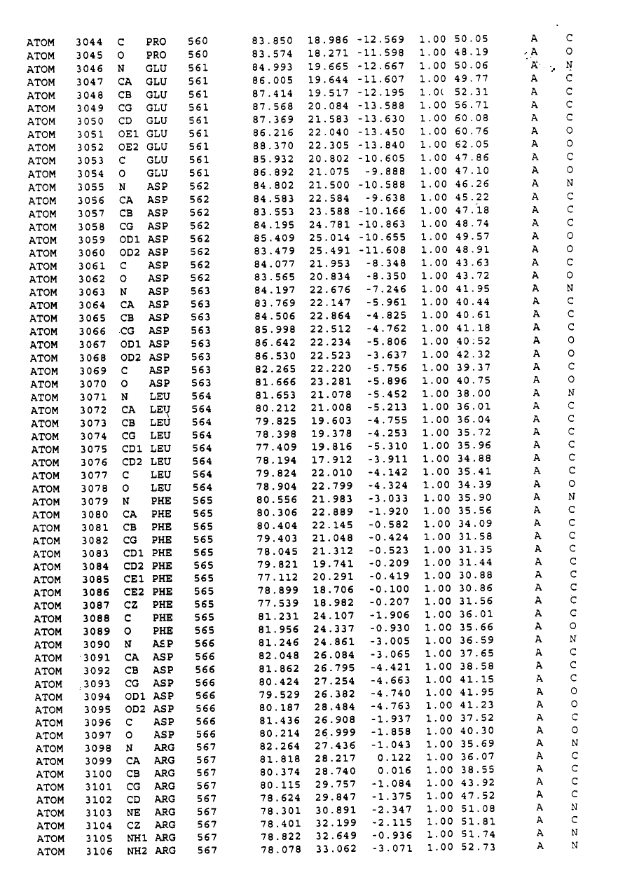

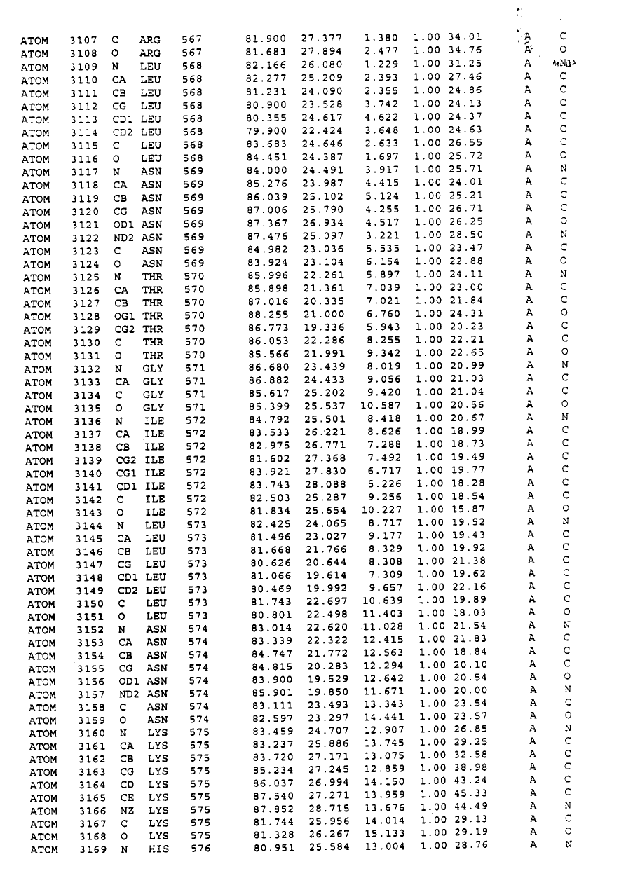

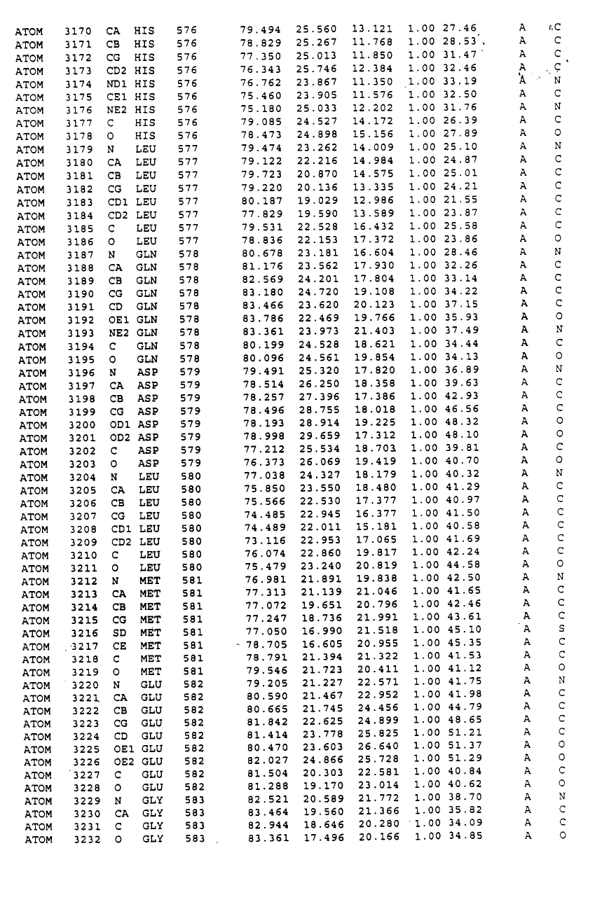

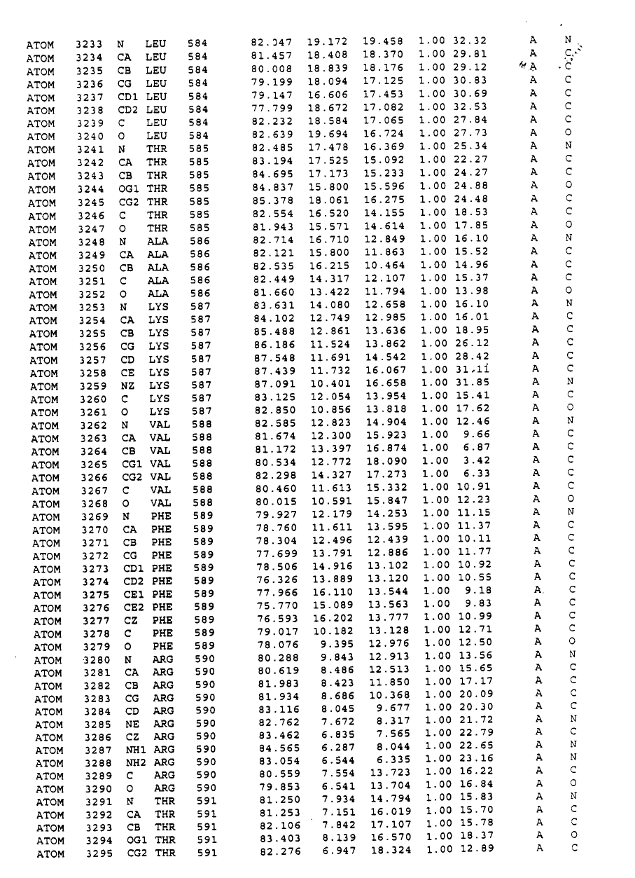

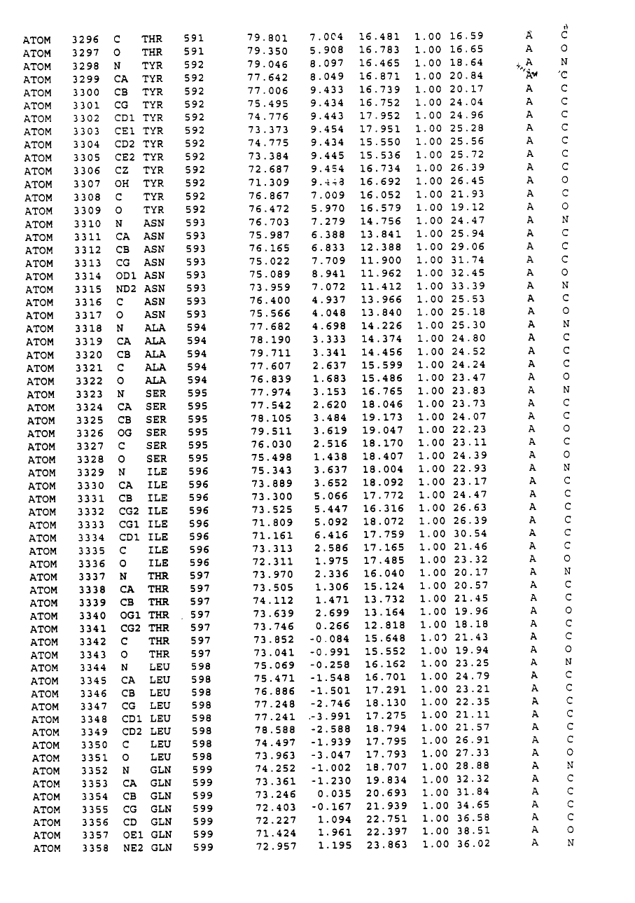

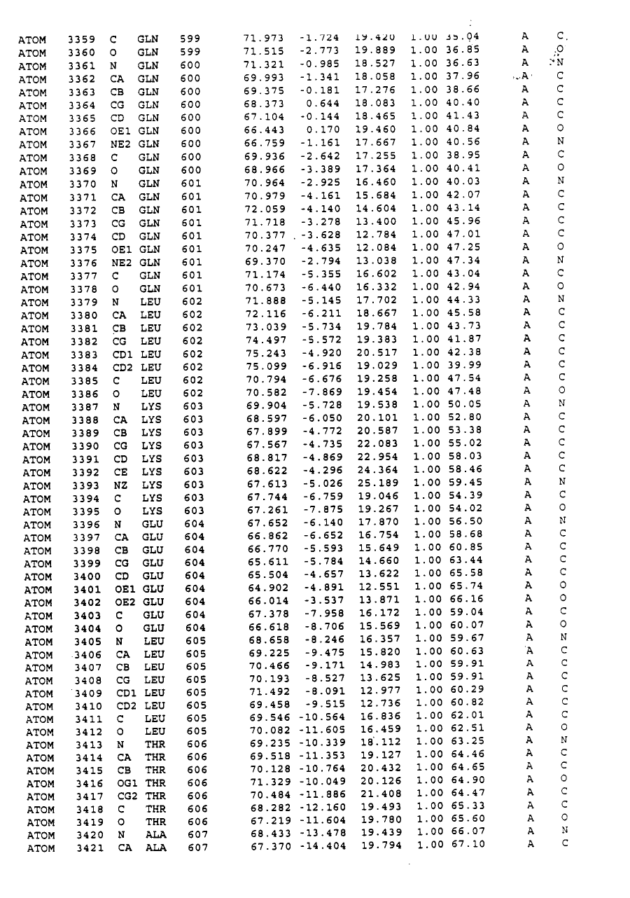

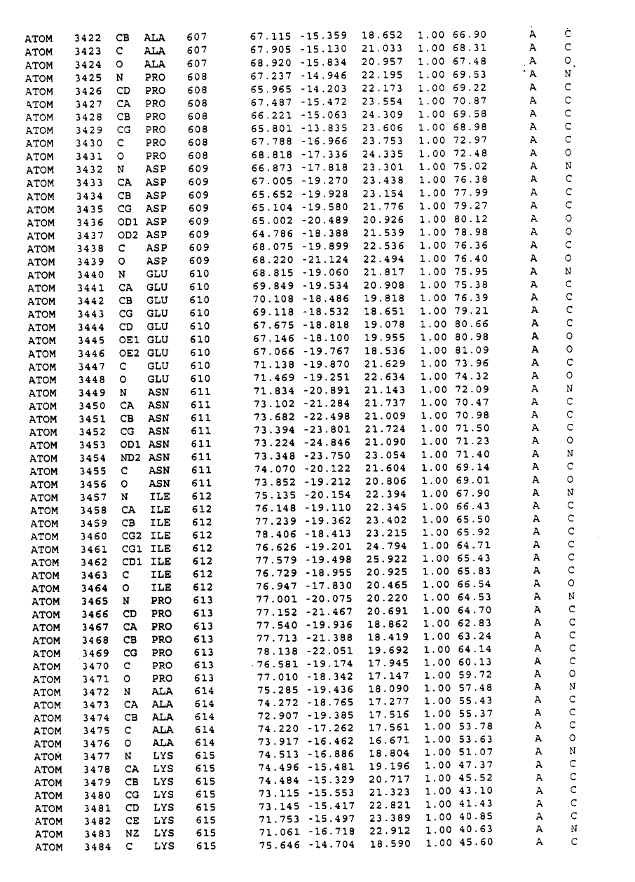

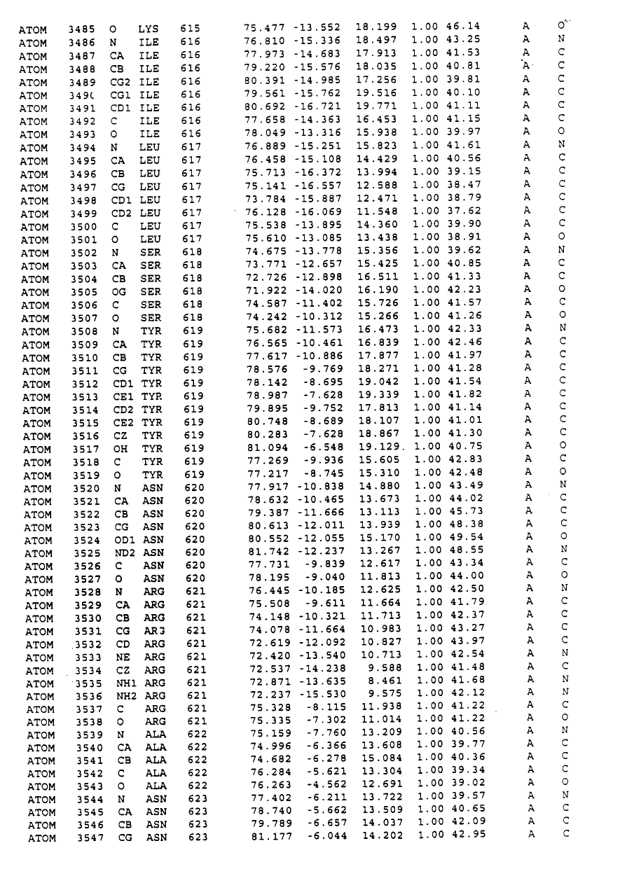

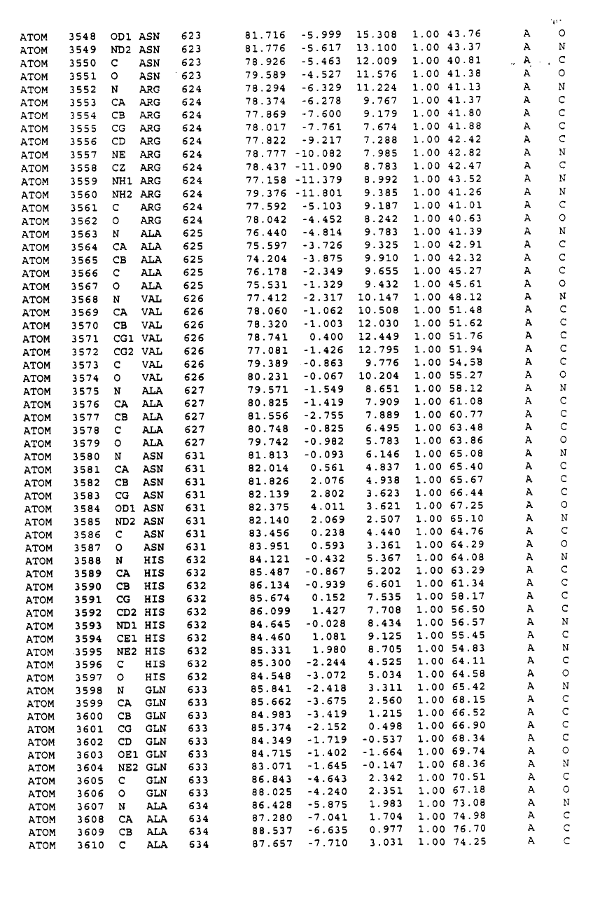

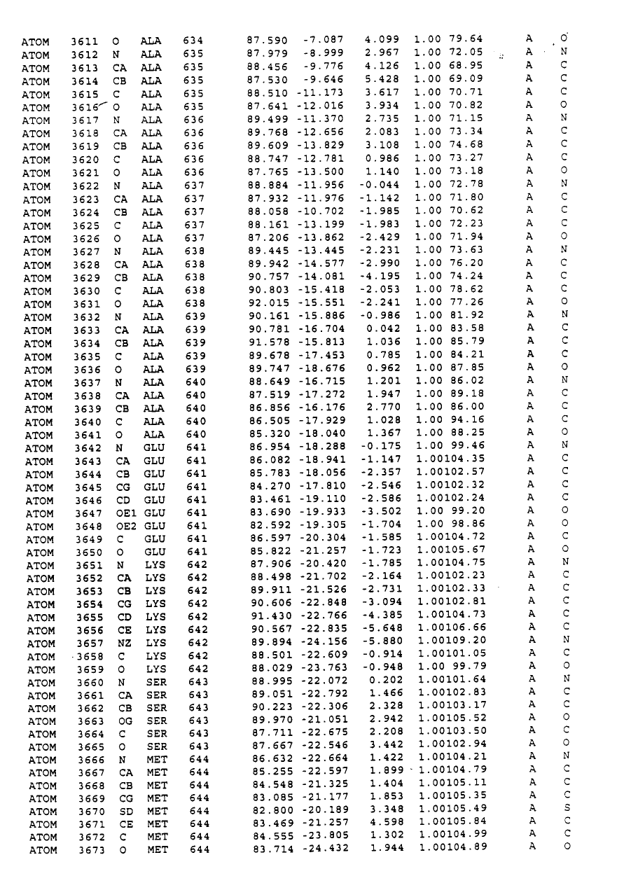

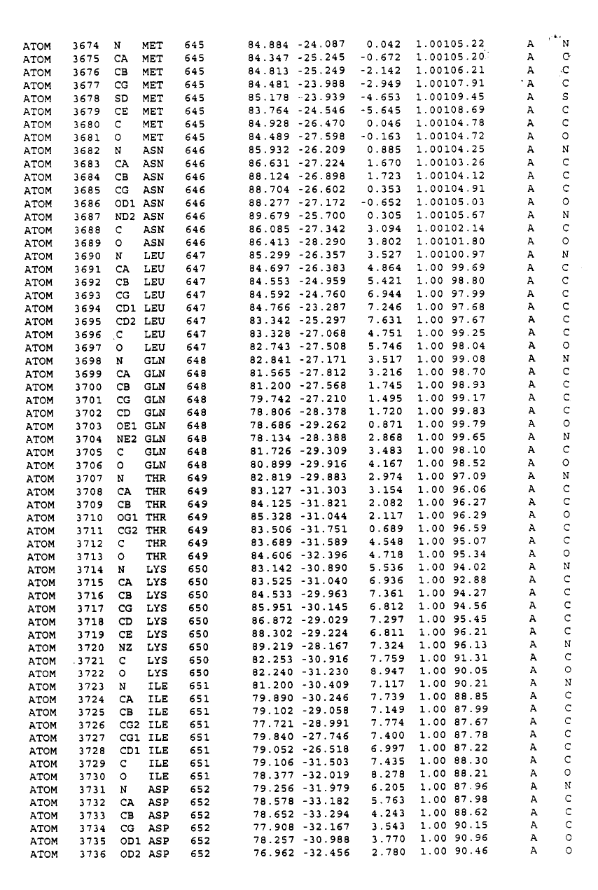

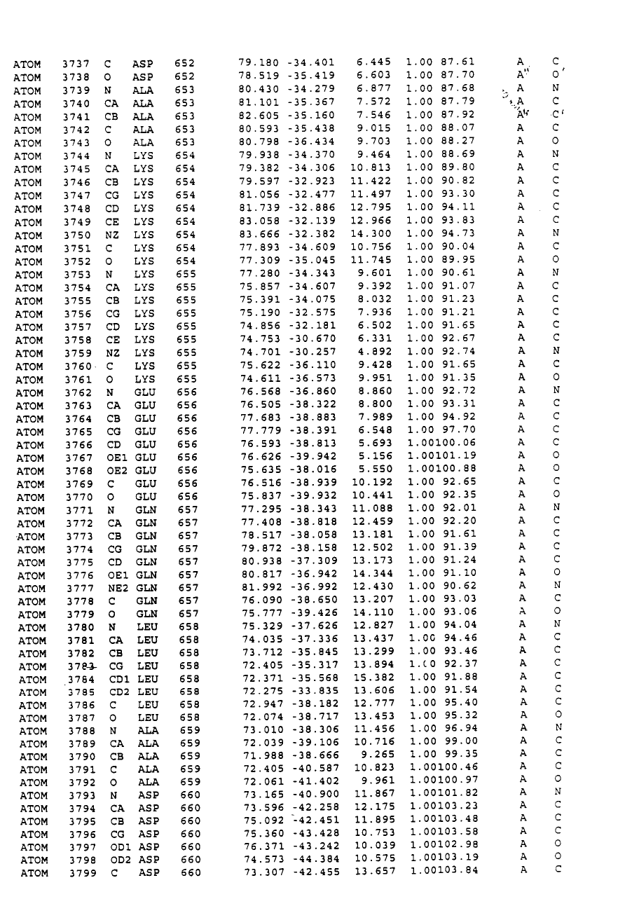

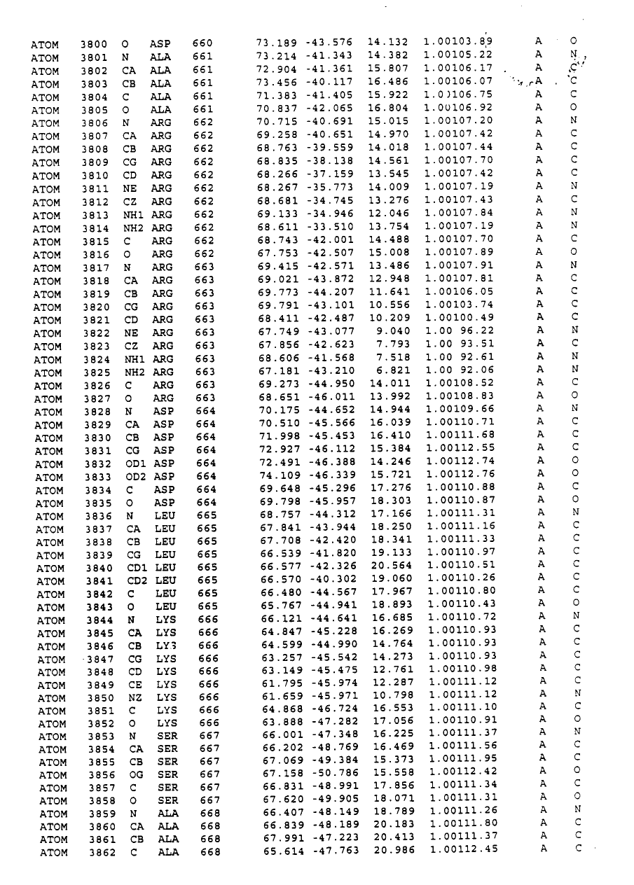

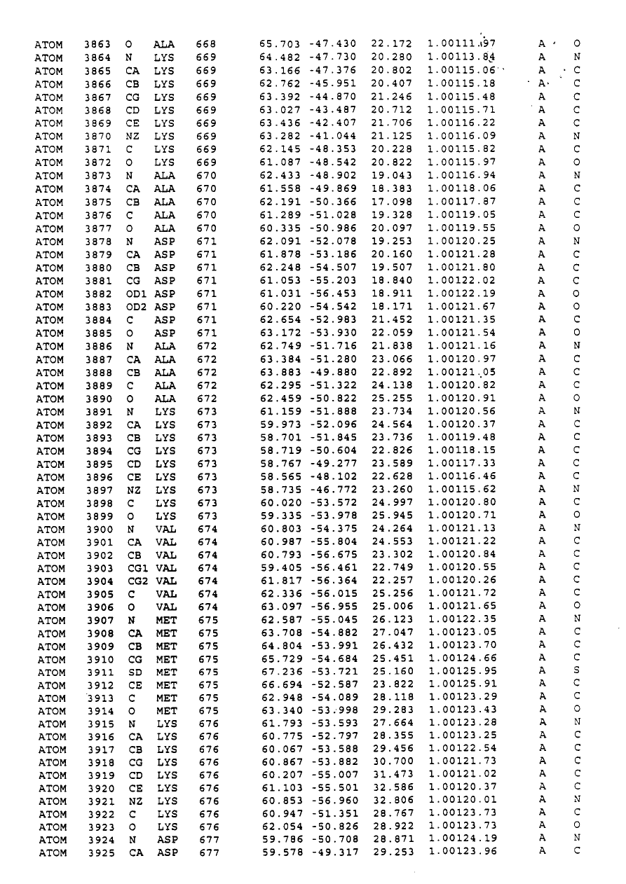

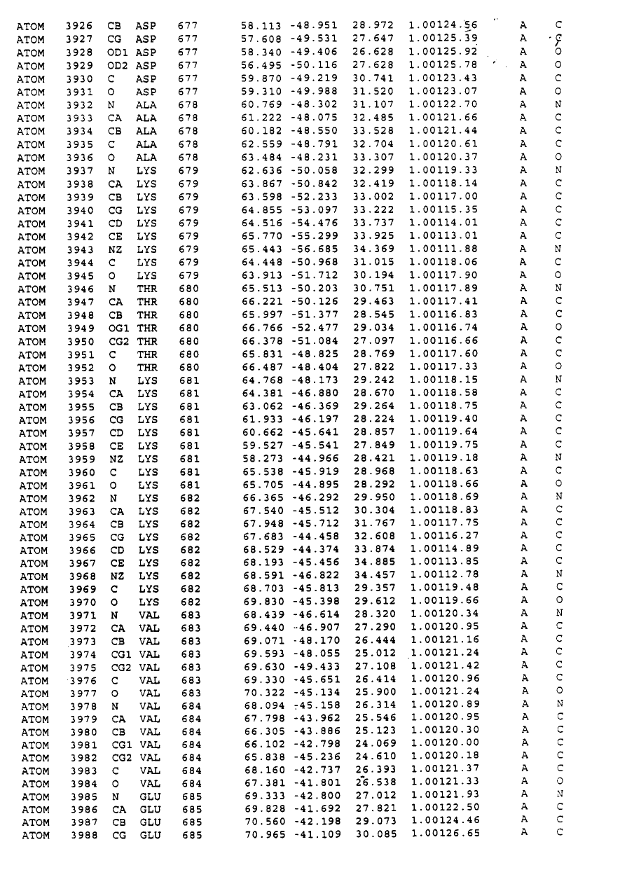

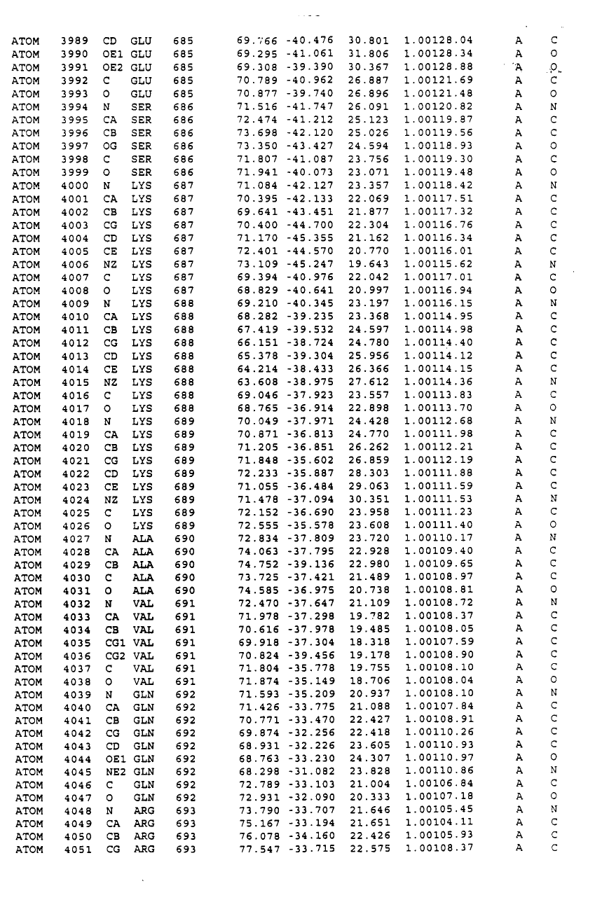

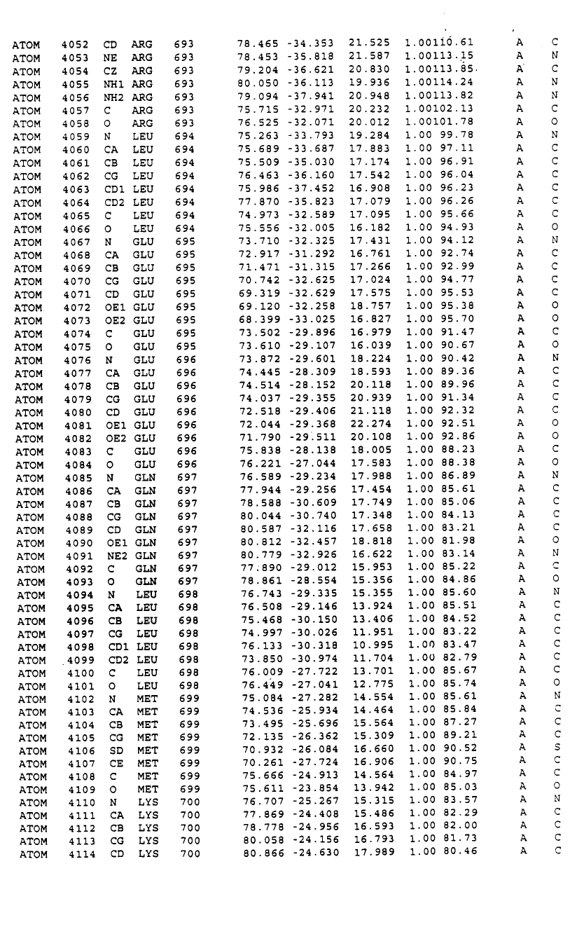

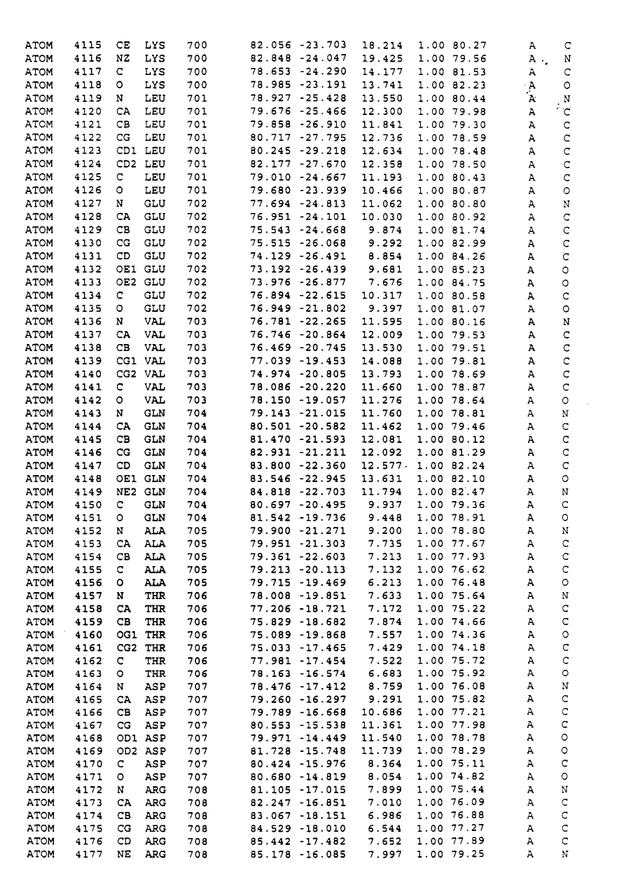

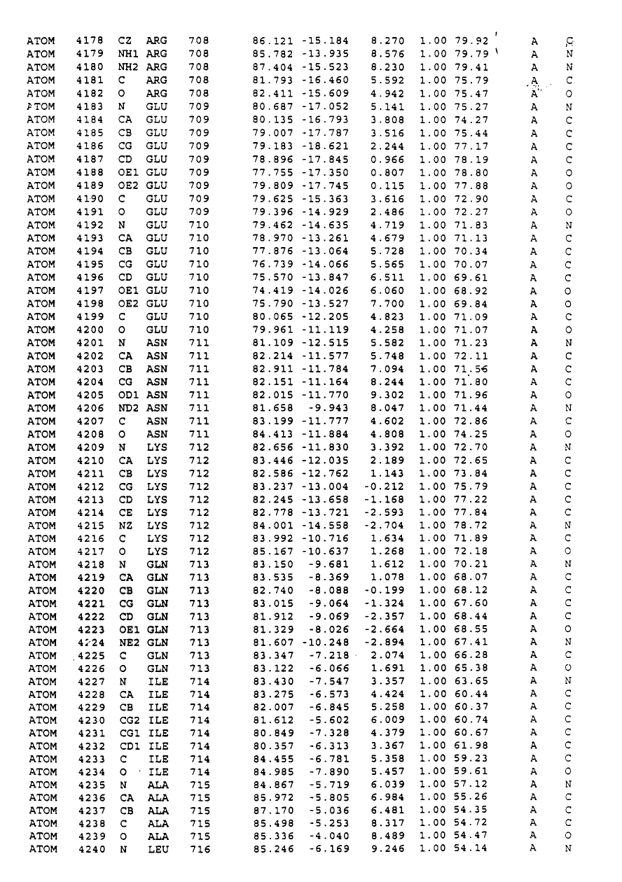

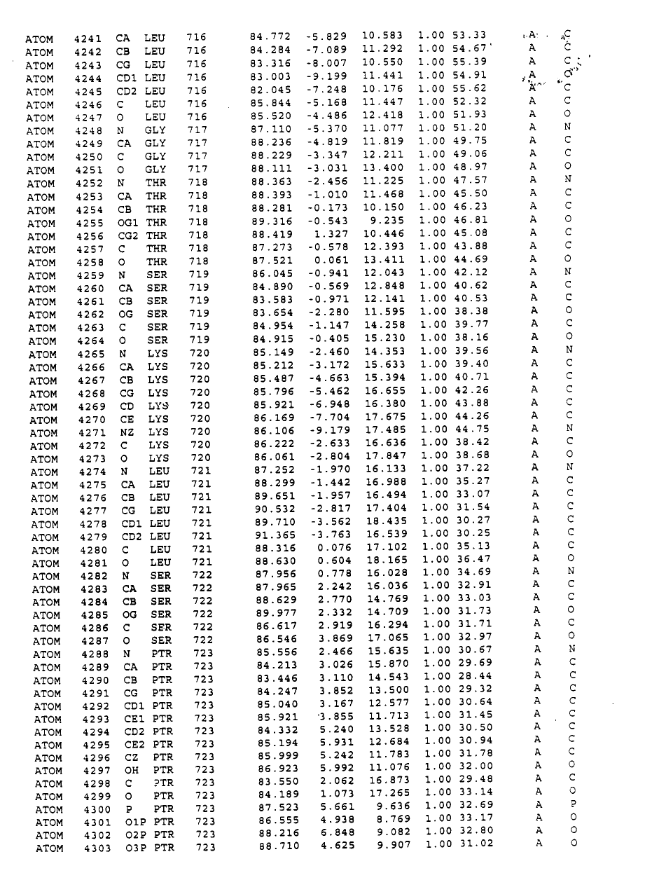

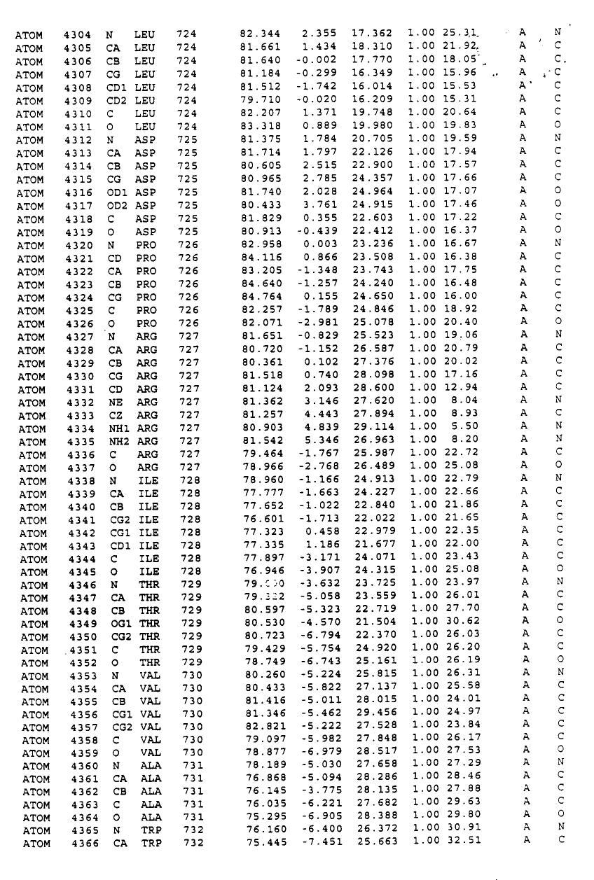

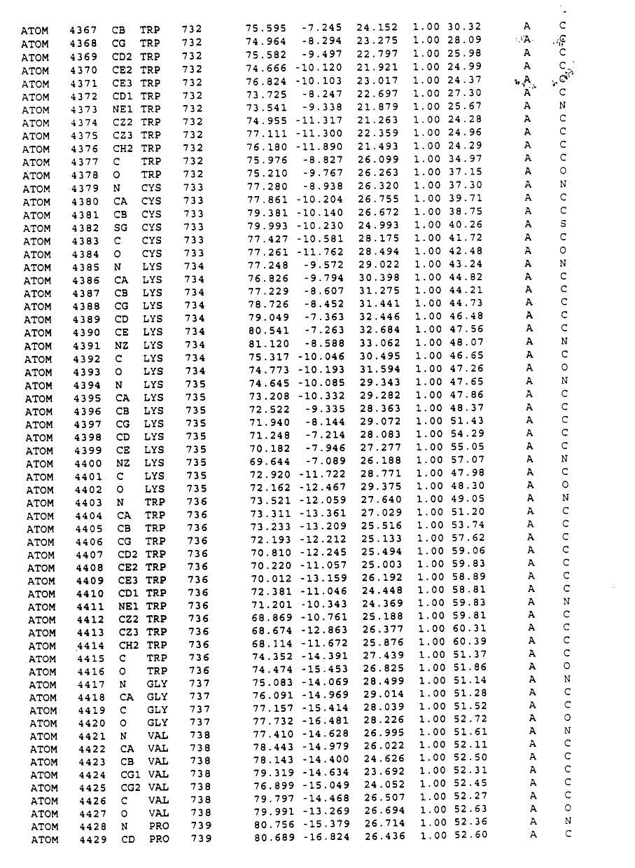

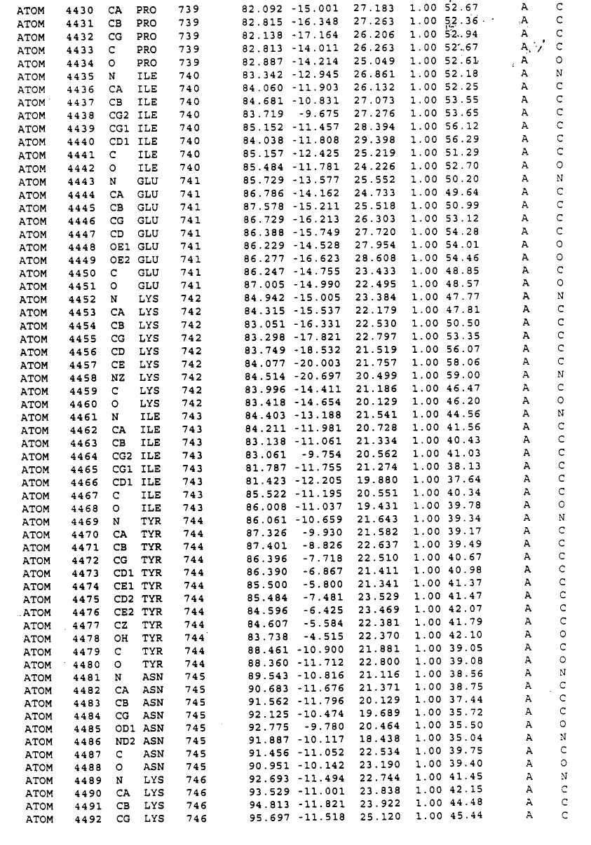

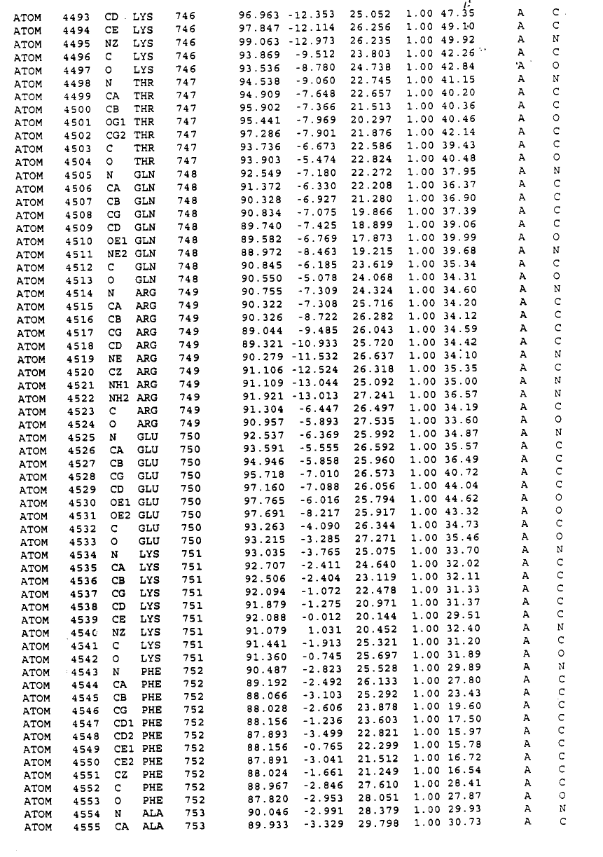

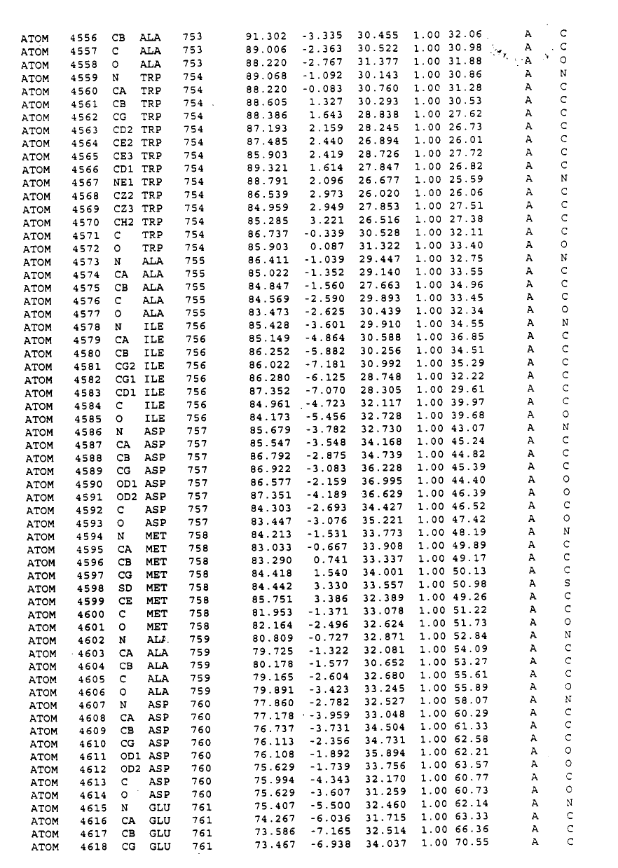

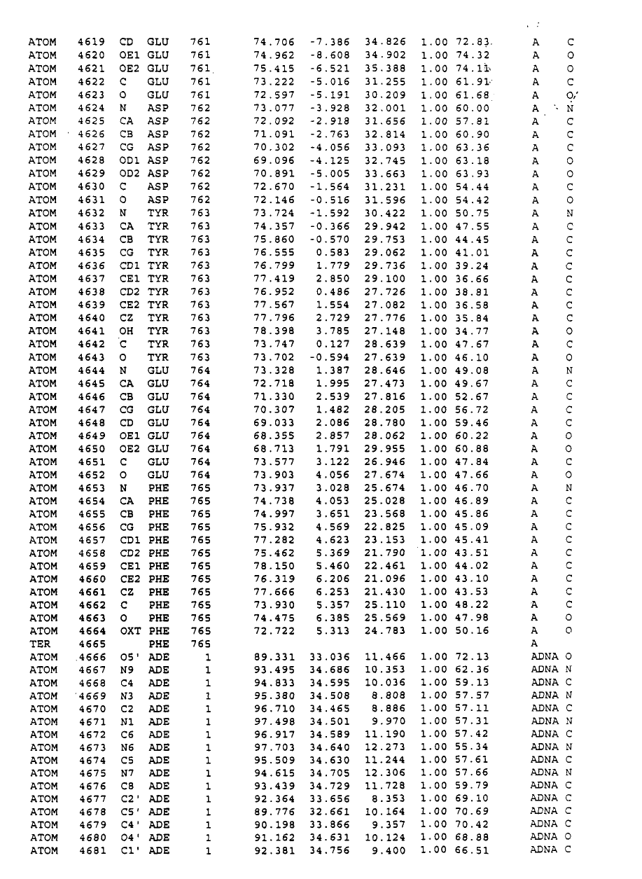

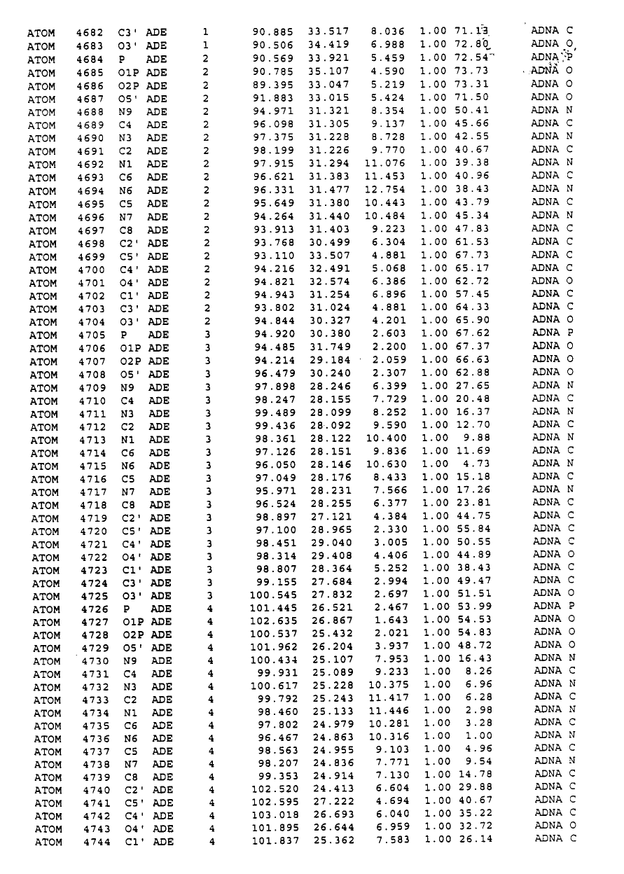

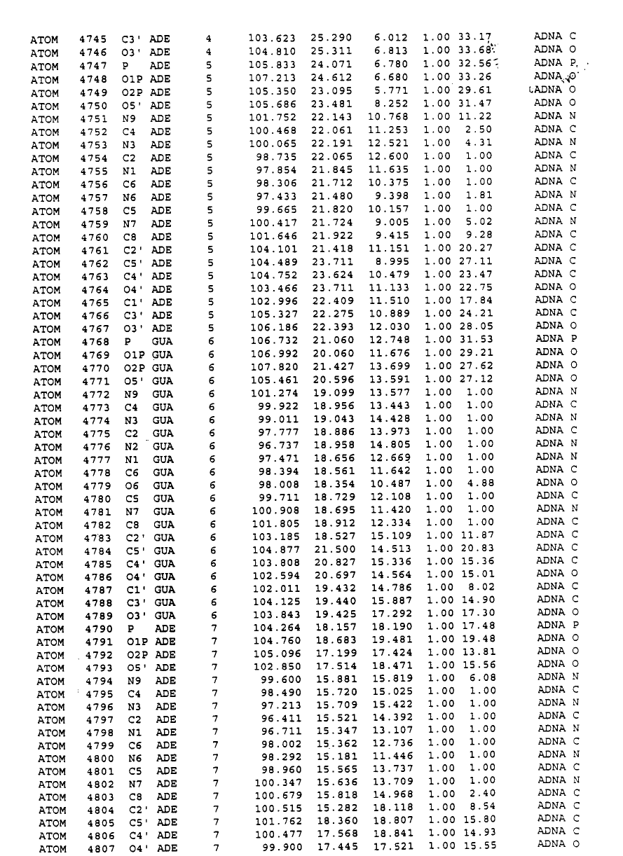

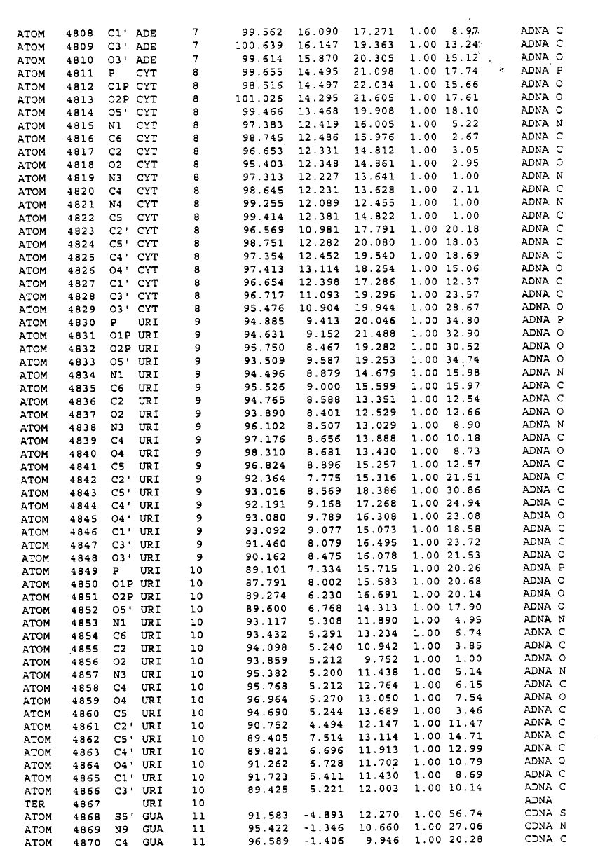

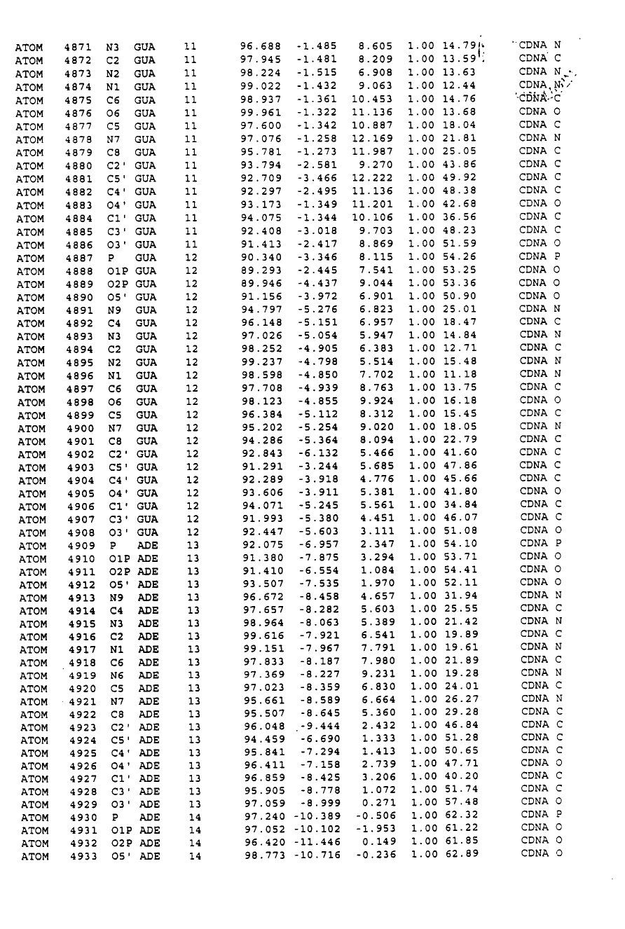

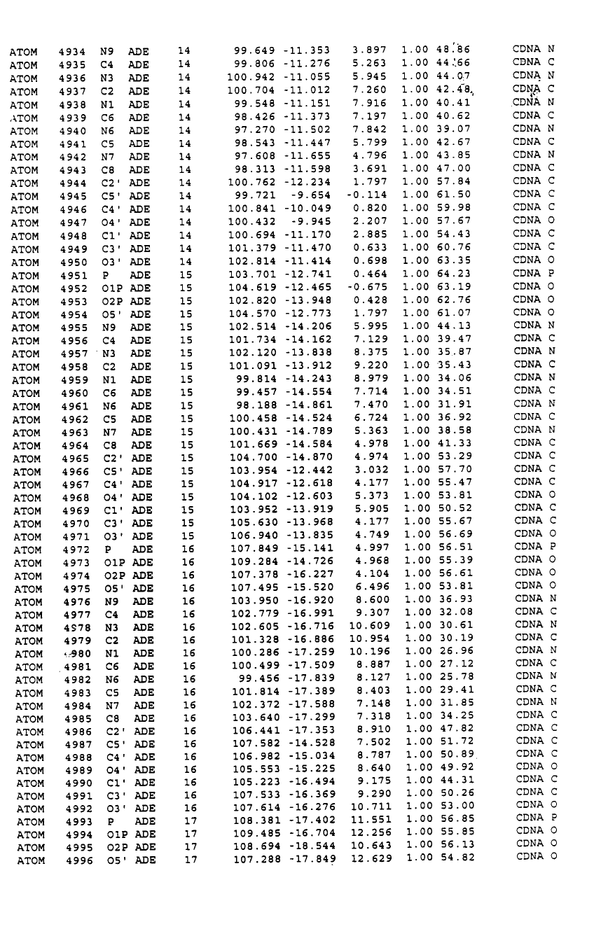

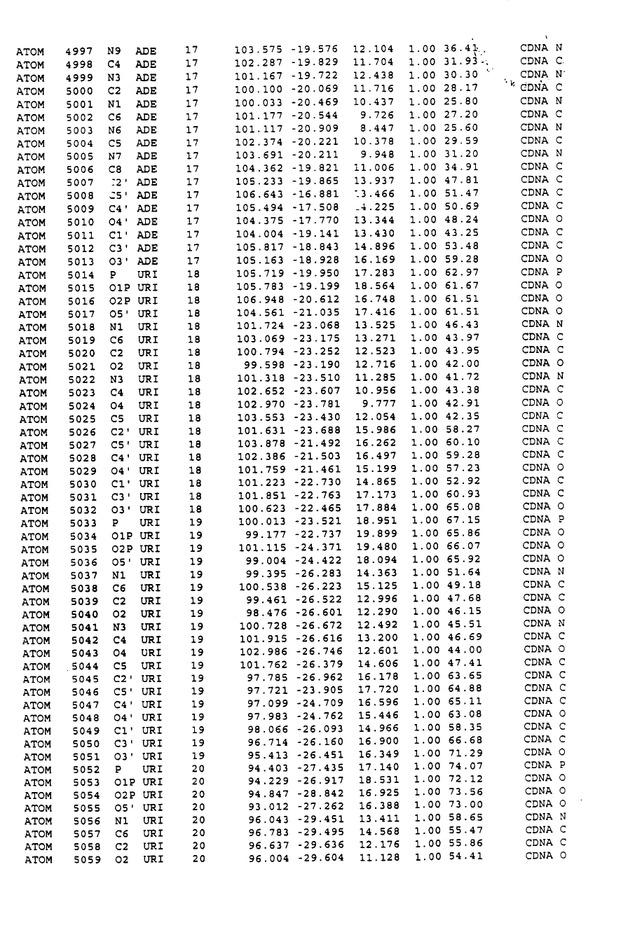

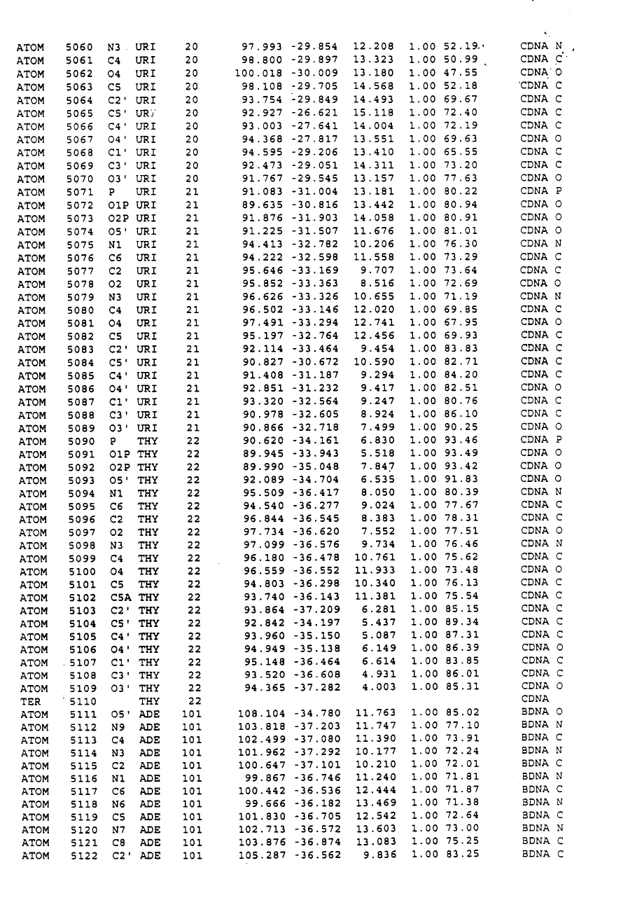

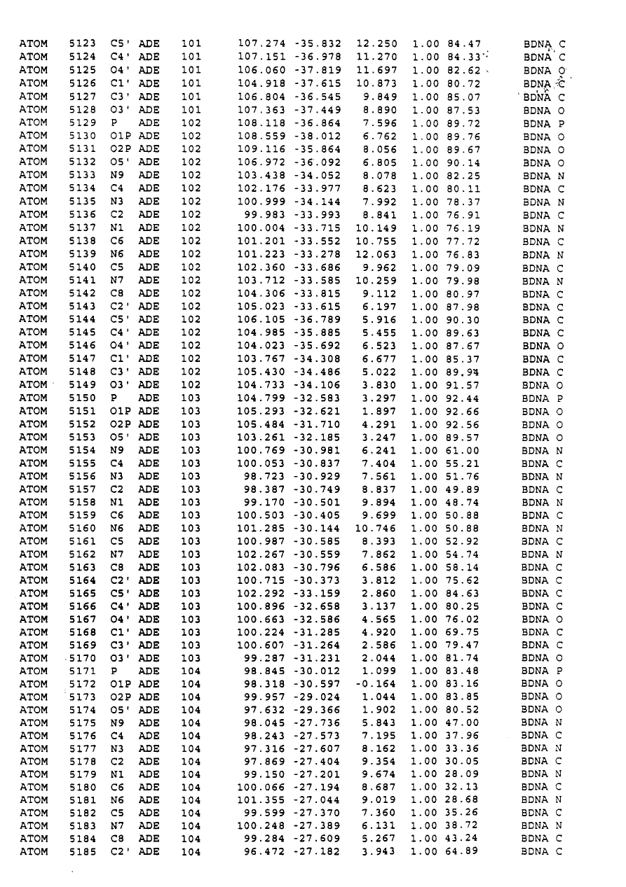

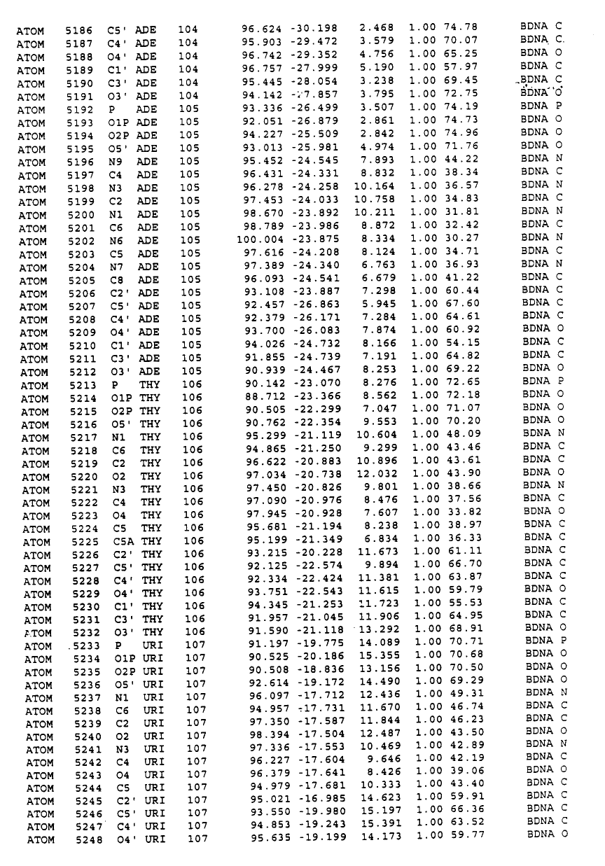

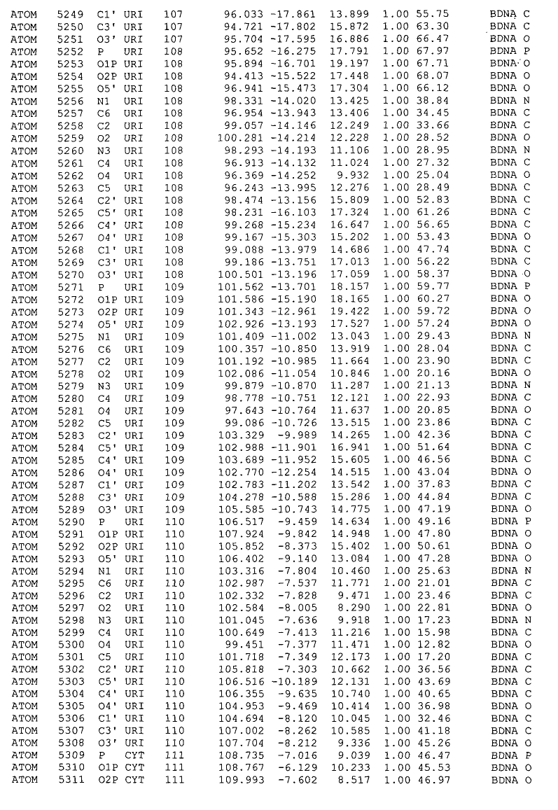

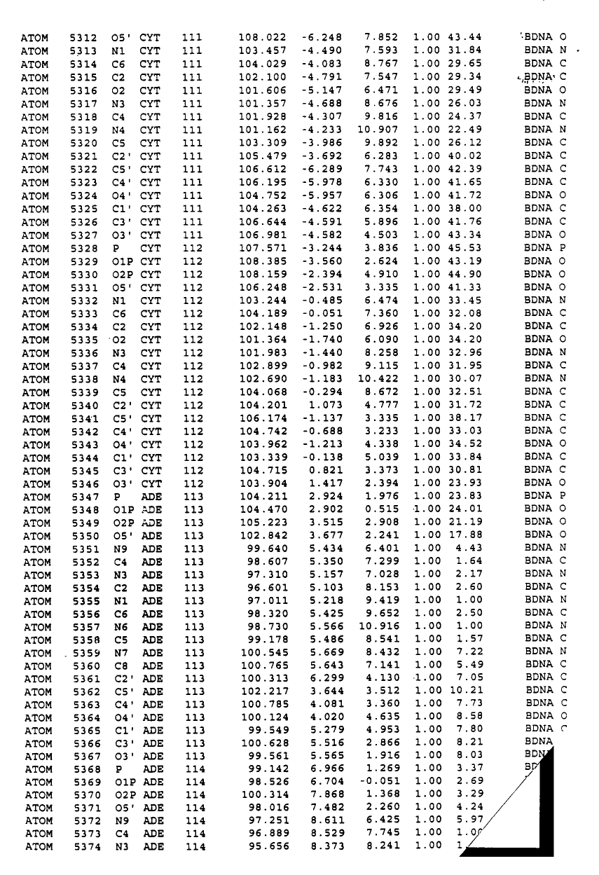

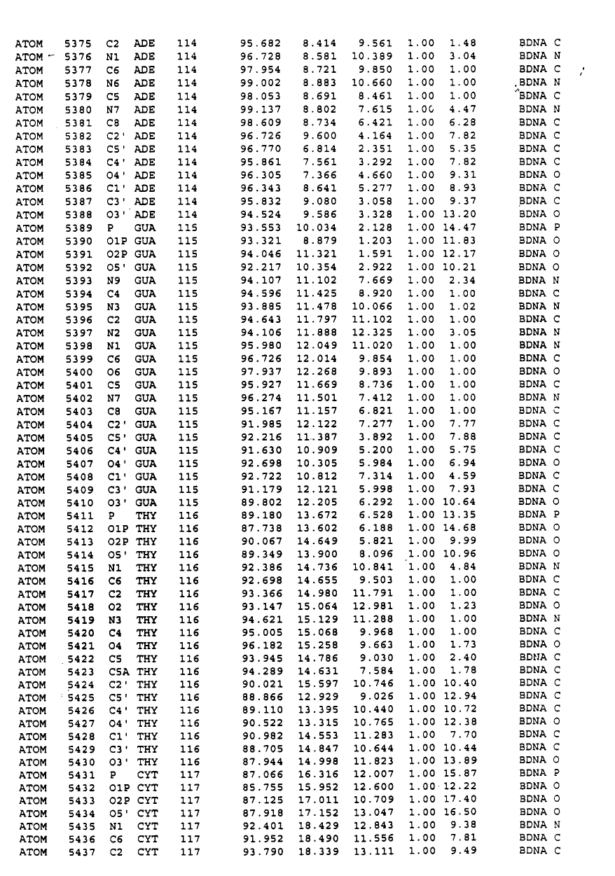

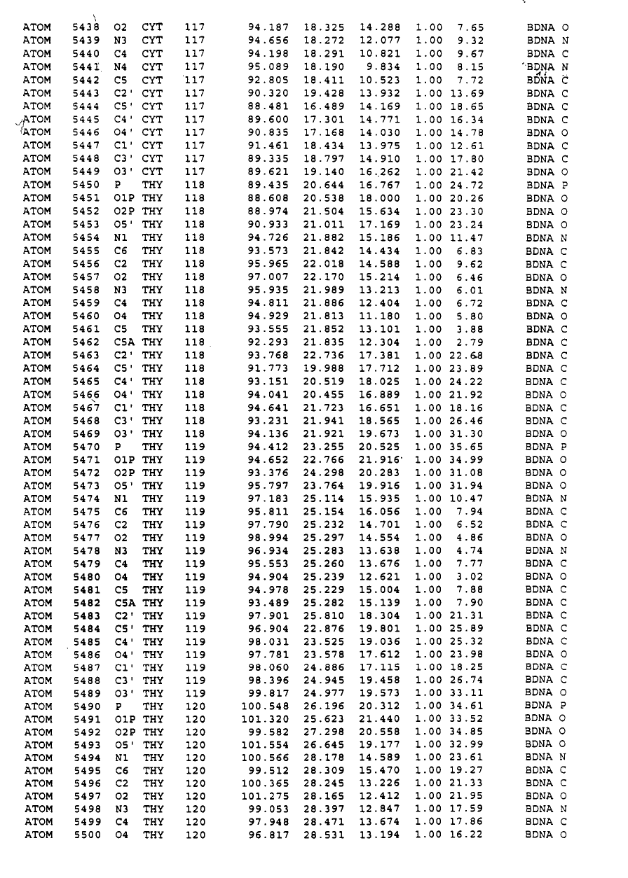

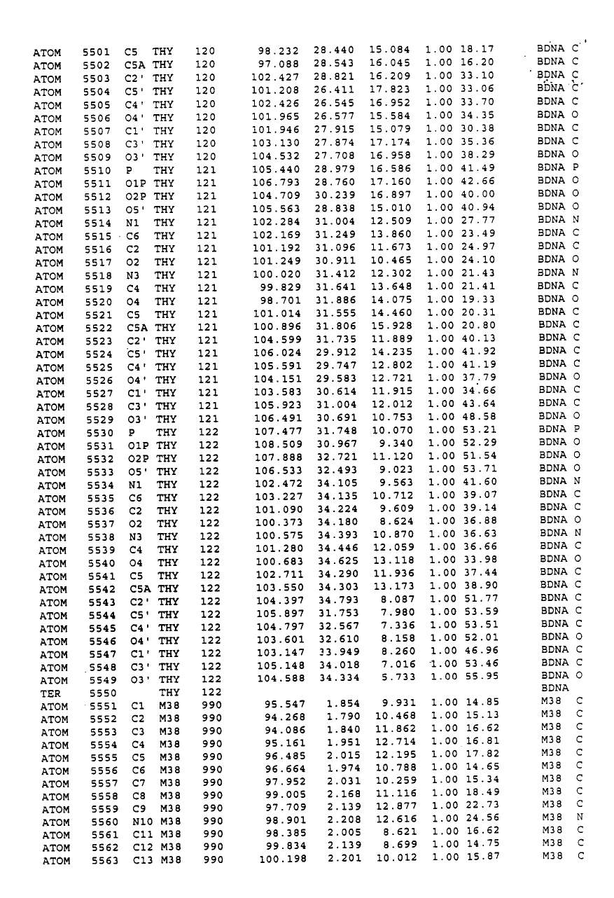

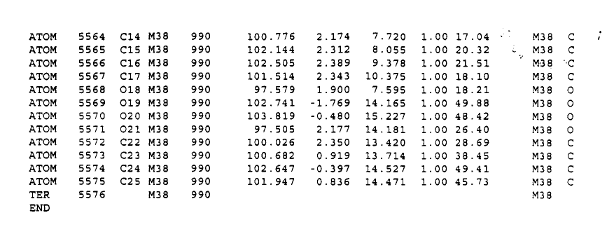

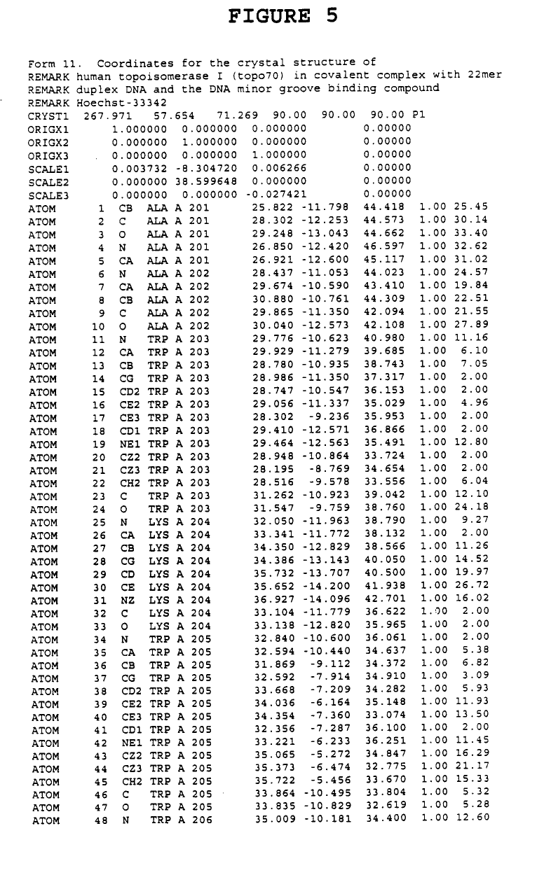

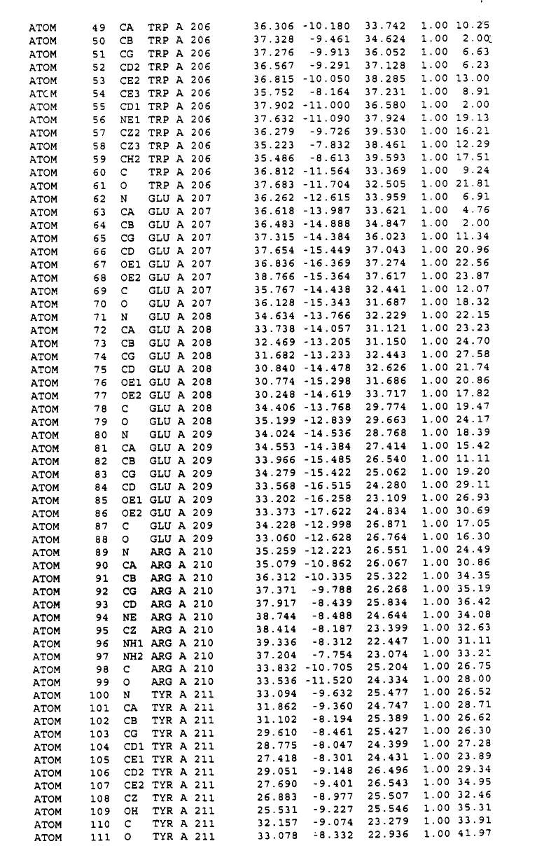

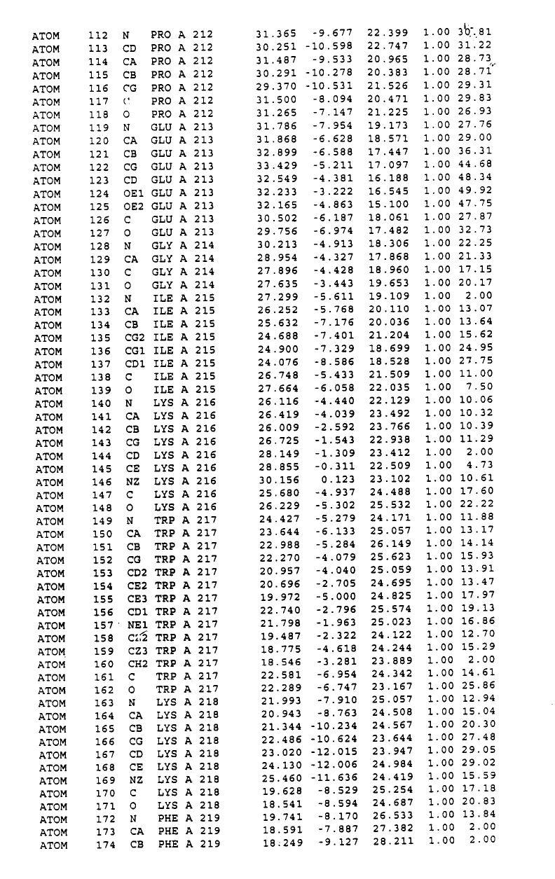

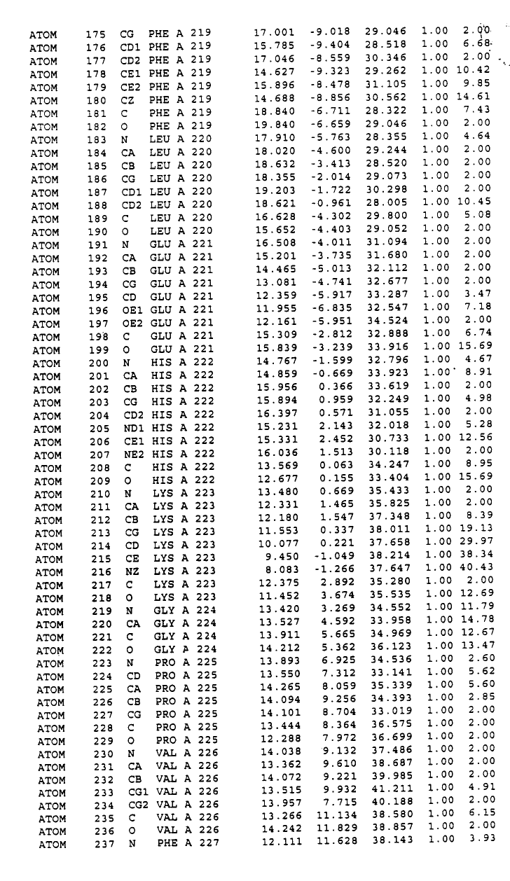

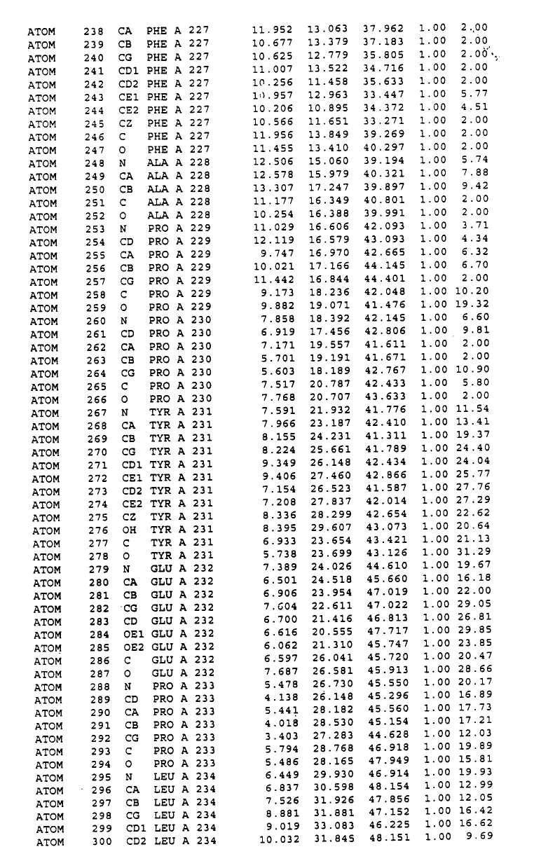

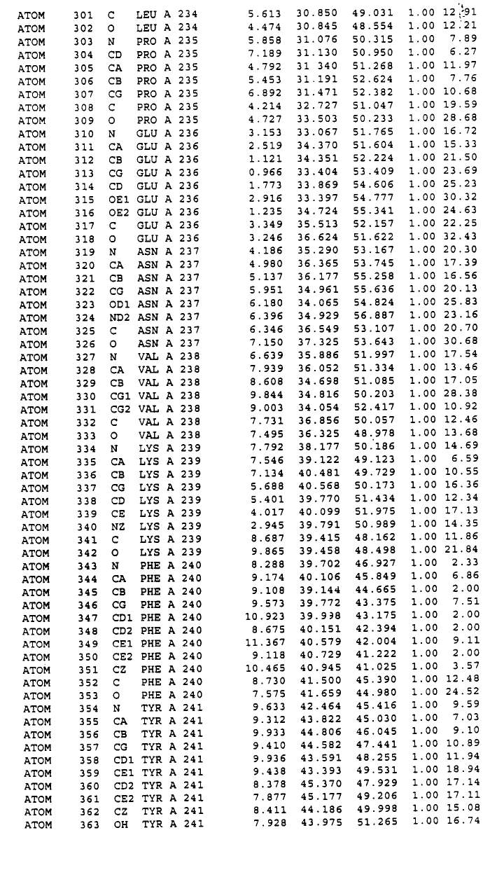

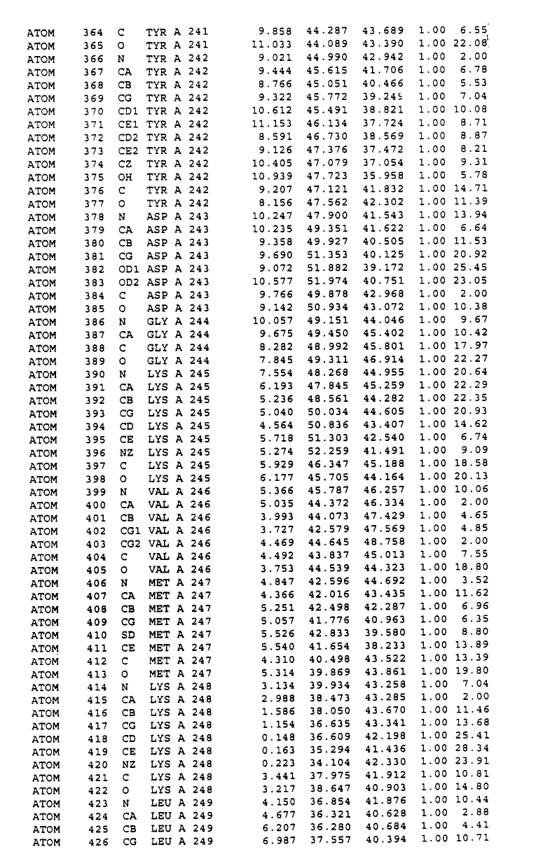

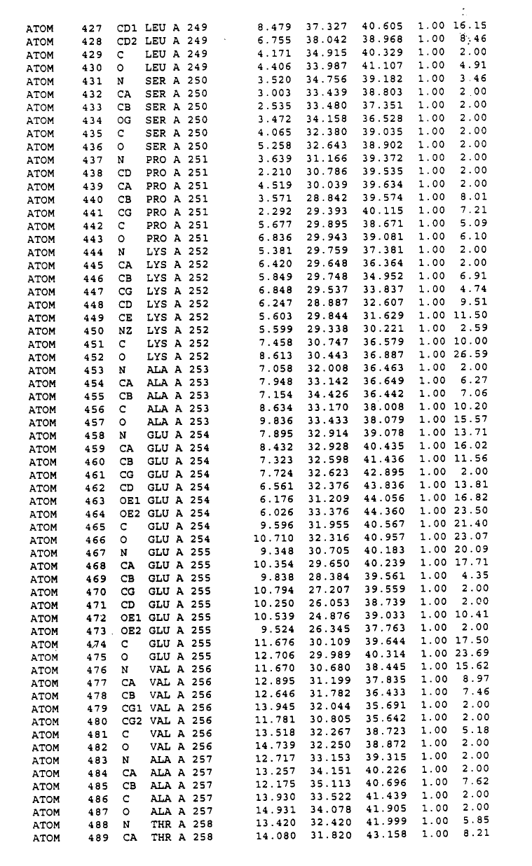

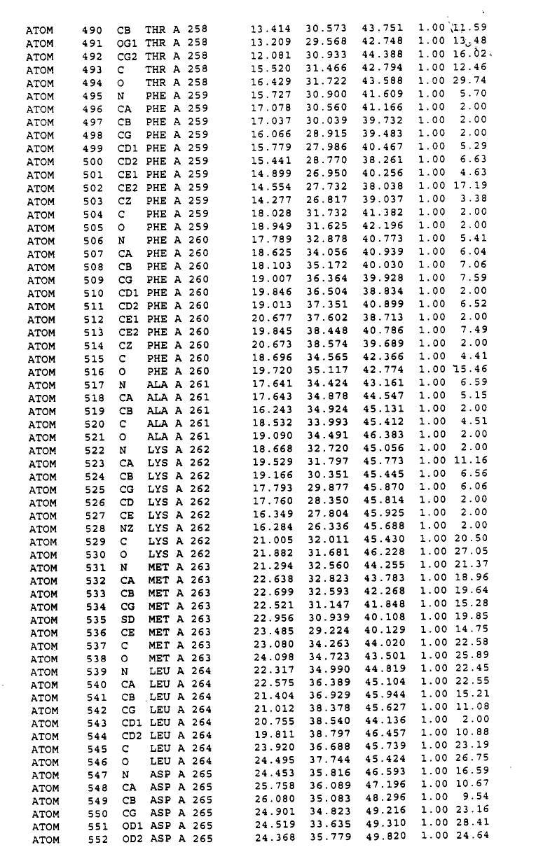

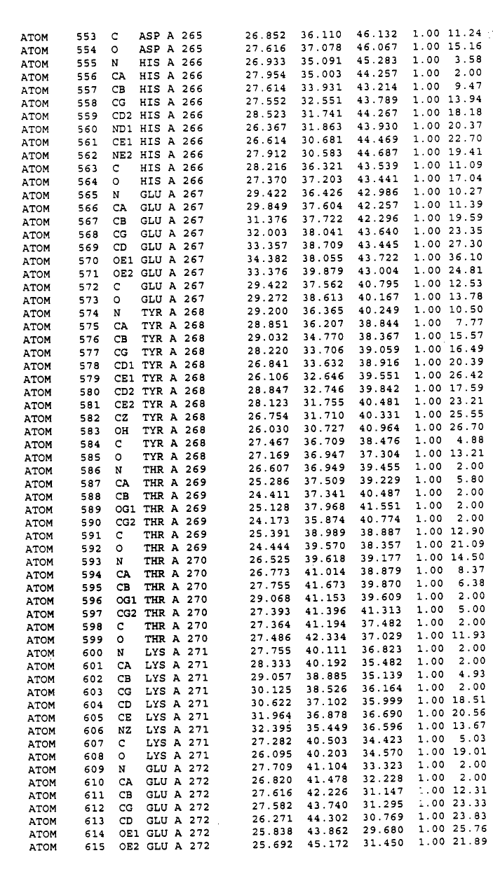

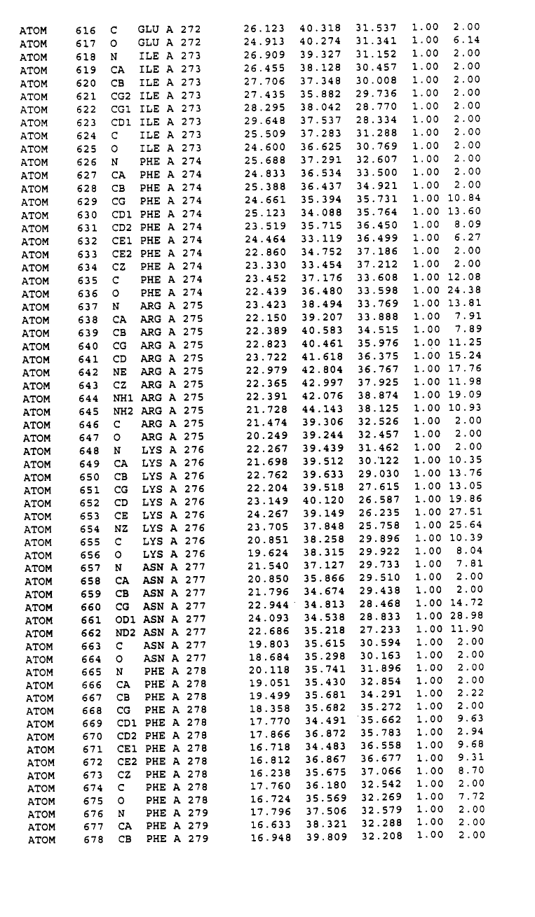

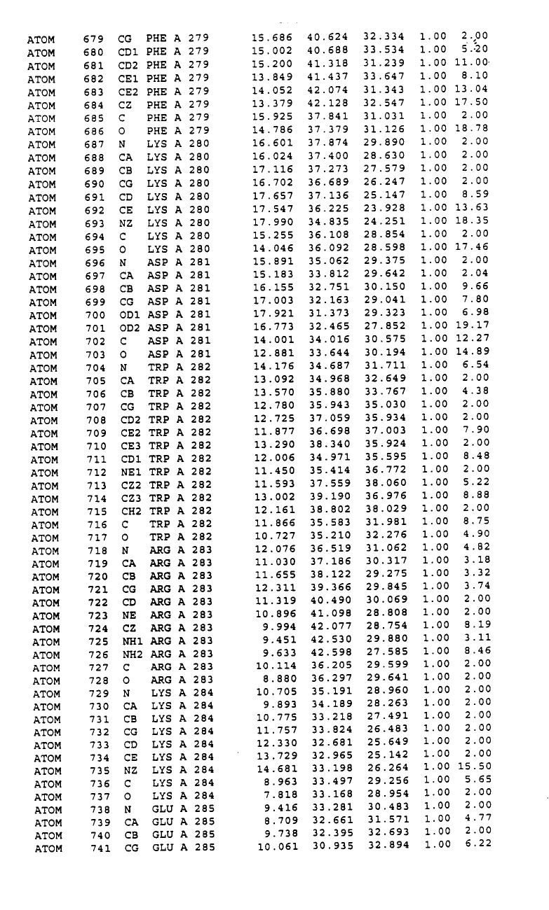

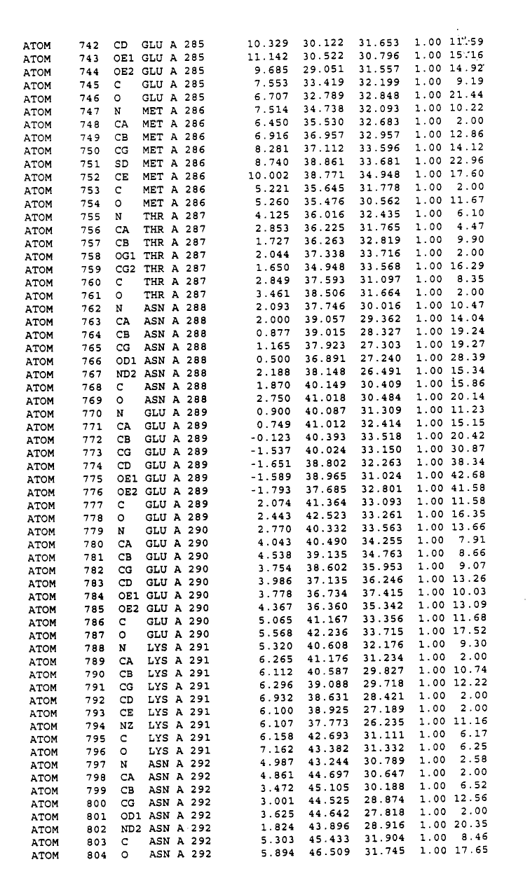

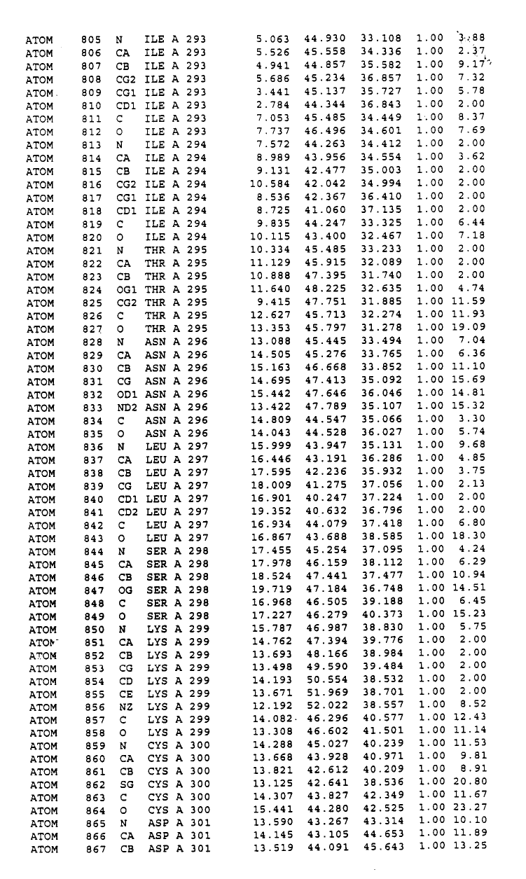

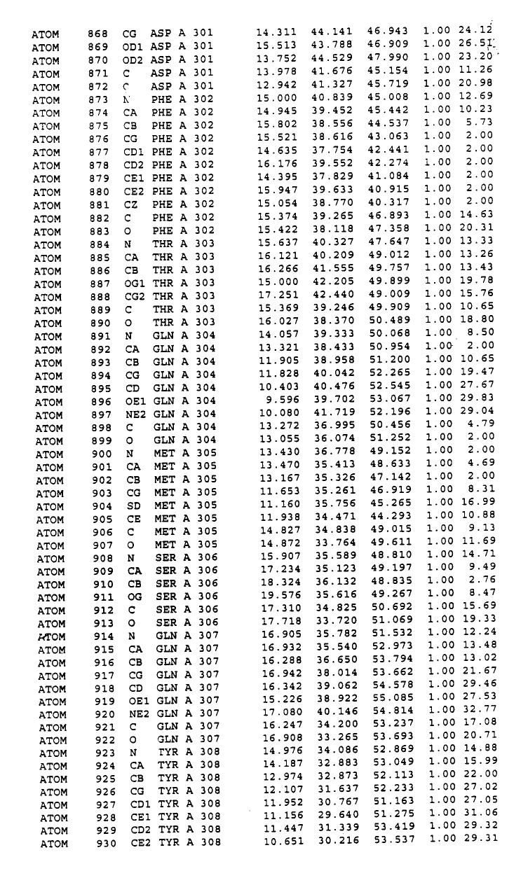

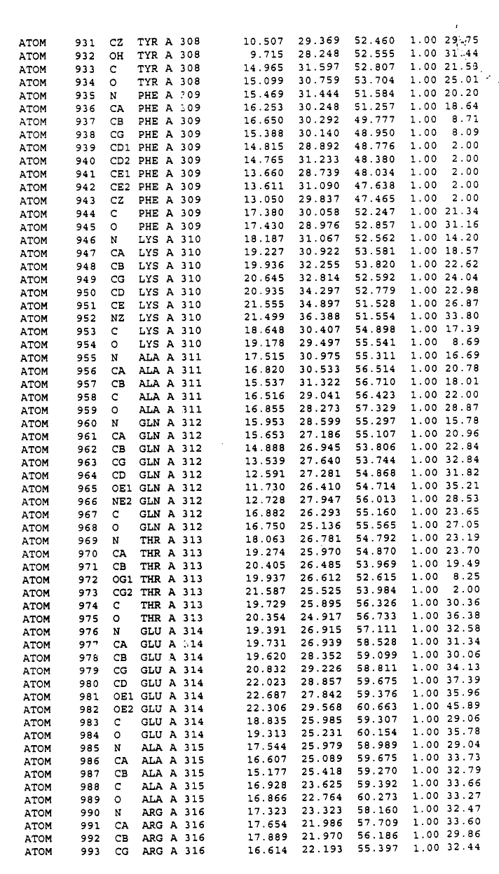

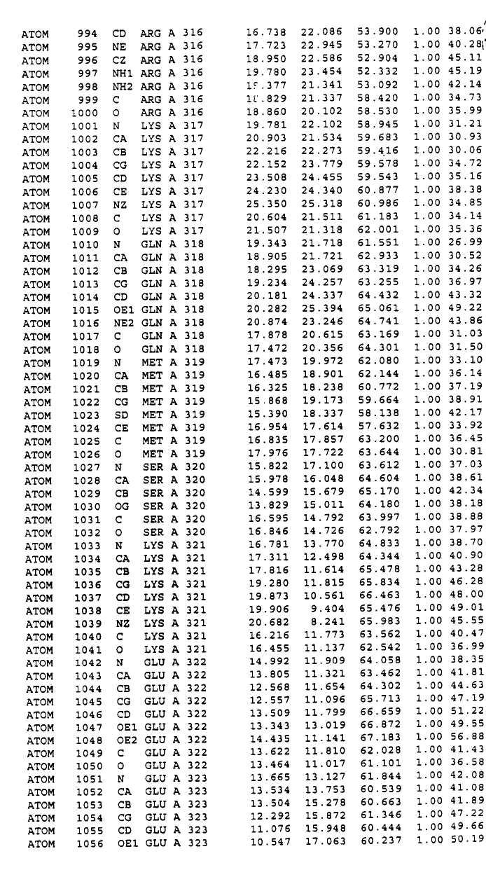

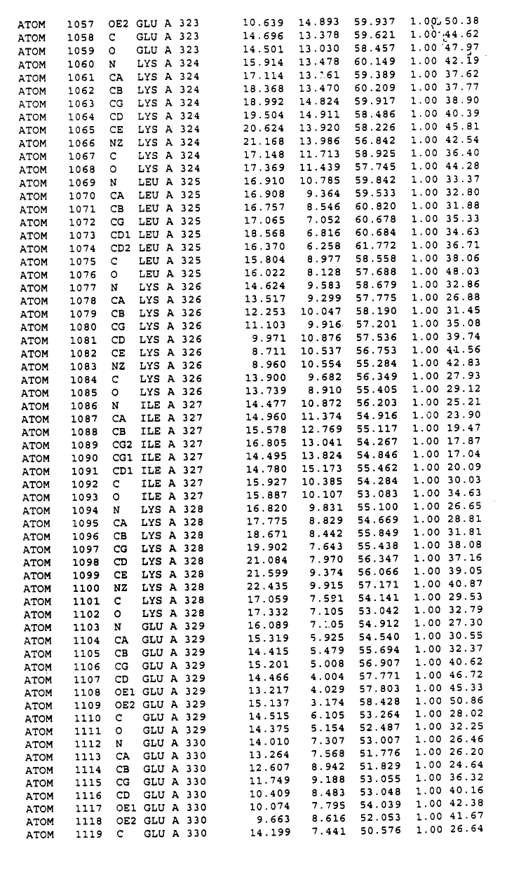

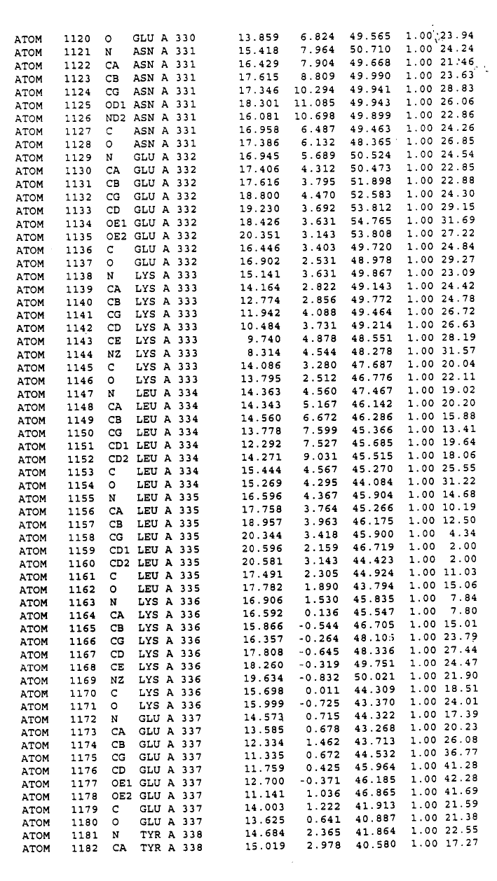

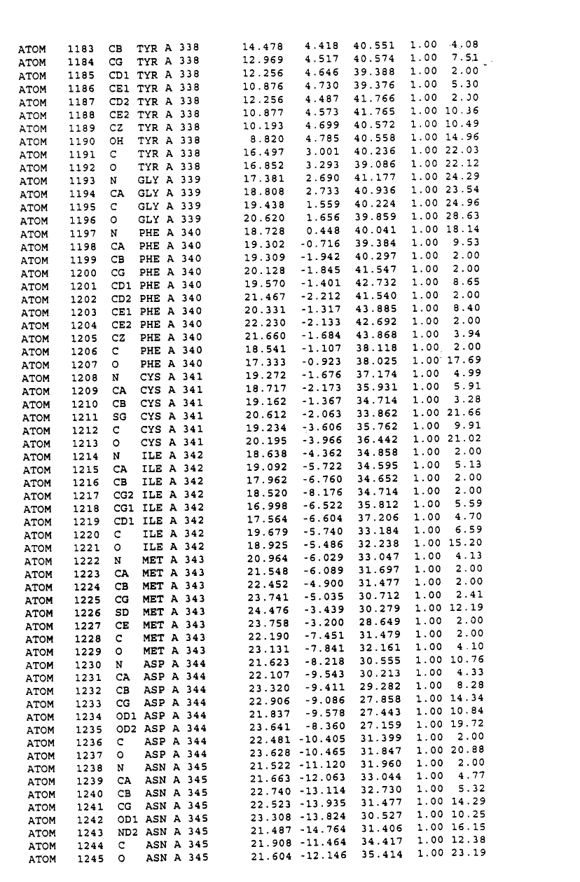

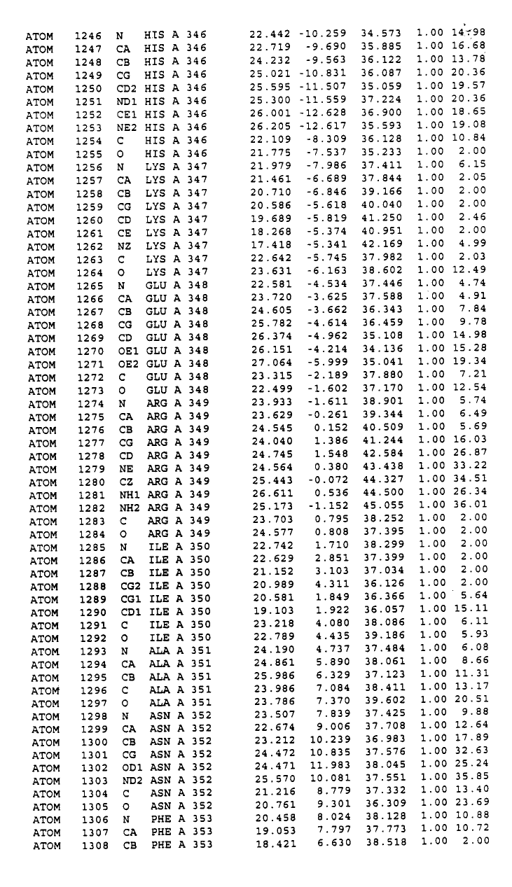

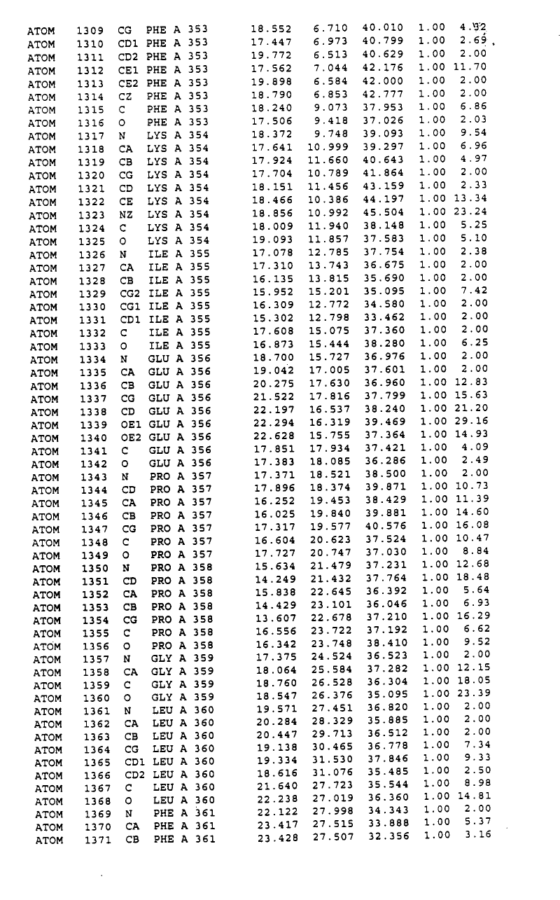

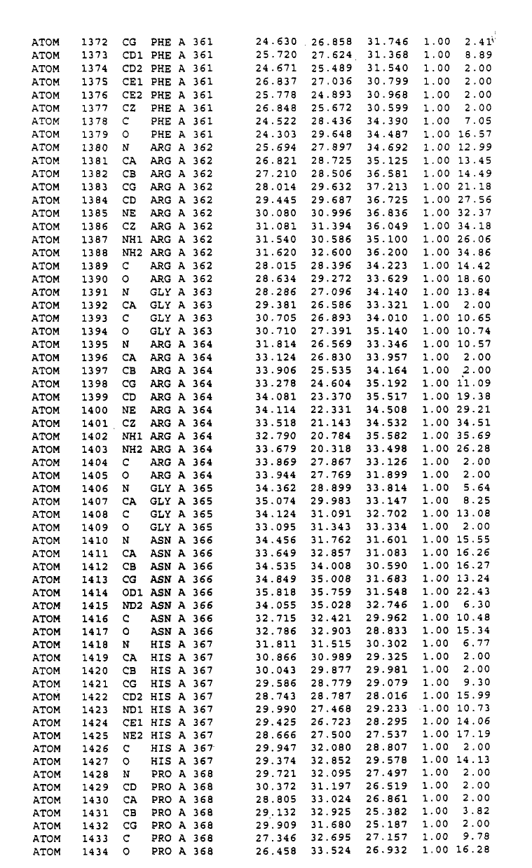

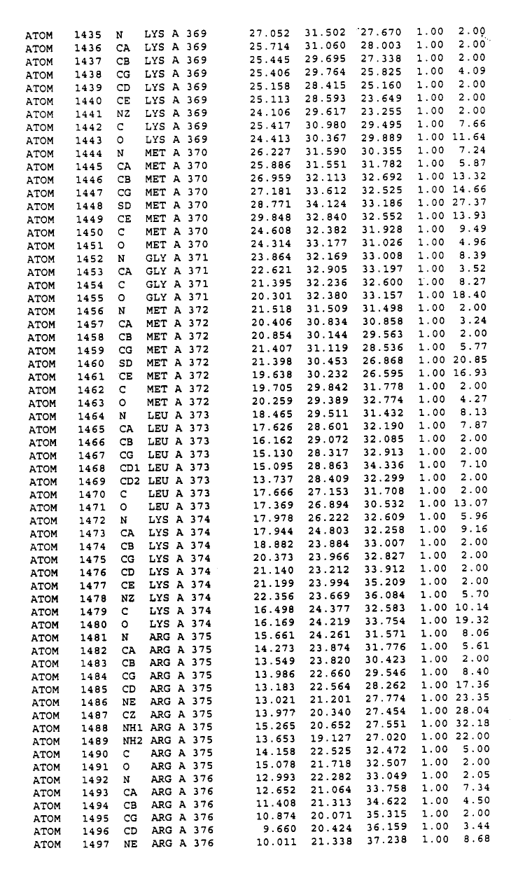

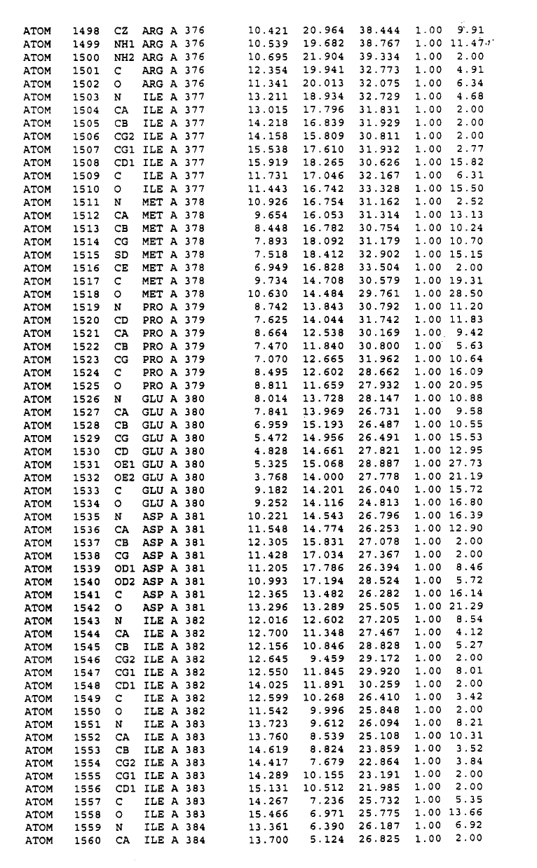

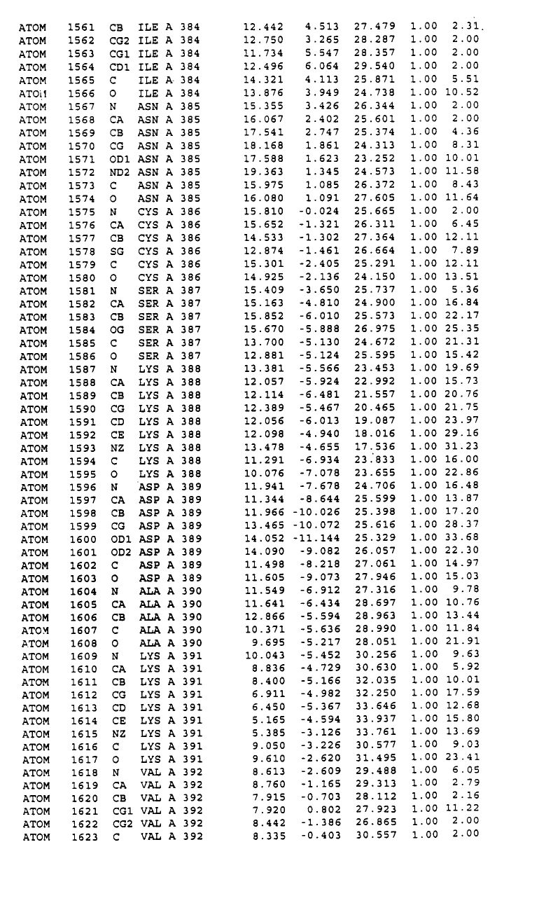

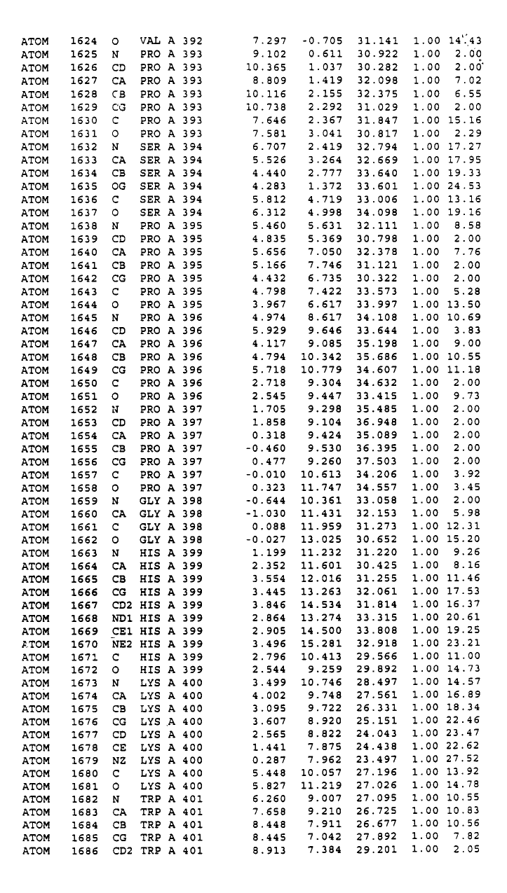

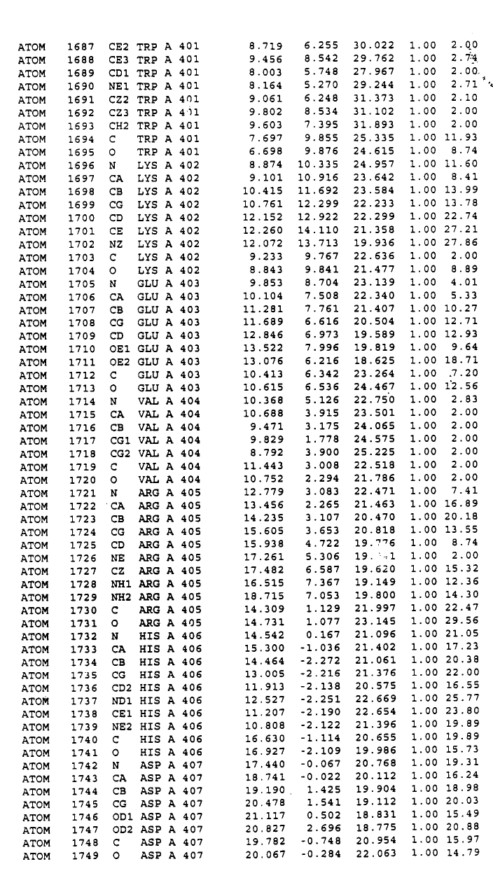

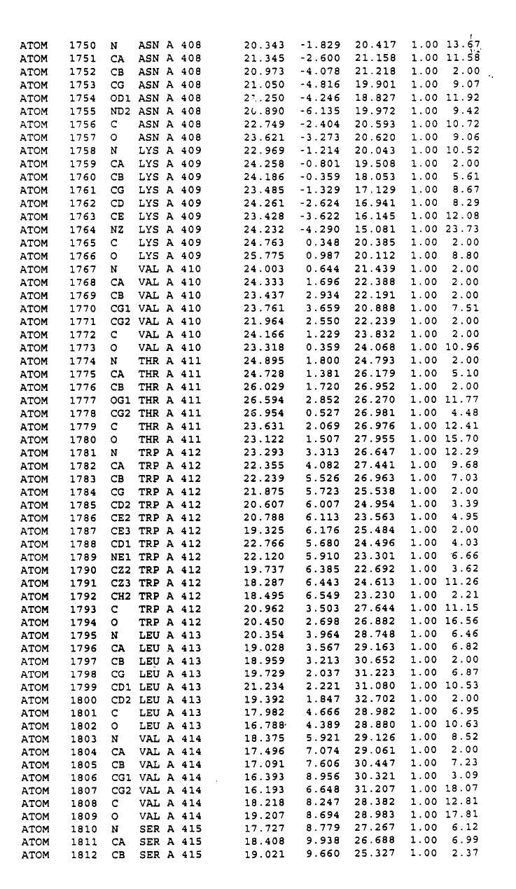

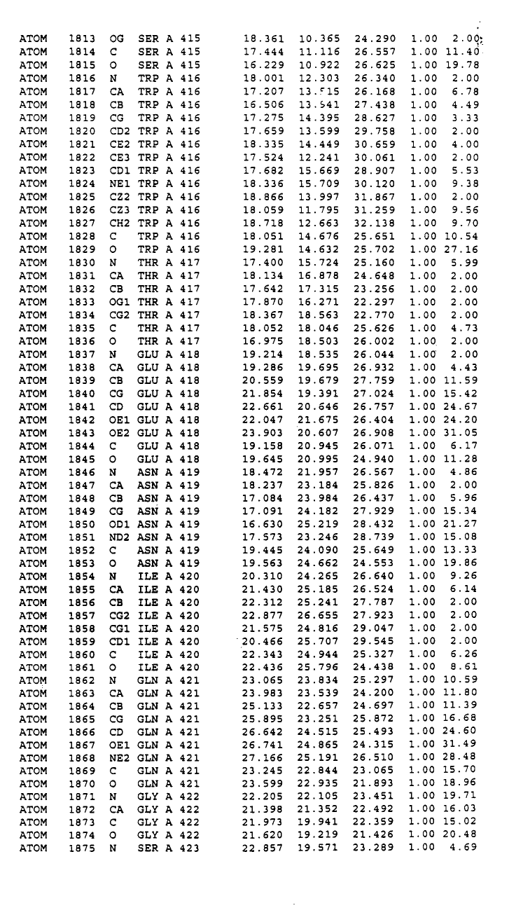

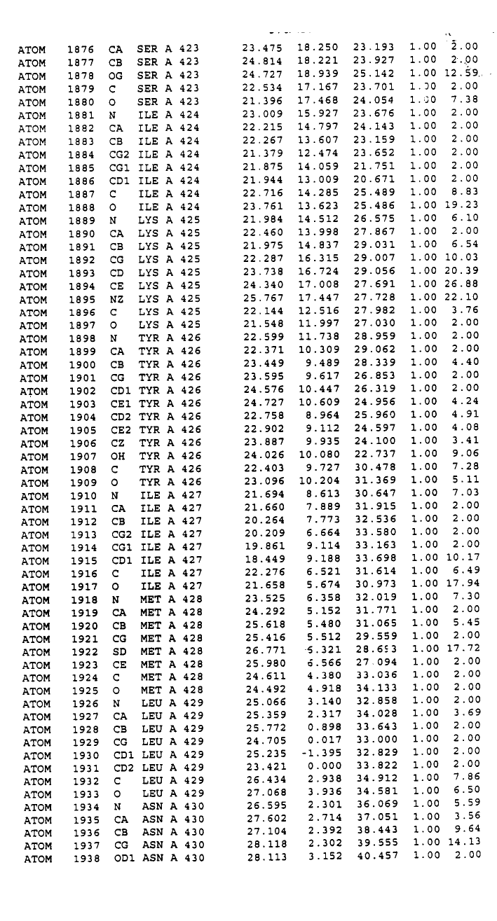

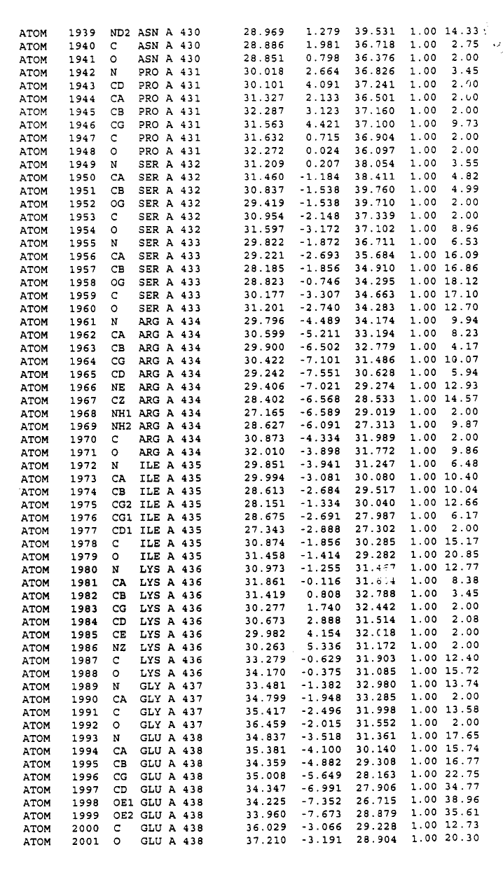

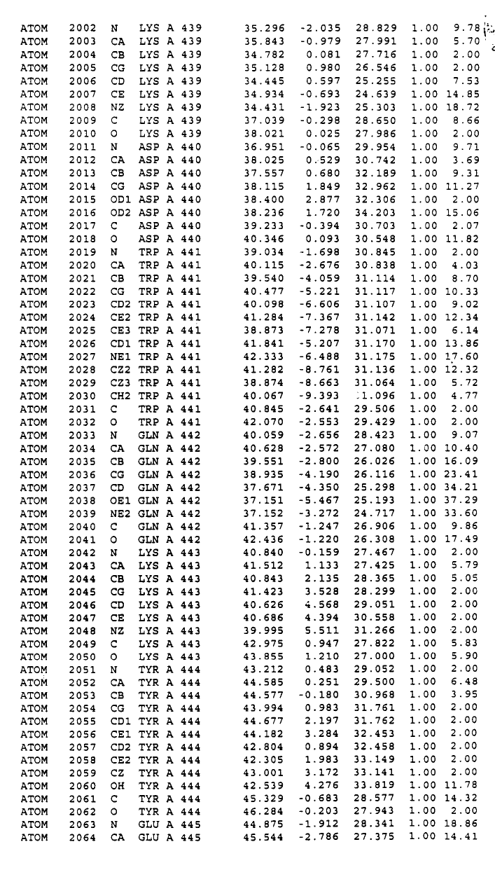

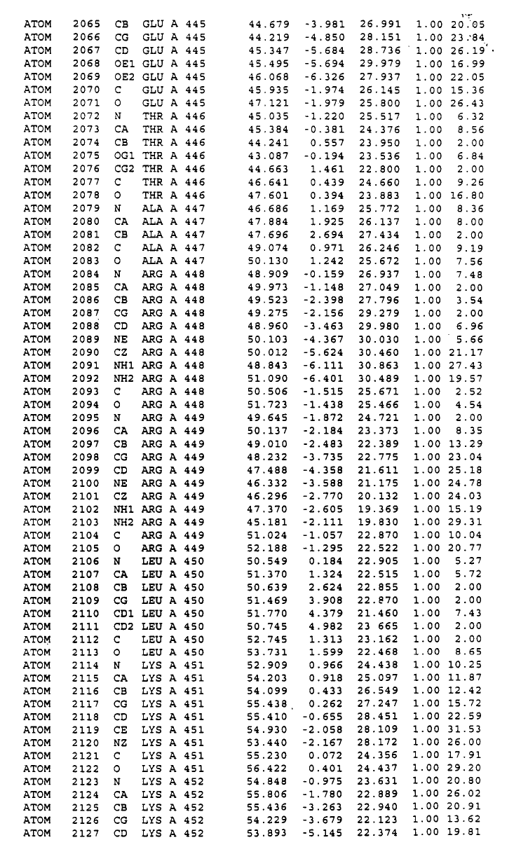

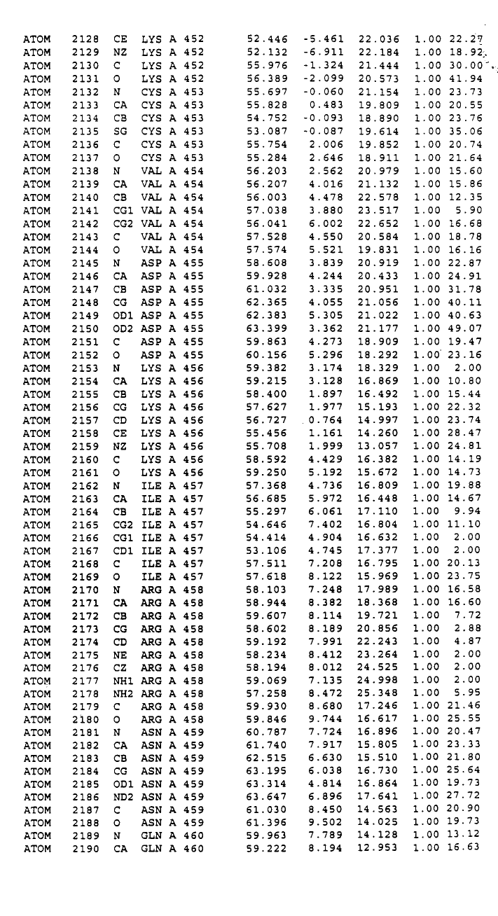

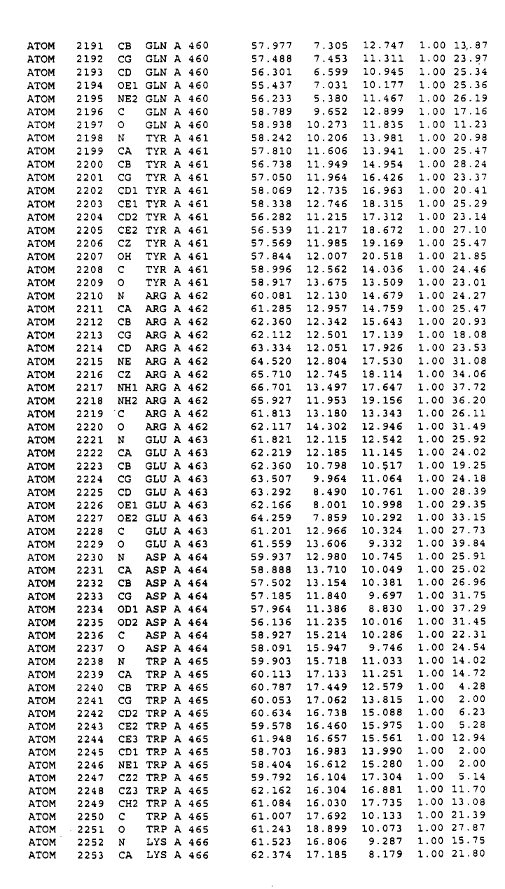

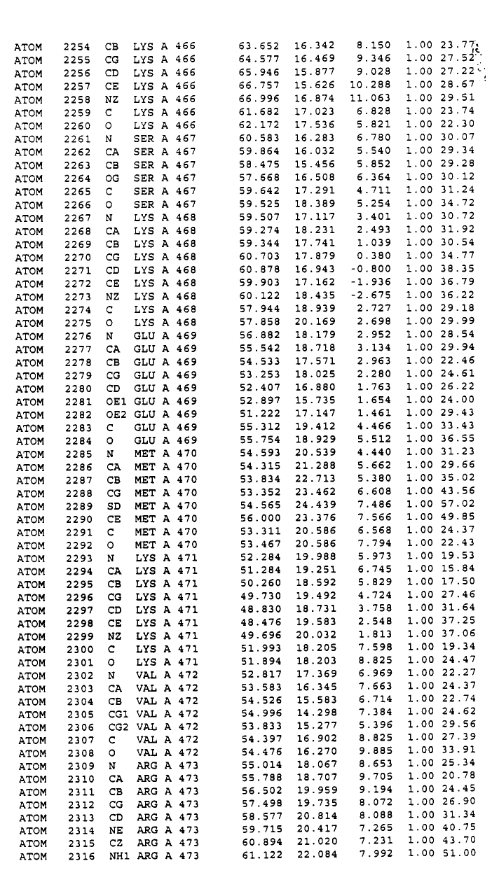

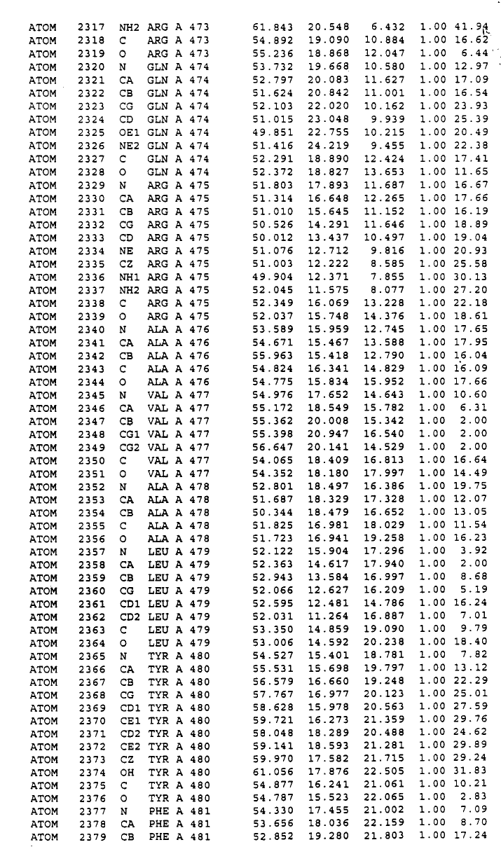

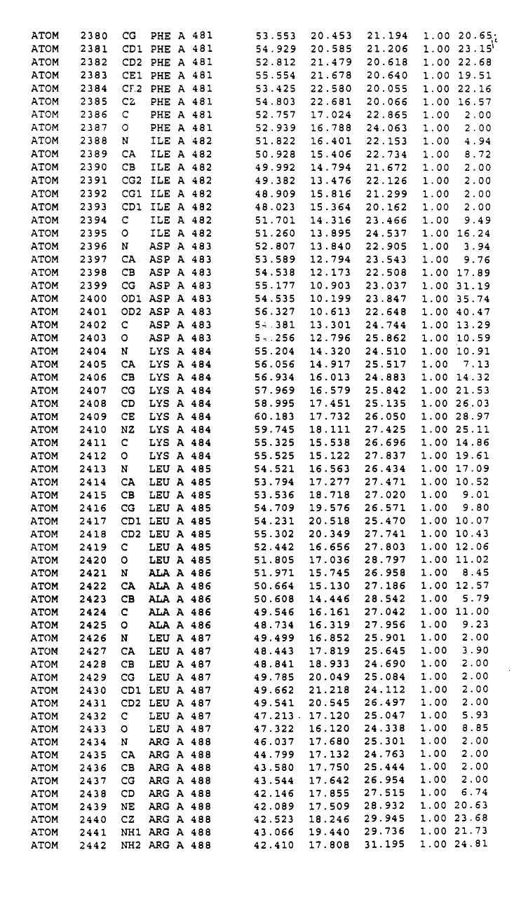

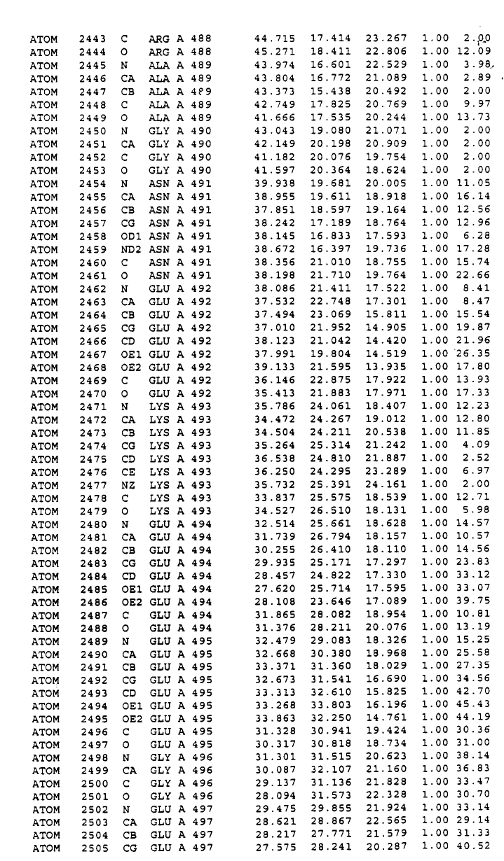

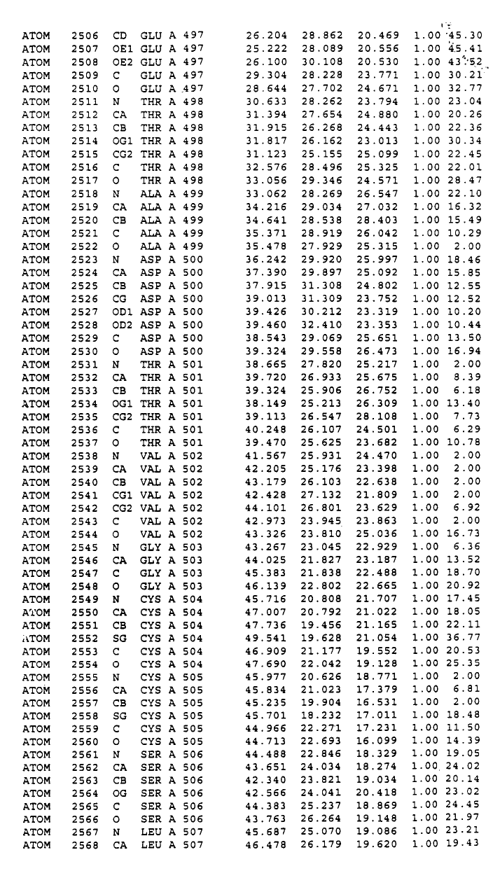

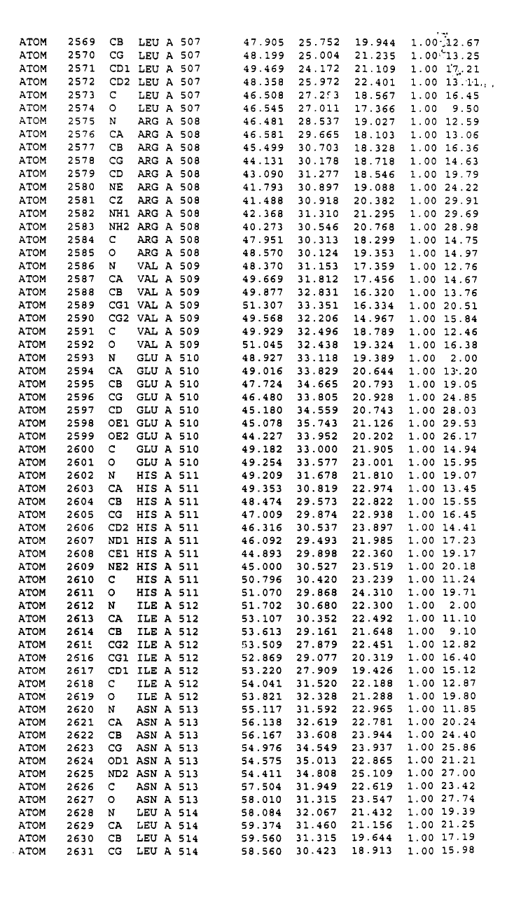

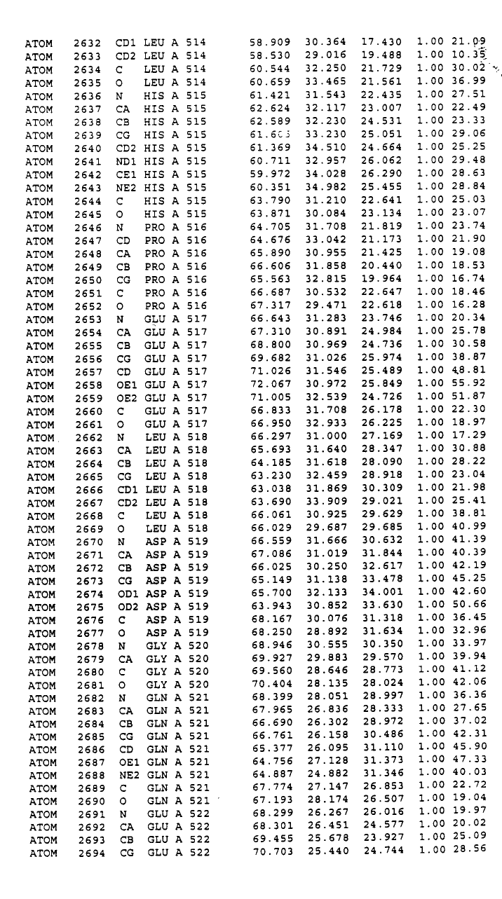

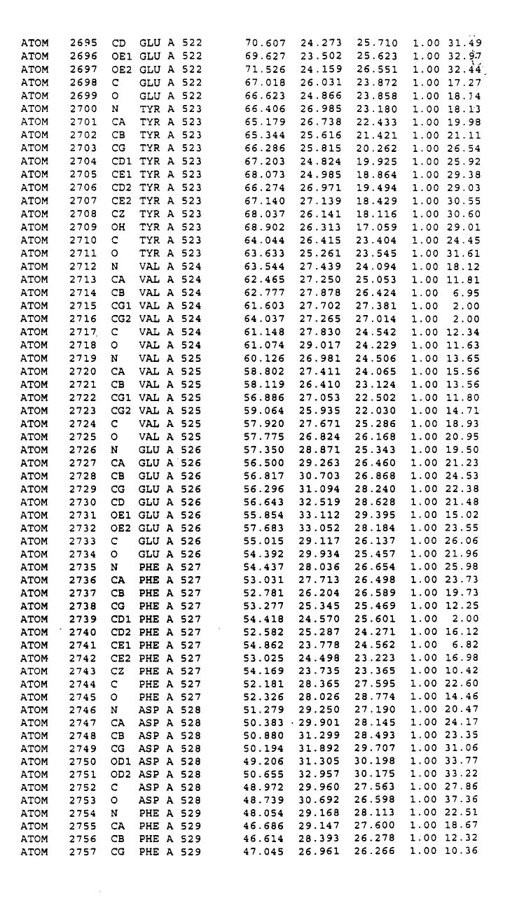

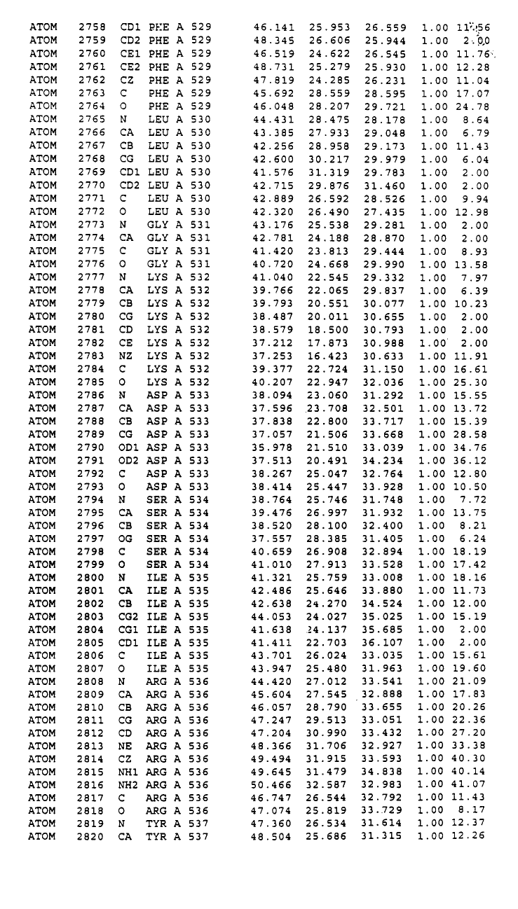

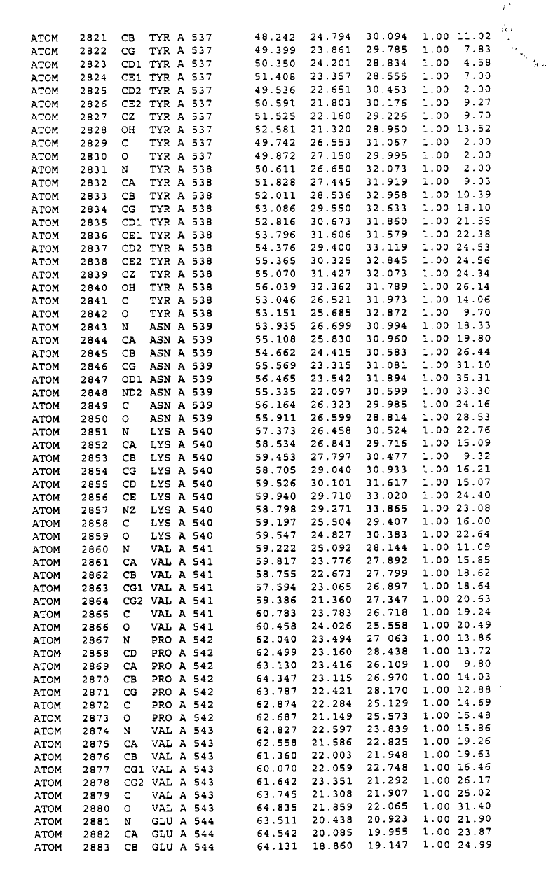

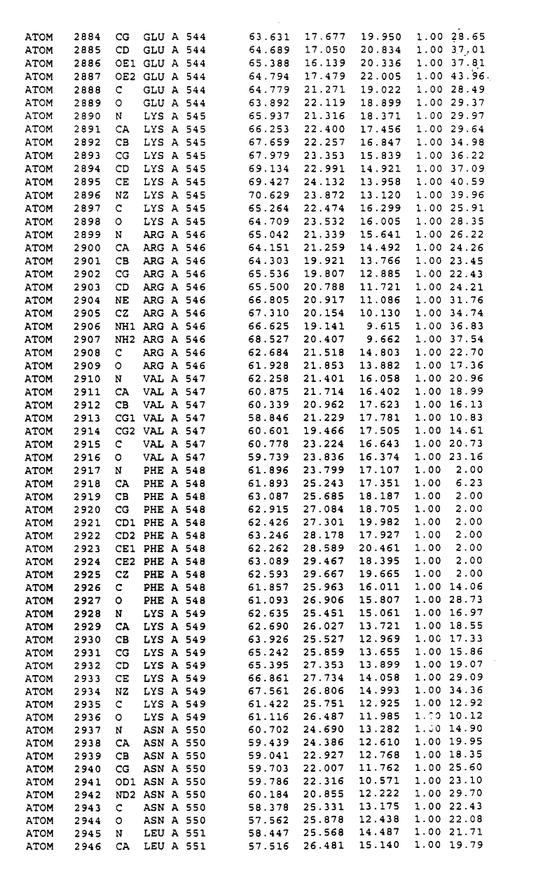

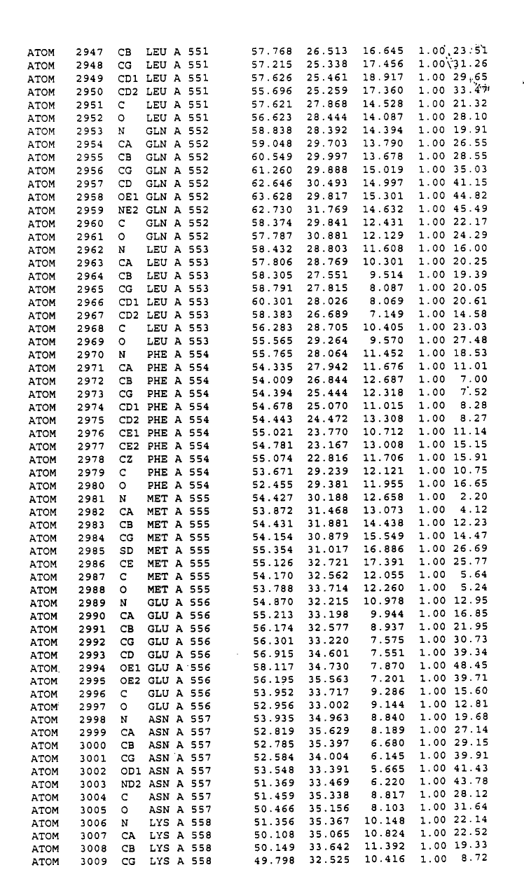

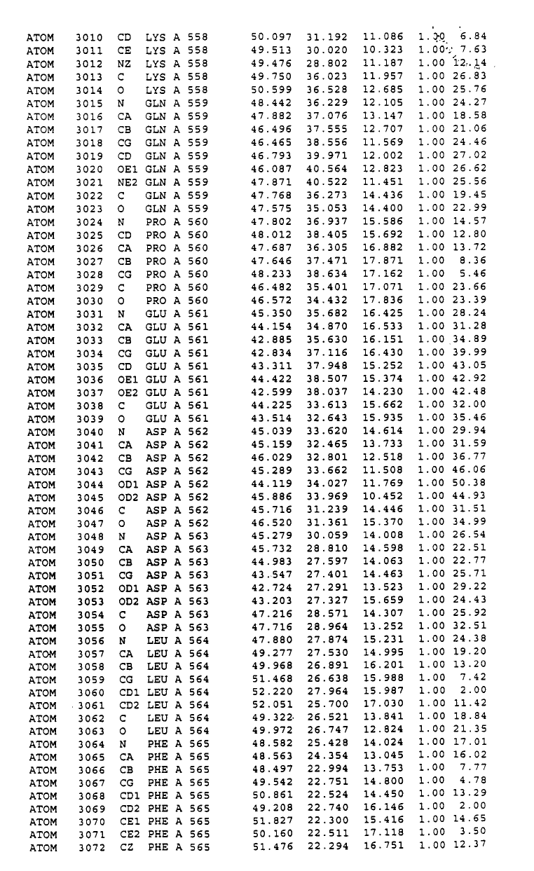

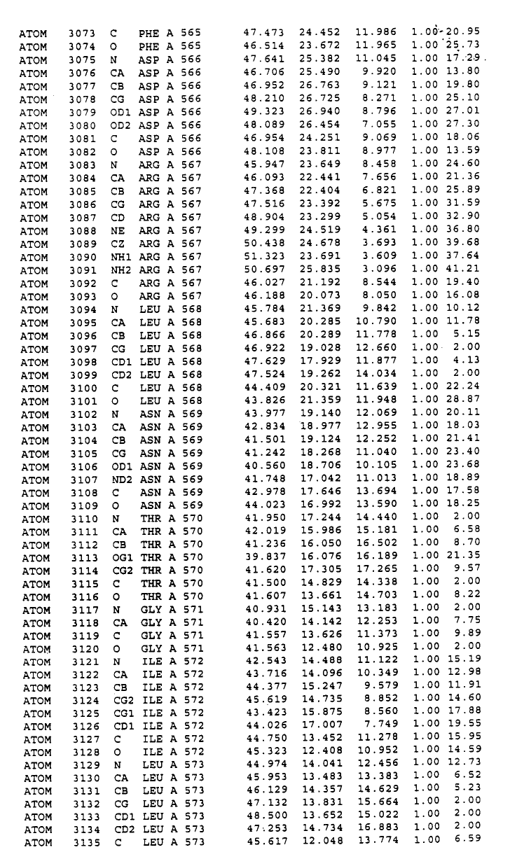

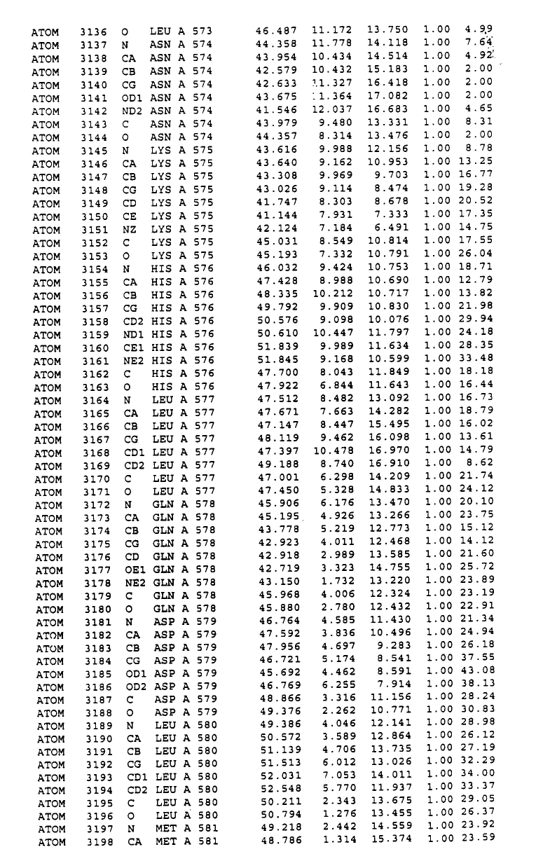

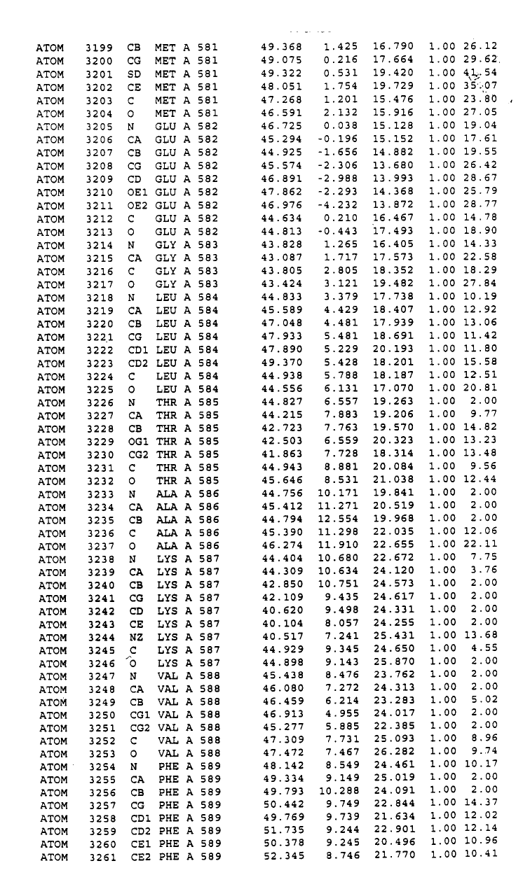

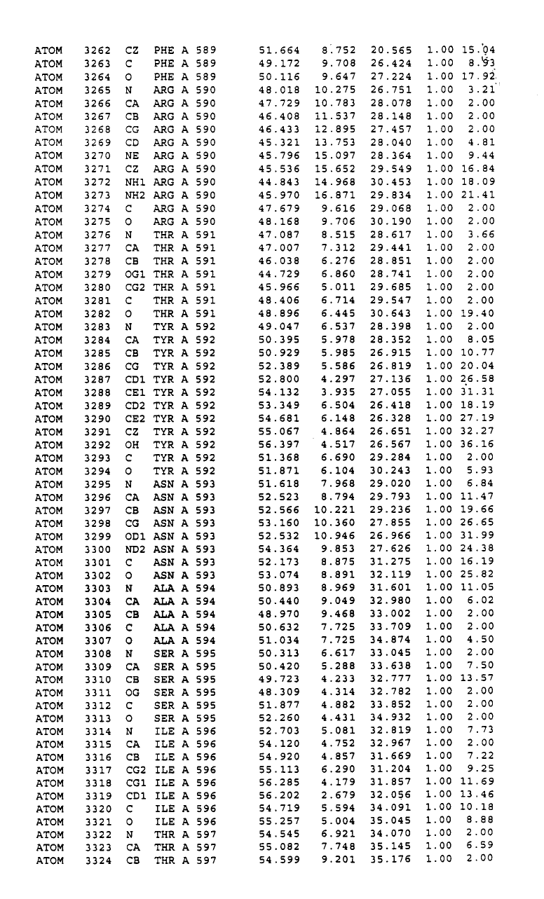

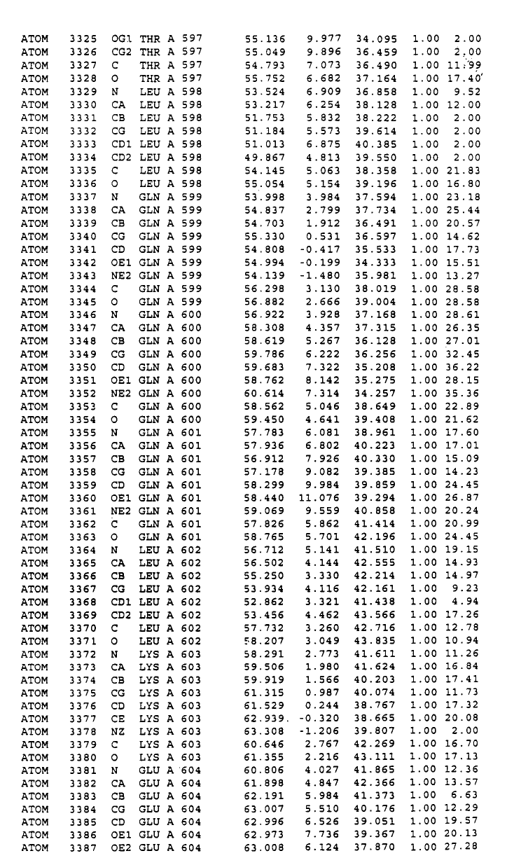

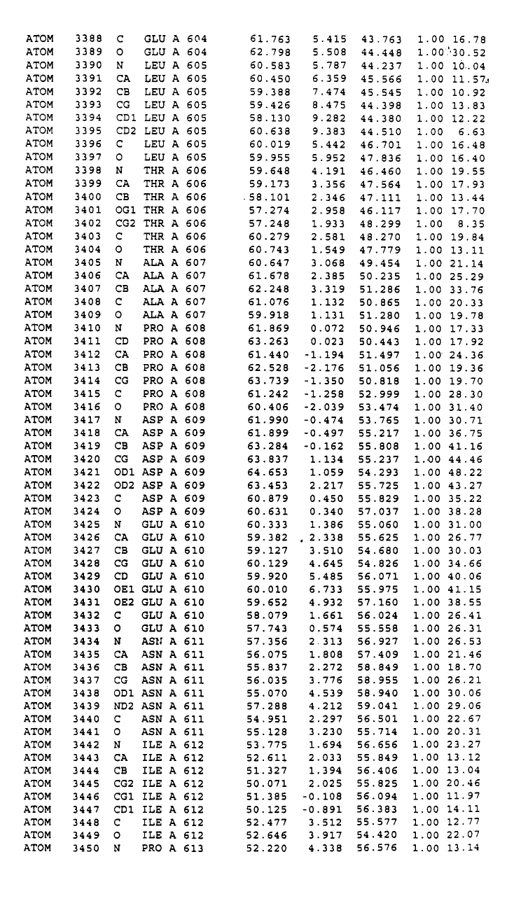

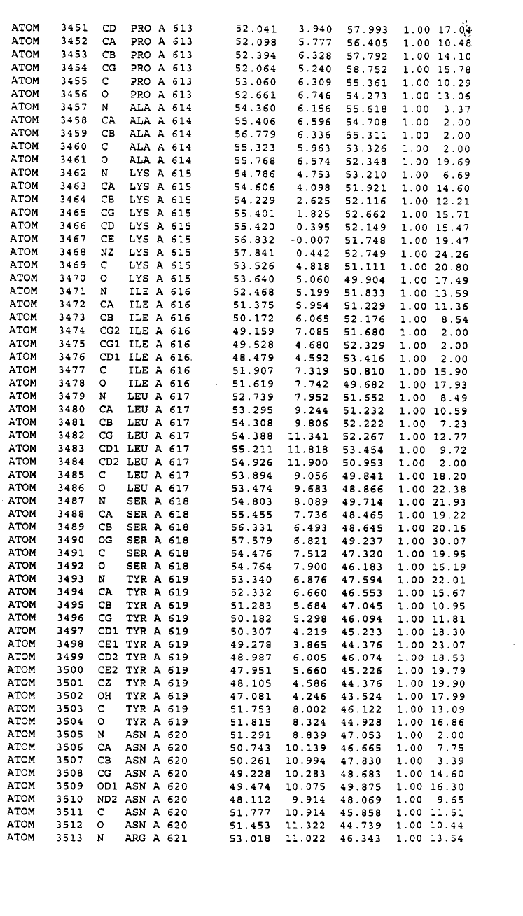

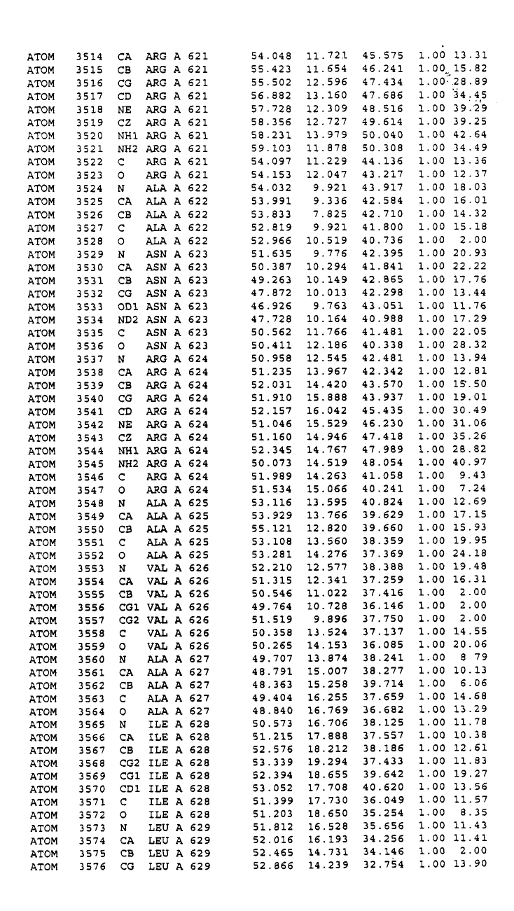

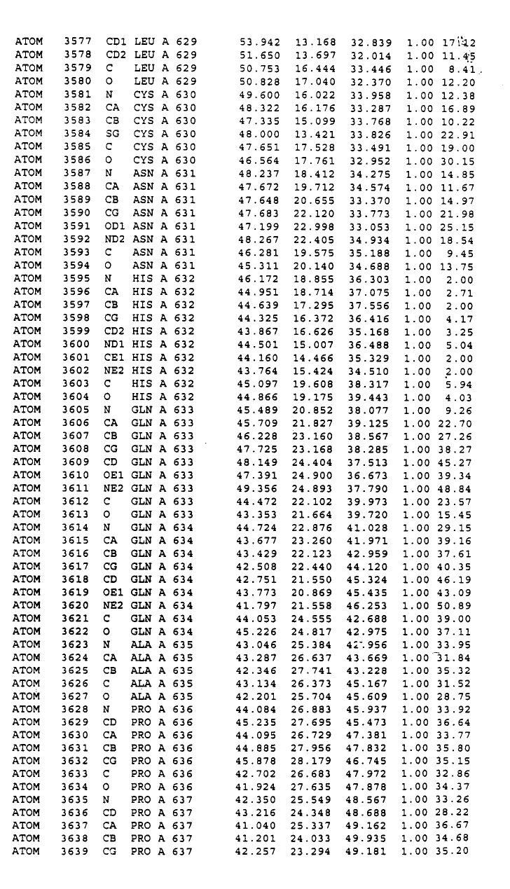

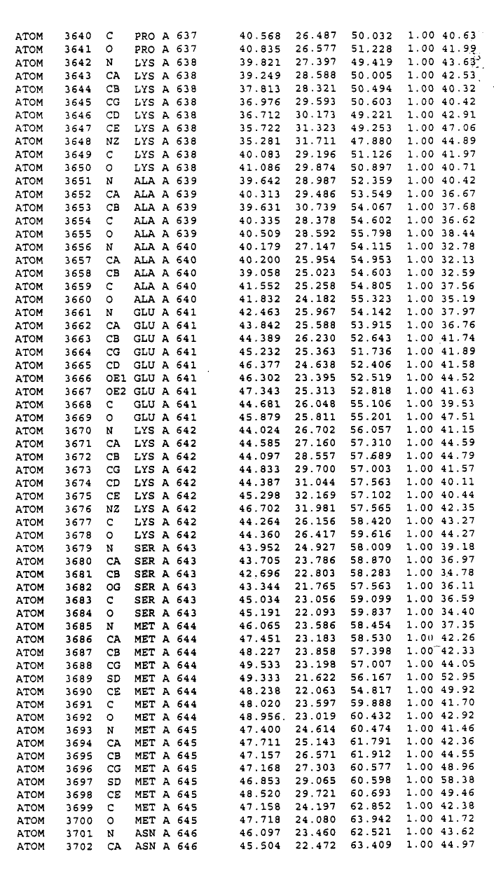

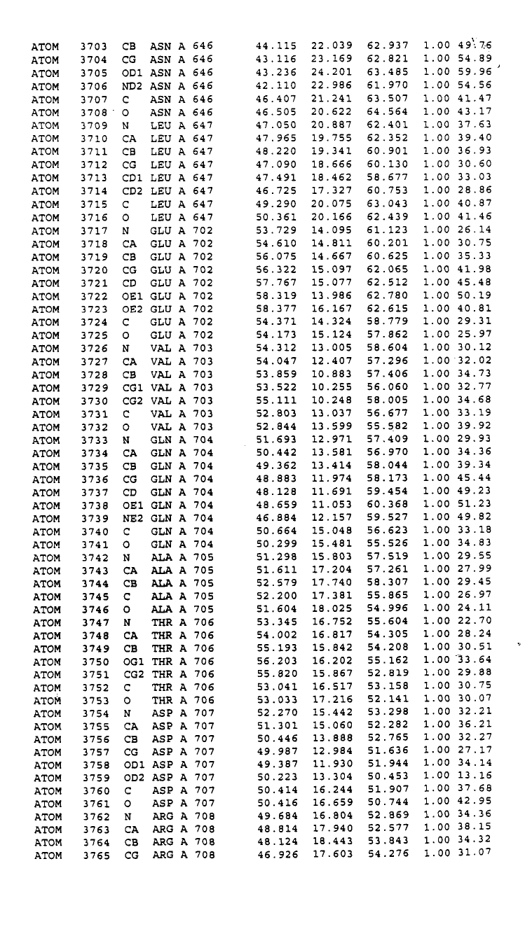

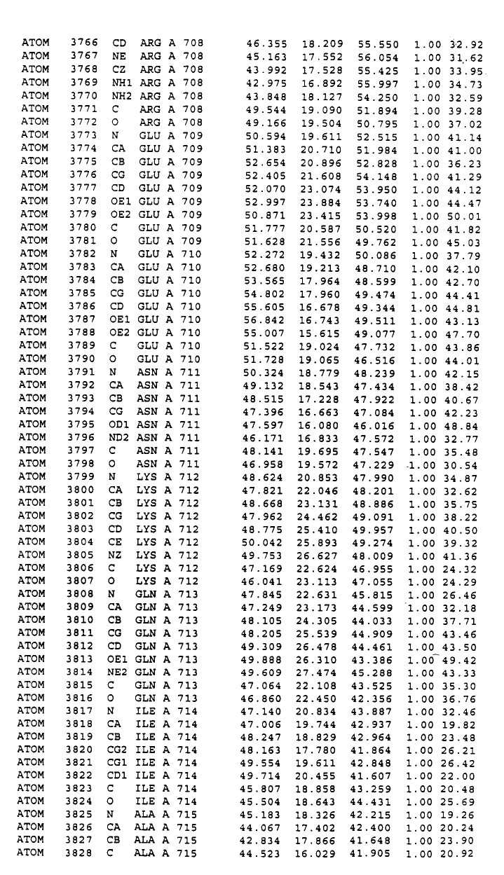

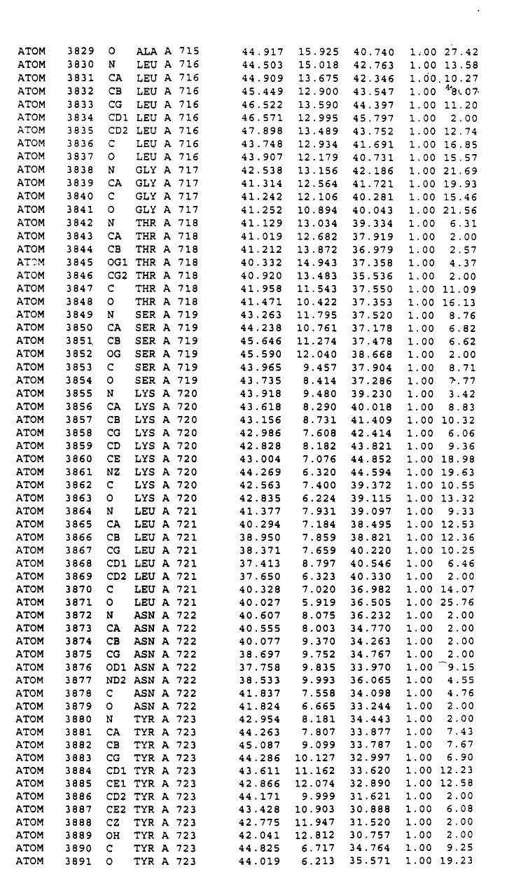

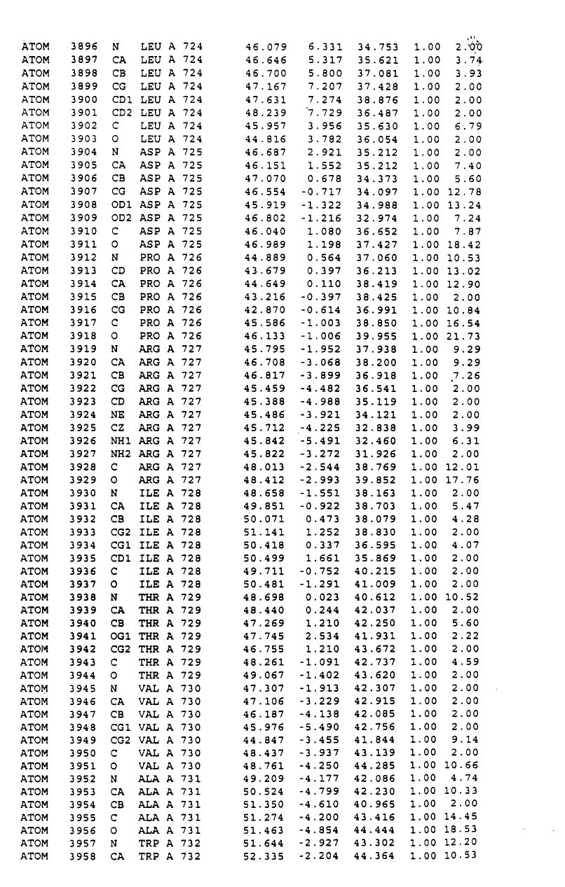

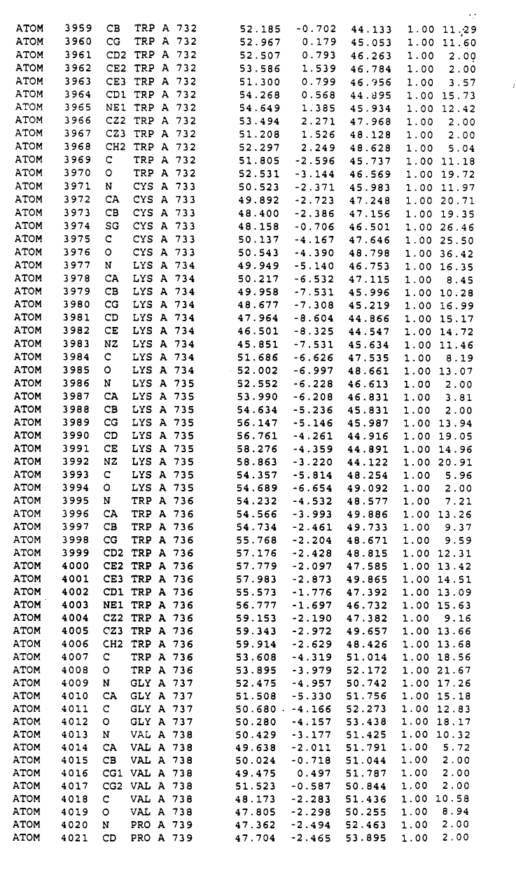

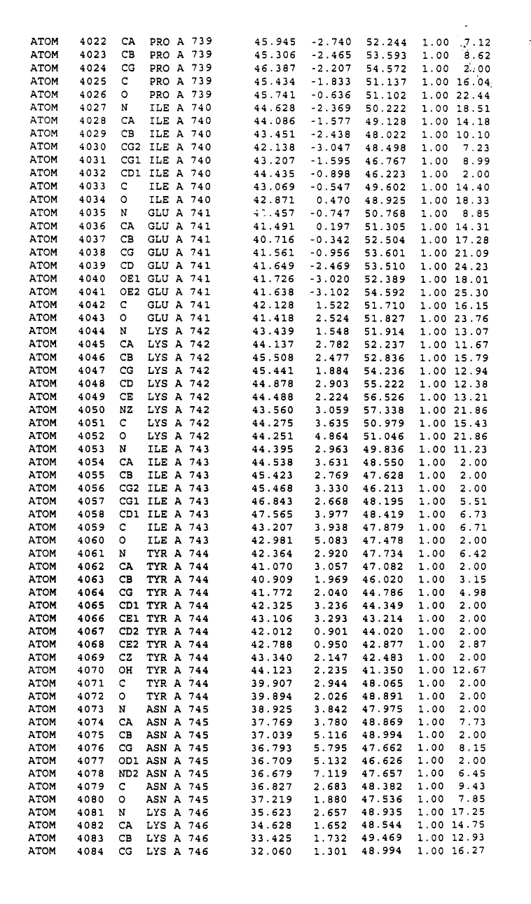

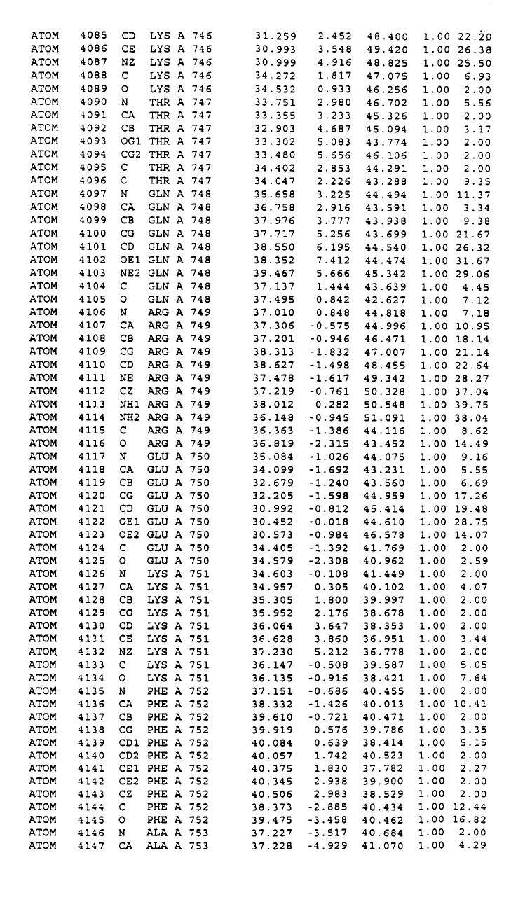

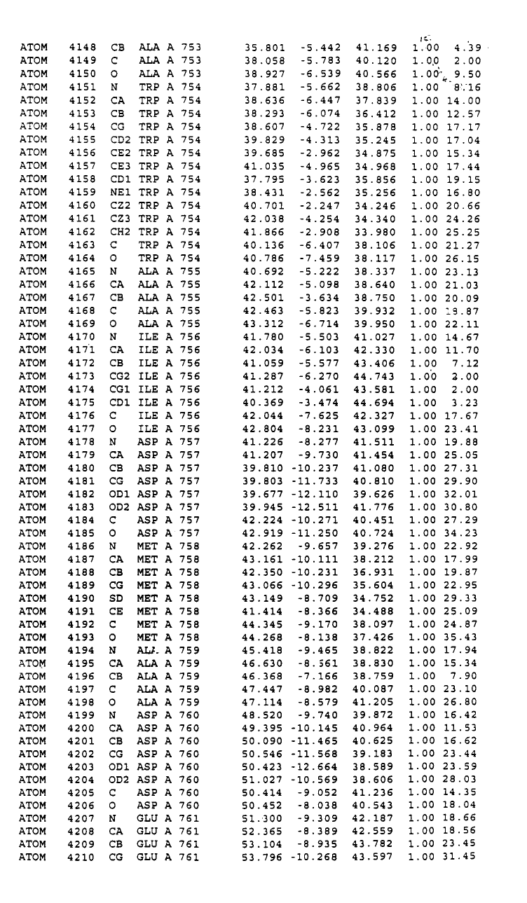

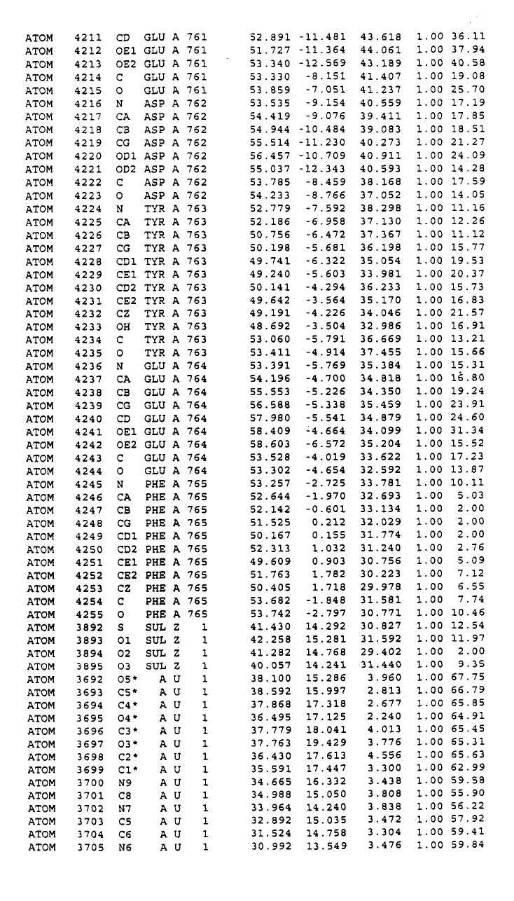

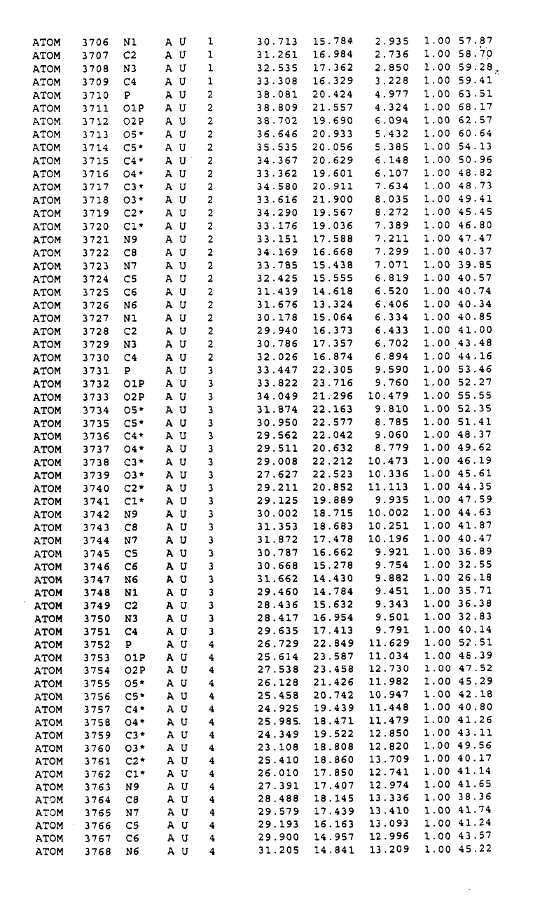

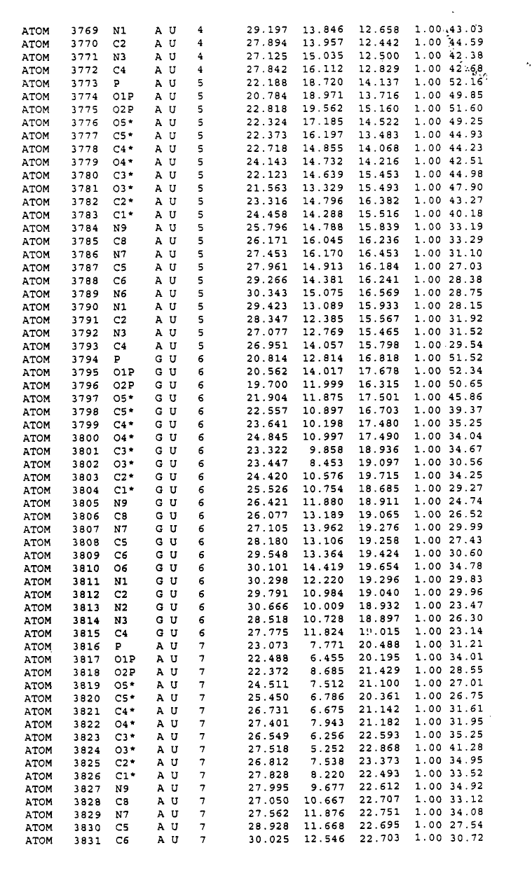

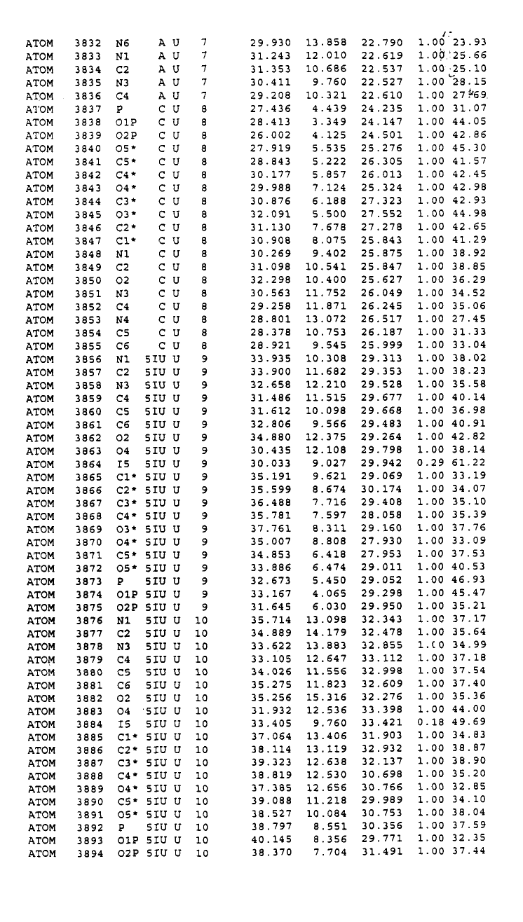

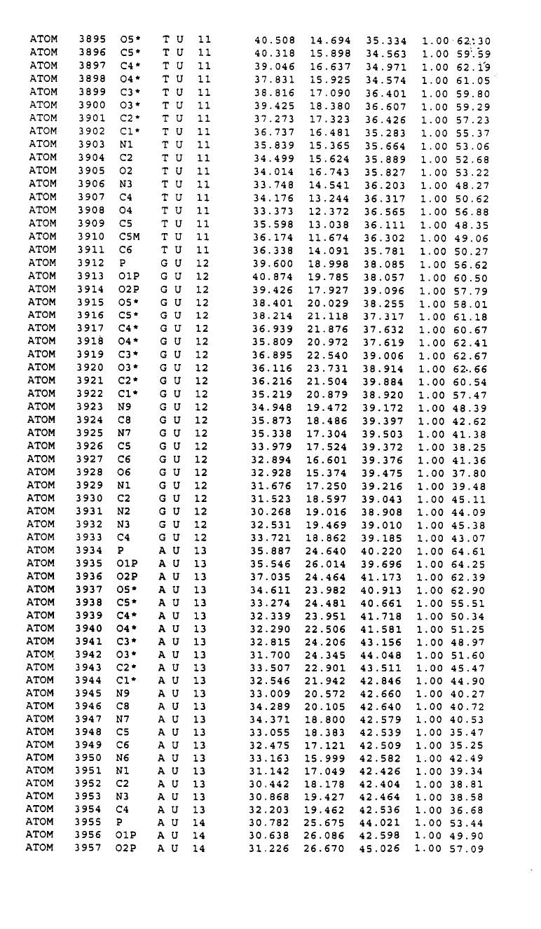

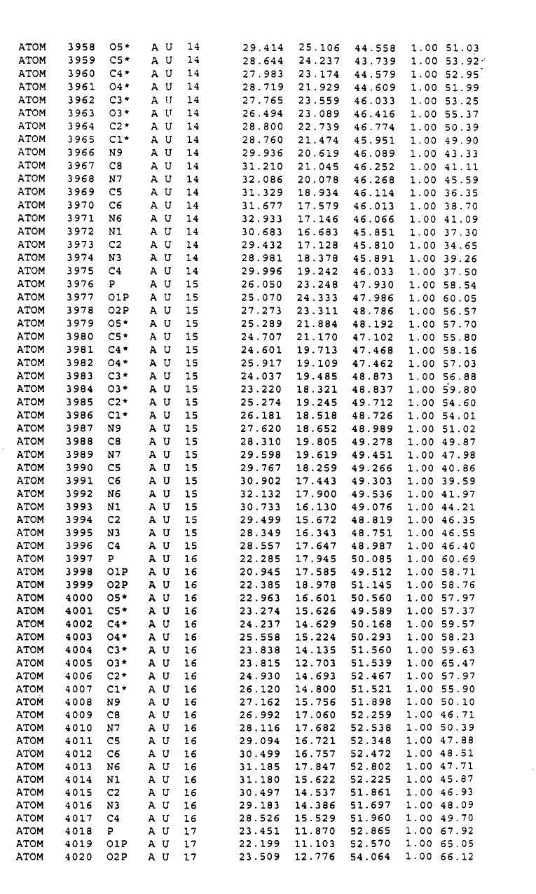

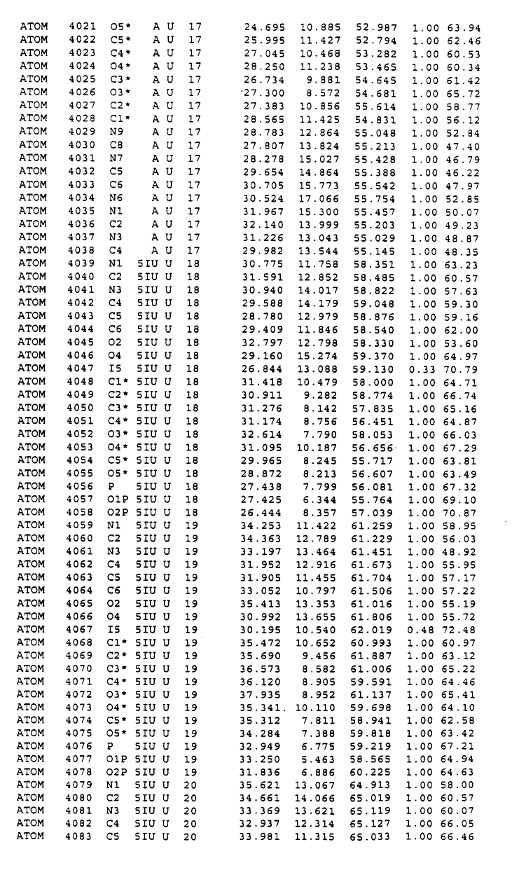

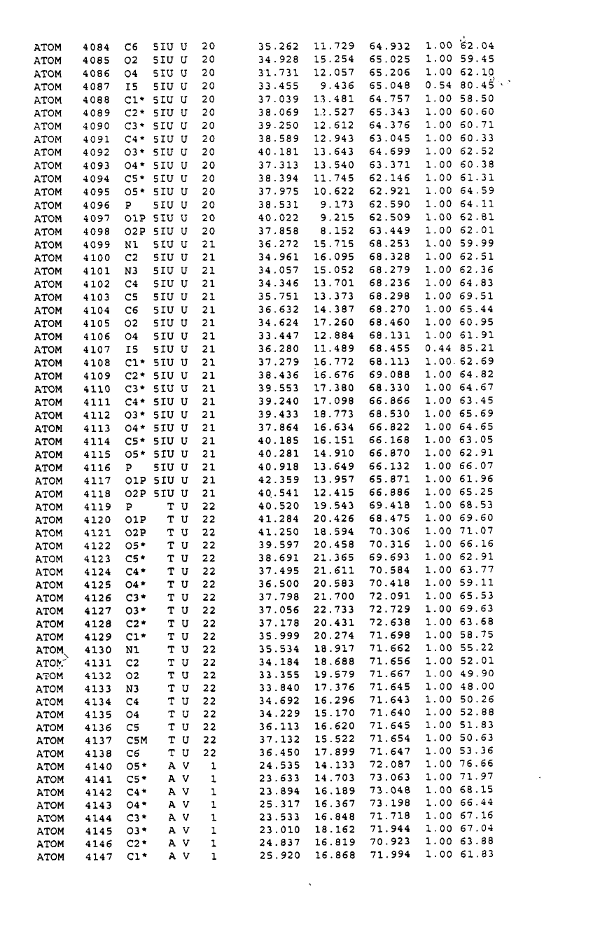

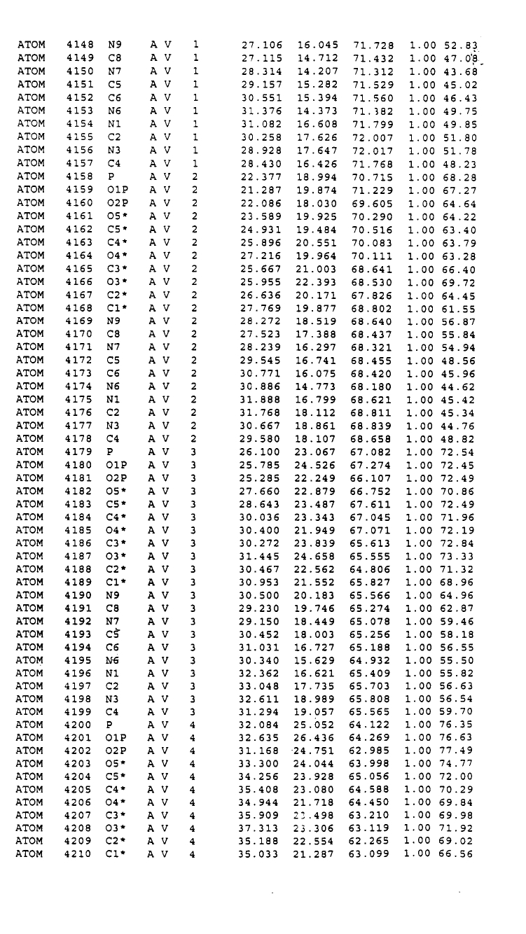

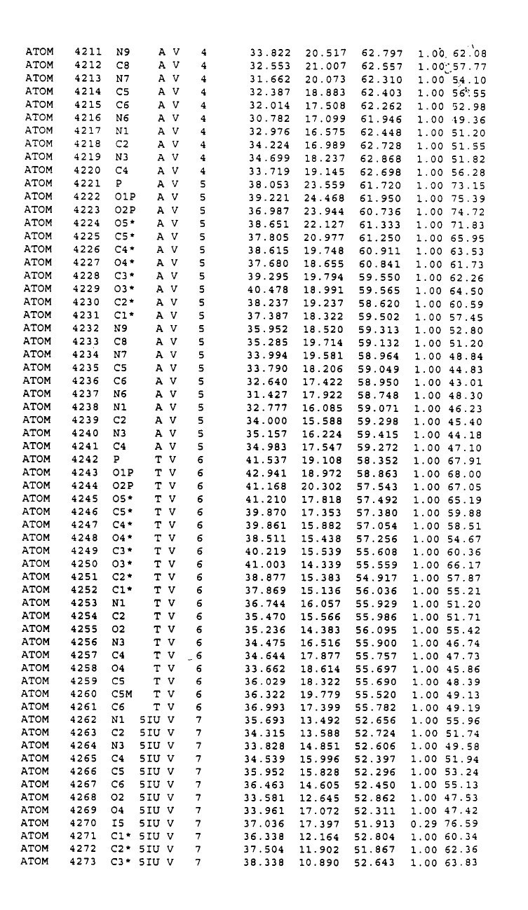

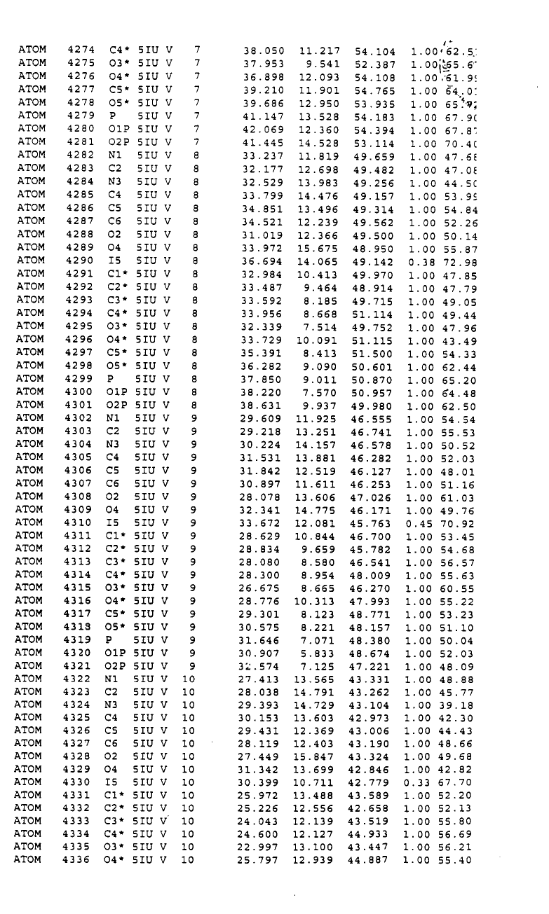

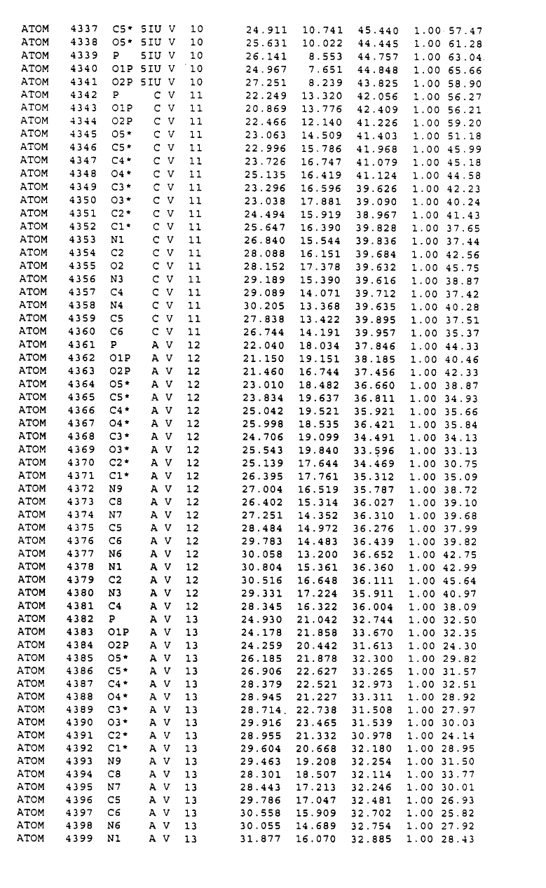

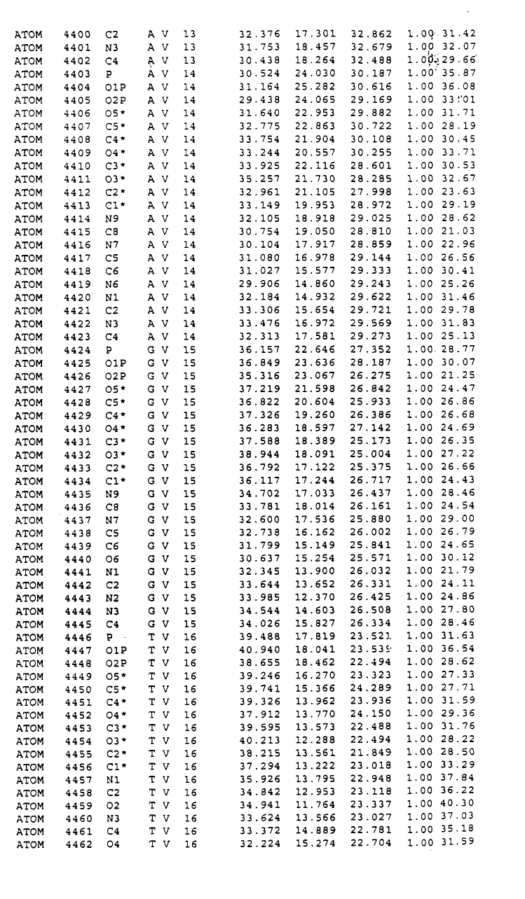

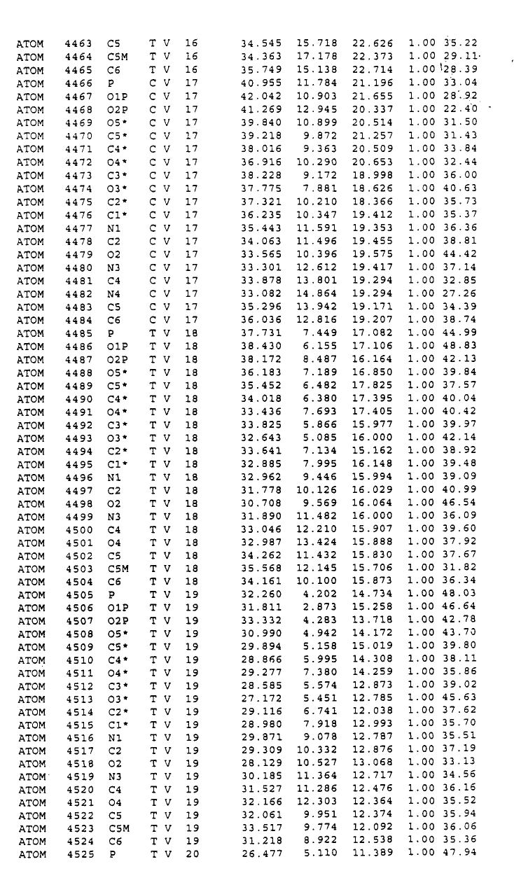

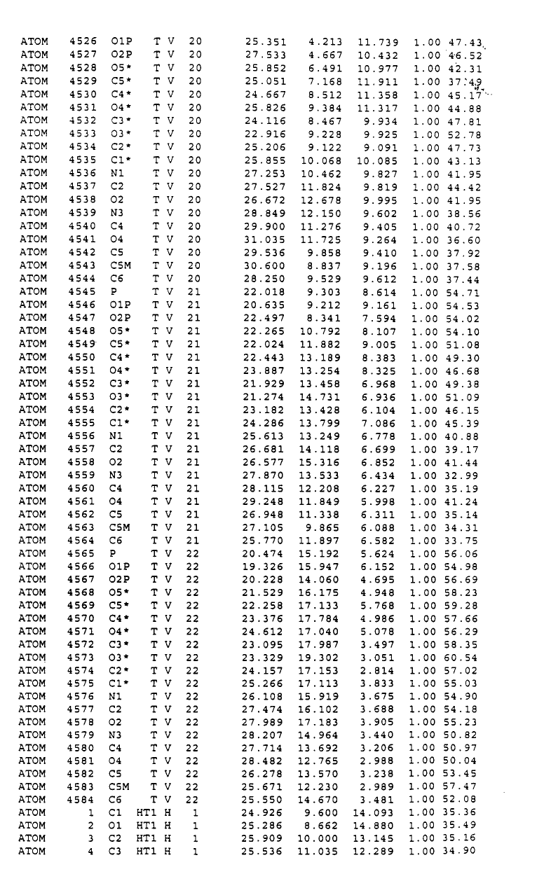

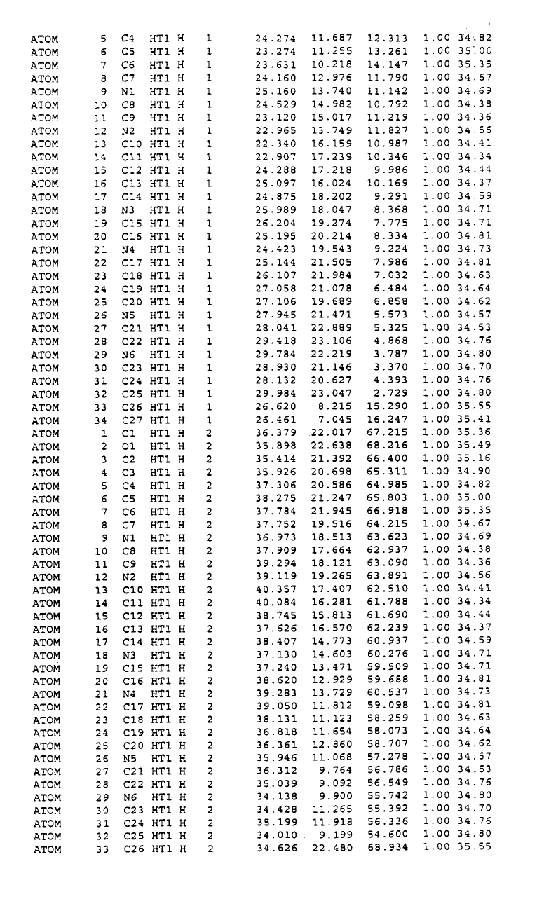

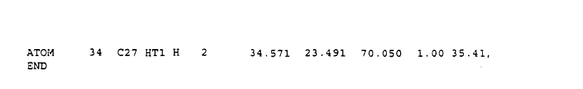

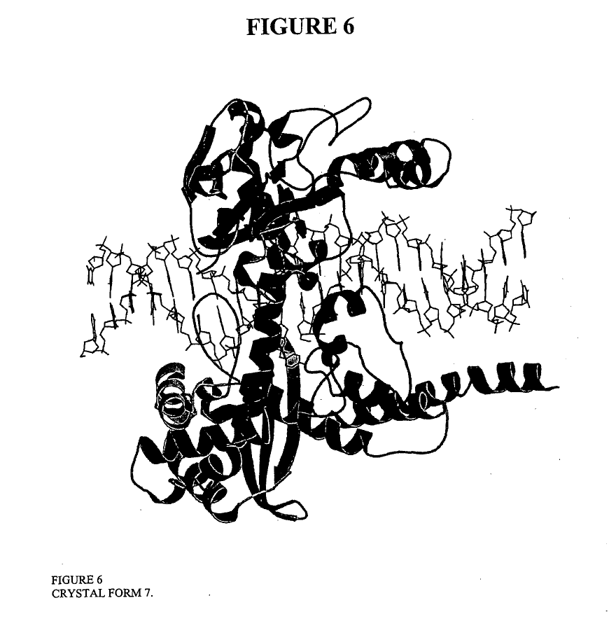

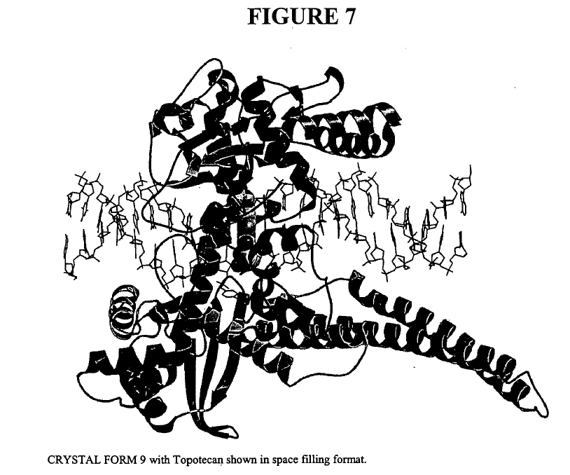

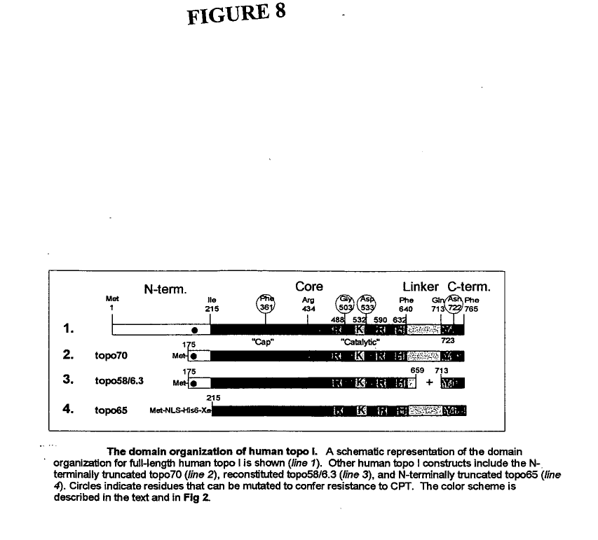

Detailed coordinate for various crystal forms are set-out in FIGS. 1-5

G. Structure Determinations

The X-ray diffraction data collected on the various crystal forms of human topoisomerase I have been obtained at the X25 beamline of the National Synchrotron Light Source (NSLS) at Brookhaven National Laboratory (BNL) in Upton, N.Y.; or at the COM-CAT beam line of the Advanced Photon Source (APS) of the Argonne National Laboratory (ANL) in Argonne, Ill.

All X-ray diffraction experiments were performed with crystals held in a gaseous nitrogen cryo stream at 100 degrees kelvin as described in, Rodgers, D. W., Structure, 2, pp. 1135-1140 (1994). X-ray diffraction data was processed using the software package HKL-2000. This software has been reported in the following reference, Otwinoski, Z. and Minor, W., Meths. Enzymol., 276, pp. 307-326 (1997)

Structure determinations have been performed using molecular replacement, Navaza, J., Acta. Crystallogr., A50, pp. 157-163 (1994), in conjunction with CNX, Brlnger, A. T., et al., Acta. Crystallogr., D54, pp. 905-921 (1998)), and XtalView crystallographic computing packages under license to Emerald BioStructures, Inc. McRee, D. E., J. Struct. Biol., 125, pp. 156-165 (1999).

H. Crystal Growth.

Oligonucleotide duplexes (22-mer Suicide Substrates) at 0.05 mM were mixed with crystallization solution (Referred to as “Crystallant”) in the drop chambers of patented clover plates described in U.S. Pat. No. 6,029,804, followed by the addition of drug compound, and then protein solution at 2-5 mg/ml (as determined by a Bradford Assay, relative to a bovine serum albumin standard). The reservoir chambers of the clover plates contained 0.4 to 1.0 ml of crystallant. After set up of the crystallization drops at room temperature, the clover chambers were sealed with crystal clear tape and incubated at 15-16 degrees C. Crystals appeared within 2-5 days but sometimes crystallization required incubation of up to 7 months. On certain occasions, the tape from one quarter of a combinatorial crystallization clover was removed, thereby exposing the crystallization drops to the outside air environment causing evaporation crystallization drops and promotion of crystal growth.

Crystallizations are preferably set up and conducted in accordance with the methods and apparatus described in U.S. Pat. No. 6,039,804. However, crystallizations could also be performed in other crystallization apparatuses that accommodate vapor diffusion techniques.

In order that the invention described herein may be more fully understood, the following examples are set forth. It should be understood that these examples are for illustrative purposes only and are not to be construed as limiting this invention in any manner.

### EXAMPLE 1

Form 7.

Crystal Structure of Topoisomerase I and Duplex DNA.

This crystal structure contains the first example of a fully active human topoisomerase I (topo70) in covalent complex with the duplex 5′-bridging phosphorthiolate DNA.

The Crystal Form 7 Crystallant was composed of 10% (w/v) PEG-8000 (Sigma, Cat.# P4463, CAS # 25322-68-3) 100 mM Tris-HCl pH 8.0, 100 mM Na/K phosphate pH 6.2, 100 mM KCl (Sigma, Cat. # P9333, CAS # 7447-40-7) 10 mM dithiothreitol (Sigma Cat. # D5545, CAS 27565-41-9). The internal Reference Code for this Crystallant is “VII-6-1 #4)”

The crystallization set up that produces Crystal Form 7 was prepared at 25 degrees C. (room temperature) in drop chambers of Combinatorial Clover Plates as follows.

A milliliter (1 ml) of Crystal Form 7 Crystallant was placed into the reservoir chamber of a Combinatorial Clover. Three microliters (3 ul) of Crystal Form 7 Crystallant was removed from the reservoir chamber and placed into one of the four surrounding Drop Chambers. One and a half microliter (1.5 ul) of 22-mer CL22-sT:CP22-A Suicide Substrate Oligonucleotide Duplex at 0.05 mM in 3 mM NaCl (Sigma Cat. # S7653, CAS 7647-14-5) was then added to the 3 ul drop of Crystal Form 7 Crystallant in the drop chamber. After allowing the Suicide Substrate to mix with the Crystallant for approximately 1 minute, 2.5 microliter (2.5 ul) of topo70 wild type (Tyr723) at 2.5 milligrams per milliliter (2.5 mg/ml) was added to the drop. After allowing the mixture of Crystallant, Suicide Substrate, and topo70 wild type to sit for approximately 1 minute. The combinatorial clover reservoir was sealed with Crystal Clear tape (Manco), and the crystallization sample was maintained at 16 degrees C. for approximately two to four weeks. Crystals typically grew between the first and third weeks after set up.

The Form 7 crystal growth specifications and the following cryopreservation specifications are based on Emerald's internal reference code of “BNL-63”

Cryopreservation of Form 7 Crystals was achieved by transferring individual Form 7 Crystals (at room temperature, using glass capillary pipettes or by looping the crystal out of its liquid crystallization drop) into a cryoprotectant solution comprised of 20 microliters of (20 ul) of Form 7 Cryoprotectant Solution [30% (v/v) PEG-400 (Sigma Cat. # P3265, CAS # 25322-68-3), 100 mM Tris-HCl pH 8.0, 100 mM Na/K phosphate pH 6.2, 100 mM KCl (Sigma, Cat. # P9333, CAS # 7447-40-7)]. The transferred crystal was incubated in the cryoprotectant solution for approximately one minute, looped up in a nylon loop of approximately 700 micrometers in diameter, and plunged into liquid nitrogen for cryopreservation.

### EXAMPLE 2

Form 8.

Crystal structure of fully active human topoisomerase I (topo70) in ternary complex with 22 mer phosphorthiolate duplex DNA and the anti-cancer compound topotecan.

The Crystal Form 8 Crystallant was composed of 15% (w/v) PEG-3000 (Fluka, Cat.# 81227, CAS # 25322-68-3) 100 mM Tris-HCl pH 7.0, 100 mM Na/K phosphate pH 6.2, 10 mM Beta-mercaptoethanol (Sigma Cat. # M6250, CAS 60-24-2). The internal Reference Code for this Crystallant is “VII-10 #23”

The crystallization set up that produces Crystal Form 8 was prepared at 25 degrees C. (room temperature) in drop chambers of Emerald's Combinatorial Clover Plates as follows. A milliliter (1 ml) of Crystal Form 8 Crystallant was placed into the reservoir chamber of a Combinatorial Clover. Two microliters (2 ul) of Crystal Form 8 Crystallant was removed from the reservoir chamber and placed into one of the four surrounding Drop Chambers. One microliter (1 ul) of 22-mer CL22-sG:CP22—C Suicide Substrate Oligonucleotide Duplex at 0.05 mM in 3 mM NaCl (Sigma Cat. # S7653, CAS 7647-14-5) was then added to the 2 ul drop of Crystal Form 8 Crystallant in the drop chamber. After allowing the Suicide Substrate to mix with the Crystallant for approximately 1 minute, 0.3 microleter (0.3 ul) of 5 mM Topotecan (obtained from the drug synthesis branch of the National Cancer Institute, NSC609699) was added to the drop. After allowing the Topotecan to mix with the Crystallant and Suicide Substrate for approximately 1 minute, I microleter (1 ul) of topo70 wild type (Tyr723) at 3 milligrams per milliliter (3 mg/ml) was added to the drop. After allowing the mixture of Crystallant, Suicide Substrate, Topotecan and topo70 wild type to sit for approximately 1 minute. The combinatorial clover reservoir was sealed with Crystal Clear tape (Manco), and the crystallization sample was maintained at 15 degrees C. for approximately 7 months. Crystals grew sometime between the first and seventh month of incubation.

The Form 8 crystal growth specifications and the following cryopreservation specifications are based on Emerald's internal reference code of “BNL-91”

Cryopreservation of Form 8 Crystals was achieved by transferring individual Form 8 Crystals (at room temperature, using glass capillary pipettes or by looping the crystal out of its liquid crystallization drop) into a cryoprotectant solution comprised of 20 microliters of (20 ul) of Form 8 Cryoprotectant Solution [30% (v/v) PEG-400 (Sigma Cat. # P3265, CAS # 25322-68-3) 100 mM Tris-HCl pH 7.0, 100 mM Na/K phosphate pH 6.2] plus 1.5 microliter (1.5 ul) of 1 mM Topotecan. The transferred crystal was incubated in the cryoprotectant solution for approximately one minute, during which time, the crystal was observed to crack and therefore a small chunk of the crystal that displayed no visible cracking was looped up in a nylon loop of approximately 300 micrometers in diameter and plunged into liquid nitrogen for cryopreservation.

### EXAMPLE 3

Form 9 with Compound Topotecan.

Crystal structure of fully active human topoisomerase I (topo70) in ternary complex with 22 mer phosphorthiolate duplex DNA and the anti-cancer compound topotecan.

This example demonstrates the utility of using the said invention to crystallize one compound in multiple crystal forms (See example 2 above).

The Crystal Form 9 Crystallant was composed of 10% (w/v) PEG-8000 (Fluka, Cat.# 81268, CAS # 25322-68-3) 100 mM MES-NaOH pH 6.4 (or alternatively ADA-NaOH pH 6.5), 200 mM lithuim sulfate (Sigma Cat. # L8158, CAS # 10102-25-7).

The internal reference code for this Crystallant is “T80P #9 or #10)”

The crystallization set up that produces Crystal Form 9 was prepared at 25 degrees C. (room temperature) in drop chambers of Emerald's Combinatorial Clover Plates as follows. A milliliter (1 ml) of Crystal Form 9 Crystallant was placed into the reservoir chamber of a Combinatorial Clover. Two microliters (2 ul) of Crystal Form 9 Crystallant was removed from the reservoir chamber and placed into one of the four surrounding Drop Chambers. One and a half microliter (1.5 ul) of 22-mer CL22-sG:CP22—C Suicide Substrate Oligonucleotide Duplex at 0.05 mM in 3 mM NaCl (Sigma Cat. # S7653, CAS 7647-14-5) was then added to the 2 ul drop of Crystal Form 9 Crystallant in the drop chamber.

After allowing the Suicide Substrate to mix with the Crystallant for approximately 1 minute, 0.3 microleter (0.3 ul) of 1 mM Topotecan (obtained from the drug synthesis branch of the National Cancer Institute, NSC609699) was added to the drop. After allowing the Topotecan to mix with the Crystallant and Suicide Substrate for approximately 1 minute, I microleter (1.5 ul) of topo70 wild type (Tyr723) at 4 milligrams per milliliter (4 mg/ml) was added to the drop. After allowing the mixture of Crystallant, Suicide Substrate, Topotecan and topo70 wild type to sit for approximately 1 minute, the combinatorial clover reservoir was sealed with Crystal Clear tape (Manco), and the crystallization sample was maintained at 16 degrees C. for approximately two to four weeks. Crystals typically grew between the first and third weeks after set up.

The Form 9 crystal growth specifications and the following cryopreservation specifications are based on Emerald's internal reference code of “Topo-104”

Cryopreservation of Form 9 Crystals was achieved by transferring individual Form 9 Crystals (at room temperature, using glass capillary pipettes or by looping the crystal out of its liquid crystallization drop) into a cryoprotectant solution comprised of 10 microliters of (10 ul) of Form 9 Cryoprotectant Solution [30% (v/v) PEG-400 (Sigma Cat. # P3265, CAS # 25322-68-3), 100 mM MES-NaOH pH 6.4 (or alternatively ADA-NaOH pH 6.5), 200 mM lithuim sulfate (Sigma Cat. # L8158, CAS # 10102-25-7)], plus I microliter (1 ul) of 1 mM Topotecan. The transferred crystal was incubated in the cryoprotectant solution for approximately one minute, during which time, the crystal was looped up in a nylon loop of approximately 300 micrometers in diameter and plunged into liquid nitrogen for cryopreservation.

### EXAMPLE 4

Form9 with Compound AG260.

Crystal structure of fully active human topoisomerase I (topo70) in ternary complex with 22 mer phosphorthiolate duplex DNA and the anti-cancer compound AG260.

This example demonstrates the utility of using said invention to crystallize and solve the three-dimensional structure of different compounds with the same crystal form. This example also demonstrates the utility of using said invention to determine the three dimensional structure of camptothecin derivative compounds such the silatecan, AG-260.

Crystals of AG260 were grown and the structure was solved exactly as detailed in EXAMPLE 3 above. Crystal unit cell parameters were determined to be similar to the FORM-9 topotecan crystal. See table 1.

### EXAMPLE 5

Form-10

Crystal structure of fully active human topoisomerase I (topo70) in ternary complex with 22 mer phosphorthiolate duplex DNA and the anti-cancer compound MJ-II-38. This example demonstrates the utility of using said invention to determine the three dimensional structure of non-camptothecin derivatives such the indenoisoquinoline compound MJ-II-38.

The Crystal Form 10 Crystallant was composed of 10% (w/v) PEG-8000 (Fluka, Cat.# 81268, CAS # 25322-68-3) 100 mM MES-NaOH pH 6.4 (or alternatively ADA-NaOH pH 6.5), 200 mM lithuim sulfate (Sigma Cat. # L8158, CAS # 10102-25-7).

The internal reference code for this Crystallant is “T80P #9 or #10

The crystallization set up that produces Crystal Form 10 was prepared at 25 degrees C. (room temperature) in drop chambers of Emerald's Combinatorial Clover Plates as follows. A milliliter (1 ml) of Crystal Form 9 Crystallant was placed into the reservoir chamber of a Combinatorial Clover. Two microliters (2 ul) of Crystal Form 9 Crystallant was removed from the reservoir chamber and placed into one of the four surrounding Drop Chambers. One and a half microliter (1.5 ul) of 22-mer CL22-sG:CP22—C Suicide Substrate Oligonucleotide Duplex at 0.05 mM in 3 mM NaCl (Sigma Cat. # S7653, CAS 7647-14-5) was then added to the 2 ul drop of Crystal Form 7 Crystallant in the drop chamber.

After allowing the Suicide Substrate to mix with the Crystallant for approximately 1 minute, 0.3 microleter (0.3 ul) of 1 mM MJ-II-38 (see FIG. 12 for the structure of MJ-II-38) in 90% (v/v) DMSO (Sigma Cat. # D5879, CAS 67-68-5) was added to the drop. After allowing the MJ-1′-38 to mix with the Crystallant and Suicide Substrate for approximately 1 minute, I microleter (1.5 ul) of topo70 wild type (Tyr723) at 4 milligrams per milliliter (4 mg/ml) was added to the drop. After allowing the mixture of Crystallant, Suicide Substrate, MJ-II-38, and topo70 wild type to sit for approximately 1 minute, the combinatorial clover reservoir was sealed with Crystal Clear tape (Manco), and the crystallization sample was maintained at 16 degrees C. for approximately two to four weeks. Crystals typically grew between the first and third weeks after set up.

NOTE: The Form 10 crystallization condition first produces large Transamerica Building shaped crystals. However, these crystals are found not to diffract X-rays to beyond 8 angstrom resolution. However, crystals with Form 9 morphology will grow out of the conditions if one of the four drop chambers of the combinatorial clover is unsealed (by removal of the tape above the drop) and evaporation is allowed to occur at 16 degrees C. over a period of two weeks. The resulting crystals that have Form 9 morphology are the Form 10 crystals.

The Form 10 crystal growth specifications and the following cryopreservation specifications are based on internal reference code of “BART-COM-CAT-From 10”

Cryopreservation of Form 10 Crystals was achieved by transferring individual Form 10 Crystals (at room temperature, using glass capillary pipettes or by looping the crystal out of its liquid crystallization drop) into a cryoprotectant solution comprised of 10 microliters of (10 ul) of Form 10 Cryoprotectant Solution [30% (v/v) PEG-400 (Sigma Cat. # P3265, CAS # 25322-68-3), 100 mM MES-NaOH pH 6.4 (or alternatively ADA-NaOH pH 6.5), 200 mM lithuim sulfate (Sigma Cat. # L8158, CAS # 10102-25-7)], plus I microliter (1 ul) of 1 mM MJ-II-38 in 90% (v/v) DMSO (Sigma Cat. # D5879, CAS 67-68-5). The transferred crystal was incubated in the cryoprotectant solution for approximately one minute, during which time, the crystal was looped up in a nylon loop of approximately 300 micrometers in diameter and plunged into liquid nitrogen for cryopreservation.

### EXAMPLE 6

Form-11

Crystal structure of fully active human topoisomerase I (topo70) in ternary complex with 22 mer phosphorthiolate duplex DNA and the DNA minor-groove binding compound hoecsht-33342.

This example demonstrates the utility of using said invention to crystallize and solve the structure of DNA binding compounds which do not bind to the active site of topoisomerase 1.

Crystals of Form-11 were grown and the structure was solved similarly as detailed in EXAMPLE 3 above.

While we have described a number of the embodiements of this invention, it is apparent that our basic examples may be altered to provide other embodiements which utilize the products and processes of this invention. Therefore, it will be appreciated that the scope of this invention is to be defined by the appended claims rather than by specific embodiements which have been represented by way of example.

### REFERENCES

### U.S. Patent Documents

U.S. Pat. No. 5,856,116
U.S. Pat. No. 5,070,192
U.S. patent application Ser. No. 09/882,274 (Burgin, SDSU patent application
U.S. Pat. No. 5,004,758
U.S. Pat. No. 6,029,804
U.S. Pat. No. 6,039,804. Issued Mar. 21, 2000.

### Foreign Patent Documents

WO 99/45379
WO 00/14105

### Other Documents

D'Arpa, P., et al., “cDNA cloning of human DNA topoisomerase I: catalytic activity of a 67.7-kDa carboxyl-terminal fragment”, Proc Natl Acad Sci USA, 85, pp. 2543-2547 (1988).
Burgin Jr., A. B., Huizenga, B. N., Nash, H. A., “A novel suicide substrate for DNA topoisomerases and site-specific recombinases.” Nucleic Acids Res., 23, pp. 2973-2979 (1995).
Stewart, L., et al., “A model for the mechanism of human topoisomerase I”, Science, 729, pp. 1534-1541 (1998).
Redinbo, M. R., Stewart, L., Kuhn, P., Champoux, J. J., Hol, W. G. J., “Crystal structures of human topoisomerase I in covalent and noncovalent complexes with DNA.” Science, 279, pp. 1504-1513 (1998).
Stewart, L., et al., “Reconstitution of human topoisomerase I by fragment complementation”, J. Mol. Biol., 269, pp. 355-372 (1997).
Champoux, J. J., “DNA Topoisomerases: structure, function, and mechanism.” Ann. Rev. Biochem., 70, pp. 369-413 (2001).
Wall, M. E., et al., “The isolation and structure of camptothecin, a novel alkaloidal leukemia and tumor inhibitor from Camptotheca acuminata.” J. Am. Chem. Soc., 88, pp. 3888-3890 (1966).
Hsiang, Y. H., et al., “Camptothecin induces protein-linked DNA breaks via mammalian DNA topoisomerase I”, J. Biol. Chem., 260, pp. 14873-14878 (1985).
Nitiss, J. L. and Wang, J. C., “DNA topoisomerase-targeting antitumor drugs can be studied in yeast”, Proc. Natl. Acad. Sci. U.S.A., 85, pp. 7501-7505 (1988).
Hertzberg, R. P., et al., “On the mechanism of topisomerase I inhibition by camptothecin: evidence for binding to an enzyme-DNA complex.” Biochem., 28, pp. 4629-4638 (1989).
Chen, A. Y. and Liu, L. F., “DNA topoisomerases: Essential enzymes and lethal targets.” Rev. Pharmacol. Toxicol., 34, pp. 191-218 (1994).
Li, X. G., et al., “Involvement of amino acids 361 to 364 of human topoiosmerase I in camptothecin resistance and enzyme catalysis.” Biochem. Pharmacol., 53, pp. 1019-1027 (1997).
Kehrer, D. F. S., et al., “Modulation of camptothecin analogs in the treatment of cancer: a review.” Anti-Cancer Drugs, 12, pp. 89-105 (2001).
Chourpa, 1., R10u, J. F., Millot, J., Pommier, Y., Manfait, M., “Modulation in kinetics of lactone ring hydrolysis of camptothecins upon interaction with topoisomerase I cleavage sites on DNA.” Biochem., 37, pp. 7284-7291 (1998).
Hsiang, Y. H., et al., “DNA topoisomerase 1-mediated DNA cleavage and cytotoxicity of camptothecin analogues”, Cancer Res, 49, pp. 4385-9 (1989).
Giovanella, B. C., et al., “DNA topoisomerase 1—targeted chemotherapy of human colon cancer in xenografts”, Science, 246, pp. 1046-8 (1989).
Sheldrick, G. M., “SHELXL-97”, pp. (1997).
Smith, J. L., Hendrickson, W. A., Honzatko, R. B., Sheriff, S., “Structural heterogeneity in protein crystals.” Biochem., 25, pp. 5018-5027 (1986).
Tamura, H., et al., “Molecular cloning of a cDNA of a camptothecin-resistant human DNA topoisomerase I and identification of mutation sites.” Nucleic Acids Res., 19, pp. 69-75 (1991).
Fertala, J., et al., “Substitutions of Asn-726 in the active siteof yeast DNA topoisomerase I define novel mechanisms of stabilizing the covalent enzymer-DNA intermediate.” J. Biol. Chem, 275, pp. 15246-15253 (2000).
Krogh, B. O., Shuman, S., “Catalytic mechanism of DNA topoisomerase IB.” Mol. Cell, 5, pp. 1034-1041 (2000).
Champoux, J. J., “Structure-based analysis of the effects of camptothecin on activities of human topoisomerase I”, Ann. N.Y. Acad. Sci., 922, pp. 56-64 (2000).
Saenger, W., “Principles of nucleic acid structure”, Springer Advanced Texts in Chemistry, pp. 556 (1984).
Rubin, E., et al., “Identification of a mutant human topoisomerase I with intact catalytic activity and resistance to 9-nitro-camptothecin”, J Biol Chem, 269, pp. 2433-2439 (1994).
Fiorani, P., et al., “Domain interactions affecting human DNA topoisomerase I catalysis and camptothecin sensitivity”, Mol Pharmacol, 56, pp. 1105-1115 (1999).
Stewart, L., et al., “A functional linker in human topoisomerase I is required for maximum sensitivity to camptothecin in a DNA relaxation assay”, J. Biol. Chem., 274, pp. 32950-32960 (1999).
Stewart, L., et al., “Biochemical and biophysical analyses of recombinant forms of human topoisomerase I”, J. Biol. Chem., 271, pp. 7593-7601 (1996).
Strumberg, D., et al., “Synthesis of cytotoxic indenoisoquinoline topoisomerase I poisons”, J Med Chem, 11, pp. 446-457 (1999).
Cushman, M., et al., “Synthesis of new indeno[1,2-c]isoquinolines: cytotoxic non-camptothecin topoisomerase I inhibitors”, J Med Chem, 5, pp. 3688-3698 (2000).
Rodgers, D. W., “Cryocrystallography”, Structure, 2, pp. 1135-1140 (1994).
Otwinoski, Z. and Minor, W., “Processing of X-ray diffraction data in oscillation mode.” Meths. Enzymol., 276, pp. 307-326 (1997).
Navaza, J., “AMORE: an automated package for molecular replacement.” Acta. Crystallogr., A50, pp. 157-163 (1994).
Brünger, A. T., et al., “Crystallography and NMR systems (CNS): A new software system for macromolecular structure determination.” Acta. Crystallogr., D54, pp. 905-921 (1998).
McRee, D. E., “XtalView/Xfit-A Versatile Program for Manipulating Atomic Coordinates and Electron Density.” J. Struct. Biol., 125, pp. 156-165 (1999).

1. A crystal composition comprising a ternary complex of a compound, a protein, and a poly-nucleic acid wherein the protein is covalently linked to a phosphorous of the poly-nucleic acid.

2. A crystal composition of claim 1, wherein the compound is an inhibitor of a topoisomerase.

3. A crystal composition comprising a complex of a compound and topoisomerase covalently linked to a poly-nucleic acid substrate.

4. A crystal composition of claim 3 wherein the protein is a eukaryotic topoisomerase.

5. A crystal composition of claim 3, wherein the nucleic acid is duplex DNA.

6. A crystal composition of claim 3, wherein the nucleic acid is duplex DNA.

7. A crystal composition comprising a complex of a compound and human topoisomerase I covalently linked to a duplex DNA substrate.

8. A crystal composition of claim 7, wherein the compound is an inhibitor of a topoisomerase.

9. The crystal composition of claim 7 wherein the crystal structure is crystal Form 7, Form 8, Form 9, Form 10, or Form 11.

10-27. (Canceled)
